SEQUENCE LISTING (41 sequences)

SEQ ID NO: 1 - DNA, 24 bp, Mus musculus
ctcaggaggagcaatgatcttgat

SEQ ID NO: 2 - DNA, 20 bp, Mus musculus
taccaccatgtacccaggca

SEQ ID NO: 3 - DNA, 20 bp, Mus musculus
aagaacacgcttgggaatgg

SEQ ID NO: 4 - DNA, 17 bp, Mus musculus
ctgcacctgctgcggac

SEQ ID NO: 5 - DNA, 20 bp, Mus musculus
acacgtttgggaatggacac

SEQ ID NO: 6 - DNA, 20 bp, Mus musculus
ccatgggaagatgttctggg

SEQ ID NO: 7 - DNA, 22 bp, Mus musculus
cttaagtaccaggtgctggacg

SEQ ID NO: 8 - DNA, 20 bp, Mus musculus
ggtgggtagcgcatcaacac

SEQ ID NO: 9 - DNA, 23 bp, Mus musculus
tgggtgtgaagggaaataaggag

SEQ ID NO: 10 - DNA, 20 bp, Mus musculus
atttgagccttgggggtcag

SEQ ID NO: 11 - DNA, 20 bp, Mus musculus
agcacactgctggtcatcaa

SEQ ID NO: 12 - DNA, 21 bp, Mus musculus
ttggcaaaagccttcttccgc

SEQ ID NO: 13 - DNA, 22 bp, Mus musculus
cttgctgacctggcagtgtatg

SEQ ID NO: 14 - DNA, 20 bp, Mus musculus
tgtgagtgtcgcatcaggtc

SEQ ID NO: 15 - DNA, 20 bp, Homo sapiens
gcgagaagatgacccagatc

SEQ ID NO: 16 - DNA, 19 bp, Homo sapiens
ccagtggtacggccagagg

SEQ ID NO: 17 - DNA, 23 bp, Homo sapiens
aaccaggagttaagacagcgctt

SEQ ID NO: 18 - DNA, 18 bp, Homo sapiens
ctgcaccctctgcggact

SEQ ID NO: 19 - DNA, 20 bp, Homo sapiens
gaatggggtgatgagcagtt

SEQ ID NO: 20 - DNA, 19 bp, Homo sapiens
cagaagggcaggatacagc

SEQ ID NO: 21 - DNA, 20 bp, Homo sapiens
cctaacccttttgtcgcctg

SEQ ID NO: 22 - DNA, 19 bp, Homo sapiens
caggtaggccacggtgtgt

SEQ ID NO: 23 - DNA, 23 bp, Homo sapiens
tcctatgaagggctagtaaccaa

SEQ ID NO: 24 - DNA, 21 bp, Homo sapiens
tccacgggtcaccaatataaa

SEQ ID NO: 25 - DNA, 20 bp, Homo sapiens
aaccttactcgccccagtct

SEQ ID NO: 26 - DNA, 20 bp, Homo sapiens
cagaaggacacttggcttcc

SEQ ID NO: 27 - DNA, 20 bp, Homo sapiens
tctttcggggagatcttgtg

SEQ ID NO: 28 - DNA, 20 bp, Homo sapiens
tgagacccatttcaggcttc

SEQ ID NO: 29 - DNA, 20 bp, Homo sapiens
ctccattgtccaggctgagt

SEQ ID NO: 30 - DNA, 20 bp, Homo sapiens
ttccaggcaaatcctcaaac

SEQ ID NO: 31 - DNA, 19 bp, Homo sapiens
gagtttctccctccctctc

SEQ ID NO: 32 - DNA, 19 bp, Homo sapiens
gctccattgaaccgctcac

SEQ ID NO: 33 - DNA, 23 bp, Homo sapiens
cattgtgggttttgaaaaggtta

SEQ ID NO: 34 - DNA, 25 bp, Homo sapiens
atgaaccacgactagaggctgactt

SEQ ID NO: 35 - DNA, 23 bp, Homo sapiens
tgcacgtagtctgagtgctgcgg

SEQ ID NO: 36 - DNA, 23 bp, Homo sapiens
atgtagaggattccatctgaccc

SEQ ID NO: 37 - RNA, 20 nt, Artificial Sequence
  A synthetic RNA sequence
cguuaaucgcguauaauacg

SEQ ID NO: 38 - DNA, 19 bp, Artificial Sequence
  A synthetic oligonucletoide sequence
aaccttactcgccccagtc

SEQ ID NO: 39 - DNA, 20 bp, Artificial Sequence
  A synthetic oligonucletoide sequence
cagaaggacacttggcttcc

SEQ ID NO: 40 - DNA, 20 bp, Artificial Sequence
  A synthetic oligonucletoide sequence
tctttcggggagatcttgtg

SEQ ID NO: 41 - DNA, 20 bp, Artificial Sequence
  A synthetic oligonucletoide sequence
tgagacccatttcaggcttc

(19) United States
(12) Patent Application Publication
(10) Pub. No.: US 2022/0160705 A1
(43) Pub. Date: May 26, 2022

(54) METHODS FOR CONTROLLING PROSTAGLANDIN-MEDIATED BIOLOGICAL PROCESSES

(71) Applicant: Cornell University, Ithaca, NY (US)

(72) Inventors: Juan Rodrigo Cubillos Ruiz, New York, NY (US); Laurie H. Glimcher, West Newton, MA (US); Sahil Chopra, San Francisco, CA (US); Paolo Giovanelli, New York, NY (US)

(21) Appl. No.: 17/440,895

(22) PCT Filed: Mar. 19, 2020
(86) PCT No.: PCT/US2020/023697

§ 371 Date: Sep. 20, 2021

Related U.S. Application Data
(60) Provisional application No. 62/821,167, filed on Mar. 20, 2019.

(51) Int. Cl. A61K 31/496; A61P 25/04; A61K 31/498; A61K 31/4725; A61K 31/517; A61K 31/55; A61K 31/5377; A61K 31/4439; A61K 31/536; A61K 31/4985

(52) U.S. Cl. CPC A61K 31/496; A61P 25/04; A61K 31/498; A61K 31/4725; A61K 31/4985; A61K 31/55; A61K 31/5377; A61K 31/4439; A61K 31/536; A61K 31/517

(57) ABSTRACT

Described herein are compositions and methods for reducing prostaglandin production and pain in a mammalian or avian subject. Such compositions and methods inhibit reduce prostaglandinendoperoxide synthase 2 (Ptgs2/Cox-2) and prostaglandin E synthase (Ptges/mPGES-1) activities in the subject, but do not substantially inhibit prostaglandin-endoperoxide synthase 1 (Cox1) or prostaglandin E synthase 2 activities in the subject.

This application claims benefit of priority to the filing date of U.S. Provisional Application Ser. No. 62/821,167, filed Mar. 20, 2019, the contents of which are specifically incorporated by reference herein in their entirety.

### SEQUENCE LISTING

The instant application contains a Sequence Listing which has been submitted in ASCII format via EFS-Web and is hereby incorporated by reference in its entirety. Said ASCII copy, created on Mar. 13, 2020, is named 2020956.txt and is 8,192 bytes in size.

### BACKGROUND

The serine/threonine-protein kinase/endoribonuclease inositol-requiring enzyme 1 α (IRE1α) in humans is encoded by the ERN1 gene, and expression of the IRE1α protein is activated during endoplasmic reticulum (ER) stress. The IRE1α-XBP1 arm of the unfolded protein response (UPR) maintains endoplasmic reticulum (ER) homeostasis, and also controls UPR-independent processes such as cytokine production and lipid metabolism. Yet, the physiological consequences of IRE1α-XBP1 activation in immune cells remain largely unexplored.

### SUMMARY

As shown herein, leukocyte-intrinsic IRE1 α-XBP1 signaling drives prostaglandin biosynthesis and pain. Described herein are compositions and methods that inhibit prostaglandin biosynthesis and pain. Such compositions and methods inhibit reduce prostaglandinendoperoxide synthase 2 (Ptgs2/Cox-2) and prostaglandin E synthase (Ptges/mPGES-1) activities in the subject, but do not substantially inhibit prostaglandin-endoperoxide synthase 1 (Cox1) or prostaglandin E synthase 2 activities in the subject.

Inducible biosynthesis of prostaglandins, including PGE₂, was markedly decreased in myeloid cells with reduced or deleted IRE1α or XBP1, but not altered in the absence of the two other ER stress sensors PERK and ATF6α. IRE1α-activated XBP1 bound to and directly activated the expression of human PTGS2 and PTGES to enable PGE₂ generation. Mice selectively lacking IRE1α-XBP1 in leukocytes, or treated with pharmacological inhibitors of IRE1α, failed to induce PGE₂ production upon challenge with inflammatory stimuli and exhibited reduced behavioral pain responses in multiple PGE₂-dependent models of pain. IRE1α-XBP1 signaling as a key mediator of prostaglandin biosynthesis. Modulation of the IRE1α-XBP1 signaling pathway can control pain, and prostaglandin-dependent biological processes such as pregnancy, fever, vascular permeability, allergy, arthritis and immunosuppression in patients, including cancer patients.

A variety of compounds are described herein that inhibit IRE1α-XBP1 signaling, drives prostaglandin biosynthesis and pain.

### DESCRIPTION OF THE FIGURES

FIG. 1A-1G illustrate IRE1α-XBP1 activation in dendritic cells stimulated with lipopolysaccharides (LPS) and zymosan (a glucan with repeating glucose units connected by β-1,3-glycosidic linkages, which binds to TLR 2 and Dectin-1 (CLEC7A)). Ern1WT or Ern1KO bone marrow-derived dendritic cells (DC) (n=4/group) were stimulated as indicated for 6 hours. FIG. 1A illustrates Xbp1 mRNA splicing as evaluated using conventional RT-PCR assays (XBP1u, unspliced form; Xbp1s, spliced form). FIG. 1B illustrates expression levels of Xbp1s transcripts as confirmed by RT-qPCR. Data were normalized to Actb values in each case. FIG. 1C illustrates expression levels of reported regulated IRE1α-dependent decay (RIDD) target genes in wild type or IRE1α-deficient dendritic cells stimulated for 6 hours with zymosan (25 μg/ml). Data are shown as mean±s.e.m. relative to untreated Ern1WT controls. **P<0.005, ***P<0.0005. FIG. 1D illustrates expression levels of previously reported RIDD target genes in wild type or IRE1α-deficient dendritic cells stimulated for 6 hours with LPS (50 ng/ml). Data are shown as mean±s.e.m. relative to untreated Ern1WT controls. **P<0.005, ***P<0.0005. FIG. 1E illustrates dendritic cell generation from total bone marrow cells isolated from Ern1^{f/f} mice that were differentiated in vitro using GMCSF as described in the methods. Dendritic cell generation 6-7 days later was assessed by FACS using antibodies staining for CD11c and MHC-II. FIG. 1F illustrates dendritic cell generation from total bone marrow cells isolated from Ern1^{f/f} Vav1^{cre} mice that were differentiated in vitro using GMCSF, as described in the Examples. Dendritic cell generation 6-7 days later was assessed by FACS using antibodies staining for CD11c and MHC-II. FIG. 1G illustrates numbers of differentially regulated genes identified in IRE1α deficient DC treated with LPS or zymosan.

FIG. 2A-2F illustrate that IRE1α regulates the expression of Ptgs2 and Ptges. Ern1^{WT} or Ern1^{KO} dendritic cells (n=4/group) were stimulated with LPS (50 ng/ml) or zymosan (25 μg/ml) for 6 hours. FIG. 2A illustrates identification of the top ten key regulators by RNA-seq analysis. FIG. 2B illustrates expression levels of Ptgs2 upon LPS or zymosan stimulation as detected by RT-qPCR. FIG. 2C illustrates expression levels of Ptges upon LPS or zymosan stimulation as detected by RT-qPCR. FIG. 2D shows representative immunoblot analyses for Cox-2 and mPGES-1 expression in Ern1^{WT} and Ern1^{KO} dendritic cells stimulated with LPS (10 ng/ml or 100 ng/ml) or zymosan (25 μg/ml). Density of each band was normalized to its own Actin value, and numbers shown represent relative expression compared with control Ern1WT under the same condition. Data are shown as mean±s.e.m. *P<0.05, **P<0.005. FIG. 2E shows transcript levels for Ptgs1 as measured by RT-qPCR analysis in Ern1WT and Ern1KO dendritic cells stimulated with LPS (10 ng/ml or 100 ng/ml) or zymosan (25 μg/ml). FIG. 2F shows transcript levels for Ptges2 as measured by RT-qPCR analysis in Ern1^{WT} and Ern1^{KO} dendritic cells stimulated with LPS (10 ng/ml or 100 ng/ml) or zymosan (25 μg/ml). As illustrated in FIG. 2E-2F, IRE1α deficiency did not affect the constitutive expression of prostaglandin-endoperoxide synthase 1 (also known as COX1; COX3; PHS1; PCOX1; PES-1; PGHS1; PTGHS; PGG/HS; PGHS-1 and referred to as Ptgs1/Cox-1) or prostaglandin E synthase 2 (also known as GBF1; GBF-1; PGES2; C9orf15; mPGES-2, and referred to as Ptges2).

FIG. 3A-3Q illustrate that IRE1α promotes prostaglandin biosynthesis. FIG. 3A illustrates the pathway depicting the main events implicated in PGE2 biosynthesis. FIG. 3B illustrates the types of lipids in Ern1WT (n=4) or Ern1KO dendritic cells. Ern1WT (n=4) or Ern1KO DC (n=3) were stimulated with LPS (50 ng/ml) for 6 hours and lipidomic analyses were performed. Data are represented as a volcano plot with red lines indicating a 0.05 significance level. FIG. 3C illustrates PGE2 concentrations confirmed by ELISA-based assays demonstrating reduced PGE2 in supernatants from EMIKO DC upon stimulation with the indicated concentrations of LPS. FIG. 3D illustrates PGE2 concentrations confirmed by ELISA-based assays demonstrating reduced PGE2 in supernatants from Ern1KO dendritic cells at different time points after stimulation with LPS at 50 ng/ml. Two-way Anova was used where *P<0.05, **P<0.005, ***P<0.0005. For FIG. 3E-3G, murine DC of the indicated genotypes were stimulated with zymosan (25 μg/ml) for 6 hours and PGE2 was quantified by in culture supernatants by ELISA. FIG. 3E-1 illustrates PGE2 concentrations in supernatants from Ern1WT or Ern1KO dendritic cells. FIG. 3E-2 illustrates PGE2 concentrations in supernatants from Xbp1WT or Xbp1KO dendritic cells. FIG. 3F illustrates PGE2 concentrations in supernatants from Eif2 ak3WT or Eif2ak3KO dendritic cells. FIG. 3G illustrates PGE2 concentrations in supernatants from Atf6WT or Atf6KO dendritic cells. FIG. 3H illustrates XBP1s expression in untreated and zymosan-treated human monocyte-derived XBP1-deficient dendritic cells. FIG. 3I illustrates PTGS2 expression in untreated and zymosan-treated human monocyte-derived XBP1-deficient dendritic cells. FIG. 3J-1 illustrates PTGES expression in untreated and zymosan-treated human monocyte-derived XBP1-deficient dendritic cells. FIG. 3J-2 illustrates PGE2 levels in untreated and zymosan-treated human monocyte-derived XBP1-deficient dendritic cells. FIG. 3K illustrates XBP1s expression in untreated and zymosan-treated human monocyte-derived ERN1-deficient dendritic cells. FIG. 3L illustrates PGE2 levels in untreated and zymosan-treated human monocyte-derived ERN1-deficient dendritic cells. CRISPR/Cas9-based gene editing was used to ablate XBP1 (FIG. 3H-3J) or ERN1 (FIG. 3K-3L) in human monocyte-derived DC, and cells were then stimulated for 6 hours with zymosan (25 μg/ml). RT-qPCR was used to assess the indicated transcript levels (FIG. 3H-3I, 3K) and PGE2 levels were determined in the corresponding supernatants using ELISA (FIG. 3J, 3L). Data are shown as mean±s.e.m. *P<0.05, **P<0.005, ***P<0.0005. FIG. 3M-3Q illustrate that IRE1α expression in leukocytes is necessary for Cox-2-dependent prostaglandin (PGE₂, PGD₂, PGF₂α and TBX₂) production in vivo in experiments where Ern1^{f/f} (grey bars) or Ern1^{f/f} Vav1^{cre} (blue bars) mice were injected i.p. with 200 μl of PBS alone (vehicle) or with 200 μl of PBS containing 1 mg/kg zymosan, and peritoneal wash samples were collected 3 hours later. FIG. 3M graphically illustrates PGE₂ production in these Ern1^{f/f} (grey bars) or Ern1^{f/f} Vav1^{cre} (blue bars) mice. FIG. 3N graphically illustrates PGD₂ production in these Ern1^{f/f} (grey bars) or Ern1^{f/f} Vav1^{cre} (blue bars) mice. FIG. 3O graphically illustrates PGF₂α production in these Ern1^{f/f} (grey bars) or Ern1^{f/f} Vav1^{cre} (blue bars) mice. FIG. 3P graphically illustrates TBX₂ production in these Ern1^{f/f} (grey bars) or Ern1^{f/f} Vav1^{cre} (blue bars) mice. FIG. 3Q graphically illustrates 15-HETE production in these Ern1^{f/f} (grey bars) or Ern1^{f/f} Vav1^{cre} (blue bars) mice.

FIG. 4A-4J illustrate that IREα1 expression and IRE1α-XBP1 signaling is required for PGE2 synthesis by additional murine myeloid cells like neutrophils and macrophages. FIG. 4A illustrates the concentration of PGE2 secreted by neutrophils from Ern1WT and Ern1KO mice. Primary neutrophils were magnetically immunopurified from the bone marrow of either Ern1^{f/f} (Ern1WT) or Ern1^{f/f} Vav1^{cre} (Ern1KO) mice and stimulated for 6 hours with the indicated concentrations of LPS. PGE2 was measured in culture supernatants using ELISA. FIG. 4B illustrates the concentration of PGE2 secreted by macrophages derived from Ent/WT and Ern1KO mice. Total bone marrow cells isolated from Ern1^{f/f} or Ern1^{f/f} Vav1^{cre} mice and were differentiated in vitro using recombinant MCSF to generate primary macrophages, as described in Example 1. Macrophages of the indicated genotypes were stimulated with the indicated concentrations of LPS, and PGE2 was measured in culture supernatants 6 hours later. n=4 per group in all cases. FIG. 4C illustrates the concentration of PGE2 secreted by macrophages derived from Xbp1^{f/f} or Xbp1^{f/f} Vav1^{cre} mice. Total bone marrow cells isolated from Xbp1^{f/f} or Xbp1^{f/f} Vav1^{cre} mice were differentiated in vitro using recombinant MCSF to generate primary macrophages, as described in Example 1. Macrophages were stimulated with the indicated concentrations of LPS, and PGE2 was measured in culture supernatants 6 hours later. n=4 per group in all cases. Data are shown as mean±s.e.m. **P<0.005, ***P<0.0005. FIG. 4D-4G illustrate that IRE1α expression in leukocytes is necessary for PGE2 production in vivo. FIG. 4D illustrates transcript levels of Xbp1s in total leukocytes recovered from peritoneal lavages from Xbp1^{f/f} (Ern1^{WT}, black bars) or Ern1^{f/f} Vav1^{cre} (Ern1^{KO}, blue bars) mice exposed to PBS or LPS mice as measured by RT-qPCR. FIG. 4E illustrates transcript levels of Ptgs2 in total leukocytes recovered from peritoneal lavages from Xbp1^{f/f} (Ern1^{WT}, black bars) or Ern1^{f/f} Vav1^{cre} (Ern1^{KO}, blue bars) mice exposed to PBS or LPS mice as measured by RT-qPCR. FIG. 4F illustrates transcript levels of Ptges in total leukocytes recovered from peritoneal lavages of PBS or LPS from Xbp1^{f/f} (Ern1^{WT}, black bars) or Ern1^{f/f} Vav1^{cre} (Ern1^{KO}, blue bars) mice as measured by RT-qPCR. FIG. 4G illustrates PGE2 in total leukocytes recovered from peritoneal lavages from Xbp1^{f/f} (Ern1^{WT}, black bars) or Ern1^{f/f} Vav1^{cre} (Ern1^{KO}, blue bars) mice exposed to PBS or LPS mice as measured by levels in cell-free peritoneal wash supernatants were determined using mass spectrometry. At least 4 independent mice were used per group. Data are shown as mean±s.e.m. *P<0.05, **P<0.005, ***P<0.0005. FIG. 4H shows PGE2 levels in Ern1^{WT} or Ern1KO dendritic cells (n=4/group) stimulated with the indicated TLR agonists, phorbol myristate acetate (PMA) or zymosan for 6 hours and the concentration of PGE2 was determined in culture supernatants. FIG. 4I shows PGE2 levels in Ern1^{WT} or Ern1^{KO} dendritic cells treated with the ER stressor thapsigargin (TG) at 1 μM for 12 hours. FIG. 4J shows a Western blot analyzed for Cox-2 and mPGES-1 protein expression in Ern1^{WT} and Ern1^{KO} DC treated or not treated with thapsigargin (TG). Density of each band was normalized to its own Actin value, and numbers shown represent relative expression compared with control Ern1WT under the same condition. Data are shown as mean±s.e.m. *P<0.05, **P<0.005, ***P<0.0005.

FIG. 5A-5H illustrate that IRE1α-activated XBP1 (XBP1s) transactivates the PTGS2 and PTGES promoters. FIG. 5A is a schematic diagram of the promoter region of human PTGS2 showing predicted XBP1s-binding sites. (SEQ ID NOs: 38 and 39). FIG. 5B is a schematic diagram of the promoter region of human PTGES showing predicted XBP1s-binding sites. Human primary monocyte-derived DC were stimulated with zymosan in the presence or absence of the ER stressor 2-deoxy-D-glucose (2-DG), and ChIP assays were performed using anti-XBP1 s or isotype control antibodies. (SEQ ID NOs: 40 and 41). FIG. 5C shows the amount of XBP1s occupancy at the PTGS2 promoter region under the indicated conditions as determined by qPCR. FIG. 5D shows the amount of XBP1s occupancy at the PTGES promoter region under the indicated conditions as determined by qPCR. FIG. 5E shows the amount of XBP1s occupancy at the GFPT1 promoter region under the indicated conditions as determined by qPCR. FIG. 5F shows the amount of XBP1s occupancy at the pri-mIR-21 promoter region under the indicated conditions as determined by qPCR. ChIP-PCR assays were performed using 3-6 independent human donors. For FIG. 5G-5H, HEK293 cells were co-transfected with XBP1s expressing or CHOP-expressing vectors, and luciferase reporter constructs harboring the PTGS2 or PTGES promoters, along with Renilla. Luciferase activity was normalized to Renilla activity in each case. Data are representative of at least two independent experiments with similar results, using four independent technical replicates. Data are shown as mean±s.e.m *P<0.05, **P<0.005, ***P<0.0005. FIG. 5G shows the luciferase activity at the PTGS2 promoter region when XBP1 or CHOP are expressed. FIG. 5H shows the luciferase activity at the PTGES promoter region when XBP1 or CHOP are expressed.

FIG. 6A-6O illustrate that IRE1α expression in immune cells promotes pain behaviors. The results shown in FIG. 6A-6D were obtained from experiments where 0.9% v/v acetic acid (5 ml/kg) was injected intraperitoneally into Ern1^{f/f} (n=11) or Ern1^{f/f} Vav1^{cre} mice (n=12). FIG. 6A shows electrophoretically separated Xbp1 RNA illustrating Xbp1 splicing in leukocytes recovered from peritoneal lavages 30 minutes after acetic acid challenge (Xbp1u, unspliced form; Xbp1s, spliced form). FIG. 6B graphically illustrates the number of writhing behaviors in Ern1^{f/f} and Ern1^{f/f} Vav1^{cre} mice after acetic acid administration that were recorded every 5 minutes for 30 minutes. FIG. 6C graphically illustrates the number of writhing behaviors in Xbp1^{f/f} (n=10) or Xbp1^{f/f} Vav1^{cre} mice after acetic acid administration that were recorded every 5 minutes for 30 minutes. FIG. 6D graphically illustrates total ambulatory time for Ern1^{f/f} and Ern1^{f/f} Vav1^{cre} mice after acetic acid injection. FIG. 6E graphically illustrates total ambulatory counts for Ern1^{f/f} and Ern1^{f/f} Vav1^{cre} mice after acetic acid injection. The results shown in FIG. 6F-6I were obtained from experiments where a surgical incision was made in the left hind paw of Ern1^{f/f} (n=8) or Ern1^{f/f} Vav1^{cre} (n=8) mice. FIG. 6F shows electrophoretically separated Xbp1 RNA illustrating Xbp1 splicing in leukocytes sorted from the lesion 24 hours post-incision. FIG. 6G graphically illustrates spontaneous hind paw weight bearing distribution over time after surgery for Ern1^{f/f} and Ern1^{f/f} Vav1^{cre} mice. FIG. 6H graphically illustrates total weight for Ern1^{f/f} and Xbp1^{f/f} Vav1^{cre} mice over time after surgery. FIG. 6I graphically illustrates rearing activity for Ern1^{f/f} and Ern1^{f/f} Vav1^{cre} mice over time after surgery. Data are shown as mean±s.e.m. Two-way Anova was used for FIG. 6B; *P<0.05, **P<0.005, ***P<0.0005. FIG. 6J-6K illustrate CD45+ leukocyte infiltration and Cox-2 expression in the leukocytes infiltrating the paw after surgery of Ern1^{f/f} or Ern1^{f/f} Vav1^{cre} mice. FIG. 6J graphically illustrates quantification of total CD45+ cells infiltrating paw tissue in Ern1^{f/f} or Ern1^{f/f} Vav1^{cre} mice. FIG. 6K graphically illustrates the numbers of CD45+ leukocytes expressing Cox-2 in the paw 48 hours after surgery of Ern1^{f/f} or Ern1^{f/f} Vav1^{cre} mice. Data are shown as mean±s.e.m. *P<0.05. FIGS. 6L-6O illustrate levels of pro-inflammatory factors after acetic acid challenge in mice lacking IRE1α in leukocytes. 0.9% v/v acetic acid (5 ml/kg) was injected intraperitoneally into Ern1^{f/f} or Ern1^{f/f} Vav1^{cre} mice. FIG. 6L shows PGE2 levels in cell free-peritoneal lavage collected after 30 minutes from acetic acid injected mice where PGE2 levels were measured using mass spectrometry. FIG. 6M shows IL-6 transcript levels as measured in the recovered leukocytes from peritoneal lavage. FIG. 6N shows IL-1β transcript levels as measured in the recovered leukocytes from peritoneal lavage. FIG. 6O shows TNFα transcript levels as measured in the recovered leukocytes from peritoneal lavage. Each point represents a single independent mouse. Data are shown as mean±s.e.m. Two-way Anova was used for FIG. 6L; *P<0.05.

FIG. 7A-7B illustrate PGE2 production by ovarian cancer-associated dendritic cells of the indicated genotypes. FIG. 7A illustrates PGE2 concentrations in Ern1^{f/f} and Ern1^{f/f} CD11c^{cre} ovarian cancer-associated dendritic cells. FIG. 7B illustrates PGE2 concentrations in Xbp1^{f/f} and Xbp1^{f/f} CD11c^{cre} ovarian cancer-associated dendritic cells.

FIG. 8A-8D illustrate that pharmacological inhibition of IRE1α can reduce pain behaviors. FIG. 8A-1 illustrates Xbp1s mRNA levels measured in leukocytes recovered from peritoneal lavages by qRT-PCR after administration of IRE1α inhibitors KIRA6 and MKC8866. FIG. 8A-2 illustrates Ptges mRNA levels measured in leukocytes recovered from peritoneal lavages by qRT-PCR after administration of IRE1α inhibitors KIRA6 and MKC8866. FIG. 8B illustrates reduced writhing behaviors, recorded every 5 minutes for 30 minutes, in mice administered the IRE1α inhibitor KIRA6 compared to vehicle controls. FIG. 8C illustrates reduced writhing behaviors, recorded every 5 minutes for 30 minutes, in mice administered the IRE1α inhibitor MKC8866 compared to vehicle controls. For FIG. 8B-8C, wild-type C57BL/6J mice were administered i.p. with KIRA6 (25 mg/kg) or MKC8866 (20 mg/kg) 6 hours and 30 minutes prior to challenge with 0.9% v/v acetic acid (5 ml/kg). FIG. 8D illustrates that Celecoxib, a selective Cox-2 inhibitor, also decreased writhing behaviors after acetic acid injection. C57BL/6J mice (n=8/group) were administered with 20 mg/kg Celecoxib (200 μl) i.p. 6 hours and 30 minutes before 0.9% v/v acetic acid injection (5 ml/kg). Writhing behaviors were recorded every 5 minutes for 30 minutes. Data are shown as mean±s.e.m. Two-way Anova was used for statistical analysis. *P<0.05.

FIG. 9A-9F illustrate that pharmacological inhibition of IRE1α using KIRA6 reduces post-operative pain behaviors. FIG. 9A illustrates the weight distribution of mice that received KIRA6 (light grey symbols) compared to control mice that received vehicle (dark symbols). FIG. 9B illustrates the guarding scores of mice that received KIRA6 (light grey bars) compared to control mice that received vehicle (dark bars). FIG. 9C illustrates the grimace scores of mice that received KIRA6 (light grey bars) compared to control mice that received vehicle (dark bars). FIG. 9D illustrates the numbers of flinches by mice that received KIRA6 (light grey symbols) compared to control mice that received vehicle (dark symbols). FIG. 9E illustrates the numbers of rearings by mice that received KIRA6 (light grey symbols) compared to control mice that received vehicle (dark symbols). FIG. 9F illustrates the mechanical thresholds in grams of mice that received KIRA6 (light grey symbols) compared to control mice that received vehicle (dark symbols). C57BL/6J mice (n=8/group) were administered i.p. with KIRA6 (25 mg/kg) 6 hours and 30 minutes before a surgical incision was made in the left hind paw. Animals were monitored for the indicated behaviors at different time points. Data are shown as mean±s.e.m. Two-way Anova was used for (FIG. 9A-9F); *P<0.05.

FIG. 10A-10F illustrate that pharmacological inhibition of IRE1α using MKC8866 reduces post-operative pain behaviors. FIG. 10A illustrates the weight distribution of mice that received MKC8866 (light/orange symbols) compared to control mice that received vehicle (dark symbols). FIG. 10B illustrates the guarding scores of mice that received MKC8866 (light bars) compared to control mice that received vehicle (dark bars). FIG. 10C illustrates the grimace scores of mice that received MKC8866 (light/orange bars) compared to control mice that received vehicle (dark bars). FIG. 10D illustrates the numbers of flinches by mice that received MKC8866 (light symbols) compared to control mice that received vehicle (dark symbols). FIG. 10E illustrates the numbers of rearings by mice that received MKC8866 (light/orange symbols) compared to control mice that received vehicle (dark symbols). FIG. 10F illustrates the mechanical thresholds in grams of mice that received MKC8866 (light symbols) compared to control mice that received vehicle (dark symbols). C57BL/6J mice (n=8/group) were administered i.p. with MKC8866 (20 mg/kg) 6 hours and 30 minutes before a surgical incision was made in the left hind paw. Animals were monitored for the indicated behaviors at different time points. Data are shown as mean±s.e.m. Two-way Anova was used for (A-F); *P<0.05.

FIG. 11A-11F illustrate Celecoxib and post-operative pain behaviors. FIG. 11A illustrates the weight distribution of mice that received Celecoxib (light symbols) compared to control mice that received vehicle (dark symbols). FIG. 11B illustrates the guarding scores of mice that received Celecoxib (light bars) compared to control mice that received vehicle (dark bars). FIG. 11C illustrates the grimace scores of mice that received Celecoxib (light bars) compared to control mice that received vehicle (dark bars). FIG. 11D illustrates the numbers of flinches by mice that received Celecoxib (light symbols) compared to control mice that received vehicle (dark symbols). FIG. 11E illustrates the numbers of rearings by mice that received Celecoxib (light symbols) compared to control mice that received vehicle (dark symbols). FIG. 11F illustrates the mechanical thresholds in grams of mice that received Celecoxib (light symbols) compared to control mice that received vehicle (dark symbols). C57BL/6J mice (n=8/group) were administered i.p. Celecoxib (20 mg/kg) 6 hours and 30 minutes before a surgical incision was made in the left hind paw. Animals were monitored for spontaneous hind paw weight bearing distribution (FIG. 11A), grimace score (FIG. 11B), guarding score (FIG. 11C), flinches (FIG. 11D), rearing activity (FIG. 11E), and mechanical threshold (FIG. 11F). Data are shown as mean±s.e.m. Two-way Anova was used for (A-F); *P<0.05.

### DETAILED DESCRIPTION

Described herein are compositions and methods for reducing pain, and modulating processes such as hepatic lipogenesis, response to hypoxia, allergies, angiogenesis, atherosclerosis, arthritis, fever, immunosuppression, vascular permeability, and anti-tumor immunity. For example, as illustrated herein inhibition of IRE1α-XBP1s signaling can reduce Cox-2 and mPGES-1 activities in the prostaglandin biosynthetic pathway, which leads to a dramatic reduction in the production of prostaglandins such as PGE2. Moreover, targeting IRE1α or XBP1 can also lead to reduction in the expression of genes encoding cytokines like IL-6, IL-10, CXCL1 and RANTES. Hence, inhibition of IRE1α and/or XBP1 can be used to treat diseases and conditions such as pain, fever, vascular permeability, immunosuppression, and arthritis.

The serine/threonine-protein kinase/endoribonuclease inositol-requiring enzyme 1 α (IRE1α) is an enzyme that in humans is encoded by the ERN1 gene. IRE1α is a dual enzyme, containing a kinase and endoribonuclease domain. Phosphorylation of the kinase domain during times of endoplasmic reticulum (ER) stress leads to activation of the endoribonuclease domain and subsequent Xbp1 splicing. X-box binding protein 1 (XBP1) is a transcription factor containing a bZIP domain. It was first identified by its ability to bind to the Xbox, a conserved transcriptional element in the promoter of the human leukocyte antigen (HLA) DR alpha

As illustrated herein, leukocyte-intrinsic IRE1α-XBP1 signaling drives prostaglandin biosynthesis and pain. Transcriptomic analyses described herein demonstrate that induction of prostaglandin-endoperoxide synthase 2 (Ptgs2/Cox-2) and prostaglandin E synthase (Ptges/mPGES-1) was reduced in IRE1α-deficient myeloid cells undergoing endoplasmic reticulum stress. Inducible biosynthesis of prostaglandins, including PGE2, was markedly decreased in myeloid cells lacking IRE1α or XBP1, but not altered in the absence of the two other ER stress sensors PERK and ATF6a.

However, as illustrated herein, inhibition of IRE1α did not affect the expression of prostaglandin-endoperoxide synthase 1 (also known as COX1; COX3; PHS1; PCOX1; PES-1; PGHS1; PTGHS; PGG/HS; PGHS-1 and referred to as Ptgs1/Cox-1) or prostaglandin E synthase 2 (also known as GBF1; GBF-1; PGES2; C9orf15; mPGES-2, and referred to as Ptges2).

While not limited to any mechanism, IRE1α-activated XBP1 appeared to bind to and directly activate the expression of human PTGS2 and PTGES to enable PGE2 generation. Mice selectively lacking IRE1α-XBP1 in leukocytes failed to induce PGE2 upon challenge with inflammatory stimuli and demonstrated reduced behavioral pain responses in multiple PGE2-dependent models of pain.

Surprisingly, IRE1α-XBP1 as a key mediator of prostaglandin biosynthesis. Inhibition of IRE1α-XBP1 can control and reduce pain. Modulation of IRE1α-XBP1 activities can also modulate additional prostaglandin-dependent biological processes such as pregnancy, fever, vascular permeability, allergy, arthritis, and immunosuppression in cancer hosts.

The endoplasmic reticulum (ER) ensures proper folding and post-translational modification of secretory and transmembrane proteins. Physiological and pathological conditions can provoke accumulation of misfolded proteins in this cellular compartment, thus inducing ER stress and activation of the unfolded protein response (UPR). The IRE1α-XBP1 pathway is the most evolutionarily conserved arm of the UPR (Bettigole & Glimcher Annu Rev Immunol 33: 107 (2015)). When ER homeostasis is altered, the dual enzyme IRE1α undergoes oligomerization and autophosphorylation, thereby activating its endoribonuclease domain to excise a 26-nucleotide fragment from the unspliced Xbp1 mRNA. This unconventional splicing event gives rise to the functional transcription factor XBP1, which promotes expression of genes involved in enhancing the protein folding capacity of the endoplasmic reticulum. Emerging evidence indicates that IRE1α-XBP1 can also control UPR-independent cellular pathways, thus influencing processes such as hepatic lipogenesis, response to hypoxia, angiogenesis, atherosclerosis, arthritis, and anti-tumor immunity. Myeloid cells stimulated via membrane-bound Toll-like receptors (TLRs) rapidly and selectively activate IRE1α-XBP1, and this event is required for their optimal production of some pro-inflammatory cytokines. However, the precise transcriptional and metabolic programs coordinated by IRE1α-XBP1 signaling in myeloid cells under inflammatory conditions, and their physiological consequences, were previously unexplored.

### Compounds

IRE1α-XBP1 signaling inhibitors, for example, that can reduce PGE₂ production and/or that can exhibit pain reducing properties are described herein. IRE1α-XBP1 signaling can modulate processes such as hepatic lipogenesis, response to hypoxia, angiogenesis, atherosclerosis, arthritis, and anti-tumor immunity. As used herein inhibition of IRE1α-XBP1 signaling can include inhibition of IRE1α, inhibition of XBP1, or inhibition of both IRE1α and XBP1. Hence, the methods and compositions described herein can include one or more inhibitors of IRE1α and/or one or more inhibitors of XBP1. The inhibitors described herein that have unique chemical structures, unique binding mechanisms, unique inhibitory activities, and reduced off-target effects.

One aspect of the invention is a compound of formula I:

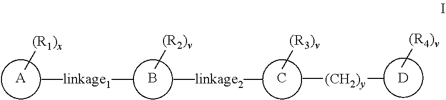

wherein:
A and B are separately each a heterocyclyl ring or a phenyl group, where the A ring has x R₁ substituents;
C is phenyl or pyridinyl;
D is heterocyclyl ring;
linkage₁ is a single bond between A and B or
linkage₁ is a C₁-C₅ alkylene, an alkenylene, an alkynylene, an alkylamido, an acyl, or an oxo(carbonyl)alkylene with a first and second terminal atom;
linkage₂ is a C₁-C₃ alkylamido, amidoalkyl, amino, urea, alkylurea, or ureaalkyl with a first and second terminal atom;
y is an integer of 0-3, and when y is 0, the linkage between the rings is a single bond;
x is an integer of 0-4 (e.g. 0-2);
v is an integer of 0-2 (e.g., 0-1);
R₁ substituents on the A ring are selected from amino, optionally substituted C₁-C₄ alkyl, optionally substituted ether, optionally substituted C₁-C₄ alkoxy, oxy, hydroxy, —NH—SO₂-phenyl-(R₅), and cyano;
R₂ substituents on the B ring are selected from amino, and optionally substituted C₁-C₄ alkyl;
R₃ substituents on the C ring are selected from halo, CF₃, optionally substituted C₁-C₄ alkyl, and optionally substituted heteroaryl; and
R₄ substituents on the D ring are selected from optionally substituted C₁-C₄ alkyl, optionally substituted C₁-C₄ alkoxy, (optionally substituted C₁-C₄ alkylene)-OH, hydroxy, optionally substituted aryl, optionally substituted benzyl, and optionally substituted benzaldehyde;
R₅ is halo; or
a pharmaceutically acceptable salt thereof.

Another aspect is a compound of formula II:

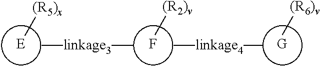

wherein:
E is phenyl;
F is phenyl, naphthalene, tetrahydronaphthalene, or a bicyclic heterocycle;
G is phenyl, or a heterocyclyl ring; heterocycle indene, dihydroindene, or benzodioxole;
linkage₃ is a C₁-C₃ alkyl, alkylamino, aminoalkyl, alkylaminoalkylene, or amino;
linkage₄ is alkylamido, amidoalkyl, alkylamidoalkylene;
R₂ is amino, or C₁-C₃ alkyl;
R₅ is halo;
R₆ is C₁-C₃ alkyl, C₁-C₃ alkoxy, or hydroxy;
x is an integer of 0-2;
v is an integer of 0-1; or
a pharmaceutically acceptable salt thereof.

The compounds of the invention include any of those described herein, including compounds shown in the Examples. In some instances, the compounds are embraced by formula I:

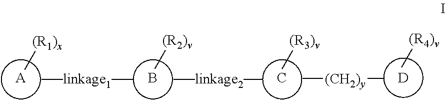

In some cases, the A ring of the compounds described herein is heteroaromatic. For example, the A ring can be a fusion of two rings. Examples of A rings include indazole, imadazopyridine, imadazopyrazine, imadazopyridazine, pyrrolopyridine, hexahydrothienopyrimidine, imidazole, pyrazole, pyrazine, pyridine, pyrimidine, and phenylpyrimidinamine. For example, the A ring can be selected from any of the following:

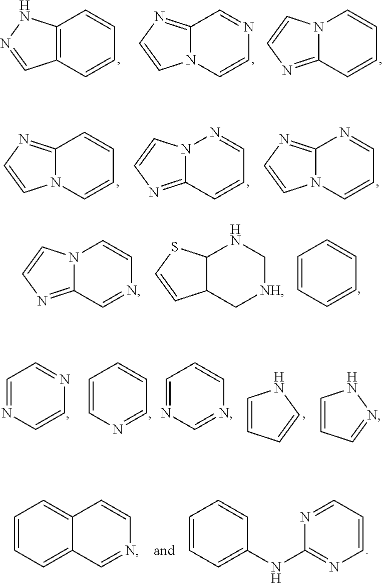

The R₁ substituents on the A ring can, for example, be selected from amino and C₁-C₃ alkyl. In some cases, the R₁ substituents on the A ring are selected from —NH₂ and —CH₃. In addition, in some cases x=0, but in other cases x=1. For example, x can in some cases be 0 when the A ring is a fusion of two rings. In other cases, x=1 or 2 when the A ring is a single, nonfused ring.

The B ring can be a single, non-fused ring. Alternatively, the B ring can be a fusion of two rings. For example, the B ring can be selected from any of the following:

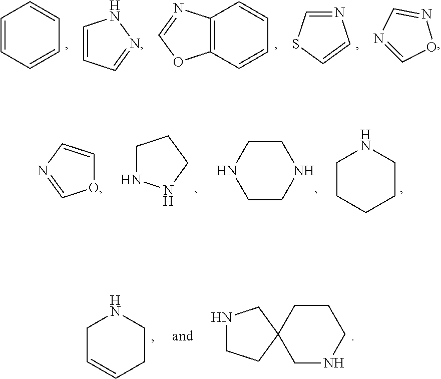

The linkage₁ can, for example, be selected from:

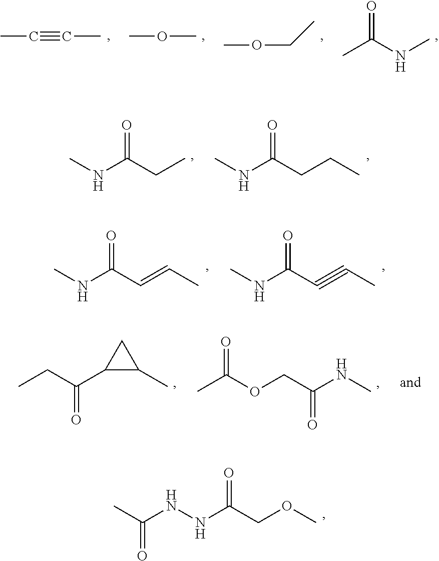

wherein a hydrogen atom on Ring A is replaced by the first terminal atom of linkage₁ and a hydrogen atom on Ring B is replaced by the second terminal atom of linkage₁.

In some cases, the C ring can be a phenyl group, and in other cases, a pyridinyl group. For example, the R₃ substituent on the C ring is CF₃.

The linkage₂ group can, for example, be selected from any of the following:

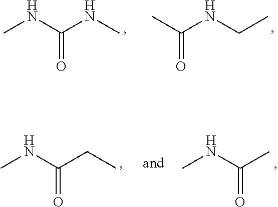

wherein a hydrogen atom on Ring B is replaced by the first terminal atom of linkage₂ and a hydrogen atom on Ring C is replaced by the second terminal atom of linkage₂, The D ring can, for example, be selected from any of the following:

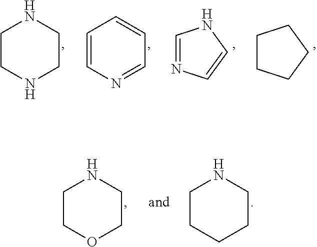

The R₄ substituents on the D ring can in some cases be selected from CH₃, CH₃CHCH₃, CH₃CH(CH₂)CH₃, and CH₃CH₂CH₃OH.

Embodiments of the invention include but are not limited to one or more compounds of formula II:

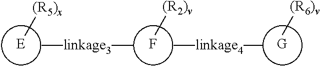

The F ring can, for example, be phenyl, naphthalene, tetrahydronaphthalene, or a bicyclic heterocycle. Such an F bicyclic heterocycle can be a spirodecane where one or two of the ring carbons is nitrogen rather than carbon. For example, an F bicyclic heterocycle can have any of the following structures:

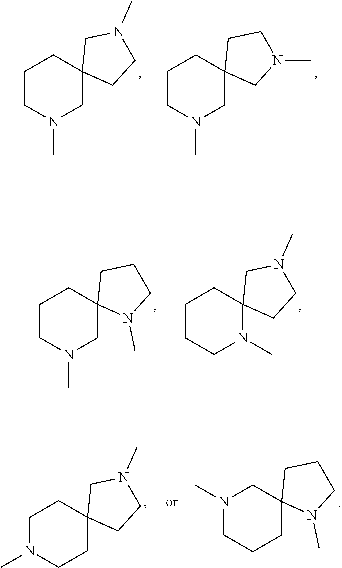

The G ring can be phenyl, a heterocycle indene, a dihydroindene, or benzodioxole.

In some cases, the A ring is heterocyclyl ring. In some cases, the A ring is a heterocyclyl that is a single non-fused ring. In other cases, the A ring is a heterocyclyl that is a fusion of two or three rings. In other cases, the A ring is a heterocyclyl that is a fusion of two rings. In some cases, the A ring of the compounds described herein is heteroaromatic. In some embodiments, the A ring is a single non-fused 5-membered heteroaryl. In some embodiments, the A ring is a single non-fused 6-membered heteroaryl. In some embodiments, the A ring is pyridinyl, pyridazinyl, pyrimidinyl, or pyrazinyl. In some embodiments, the A ring is pyridinyl. In some cases, the A ring is a heteroaryl that is a fusion of two rings. Examples of A rings include indazole, imadazopyridine, imadazopyrazine, imadazopyridazine, pyrrolopyridine, hexahydrothienopyrimidine, imidazole, pyrazole, pyrazine, pyridine, pyrimidine, phenylpyrimidinamine, quinolinyl, isoquinolinyl, tetrahydroquinolinyl, and quinazolinyl. In some embodiments, the A ring is isoquinolinyl. In some embodiments, the A ring is quinazolinyl. For example, the A ring can be selected from any of the following:

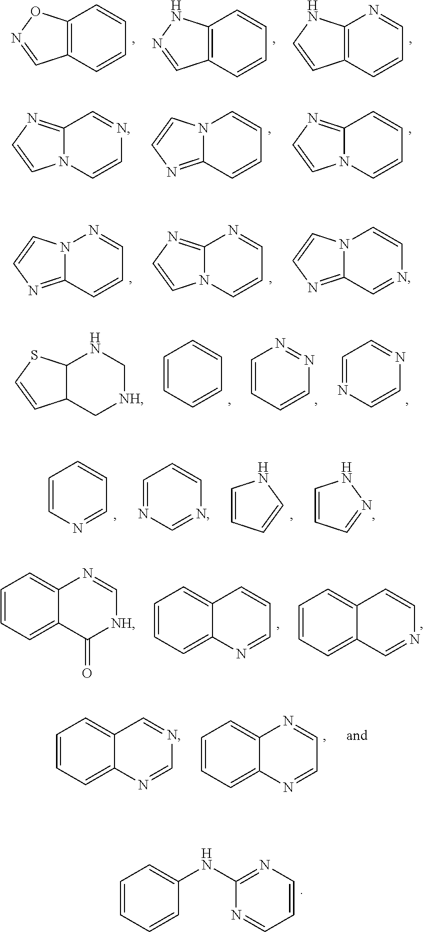

In some instances, the A ring is

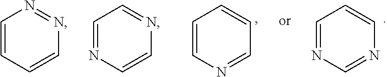

In some instances, the A ring is

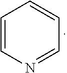

In some instances, the A ring is

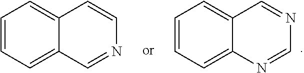 [SMILES: c1ccc2cnccc2c1 ; "or" ; c1ccc2ncncc2c1 ; "."]

In some embodiments, the R₁ substituents on the A ring are selected from amino, optionally substituted C₁-C₄ alkyl, and hydroxy. In some embodiments, the R₁ substituents on the A ring can, for example, be selected from amino and optionally substituted C₁-C₃ alkyl. In some cases, the R₁ substituents on the A ring are selected from —NH₂ and —CH₃. In addition, in some cases x=0, but in other cases x=1. In some cases, x=2. In some cases, x=3. For example, x can in some cases be 0, 1, or 2 when the A ring is a fusion of two rings. In other cases, x=1 or 2 when the A ring is a single, nonfused ring.

The B ring can be a single, non-fused ring. In some embodiments, the B ring is single, non-fused 5-membered ring. In some embodiments, the B ring is pyrazolyl, imidazolyl, or triazolyl. In some cases, the B ring is pyrazolyl. Alternatively, the B ring can be a fusion of two rings. In some embodiments, the B ring is indazolyl or benzoxazolyl. For example, the B ring can be selected from any of the following:

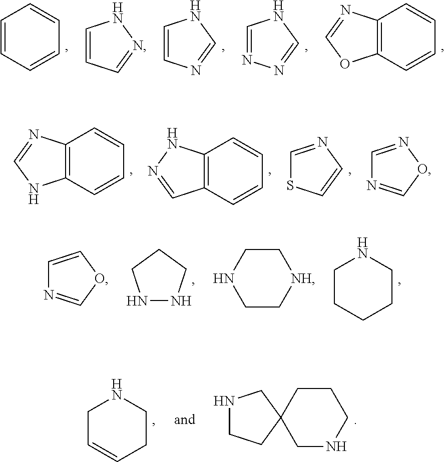

In some cases, the B ring is

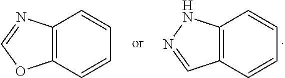 [SMILES: c1ccc2ocnc2c1 ; "or" ; c1ccc2[nH]ncc2c1 ; "."]

In some cases, the B ring is

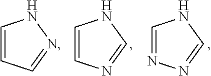

In some cases, the B ring is

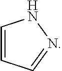

In some embodiments, R₂ substituents on the B ring are optionally substituted C₁-C₄ alkyl. In some embodiments, R₂ substituents on the B ring are —CH₃.

In some cases, the C ring can be a phenyl group, and in other cases, a pyridinyl group. In some instances, the C ring is phenyl. In some embodiments, the R₃ substituents on the C ring are selected from halo, CF₃, optionally substituted C₁-C₄ alkyl, and optionally substituted heteroaryl. In some embodiments, the R₃ substituent is halo. In some embodiments, the R₃ substituent is CF₃. In some embodiments, the R₃ substituent is optionally substituted C₁-C₄ alkyl. In some embodiments, the R₃ substituent is optionally substituted heteroaryl.

The linkage₂ group can, for example, be selected from any of the following:

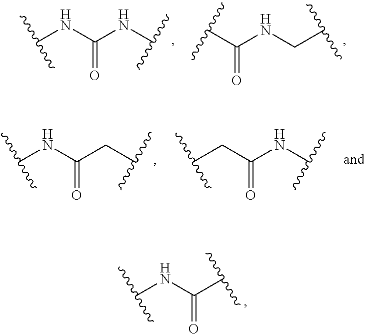 [SMILES: *NC(=O)N* ; *CNC(*)=O ; *CC(=O)N* ; *CC(=O)N* ; *NC(*)=O]

wherein a hydrogen atom on Ring B is replaced by the first terminal atom of linkage₂ and a hydrogen atom on Ring C is replaced by the second terminal atom of linkage₂. In some cases, linkage₂ is

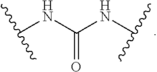 [SMILES: *NC(=O)N*]

In some cases, linkage₂ is

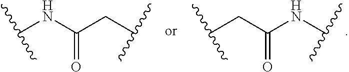 [SMILES: *CC(=O)N* ; *CC(=O)N*]

In some embodiments, D ring is a heterocyclyl ring containing at least one N atom. In some embodiments, the D ring is piperidinyl, piperazinyl, or morpholinyl. The D ring can, for example, be selected from an of the following:

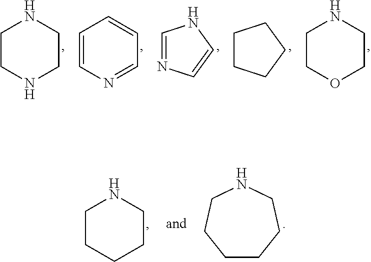

In some embodiments, Ring D is

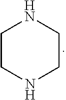

In some embodiments, the R₄ substituents on the D ring are optionally substituted C₁-C₄ alkyl. The R₄ substituents on the D ring can in some cases be selected from CH₃, CH₃CHCH₃, CH₃CH(CH₂)CH₃, and CH₃CH₂CH₃OH. In some cases, R₄ is CH₃. In some embodiments, R₄ is optionally substituted C₁-C₄ alkoxy. In some embodiments, R₄ is (optionally substituted C₁-C₄ alkylene)-OH. In some embodiments, R₄ is (optionally substituted C₁ alkylene)-OH. In some embodiments, R₄ is (optionally substituted C₂ alkylene)-OH. In some embodiments, R₄ is (optionally substituted C₃ alkylene)-OH. In some embodiments. R₄ is (optionally substituted C₄ alkylene)-OH. In some embodiments. R₄ is hydroxyl. In some embodiments. R₄ is optionally substituted aryl. In some embodiments, R₄ is phenyl. In some embodiments. R₄ is optionally substituted benzyl. In some embodiments, v is 1. In some embodiments, v is 2. In some embodiments, y is 1. In some embodiments, y is 2. In some embodiments, y is 3.

In some instances, the compounds are embraced by formula Ia:

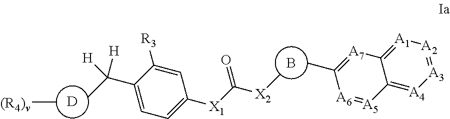

wherein:
A₁ is N, CH, or CR₁; A₂ is N, CH, or CR₁; A₃ is N, CH, or CR₁; A₄ is N, CH, or CR₁; A₅ is N, CH, or CR₁; A₆ is N, CH, or CR₁; A₇ is N CH, or CR₁;
v is an integer of 0-2;
each R₁ is NH₂ or OH; provided that the number of R₁ on the A ring does not exceed 4;
B is selected from:

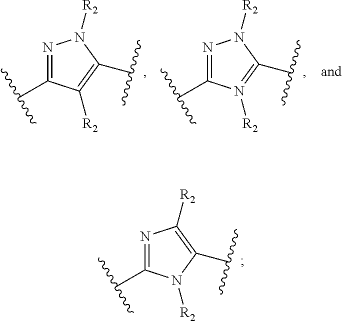

each R₂ is independently selected from H and optionally substituted C₁-C₄ alkyl;
X₁ and X₂ are each independently CH₂ or NH; with the provision that X₁ and X₂ are not each CH₂;
R₃ is selected from H, halo, CF₃, optionally substituted C₁-C₄ alkyl, and optionally substituted heteroaryl;
D is heterocyclyl ring containing at least one N atom;
each R₄ is selected from H, optionally substituted C₁-C₄ alkyl, optionally substituted C₁-C₄ alkoxy, (optionally substituted C₁-C₄ alkylene)-OH, hydroxy, optionally substituted aryl, and optionally substituted benzyl; or
a pharmaceutically acceptable salt thereof.

In some embodiments, A₁ is CH or CR₁; A₂ is N; A₃ is CH or CR₁; A₄ is N, CH, or CR₁; A₅ is CH or CR₁; A₆ is CH or CR₁; and A₇ is CH or CR₁. In some embodiments. A₁ is CH or CR₁; A₂ is N; A₃ is CH or CR₁; A₄ is N; A₅ is CH or CR₁; A₆ is CH or CR₁; and A₇ is CH or CR₁. In some embodiments, A₁ is CH or CR₁; A₂ is N; A₃ is CH or CR₁; A₄ is CH or CR₁; A₅ is CH or CR₁; A₆ is CH or CR₁; and A₇ is CH or CR₁. In some embodiments, A₁ is CH; A₂ is N; A₃ is CR₁; A₄ is N; A₅ is CH; A₆ is CH; and A₇ is CH. In some embodiments, A₁ is CH; A₂ is N; A₃ is CR₁; A₄ is CR₁; A₅ is CH; A₆ is CH; and A₇ is CH.

In some embodiments, A₁ is CH or CR₁; A₂ is N; A₃ is CH or CR₁; A₄ is N; A₅ is CH; A₆ is CH; and A₇ is CH. In some embodiments, A₁ is CH or CR₁; A₂ is N; A₃ is CH or CR₁; A₄ is CH or CR₁; A₅ is CH; A₆ is CH; and A₇ is CH.

In some embodiments, A₁ is N. In some embodiments, A₁ is CH. In some embodiments. A₁ is CR₁, and R₁ is OH. In some embodiments, A₁ is CR₁, and R₁ is NH₂. In some embodiments. A₂ is N. In some embodiments, A₂ is CH. In some embodiments, A₂ is CR₁, and R₁ is OH. In some embodiments. A₂ is CR₁, and R₁ is NH₂. In some embodiments, A₃ is N. In some embodiments, A₃ is CH. In some embodiments, A₃ is CR₁, and R₁ is OH. In some embodiments. A₃ is CR₁, and R₁ is NH₂. In some embodiments, A₄ is N. In some embodiments, A₄ is CH. In some embodiments. A₄ is CR₁, and R₁ is OH. In some embodiments, A₄ is CR₁, and R₁ is NH₂. In some embodiments. A₅ is N. In some embodiments, A₅ is CH. In some embodiments. A₅ is CR₁, and R₁ is OH. In some embodiments, A₅ is CR₁, and R₁ is NH₂. In some embodiments, A₆ is N. In some embodiments, A₆ is CH. In some embodiments, A₆ is CR₁, and R₁ is OH. In some embodiments. A₆ is CR₁, and R₁ is NH₂. In some embodiments, A₇ is N. In some embodiments, A₇ is CH. In some embodiments, A₇ is CR₁, and R₁ is OH. In some embodiments, A₇ is CR₁, and R₁ is NH₂.

In some embodiments, B is

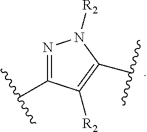

In some embodiments, B is

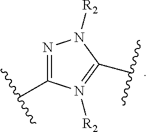

In some embodiments, B is

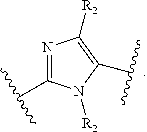

In some embodiments, each R₂ is H. In some embodiments, each R₂ is optionally substituted C₁-C₄ alkyl. In some embodiments, each R₂ is methyl.

In some embodiments, X₁ and X₂ are each NH. In some embodiments, X₁ is CH₂ and X₂ is NH. In some embodiments, X₁ is NH and X₂ is CH₂. In some embodiments. R₃ is H. In some embodiments. R₃ is halo. In some embodiments. R₃ is CF₃. In some embodiments, R₃ is optionally substituted C₁-C₄ alkyl. In some embodiments. R₃ is optionally substituted heteroaryl.

In some embodiments, D is selected from:

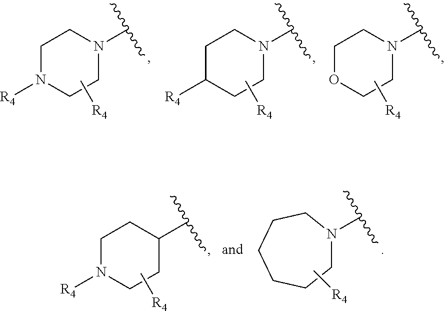

In some embodiments, D is

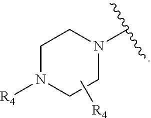

In some embodiments, D is

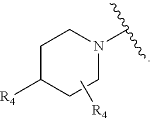

In some embodiments, D is

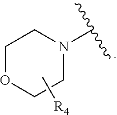

In some embodiments, D is

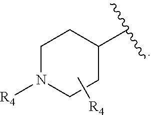

In some embodiments, D is

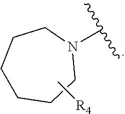

In some embodiments, v is 0. In some embodiments, v is 1. In some embodiments, R₄ is H. In some embodiments, R₄ is optionally substituted C₁-C₄ alkyl. In some embodiments. R₄ is methyl (Me), ethyl (Et), propyl or isopropyl (i-Pr). In some embodiments. R₄ is optionally substituted C₁-C₄ alkylene-OH. In some embodiments, R₄ is optionally substituted C₁ alkylene-OH. In some embodiments, R₄ is optionally substituted C₂ alkylene-OH. In some embodiments, R₄ is optionally substituted C₃ alkylene-OH. In some embodiments, R₄ is optionally substituted C₄ alkylene-OH. In some embodiments. R₄ is hydroxyl. In some embodiments. R₄ is optionally substituted aryl. In some embodiments, R₄ is phenyl. In some embodiments, R₄ is optionally substituted benzyl. In some embodiments, v is 2. In some embodiments, at least one R₄ is H. In some embodiments, at least one R₄ is optionally substituted C₁-C₄ alkyl. In some embodiments, at least one R₄ is Me, Et, or i-Pr. In some embodiments, at least one R₄ is optionally substituted C₁-C₄ alkylene)-OH. In some embodiments, at least one R₄ is hydroxyl. In some embodiments, at least one R₄ is optionally substituted aryl. In some embodiments, at least one R₄ is optionally substituted benzyl.

In some instances, the compounds are embraced by formula Ib:

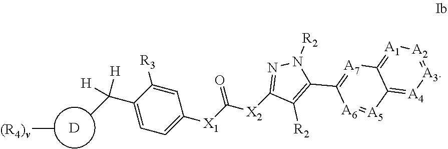

In some instances, the compounds are embraced by formula Ic:

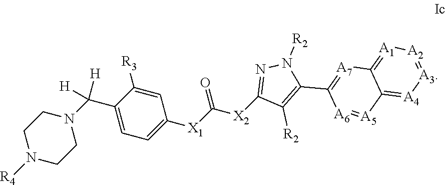

In some instances, the compounds are embraced by formula Id:

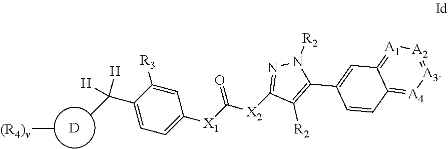

In some instances, the compounds are embraced by formula Ie:

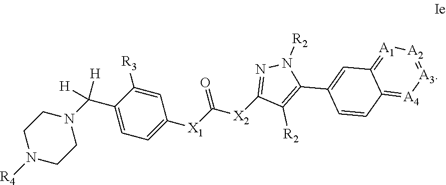

The compounds include any of those described herein, including compounds shown in the Examples. In some instances, the compounds are embraced by Formula III:

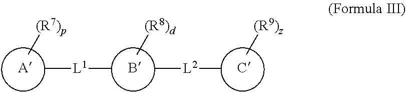

wherein:
the A′ ring is a heterocyclyl or aryl;
p is an integer of 0-2;
R⁷ is independently amino, C₁-C₄ alkyl, C₁-C₄ alkoxy, hydroxy, C₁-C₄ hydroxyalkyl, arylsulfonyl, cyano, halogen, or trifluoromethyl;
L¹ is a single bond, C₁-C₃ alkyl, C₂-C₃ alkenyl or C₂-C₃ alkynyl;
the B′ ring is a heterocyclyl or aryl;
d is an integer of 0-1;
R⁸ is independently amino, C₁-C₄ alkyl, halogen or trifluoromethyl;
L² is amino, urea, amido, alkylamido, alkenylamido, amidoalkyl, amidoalkenyl, alkylurea, or alkenylurea;
the C′ ring is a heterocyclyl or aryl;
z is an integer of 0-2;
R⁹ is independently amino, C₁-C₄ alkyl, C₁-C₄ alkoxy, hydroxy, C₁-C₄ hydroxyalkyl, cyano, halogen, trifluoromethyl, difluoromethyl, monofluoroalkyl, benzyl, dialkylaminosulfonyl, alkylsulfonyl, boronic ester, boronic acid, dialkylphosphine, C₁-C₄ alkylcarboxyl, dialkylamido, cycloalkylalkyl, or heterocyclylalkyl;
or a pharmaceutically acceptable salt thereof.
L¹ in compounds of the Formula III can be a single bond.
L¹ in compounds of the Formula III can be C₁-C₃ alkyl, C₂-C₃ alkenyl or C₂-C₃ alkynyl; and L² is a urea, alkylurea, or alkenylurea.

In some instances, the compounds are embraced by Formula IV,

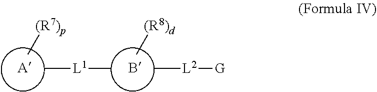

wherein:
the A′ ring is a heterocyclyl or aryl;
p is an integer of 0-2;
R⁷ is independently amino, C₁-C₄ alkyl, C₁-C₄ alkoxy, hydroxy, C₁-C₄ hydroxyalkyl, arylsulfonyl, cyano, halogen, or trifluoromethyl;
L¹ is a single bond, C₁-C₃ alkyl, C₂-C₃ alkenyl or C₂-C₃ alkynyl;
the B′ ring is a heterocyclyl or aryl;
d is an integer of 0-1;
R⁸ is independently amino, C₁-C₄ alkyl, halogen or trifluoromethyl;
L² is amino, urea, amido, alkylamido, alkenylamido, amidoalkyl, amidoalkenyl, alkylurea, or alkenylurea;
G is dialkylamino or H;
or a pharmaceutically acceptable salt thereof.

In some instances, the compounds are embraced by Formula V,

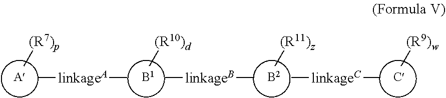

wherein:
the A′ ring is a heterocyclyl or aryl;
p is an integer of 0-2;
R⁷ is independently amino, C₁-C₄ alkyl, C₁-C₄ alkoxy, hydroxy, C₁-C₄ hydroxyalkyl, arylsulfonyl, cyano, halogen, trifluoromethyl or a group having the structure

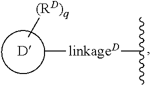

wherein the D′ ring is a heterocyclyl;
q is an integer of 0-2;
R^{D} is amino, C₁-C₄ alkyl, C₁-C₄ alkoxy, hydroxy, C₁-C₄ hydroxyalkyl, arylsulfonyl, cyano, halogen, or trifluoromethyl; and
the linkage^{D} is a single bond, amino or C₁-C₃ alkyl;
the B¹ ring is a heterocyclyl or aryl;
d is an integer of 0-1;
R¹⁰ is independently amino, C₁-C₃ alkyl, halogen or trifluoromethyl;
the B² ring is phenyl, pyridinyl, naphthyl or a bicyclic heterocyclyl;
z is an integer of 0-1;
R¹¹ is independently amino, C₁-C₄ alkyl, halogen or trifluoromethyl;
the C′ ring is a heterocyclyl ring;
w is an integer of 0-2;
R⁹ is independently C₁-C₄ alkyl, C₁-C₄ alkoxy, C₁-C₄ hydroxyalkyl, hydroxy, aryl, benzyl, benzaldehyde, halogen, cyano, amino, heterocyclyl, heterocyclylalkyl, cycloalkyl, cycloalkylalkyl, trifluoromethyl, difluoromethyl, monofluoroalkyl, dialkylaminosulfonyl, alkylsulfonyl, dialkylphosphine, C₁-C₄ alkylcarboxyl, dialkylamido, or dialkylamino;
the linkage^{A} is a single bond, is a C₁-C₅ alkyl, alkenyl, alkynyl, alkylamido, acyl, or oxo(carbonyl)alkyl;
the linkage^{B} is alkylamido, alkenylamido, amidoalkyl, amidoalkenyl, urea, alkylurea, or alkenylurea;
the linkage^{C} is CH or (CH₂)ₙ, where n is an integer of 0-3, and when n is 0, the linkage between the B² ring and the C ring is a single bond; and
or a pharmaceutically acceptable salt thereof.

In compounds of Formula V, p can be 1-2; and at least one of R⁷ can be

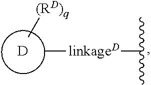

In compounds of Formula V, w can be 1-2; and at least one of R⁹ can be heterocyclyl, heterocyclylalkyl, cycloalkyl or cycloalkylalkyl.

In compounds of Formula V, if linkage^{A} is alkynyl and linkage^{B} is urea, then A can be aryl.

In compounds of Formula V, at least one of p, d, z, and w can be other than 0.

In the compounds disclosed herein, the A′ ring can be heteroaromatic. The A′ ring can be indazole, imadazopyridine, imadazopyrazine, imadazopyridazine, pyrrolopyridine, hexahydrothienopyrimidine, imidazole, pyrazole, pyrazine, pyridine, pyrimidine, phenylpyrimidinamine, quinolinyl, isoquinolinyl, tetrahydroquinolinyl or quinazolinyl. The A′ ring can be a single, non-fused ring. The A′ ring can be a fusion of two rings. The A′ ring can in some cases include a phenyl. Examples of A′ rings include indazole, imadazopyridine, imadazopyrazine, imadazopyridazine, pyrrolopyridine, hexahydrothienopyrimidine, imidazole, pyrazole, pyrazine, pyridine, pyrimidine, and phenylpyrimidinamine. For example, the A′ ring can be:

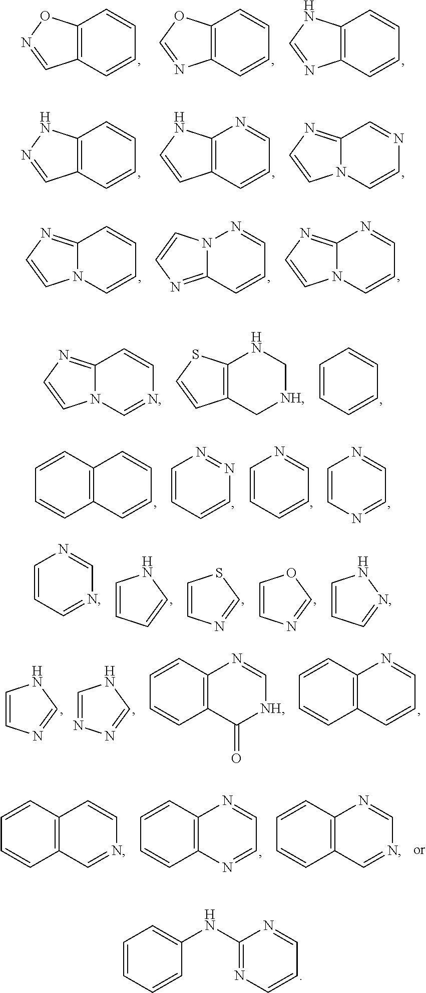

The A′ ring can be:

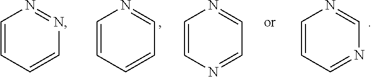

The A′ ring can be:

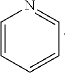

The A′ ring can be:

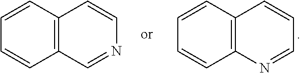 [SMILES: c1ccc2cnccc2c1 ; c1ccc2ncccc2c1]

The A′ ring can be:

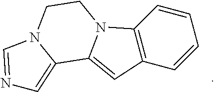 [SMILES: c1ccc2c(c1)cc1n2CCn2cncc2-1]

The R⁷ substituents on the A′ ring can, for example, be selected from amino and C₁-C₄ alkyl. The R⁷ substituents on the A′ ring can, for example, be selected from amino and C₁-C₃ alkyl. The R⁷ substituents on the A′ ring can be selected from —NH₂ and —CH₃. In addition, p can be 0. Or p can be 1. For example, p can be 0 when the A′ ring is a fusion of two rings. Or p can be 1 or 2 when the A′ ring is a single, non-fused ring.

R₇ on the A′ ring can be independently amino, C₁-C₄ alkyl, C₁-C₄ alkoxy, hydroxy, C₁-C₄ hydroxyalkyl, arylsulfonyl, cyano, halogen, trifluoromethyl or a group having the structure

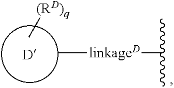

wherein the D′ ring is a heterocyclyl; q is an integer of 0-2; R^{D} is amino, C₁-C₄ alkyl. C₁-C₄ alkoxy, hydroxy, C₁-C₄ hydroxyalkyl, arylsulfonyl, cyano, halogen, or trifluoromethyl; and the linkage^{D} is a single bond, amino or C₁-C₃ alkyl. The linkage^{d} can be a single bond. The linkage^{d} can be a methylene.

R⁷ on the A′ ring can be independently amino, C₁-C₄ alkyl, hydroxy or halogen. Or R⁷ can be independently amino or substituted C₁-C₄ alkyl. Or R⁷ can be independently amino or unsubstituted C₁-C₄ alkyl. R⁷ can be amino. Or R⁷ can be unsubstituted C₁-C₄ alkyl.

The B¹ ring can be a single, non-fused ring. Alternatively, the B¹ ring can be a fusion of two rings. For example, the B¹ ring can be selected from any of the following:

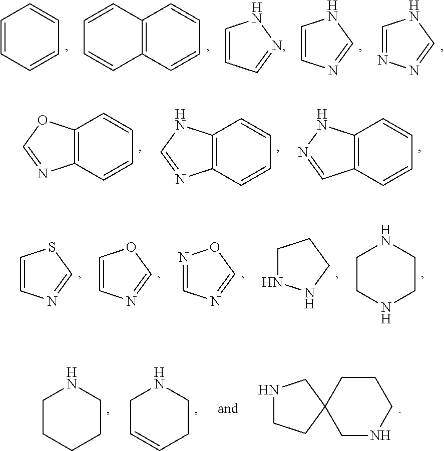

The B¹ ring can be:

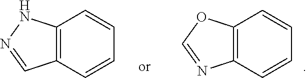 [SMILES: c1ccc2[nH]ncc2c1 ; c1ccc2ocnc2c1]

The B¹ ring can be:

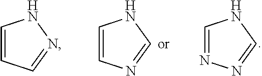 [SMILES: c1cn[nH]c1 ; c1c[nH]cn1 ; c1nnc[nH]1]

The B¹ ring can be:

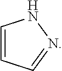

The B¹ ring can, for example, be phenyl, naphthalene, tetrahydronaphthalene, or a bicyclic heterocycle. Such B¹ ring bicyclic heterocycle can be a spirodecane where one or two of the ring carbons is nitrogen rather than carbon. For example, a B¹ ring bicyclic heterocycle can have any of the following structures:

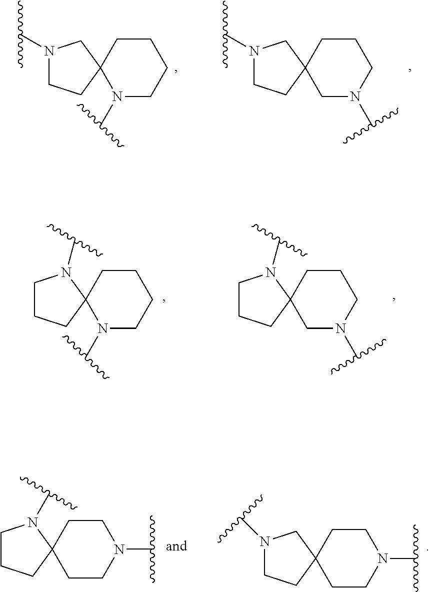 [SMILES: *N1CCC2(CCCCN2*)C1 ; *N1CCCC2(CCN(*)C2)C1 ; *N1CCCCC12CCCN2* ; *N1CCCC2(CCCN2*)C1 ; *N1CCC2(CCCN2*)CC1 ; *N1CCC2(CC1)CCN(*)C2]

R¹⁰ on the B¹ ring can be independently amino, C₁-C₄ alkyl, halogen or trifluoromethyl. Or R¹⁰ can be independently amino, C₁-C₄ alkyl, or trifluoromethyl. Or R¹⁰ can be independently C₁-C₄ alkyl or trifluoromethyl. Or R¹⁰ can be unsubstituted C₁-C₄ alkyl. Or R¹⁰ can be substituted C₁-C₄ alkyl.

The R¹⁰ substituents on the B¹ ring can be optionally substituted C₁-C₄ alkyl. Or the R¹⁰ substituents on the B¹ ring can be optionally substituted C₁-C₃ alkyl. Or the R¹⁰ substituents on the B¹ ring can be methyl. Or the R¹⁰ substituents on the B¹ ring can be optionally substituted linear C₁-C₄ alkyl. Or the R¹⁰ substituents on the B¹ ring can be unsubstituted. Or the R¹⁰ substituents on the B¹ ring can be amino. Or the R₁₀ substituents on the B¹ ring can be trifluoromethyl. Or the R¹⁰ substituents on the B¹ ring can be halogen.

The B¹ ring can be heteroaromatic. The B¹ ring can be indazole, imadazopyridine, imadazopyrazine, imadazopyridazine, pyrrolopyridine, hexahydrothienopyrimidine, imidazole, pyrazole, pyrazine, pyridine, pyrimidine, phenylpyrimidinamine, quinolinyl, isoquinolinyl, tetrahydroquinolinyl or quinazolinyl. The B¹ ring can be pyrazolyl, imidazolyl, or triazolyl. The B¹ ring can be a single, non-fused ring. The B¹ ring can be a fusion of two rings. Or the B¹ ring can be phenyl.

The B¹ ring can be a single, non-fused ring. Alternatively, the B¹ ring can be a fusion of two rings. For example, the B¹ ring can be selected from any of the following:

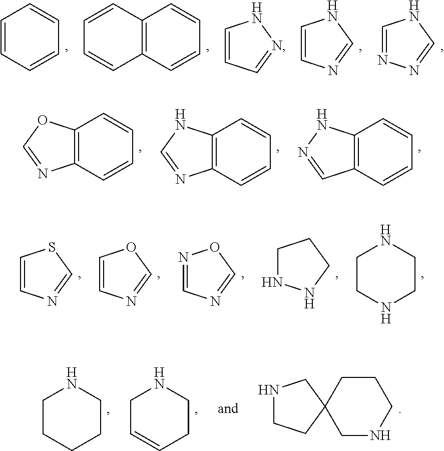

The B¹ ring can be:

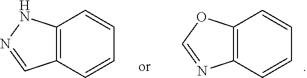 [SMILES: c1ccc2[nH]ncc2c1 ; c1ccc2ocnc2c1]

The B¹ ring can be:

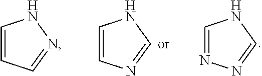 [SMILES: c1cn[nH]c1 ; c1c[nH]cn1 ; c1nnc[nH]1]

The B¹ ring can be:

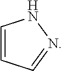

The B¹ ring can, for example, be phenyl, naphthalene, tetrahydronaphthalene, or a bicyclic heterocycle. Such B¹ ring bicyclic heterocycle can be a spirodecane where one or two of the ring carbons is nitrogen rather than carbon. For example, a B¹ ring bicyclic heterocycle can have any of the following structures:

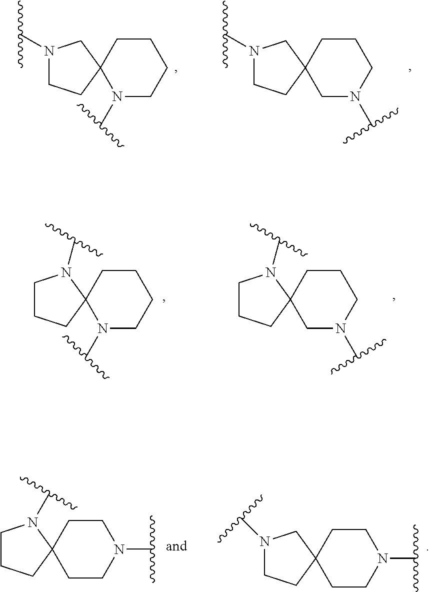 [SMILES: *N1CCC2(CCCCN2*)C1 ; *N1CCCC2(CCN(*)C2)C1 ; *N1CCCCC12CCCN2* ; *N1CCCC2(CCCN2*)C1 ; *N1CCC2(CCCN2*)CC1 ; *N1CCC2(CC1)CCN(*)C2]

R¹⁰ on the B¹ ring can be independently amino, C₁-C₄ alkyl, halogen or trifluoromethyl. Or R¹⁰ can be independently amino, C₁-C₄ alkyl, or trifluoromethyl. Or R¹⁰ can be independently C₁-C₄ alkyl or trifluoromethyl. Or R¹⁰ can be unsubstituted C₁-C₄ alkyl. Or R¹⁰ can be substituted C₁-C₄ alkyl.

R¹⁰ substituents on the B¹ ring can optionally be substituted C₁-C₄ alkyl. Or R¹⁰ substituents on the B¹ ring can optionally be substituted C₁-C₃ alkyl. Or R¹⁰ substituents on the B¹ ring can be methyl. R¹⁰ substituents on the B¹ ring can optionally be substituted linear C₁-C₄ alkyl. R¹⁰ substituents on the B¹ ring can be unsubstituted. R¹⁰ substituents on the B¹ ring can be amino. R¹⁰ substituents on the B¹ ring can be trifluoromethyl. R¹⁰ substituents on the B¹ ring can be halogen.

B² can be a phenyl, pyridinyl, naphthyl or a bicyclic heterocyclyl. The B² ring can be a phenyl group. Or the B² ring can be a pyridinyl group. The B² ring can be a benzimidazole group. The B² ring can be a naphthylene group. The R¹¹ substituent on the B² ring can be CF₃. The B² ring can be pyridinyl. The B² ring can be napthyl. The B² ring can be bicyclic heterocyclyl.

R¹¹ on the B² ring can be independently amino, C₁-C₄ alkyl, halogen or trifluoromethyl. R¹¹ can be independently amino, C₁-C₄ alkyl, or trifluoromethyl. R¹¹ can be independently C₁-C₄ alkyl or trifluoromethyl. R¹¹ can be unsubstituted C₁-C₄ alkyl. R¹¹ can be substituted C₁-C₄ alkyl.

R¹¹ substituents on the B² ring can be optionally substituted C₁-C₅ alkyl. R¹¹ substituents on the B² ring can be optionally substituted C₁-C₃ alkyl. R¹¹ substituents on the B² ring can be methyl. R¹¹ substituents on the B² ring can be optionally substituted linear C₁-C₄ alkyl. R¹¹ substituents on the B² ring can be unsubstituted. R¹¹ substituents on the B² ring can be amino. R¹¹ substituents on the B² ring can be trifluoromethyl. R¹¹ substituents on the B² ring can be halogen.

R¹¹ on the B² ring can be independently amino, C₁-C₄ alkyl, halogen or trifluoromethyl. R¹¹ can be independently amino, C₁-C₄ alkyl, or trifluoromethyl. R¹¹ can be independently C₁-C₄ alkyl or trifluoromethyl. R¹¹ can be unsubstituted C₁-C₄ alkyl. R¹¹ can be substituted C₁-C₄ alkyl.

Linkage^{A} can be methylene or acetylene. Linkage² can be:

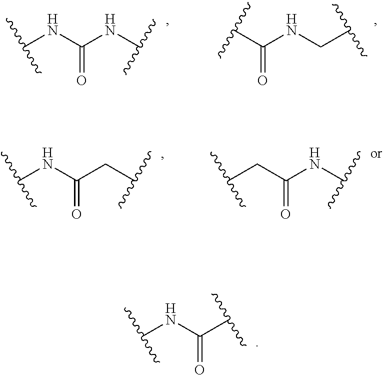 [SMILES: *NC(=O)N* ; *CNC(*)=O ; *CC(=O)N* ; *CC(=O)N* ; *NC(*)=O]

Linkage^{A} can, for example, be selected from:

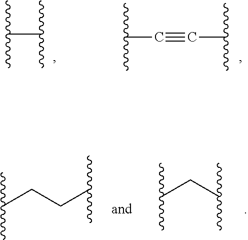

Linkage^{A} can also be amino, amido, alkylamido, alkenylamido, amidoalkyl, or amidoalkenyl. Linkage^{A} can also be acylamido, acylamido, acylamidoalkyl, or acylamidoalkenyl. Linkage^{A} can also be amidoalkylamido, amidoalkenlamido, hydrazinyl, hydrazidyl, alkylhydrazinyl, alkylhydrazidyl, N-acylhydrazide. N-acylhydrazidyl, hydrazodicarbonyl, oxalamidyl, N-alkyl-oxalamidyl, acylurea, or dialkyldiamido, each of which may be optionally substituted. Linkage^{A} can contain at least one urea, amido, amino, alkyl, alkenyl, hydrazinyl, hydrazidyl, carbonyl, ester, and ether units, any of which may be optionally substituted. Linkage^{A} can contain at least two of urea, amido, amino, alkyl, alkenyl, hydrazinyl, hydrazidyl, carbonyl, ester, and ether units, any of which may be optionally substituted. Linkage^{A} can contain at least three of urea, amido, amino, alkyl, alkenyl, hydrazinyl, hydrazidyl, carbonyl, ester, and ether units, any of which may be optionally substituted. Linkage^{A} can contain at least four of urea, amido, amino, alkyl, alkenyl, hydrazinyl, hydrazidyl, carbonyl, ester, and ether units, any of which may be optionally substituted. Linkage¹ can also be a carbonyl. Linkage^{A} can also be an alkoxy, alkylthio, sulfone or a thio.

Linkage^{B} can be alkylamido, alkenylamido, amidoalkyl, or amidoalkenyl. Linkage^{B} can be alkenylamido or amidoalkenyl.

The linkage^{B} group can, for example, be selected from any of the following:

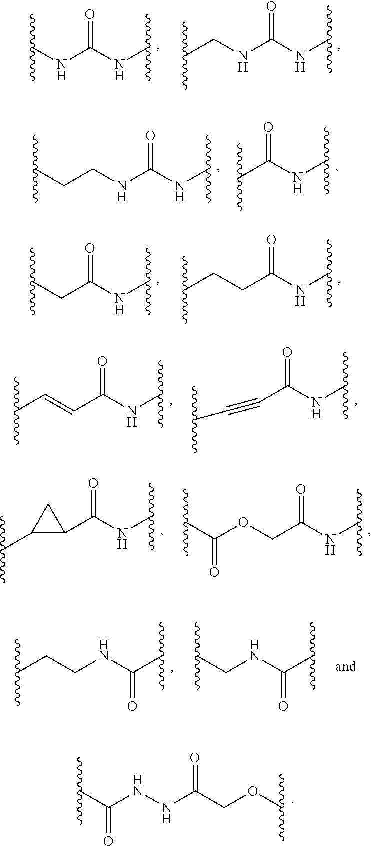

Linkage^{B} can also be amino, amido, alkylamido, alkenylamido, amidoalkyl, or amidoalkenyl. Linkage^{B} can also be acylamido, acylamido, acylamidoalkyl, or acylamidoalkenyl. Linkage^{B} can also be amidoalkylamido, amidoalkenlamido, hydrazinyl, hydrazidyl, alkylhydrazinyl, alkylhydrazidyl, N-acylhydrazide, N-acylhydrazidyl, hydrazodicarbonyl, oxalamidyl. N-alkyl-oxalamidyl, acylurea, or dialkyldiamido, each of which may be optionally substituted. Linkage^{B} can contain at least one urea, amido, amino, alkyl, alkenyl, hydrazinyl, hydrazidyl, carbonyl, ester, and ether units, any of which may be optionally substituted. Linkage^{B} can contain at least two of urea, amido, amino, alkyl, alkenyl, hydrazinyl, hydrazidyl, carbonyl, ester, and ether units, any of which may be optionally substituted. Linkage^{B} can contain at least three of urea, amido, amino, alkyl, alkenyl, hydrazinyl, hydrazidyl, carbonyl, ester, and ether units, any of which may be optionally substituted. Linkage^{B} can contain at least four of urea, amido, amino, alkyl, alkenyl, hydrazinyl, hydrazidyl, carbonyl, ester, and ether units, any of which may be optionally substituted. Linkage^{B} can be a bond. Linkage^{B} can also be a carbonyl. Linkage^{B} can also be a alkylthio, sulfone or a thio.

Linkage^{A} can be a single bond.

Linkage^{A} can be methylene or acetylene.

Linkage^{A} group can, for example, be selected from any of the following:

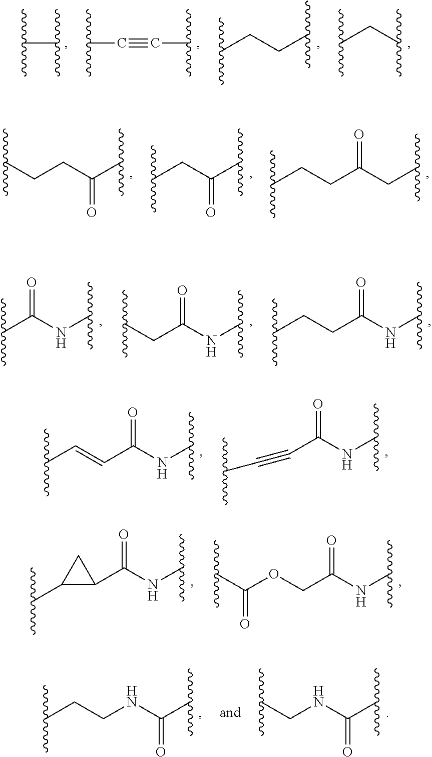

The linkage^{B} group can, for example, be selected from any of the following:

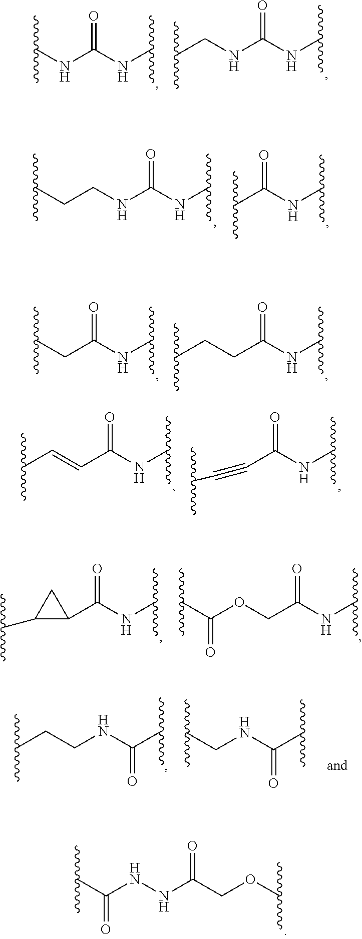

The linkage^{B} can for example be

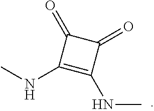

If the linkage^{A} is alkynyl and linkage^{B} is urea, then A can be aryl. Linkage^{A} can be other than alkynyl. Linkage^{B} can be other than urea.

Linkage^{B} can be

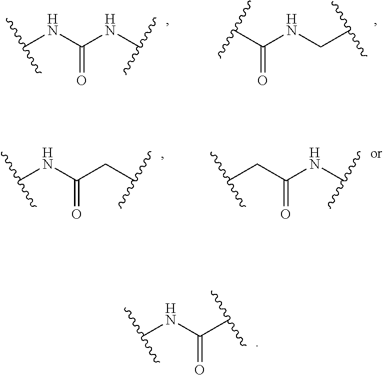 [SMILES: *NC(=O)N* ; *CNC(*)=O ; *CC(=O)N* ; *CC(=O)N* ; *NC(*)=O]

Or linkage^{B} can be

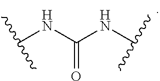 [SMILES: *NC(=O)N*]

Linkage^{B} can be alkylamido, alkenylamido, amidoalkyl, or amidoalkenyl. Linkage^{B} can be alkenylamido or amidoalkenyl.

Linkage^{C} can be methylene.

Linkage^{C} can be a —CH— unit linked to ring C′ via a double bond.

The C′ ring can be heteroaromatic. The C′ ring can be indazole, imadazopyridine, imadazopyrazine, imadazopyridazine, pyrrolopyridine, hexahydrothienopyrimidine, imidazole, pyrazole, pyrazine, pyridine, pyrimidine, phenylpyrimidinamine, quinolinyl, isoquinolinyl, tetrahydroquinolinyl or quinazolinyl.

The C′ ring can be pyrazolyl, imidazolyl, or triazolyl.

The C′ ring can be a single, non-fused ring. The C′ ring can be a fusion of two rings. The C′ ring can be phenyl. The C′ ring can be a heterocyclyl ring containing at least one N atom. The C′ ring can be piperidinyl, piperazinyl, or morpholinyl.

The C′ ring can be a phenyl group. Or the C′ ring can be a pyridinyl group. The R⁹ substituent on the C′ phenyl ring can be CF₃.

The C′ ring can be a heterocyclyl or aryl ring.

The C′ ring can be a heterocyclyl ring.

The C′ ring can, for example, be selected from the following:

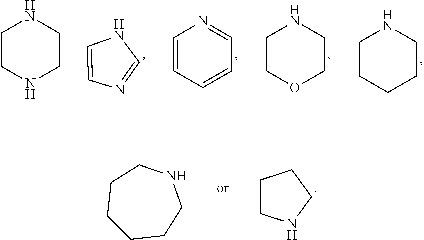

The C′ ring can be a substituted heterocycle. For example, the C′ ring can be

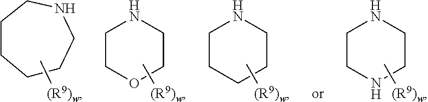

The C′ ring can be

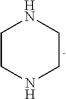

The R⁹ substituents on the C′ ring can be selected from CH₃, CH₃CHCH₃, CH₃CH(CH₂)CH₃, and CH₃CH₂CH₃OH.

The R⁹ substituents on the C′ ring can be independently amino, C₁-C₄ alkyl, C₁-C₄ alkoxy, hydroxy, C₁-C₄ hydroxyalkyl, cyano, halogen, trifluoromethyl, difluoromethyl, monofluoroalkyl, benzyl, dialkylaminosulfonyl, alkylsulfonyl, boronic ester, boronic acid, dialkylphosphine, C₁-C₄ alkykarboxyl, dialkylamido, cycloalkyl, cycloalkylalkyl, heterocyclyl or heterocyclylalkyl. R⁹ on the C′ ring can be independently C₁-C₄ alkyl, C₁-C₄ alkoxy, hydroxy, aryl, or benzyl. R⁹ can be substituted C₁-C₄ alkyl. R⁹ can be unsubstituted C₁-C₄ alkyl. R⁹ on the C′ ring can be non-aromatic heterocyclyl and aromatic heterocyclyl.

The R⁹ substituents on the C′ ring can optionally be substituted C₁-C₄ alkyl. The R⁹ substituents on the C′ ring can be selected from CH₃, CH₃CHCH₃, CH₃CH(CH₂)CH₃, and CH₃CH₂CH₃OH. R⁹ can be CH₃. R⁹ can optionally be substituted C₁-C₄ alkoxy. R⁹ can be (optionally substituted C₁-C₄alkylene)-OH. R⁹ can be (optionally substituted C₄alkylene)-OH. R⁹ can be (optionally substituted C₂alkylene)-OH. R⁹ can be (optionally substituted C₃alkylene)-OH. R⁹ can be (optionally substituted C₄alkylene)-OH. R⁹ can be hydroxyl. R⁹ can be optionally substituted aryl. R⁹ can be phenyl. R⁹ can be optionally substituted benzyl.

R⁹ can also be nitro, arylsulfonamido, amido, alkenyl, alkynyl, alkylsulfonyl, heterocycylcarbonyl, cycloalkylcarbonyl, trifluoromethoxy, alkylthio, and acetamido.

The D′ ring can be heteroaromatic. Or the D′ ring can be indazole, imadazopyridine, imadazopyrazine, imadazopyridazine, pyrrolopyridine, hexahydrothienopyrimidine, imidazole, pyrazole, pyrazine, pyridine, pyrimidine, phenylpyrimidinamine, quinolinyl, isoquinolinyl, tetrahydroquinolinyl or quinazolinyl. The D′ ring can be a single, non-fused ring. The D′ ring can be a fusion of two rings.

Examples of D′ rings include indazole, imadazopyridine, imadazopyrazine, imadazopyridazine, pyrrolopyridine, hexahydrothienopyrimidine, imidazole, pyrazole, pyrazine, pyridine, pyrimidine, and phenylpyrimidinamine. For example, the D′ ring can be:

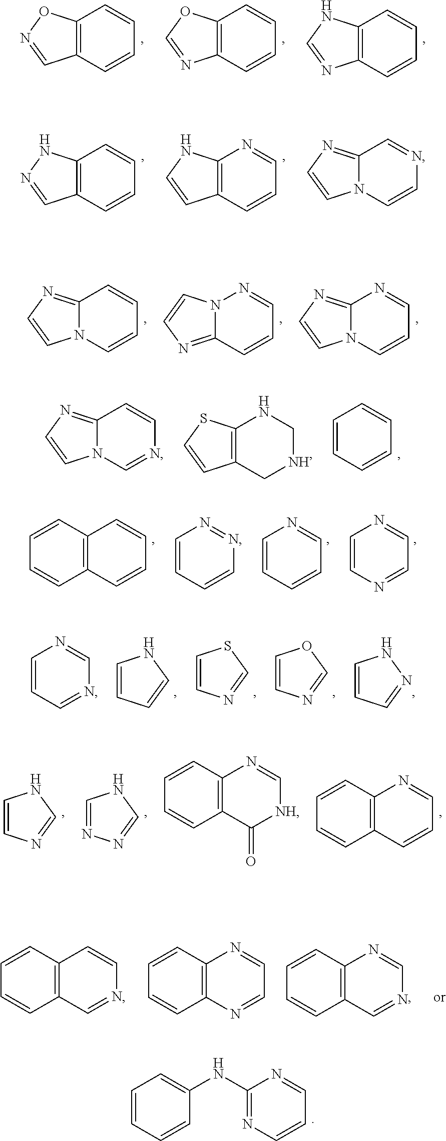

The D′ ring can be:

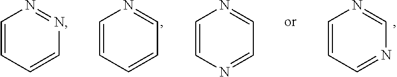

The D′ ring can be:

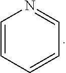

The D′ ring can be:

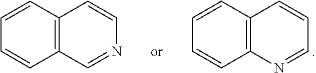 [SMILES: c1ccc2cnccc2c1 ; c1ccc2ncccc2c1]

The R^{D} substituents on the D′ ring can, for example, be selected from amino and C₁-C₄ alkyl. The R^{D} substituents on the D′ ring can, for example, be selected from amino and C₁-C₃ alkyl. The R substituents on the D′ ring are selected from —NH₂ and —CH₃. In addition, v can be 0 or q can be 1. For example, q can be 0 when the A′ ring is a fusion of two rings. Or q can be 2 when the D ring is a single, non-fused ring.

p can be 0. Or p can be 1. Or p can be 2. d can be 0. Or d can be 1. z can be 0. z can be 1. z can be 2. q can be 0. q can be 1. q can be 2. w can be 0. w can be 1. w can be 2.

The C′ ring can be phenyl, a heterocycle indene, a dihydroindene, or benzodioxole. The B² ring can be phenyl, a heterocycle indene, a dihydroindene, or benzodioxole.

All structures encompassed within a claim are “chemically feasible”, by which is meant that the structure depicted by any combination or subcombination of optional substituents meant to be recited by the claim is physically capable of existence with at least some stability as can be determined by the laws of structural chemistry and by experimentation. Structures that are not chemically feasible are not within a claimed set of compounds.

In some instances, the compounds encompassed by the various formulae presented herein are the compounds as shown in Tables 1-7.

TABLE 1

| Compound | |
|---|---|
| No. | Structure |
| A1 | 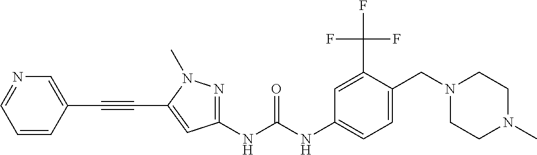 [SMILES: CN1CCN(Cc2ccc(NC(=O)Nc3cc(C#Cc4cccnc4)n(C)n3)cc2C(F)(F)F)CC1] |
| A2 | 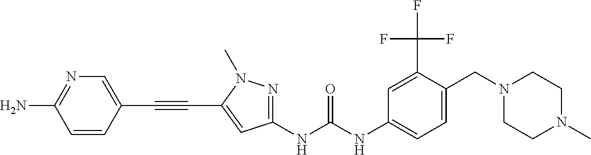 [SMILES: CN1CCN(Cc2ccc(NC(=O)Nc3cc(C#Cc4ccc(N)nc4)n(C)n3)cc2C(F)(F)F)CC1] |
| A3 | 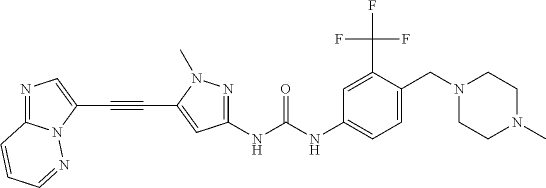 [SMILES: CN1CCN(Cc2ccc(NC(=O)Nc3cc(C#Cc4cnc5cccnn45)n(C)n3)cc2C(F)(F)F)CC1] |
| A4 | 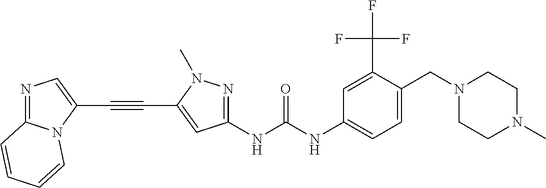 [SMILES: CN1CCN(Cc2ccc(NC(=O)Nc3cc(C#Cc4cnc5ccccn45)n(C)n3)cc2C(F)(F)F)CC1] |
| A5 | 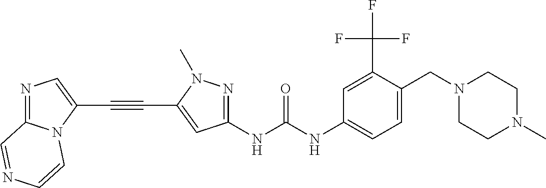 [SMILES: CN1CCN(Cc2ccc(NC(=O)Nc3cc(C#Cc4cnc5cnccn45)n(C)n3)cc2C(F)(F)F)CC1] |
| A6 | 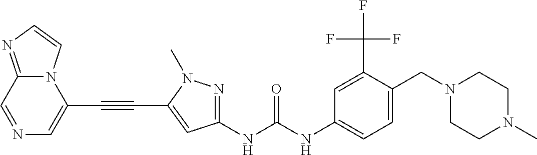 [SMILES: CN1CCN(Cc2ccc(NC(=O)Nc3cc(C#Cc4cncc5nccn45)n(C)n3)cc2C(F)(F)F)CC1] |
| A7 | 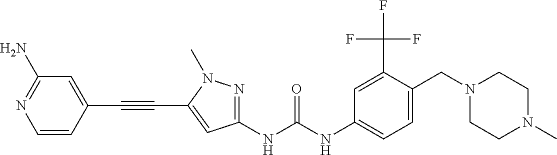 [SMILES: CN1CCN(Cc2ccc(NC(=O)Nc3cc(C#Cc4ccnc(N)c4)n(C)n3)cc2C(F)(F)F)CC1] |
| A8 | 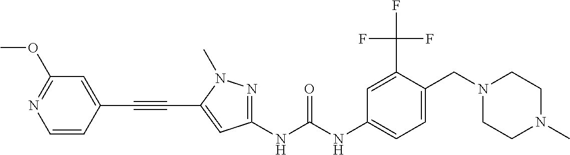 |
| A9 | 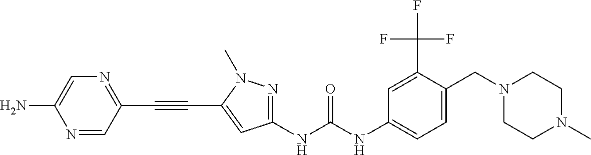 [SMILES: CN1CCN(Cc2ccc(NC(=O)Nc3cc(C#Cc4cnc(N)cn4)n(C)n3)cc2C(F)(F)F)CC1] |
| A10 | 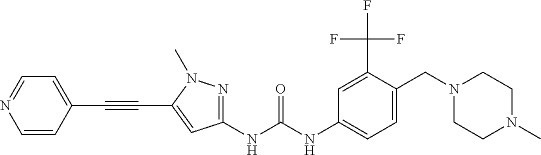 [SMILES: CN1CCN(Cc2ccc(NC(=O)Nc3cc(C#Cc4ccncc4)n(C)n3)cc2C(F)(F)F)CC1] |
| A11 | 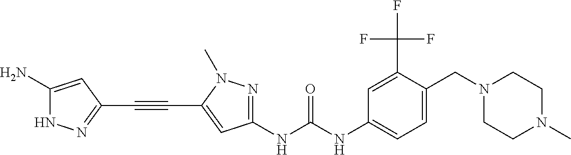 [SMILES: CN1CCN(Cc2ccc(NC(=O)Nc3cc(C#Cc4cc(N)[nH]n4)n(C)n3)cc2C(F)(F)F)CC1] |
| A12 | 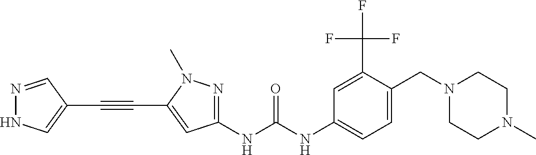 [SMILES: CN1CCN(Cc2ccc(NC(=O)Nc3cc(C#Cc4cn[nH]c4)n(C)n3)cc2C(F)(F)F)CC1] |
| A13 | 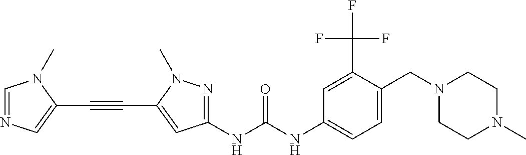 [SMILES: CN1CCN(Cc2ccc(NC(=O)Nc3cc(C#Cc4cncn4C)n(C)n3)cc2C(F)(F)F)CC1] |
| A14 | 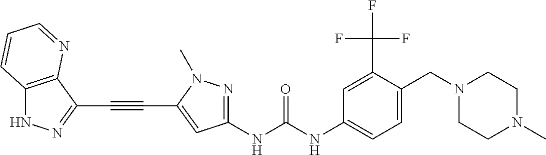 [SMILES: CN1CCN(Cc2ccc(NC(=O)Nc3cc(C#Cc4n[nH]c5cccnc45)n(C)n3)cc2C(F)(F)F)CC1] |
| A15 | 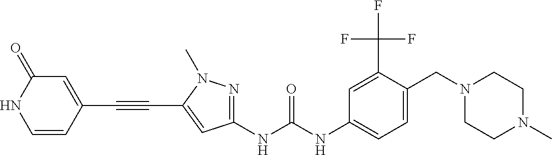 [SMILES: CN1CCN(Cc2ccc(NC(=O)Nc3cc(C#Cc4cc[nH]c(=O)c4)n(C)n3)cc2C(F)(F)F)CC1] |
| A16 | 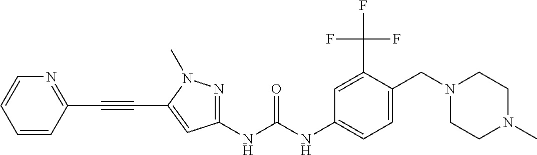 [SMILES: CN1CCN(Cc2ccc(NC(=O)Nc3cc(C#Cc4ccccn4)n(C)n3)cc2C(F)(F)F)CC1] |
| A17 | 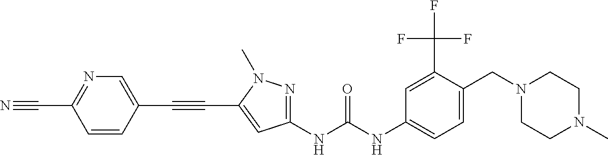 [SMILES: CN1CCN(Cc2ccc(NC(=O)Nc3cc(C#Cc4ccc(C#N)nc4)n(C)n3)cc2C(F)(F)F)CC1] |
| A18 | 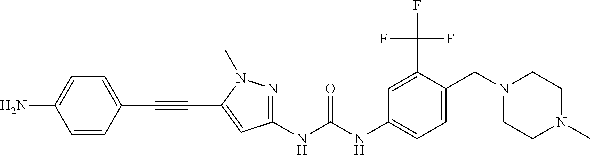 [SMILES: CN1CCN(Cc2ccc(NC(=O)Nc3cc(C#Cc4ccc(N)cc4)n(C)n3)cc2C(F)(F)F)CC1] |
| A19 | 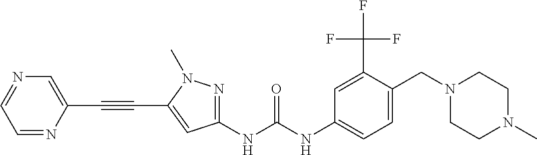 [SMILES: CN1CCN(Cc2ccc(NC(=O)Nc3cc(C#Cc4cnccn4)n(C)n3)cc2C(F)(F)F)CC1] |
| A20 | 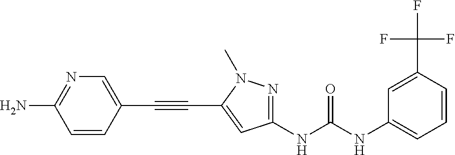 [SMILES: Cn1nc(NC(=O)Nc2cccc(C(F)(F)F)c2)cc1C#Cc1ccc(N)nc1] |
| A21 | 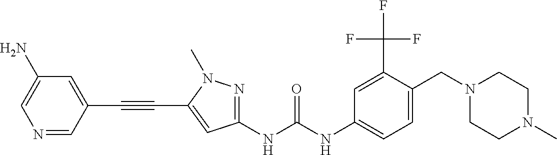 [SMILES: CN1CCN(Cc2ccc(NC(=O)Nc3cc(C#Cc4cncc(N)c4)n(C)n3)cc2C(F)(F)F)CC1] |
| A22 | 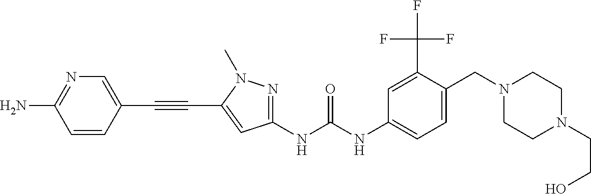 [SMILES: Cn1nc(NC(=O)Nc2ccc(CN3CCN(CCO)CC3)c(C(F)(F)F)c2)cc1C#Cc1ccc(N)nc1] |
| A23 | 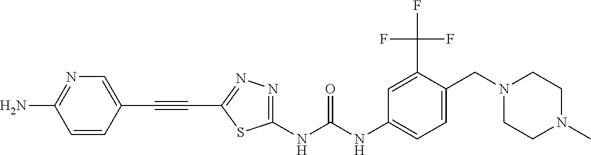 [SMILES: CN1CCN(Cc2ccc(NC(=O)Nc3nnc(C#Cc4ccc(N)nc4)s3)cc2C(F)(F)F)CC1] |
| A24 | 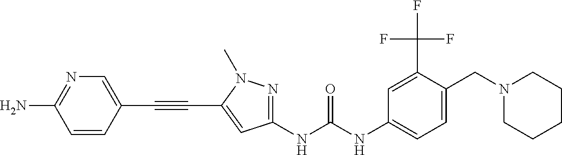 [SMILES: Cn1nc(NC(=O)Nc2ccc(CN3CCCCC3)c(C(F)(F)F)c2)cc1C#Cc1ccc(N)nc1] |
| A25 | 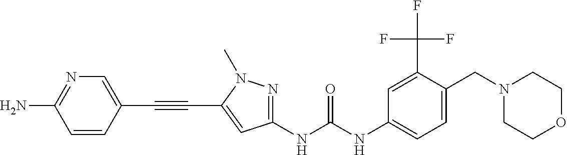 [SMILES: Cn1nc(NC(=O)Nc2ccc(CN3CCOCC3)c(C(F)(F)F)c2)cc1C#Cc1ccc(N)nc1] |

TABLE 2

| Compound | |
|---|---|
| No. | Structure |
| B1 | 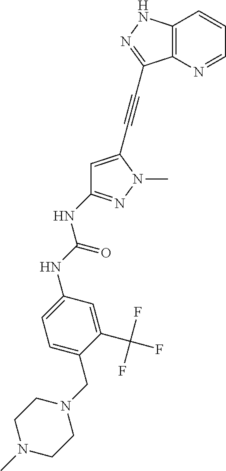 [SMILES: CN1CCN(Cc2ccc(NC(=O)Nc3cc(C#Cc4n[nH]c5cccnc45)n(C)n3)cc2C(F)(F)F)CC1] |
| B2 | 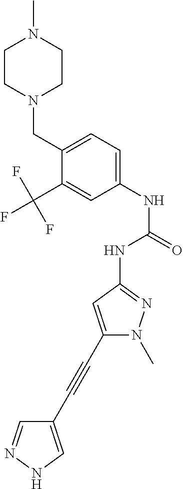 [SMILES: CN1CCN(Cc2ccc(NC(=O)Nc3cc(C#Cc4cn[nH]c4)n(C)n3)cc2C(F)(F)F)CC1] |
| B3 | 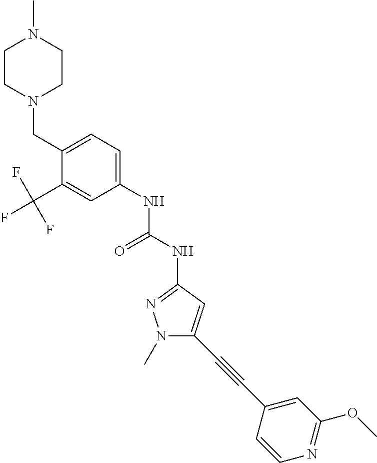 [SMILES: COc1cc(C#Cc2cc(NC(=O)Nc3ccc(CN4CCN(C)CC4)c(C(F)(F)F)c3)nn2C)ccn1] |
| B4 | 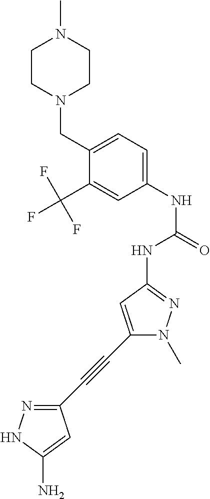 [SMILES: CN1CCN(Cc2ccc(NC(=O)Nc3cc(C#Cc4cc(N)[nH]n4)n(C)n3)cc2C(F)(F)F)CC1] |
| B5 | 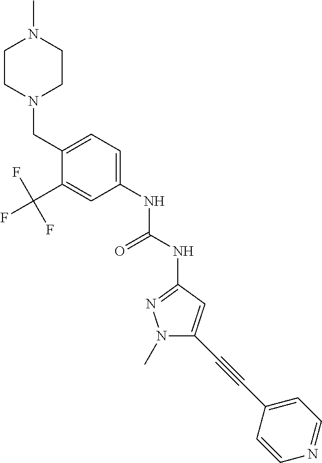 [SMILES: CN1CCN(Cc2ccc(NC(=O)Nc3cc(C#Cc4ccncc4)n(C)n3)cc2C(F)(F)F)CC1] |
| B6 | 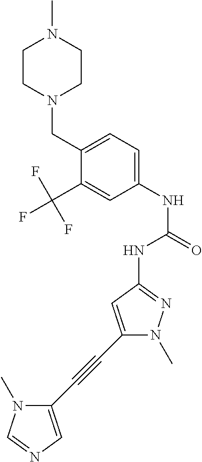 [SMILES: CN1CCN(Cc2ccc(NC(=O)Nc3cc(C#Cc4cncn4C)n(C)n3)cc2C(F)(F)F)CC1] |
| B7 | 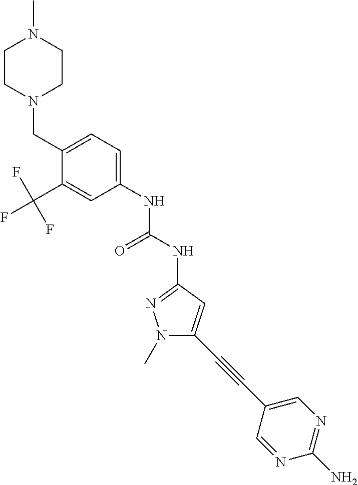 [SMILES: CN1CCN(Cc2ccc(NC(=O)Nc3cc(C#Cc4cnc(N)nc4)n(C)n3)cc2C(F)(F)F)CC1] |
| B8 | 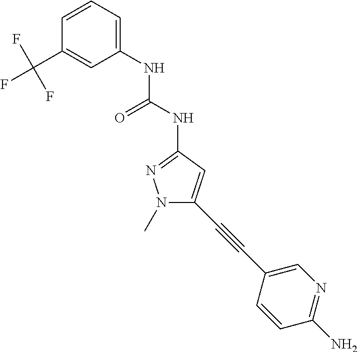 [SMILES: Cn1nc(NC(=O)Nc2cccc(C(F)(F)F)c2)cc1C#Cc1ccc(N)nc1] |
| B9 | 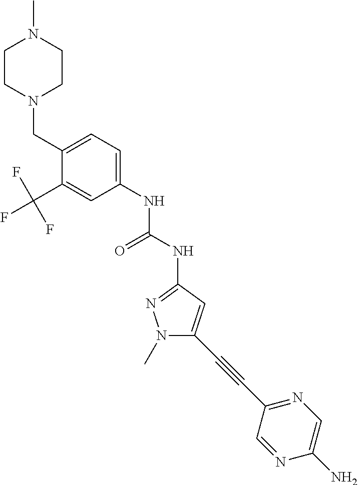 [SMILES: CN1CCN(Cc2ccc(NC(=O)Nc3cc(C#Cc4cnc(N)cn4)n(C)n3)cc2C(F)(F)F)CC1] |
| B10 | 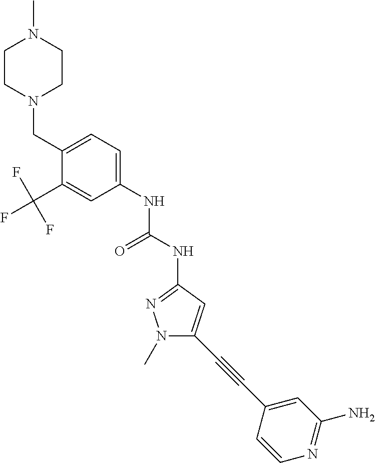 [SMILES: CN1CCN(Cc2ccc(NC(=O)Nc3cc(C#Cc4ccnc(N)c4)n(C)n3)cc2C(F)(F)F)CC1] |
| B11 | 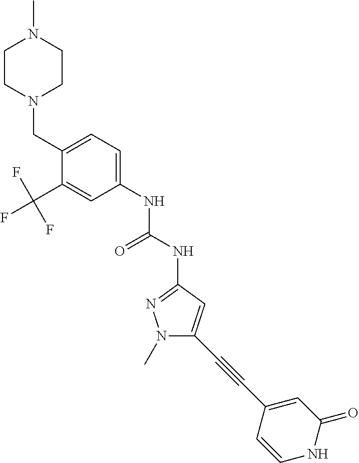 [SMILES: CN1CCN(Cc2ccc(NC(=O)Nc3cc(C#Cc4cc[nH]c(=O)c4)n(C)n3)cc2C(F)(F)F)CC1] |
| B12 | 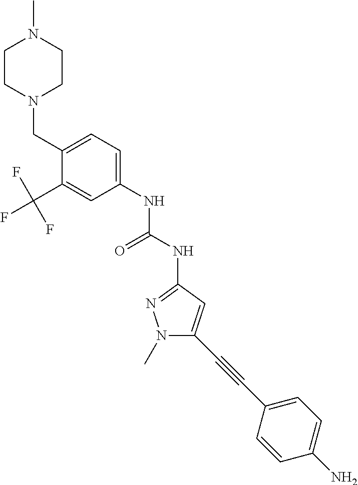 [SMILES: CN1CCN(Cc2ccc(NC(=O)Nc3cc(C#Cc4ccc(N)cc4)n(C)n3)cc2C(F)(F)F)CC1] |
| B13 | 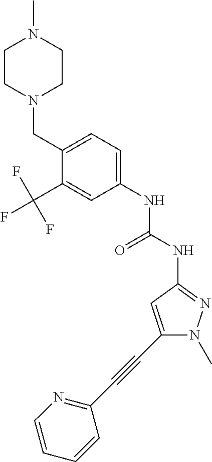 [SMILES: CN1CCN(Cc2ccc(NC(=O)Nc3cc(C#Cc4ccccn4)n(C)n3)cc2C(F)(F)F)CC1] |
| B14 | 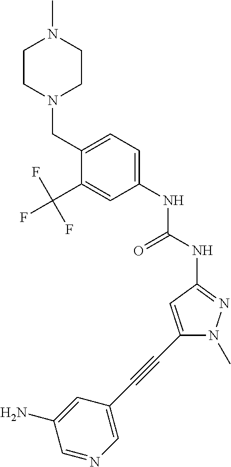 [SMILES: CN1CCN(Cc2ccc(NC(=O)Nc3cc(C#Cc4cncc(N)c4)n(C)n3)cc2C(F)(F)F)CC1] |
| B15 | 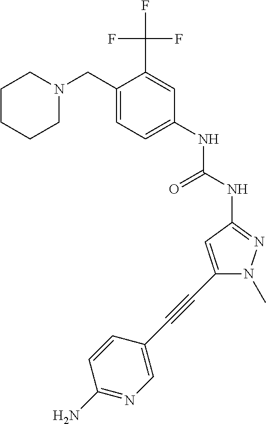 [SMILES: Cn1nc(NC(=O)Nc2ccc(CN3CCCCC3)c(C(F)(F)F)c2)cc1C#Cc1ccc(N)nc1] |
| B16 | 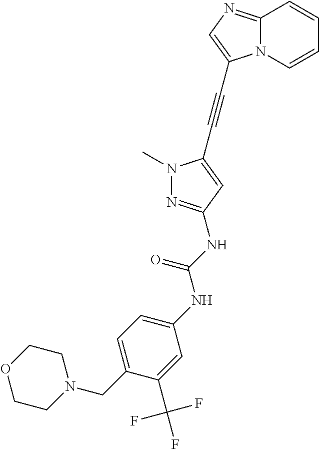 [SMILES: Cn1nc(NC(=O)Nc2ccc(CN3CCOCC3)c(C(F)(F)F)c2)cc1C#Cc1cnc2ccccn12] |
| B17 | 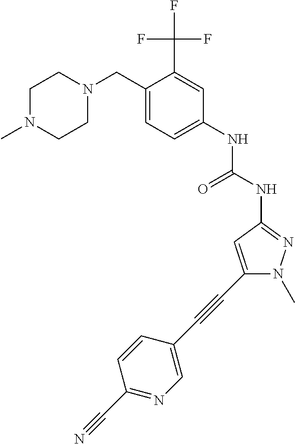 |
| B18 | 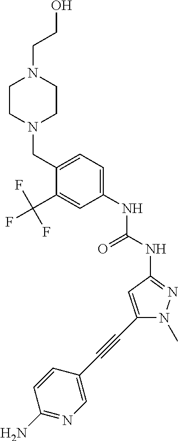 [SMILES: Cn1nc(NC(=O)Nc2ccc(CN3CCN(CCO)CC3)c(C(F)(F)F)c2)cc1C#Cc1ccc(N)nc1] |
| B19 | 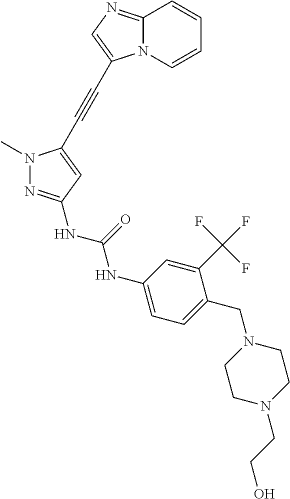 [SMILES: Cn1nc(NC(=O)Nc2ccc(CN3CCN(CCO)CC3)c(C(F)(F)F)c2)cc1C#Cc1cnc2ccccn12] |
| B20 | 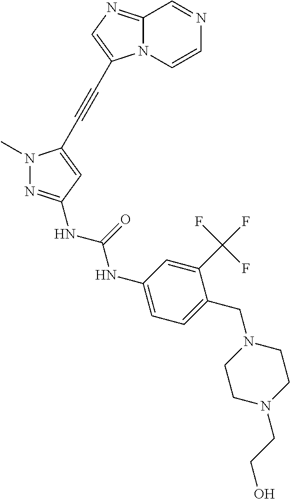 [SMILES: Cn1nc(NC(=O)Nc2ccc(CN3CCN(CCO)CC3)c(C(F)(F)F)c2)cc1C#Cc1cnc2cnccn12] |
| B21 | 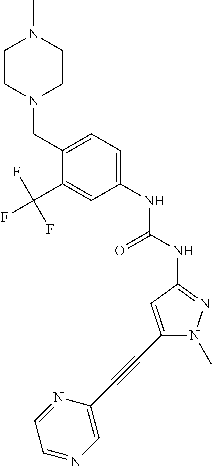 [SMILES: CN1CCN(Cc2ccc(NC(=O)Nc3cc(C#Cc4cnccn4)n(C)n3)cc2C(F)(F)F)CC1] |
| B22 | 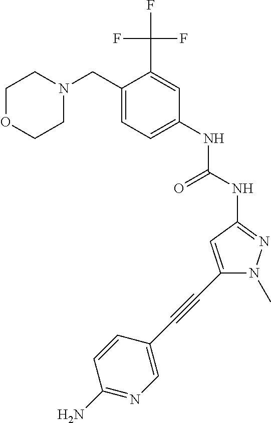 [SMILES: Cn1nc(NC(=O)Nc2ccc(CN3CCOCC3)c(C(F)(F)F)c2)cc1C#Cc1ccc(N)nc1] |
| B23 | 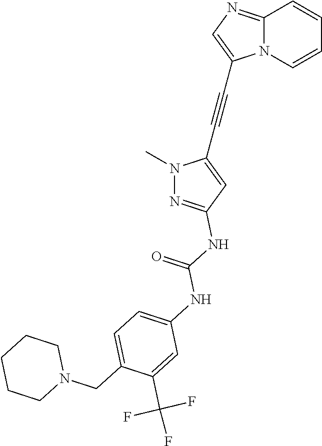 [SMILES: Cn1nc(NC(=O)Nc2ccc(CN3CCCCC3)c(C(F)(F)F)c2)cc1C#Cc1cnc2ccccn12] |
| B24 | 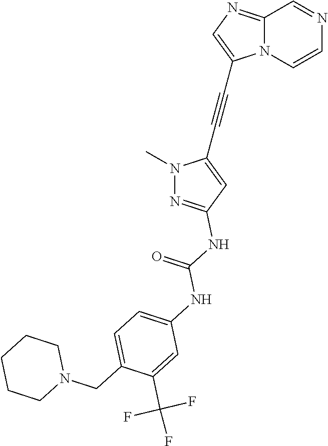 [SMILES: Cn1nc(NC(=O)Nc2ccc(CN3CCCCC3)c(C(F)(F)F)c2)cc1C#Cc1cnc2cnccn12] |
| B25 | 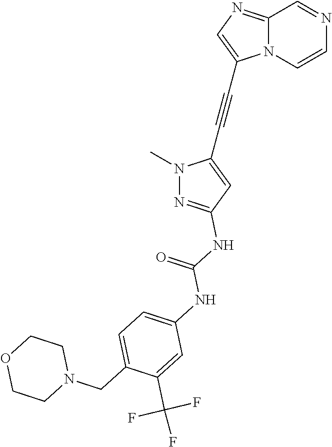 [SMILES: Cn1nc(NC(=O)Nc2ccc(CN3CCOCC3)c(C(F)(F)F)c2)cc1C#Cc1cnc2cnccn12] |
| B26 | 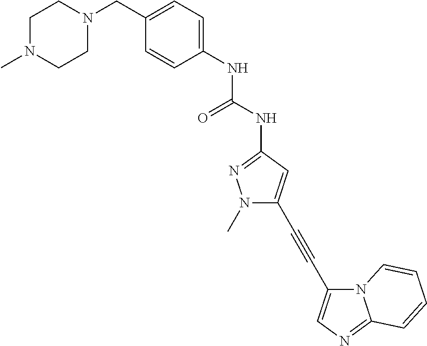 [SMILES: CN1CCN(Cc2ccc(NC(=O)Nc3cc(C#Cc4cnc5ccccn45)n(C)n3)cc2)CC1] |
| B27 | 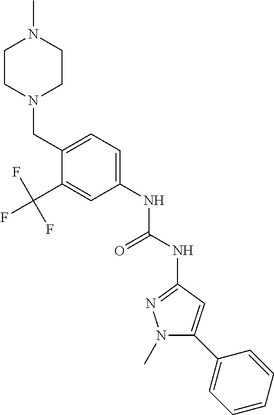 [SMILES: CN1CCN(Cc2ccc(NC(=O)Nc3cc(-c4ccccc4)n(C)n3)cc2C(F)(F)F)CC1] |
| B28 | 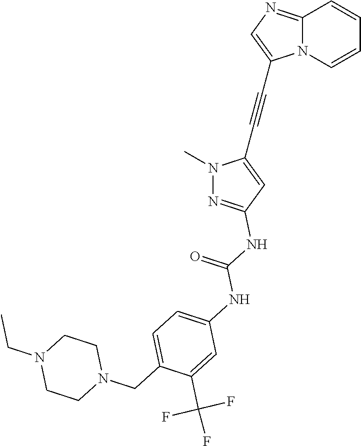 [SMILES: CCN1CCN(Cc2ccc(NC(=O)Nc3cc(C#Cc4cnc5ccccn45)n(C)n3)cc2C(F)(F)F)CC1] |
| B29 | 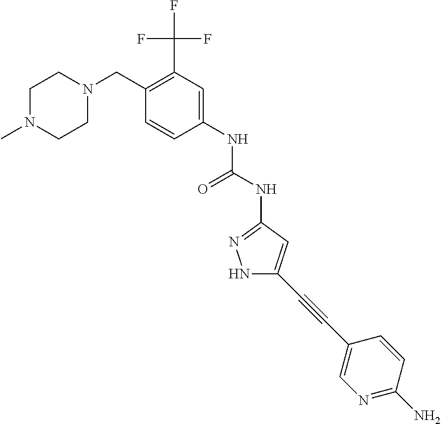 [SMILES: CN1CCN(Cc2ccc(NC(=O)Nc3cc(C#Cc4ccc(N)nc4)[nH]n3)cc2C(F)(F)F)CC1] |
| B30 | 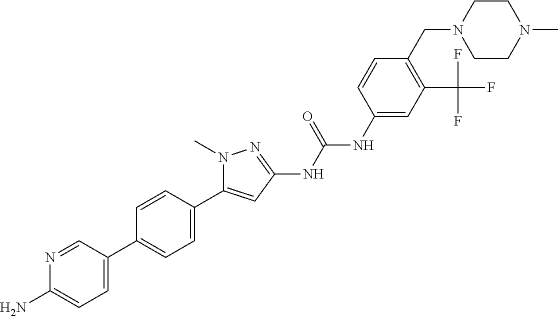 [SMILES: CN1CCN(Cc2ccc(NC(=O)Nc3cc(-c4ccc(-c5ccc(N)nc5)cc4)n(C)n3)cc2C(F)(F)F)CC1] |
| B31 | 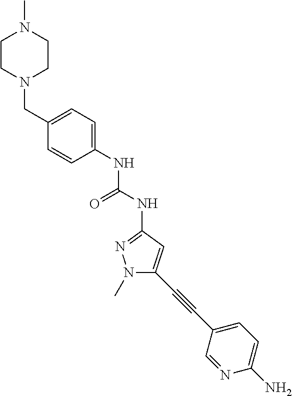 [SMILES: CN1CCN(Cc2ccc(NC(=O)Nc3cc(C#Cc4ccc(N)nc4)n(C)n3)cc2)CC1] |
| B32 | 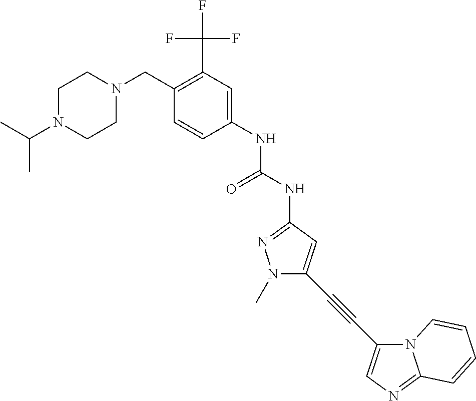 [SMILES: CC(C)N1CCN(Cc2ccc(NC(=O)Nc3cc(C#Cc4cnc5ccccn45)n(C)n3)cc2C(F)(F)F)CC1] |
| B33 | 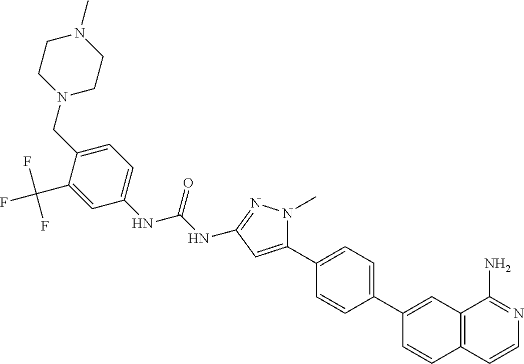 [SMILES: CN1CCN(Cc2ccc(NC(=O)Nc3cc(-c4ccc(-c5ccc6ccnc(N)c6c5)cc4)n(C)n3)cc2C(F)(F)F)CC1] |
| B34 | 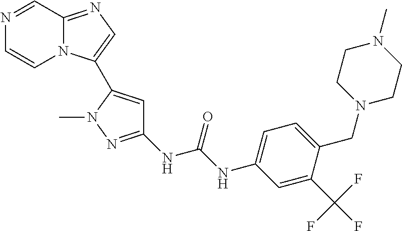 [SMILES: CN1CCN(Cc2ccc(NC(=O)Nc3cc(-c4cnc5cnccn45)n(C)n3)cc2C(F)(F)F)CC1] |
| B35 | 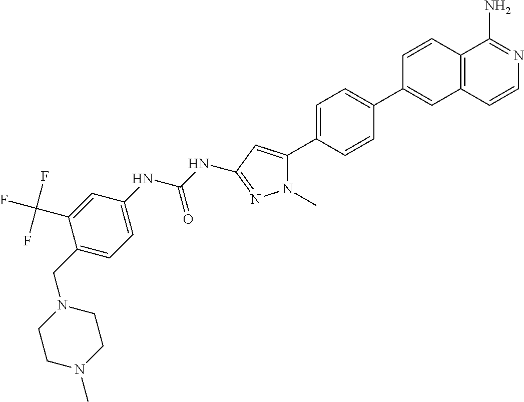 [SMILES: CN1CCN(Cc2ccc(NC(=O)Nc3cc(-c4ccc(-c5ccc6c(N)nccc6c5)cc4)n(C)n3)cc2C(F)(F)F)CC1] |
| B36 | 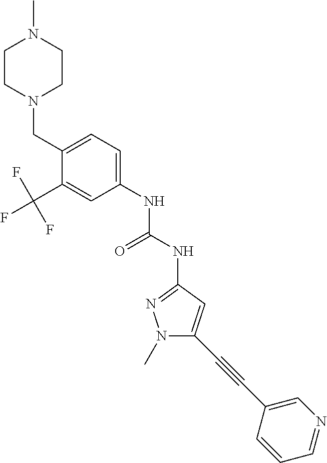 [SMILES: CN1CCN(Cc2ccc(NC(=O)Nc3cc(C#Cc4cccnc4)n(C)n3)cc2C(F)(F)F)CC1] |
| B37 | 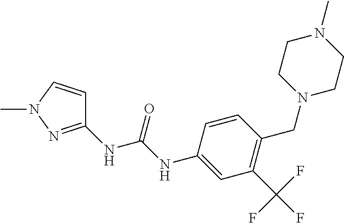 [SMILES: CN1CCN(Cc2ccc(NC(=O)Nc3ccn(C)n3)cc2C(F)(F)F)CC1] |
| B38 | 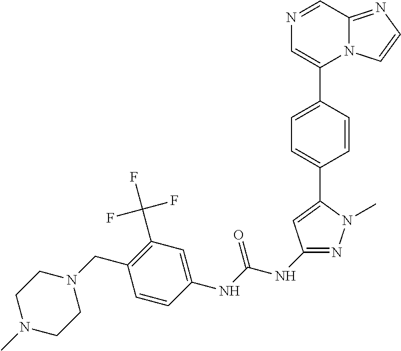 [SMILES: CN1CCN(Cc2ccc(NC(=O)Nc3cc(-c4ccc(-c5cncc6nccn56)cc4)n(C)n3)cc2C(F)(F)F)CC1] |
| B39 | 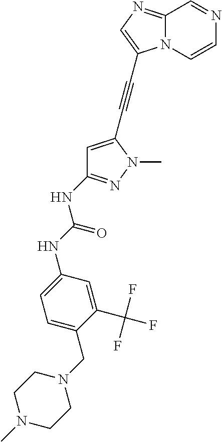 [SMILES: CN1CCN(Cc2ccc(NC(=O)Nc3cc(C#Cc4cnc5cnccn45)n(C)n3)cc2C(F)(F)F)CC1] |
| B40 | 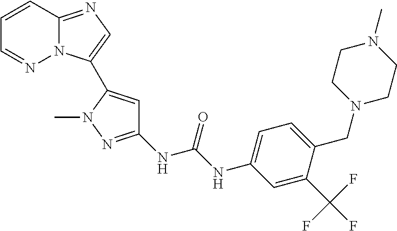 [SMILES: CN1CCN(Cc2ccc(NC(=O)Nc3cc(-c4cnc5cccnn45)n(C)n3)cc2C(F)(F)F)CC1] |
| B41 | 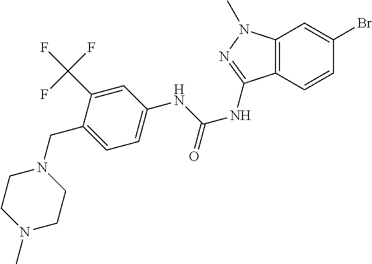 [SMILES: CN1CCN(Cc2ccc(NC(=O)Nc3nn(C)c4cc(Br)ccc34)cc2C(F)(F)F)CC1] |
| B42 | 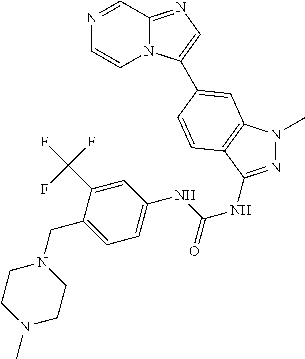 [SMILES: CN1CCN(Cc2ccc(NC(=O)Nc3nn(C)c4cc(-c5cnc6cnccn56)ccc34)cc2C(F)(F)F)CC1] |
| B43 | 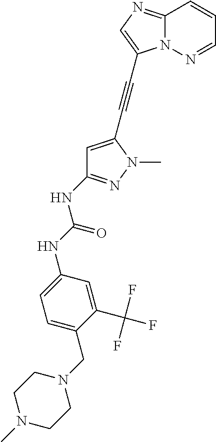 [SMILES: CN1CCN(Cc2ccc(NC(=O)Nc3cc(C#Cc4cnc5cccnn45)n(C)n3)cc2C(F)(F)F)CC1] |
| B44 | 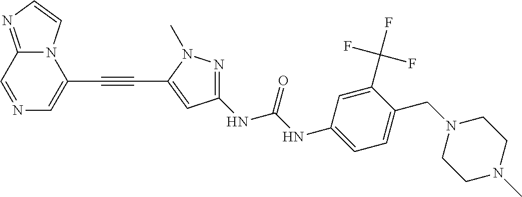 [SMILES: CN1CCN(Cc2ccc(NC(=O)Nc3cc(C#Cc4cncc5nccn45)n(C)n3)cc2C(F)(F)F)CC1] |
| B45 | 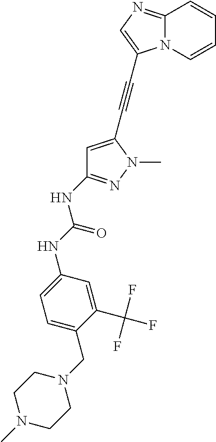 [SMILES: CN1CCN(Cc2ccc(NC(=O)Nc3cc(C#Cc4cnc5ccccn45)n(C)n3)cc2C(F)(F)F)CC1] |
| B46 | 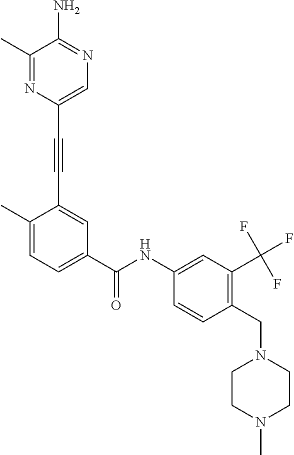 [SMILES: Cc1ccc(C(=O)Nc2ccc(CN3CCN(C)CC3)c(C(F)(F)F)c2)cc1C#Cc1cnc(N)c(C)n1] |
| B47 | 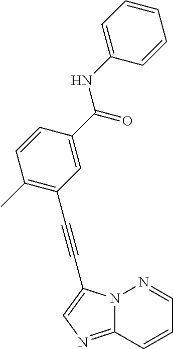 [SMILES: Cc1ccc(C(=O)Nc2ccccc2)cc1C#Cc1cnc2cccnn12] |
| B48 | 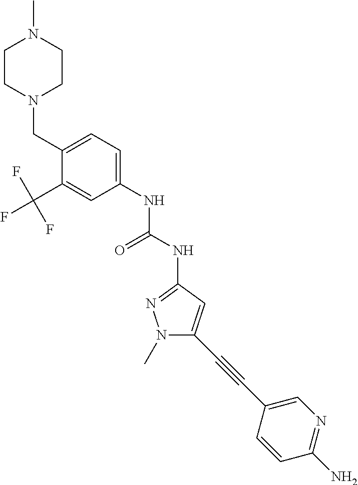 [SMILES: CN1CCN(Cc2ccc(NC(=O)Nc3cc(C#Cc4ccc(N)nc4)n(C)n3)cc2C(F)(F)F)CC1] |
| B49 | 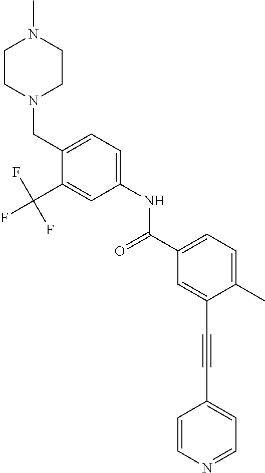 [SMILES: Cc1ccc(C(=O)Nc2ccc(CN3CCN(C)CC3)c(C(F)(F)F)c2)cc1C#Cc1ccncc1] |
| B50 | 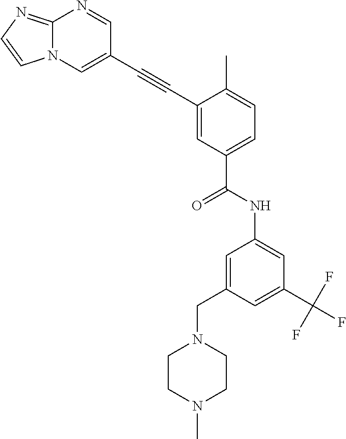 [SMILES: Cc1ccc(C(=O)Nc2cc(CN3CCN(C)CC3)cc(C(F)(F)F)c2)cc1C#Cc1cnc2nccn2c1] |
| B51 | 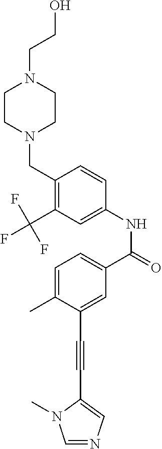 [SMILES: Cc1ccc(C(=O)Nc2ccc(CN3CCN(CCO)CC3)c(C(F)(F)F)c2)cc1C#Cc1cncn1C] |
| B52 | 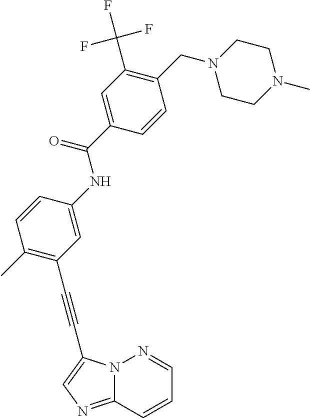 |
| B53 | 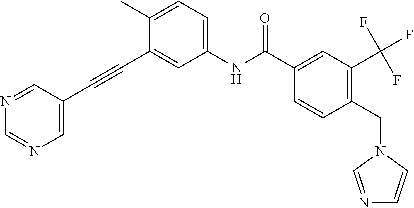 [SMILES: Cc1ccc(NC(=O)c2ccc(Cn3ccnc3)c(C(F)(F)F)c2)cc1C#Cc1cncnc1] |
| B54 | 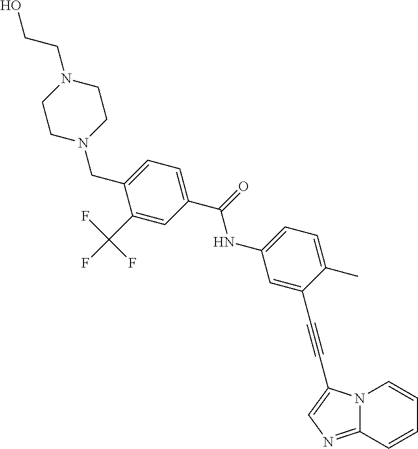 [SMILES: Cc1ccc(NC(=O)c2ccc(CN3CCN(CCO)CC3)c(C(F)(F)F)c2)cc1C#Cc1cnc2ccccn12] |
| B55 | 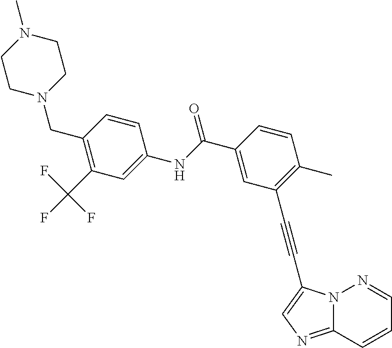 [SMILES: Cc1ccc(C(=O)Nc2ccc(CN3CCN(C)CC3)c(C(F)(F)F)c2)cc1C#Cc1cnc2cccnn12] |
| B56 | 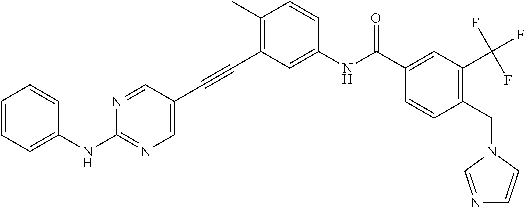 [SMILES: Cc1ccc(NC(=O)c2ccc(Cn3ccnc3)c(C(F)(F)F)c2)cc1C#Cc1cnc(Nc2ccccc2)nc1] |

TABLE 3

| Compound | | |
|---|---|---|
| No. | Structure | Name |
| 1 | 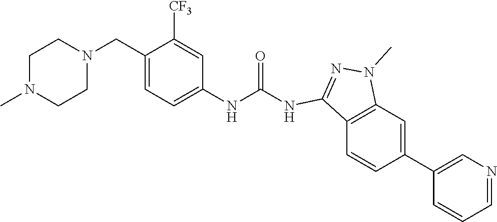 [SMILES: CN1CCN(Cc2ccc(NC(=O)Nc3nn(C)c4cc(-c5cccnc5)ccc34)cc2C(F)(F)F)CC1] | 1-(1-methyl-6-(pyridin-3-yl)-1H- indazol-3-yl)-3-(4-((4- methylpiperazin-1-yl)methyl)-3- (trifluoromethyl)phenyl)urea |
| 2 | 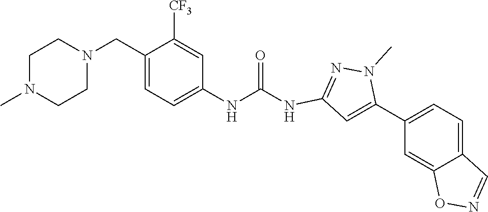 [SMILES: CN1CCN(Cc2ccc(NC(=O)Nc3cc(-c4ccc5cnoc5c4)n(C)n3)cc2C(F)(F)F)CC1] | 1-(5-(benzo[d]isoxazol-6-yl)-1- methyl-1H-pyrazol-3-yl)-3-(4-((4- methylpiperazin-1-yl)methyl)-3- (trifluoromethyl)phenyl)urea |
| 3 | 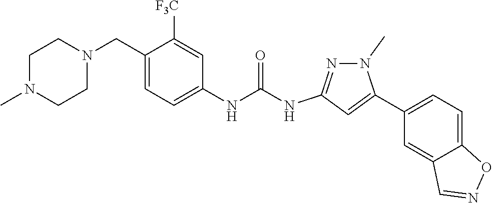 [SMILES: CN1CCN(Cc2ccc(NC(=O)Nc3cc(-c4ccc5oncc5c4)n(C)n3)cc2C(F)(F)F)CC1] | 1-(5-(benzo[d]isoxazol-5-yl)-1- methyl-1H-pyrazol-3-yl)-3-(4-((4- methylpiperazin-1-yl)methyl)-3- (trifluoromethyl)phenyl)urea |
| 4 | 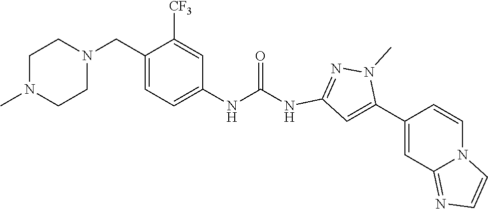 [SMILES: CN1CCN(Cc2ccc(NC(=O)Nc3cc(-c4ccn5ccnc5c4)n(C)n3)cc2C(F)(F)F)CC1] | 1-(5-(imidazo[1,2-a]pyridin-7-yl)- 1-methyl-1H-pyrazol-3-yl)-3-(4- ((4-methylpiperazin-1-yl)methyl)- 3-(trifluoromethyl)phenyl)urea |
| 5 | 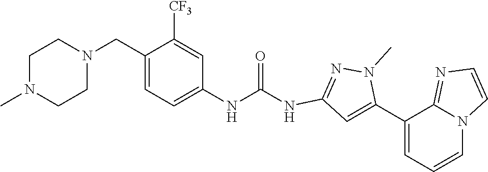 [SMILES: CN1CCN(Cc2ccc(NC(=O)Nc3cc(-c4cccn5ccnc45)n(C)n3)cc2C(F)(F)F)CC1] | 1-(5-(imidazo[1,2-a]pyridin-8-yl)- 1-methyl-1H-pyrazol-3-yl)-(3-(4- ((4-methylpiperazin-1-yl)methyl)- 3-(trifluoromethyl)phenyl)urea |
| 6 | 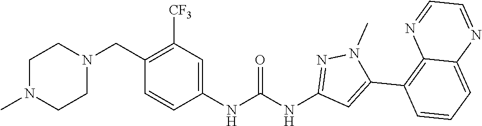 [SMILES: CN1CCN(Cc2ccc(NC(=O)Nc3cc(-c4cccc5nccnc45)n(C)n3)cc2C(F)(F)F)CC1] | 1-(1-methyl-5-(quinoxalin-5-yl)- 1H-pyrazol-3-yl)-3-(4-((4- methylpiperazin-1-yl)methyl)-3- (trifluoromethyl)phenyl)urea |
| 7 | 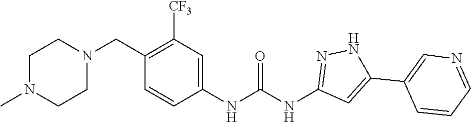 [SMILES: CN1CCN(Cc2ccc(NC(=O)Nc3cc(-c4cccnc4)[nH]n3)cc2C(F)(F)F)CC1] | 1-(4-((4-methylpiperazin-1- yl)methyl)-3- (trifluoromethyl)phenyl)-3-(5- (pyridin-3-yl)-1H-pyrazol-3- yl)urea |
| 8 | 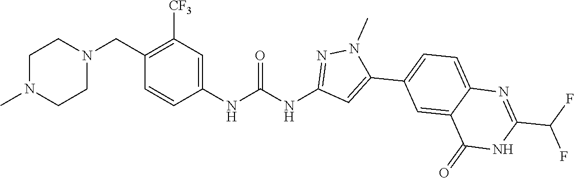 [SMILES: CN1CCN(Cc2ccc(NC(=O)Nc3cc(-c4ccc5nc(C(F)F)[nH]c(=O)c5c4)n(C)n3)cc2C(F)(F)F)CC1] | 1-(5-(2-(difluoromethyl)-4-oxo- 3,4-dihydroquinazolin-6-yl)-1- methyl-1H-pyrazol-3-yl)-3-(4-((4- methylpiperazin-1-yl)methyl)-3- (trifluoromethyl)phenyl)urea |
| 9 | 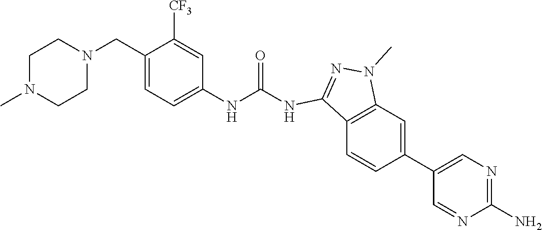 [SMILES: CN1CCN(Cc2ccc(NC(=O)Nc3nn(C)c4cc(-c5cnc(N)nc5)ccc34)cc2C(F)(F)F)CC1] | 1-(6-(2-aminopyrimidin-5-yl)-1- methyl-1H-indazol-3-yl)-3-(4-((4- methylpiperazin-1-yl)methyl)-3- (trifluoromethyl)phenyl)urea |
| 10 | 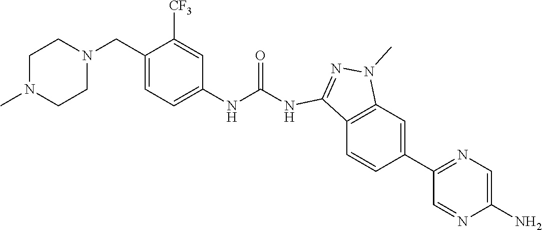 [SMILES: CN1CCN(Cc2ccc(NC(=O)Nc3nn(C)c4cc(-c5cnc(N)cn5)ccc34)cc2C(F)(F)F)CC1] | 1-(6-(5-aminopyrazin-2-yl)-1- methyl-1H-indazol-3-yl)-3-(4-((4- methylpiperazin-1-yl)methyl)-3- (trifluoromethyl)phenyl)urea |
| 11 | 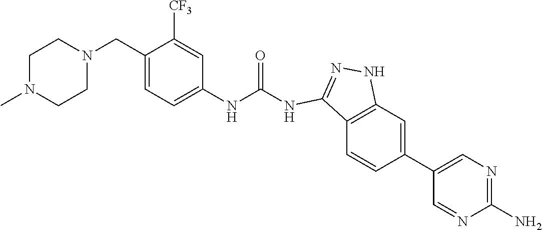 [SMILES: CN1CCN(Cc2ccc(NC(=O)Nc3n[nH]c4cc(-c5cnc(N)nc5)ccc34)cc2C(F)(F)F)CC1] | 1-(6-(2-aminopyrimidin-5-yl)-1H- indazol-3-yl)-3-(4-((4- methylpiperazin-1-yl)methyl)-3- (trifluoromethyl)phenyl)urea |
| 12 | 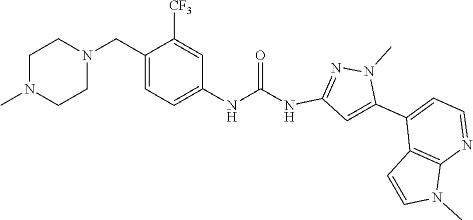 [SMILES: CN1CCN(Cc2ccc(NC(=O)Nc3cc(-c4ccnc5c4ccn5C)n(C)n3)cc2C(F)(F)F)CC1] | 1-(1-methyl-5-(1-methyl-1H- pyrrolo[2,3-b]pyridin-4-yl)-1H- pyrazol-3-yl)-3-(4-((4- methylpiperazin-1-yl)methyl)-3- (trifluoromethyl)phenyl)urea |
| 13 | 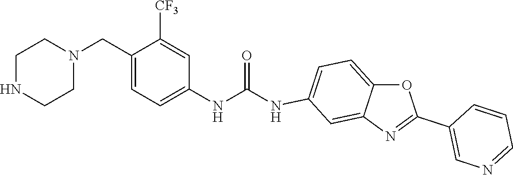 [SMILES: O=C(Nc1ccc(CN2CCNCC2)c(C(F)(F)F)c1)Nc1ccc2oc(-c3cccnc3)nc2c1] | 1-(4-(piperazin-1-ylmethyl)-3- (trifluoromethyl)phenyl)-3-(2- (pyridin-3-yl)benzo[d]oxazol-5- yl)urea |
| 14 | 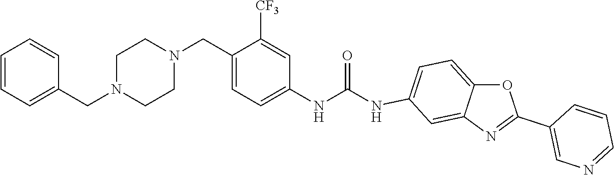 [SMILES: O=C(Nc1ccc(CN2CCN(Cc3ccccc3)CC2)c(C(F)(F)F)c1)Nc1ccc2oc(-c3cccnc3)nc2c1] | 1-(4-((4-benzylpiperazin-1- yl)methyl)-3- (trifluoromethyl)phenyl)-3-(2- (pyridin-3-yl)benzo[d]oxazol-5- yl)urea |
| 15 | 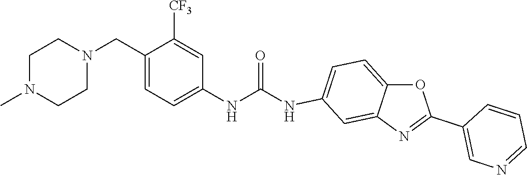 [SMILES: CN1CCN(Cc2ccc(NC(=O)Nc3ccc4oc(-c5cccnc5)nc4c3)cc2C(F)(F)F)CC1] | 1-(4-((4-methylpiperazin-1- yl)methyl)-3- (trifluoromethyl)phenyl)-3-(2- (pyridin-3-yl)benzo[d]oxazol-5- yl)urea |
| 16 | 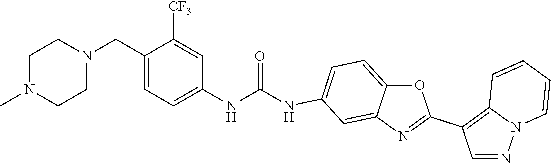 [SMILES: CN1CCN(Cc2ccc(NC(=O)Nc3ccc4oc(-c5cnn6ccccc56)nc4c3)cc2C(F)(F)F)CC1] | 1-(4-((4-methylpiperazin-1- yl)methyl)-3- (trifluoromethyl)phenyl)-3-(2- (pyrazolo[1,5-a]pyridin-3- yl)benzo[d]oxazol-5-yl)urea |
| 17 | 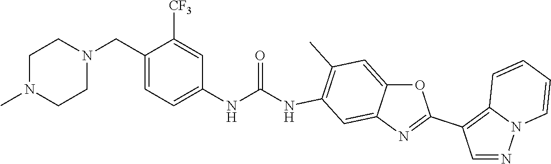 [SMILES: Cc1cc2oc(-c3cnn4ccccc34)nc2cc1NC(=O)Nc1ccc(CN2CCN(C)CC2)c(C(F)(F)F)c1] | 1-(6-methyl-2-(pyrazolo[1,5- a]pyridin-3-yl)benzo[d]oxazol-5- yl)-3-(4-((4-methylpiperazin-1- yl)methyl)-3- (trifluoromethyl)phenyl)urea |
| 18 | 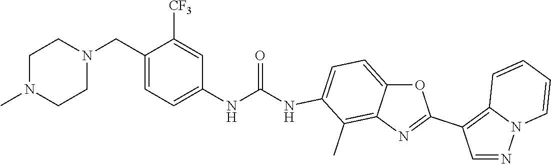 [SMILES: Cc1c(NC(=O)Nc2ccc(CN3CCN(C)CC3)c(C(F)(F)F)c2)ccc2oc(-c3cnn4ccccc34)nc12] | 1-(4-methyl-2-(pyrazolo[1,5- a]pyridin-3-yl)benzo[d]oxazol-5- yl)-3-(4-((4-methylpiperazin-1- yl)methyl)-3- (trifluoromethyl)phenyl)urea |
| 19 | 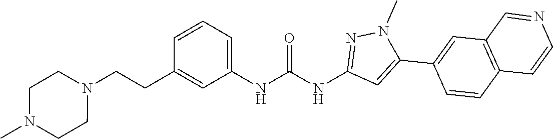 [SMILES: CN1CCN(CCc2cccc(NC(=O)Nc3cc(-c4ccc5ccncc5c4)n(C)n3)c2)CC1] | 1-(5-(isoquinolin-7-yl)-1-methyl- 1H-pyrazol-3-yl)-3-(3-(2-(4- methylpiperazin-1- yl)ethyl)phenyl)urea |
| 20 | 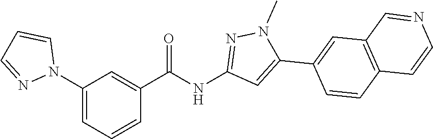 [SMILES: Cn1nc(NC(=O)c2cccc(-n3cccn3)c2)cc1-c1ccc2ccncc2c1] | N-(5-(isoquinolin-7-yl)-1-methyl- 1H-pyrazol-3-yl)-3-(1H-pyrazol- 1-yl)benzamide |
| 21 | 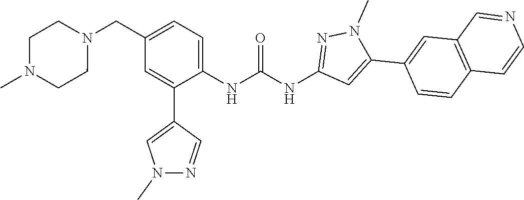 [SMILES: CN1CCN(Cc2ccc(NC(=O)Nc3cc(-c4ccc5ccncc5c4)n(C)n3)c(-c3cnn(C)c3)c2)CC1] | 1-(5-Isoquinolin-7-yl)-1-methyl- 1H-pyrazol-3-yl)-3-(2-(1-methyl- 1H-pyrazol-4-yl)-4-((4- methylpiperazin-1- yl)methyl)phenyl)urea |
| 22 | 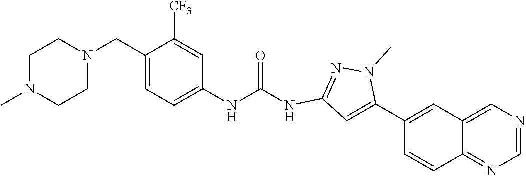 [SMILES: CN1CCN(Cc2ccc(NC(=O)Nc3cc(-c4ccc5ncncc5c4)n(C)n3)cc2C(F)(F)F)CC1] | 1-(1-methyl-5-(quinazolin-6-yl)- 1H-pyrazol-3-yl)-3-(4-((4- methylpiperazin-1-yl)methyl)-3- (triflueromethyl)phenyl)urea |
| 23 | 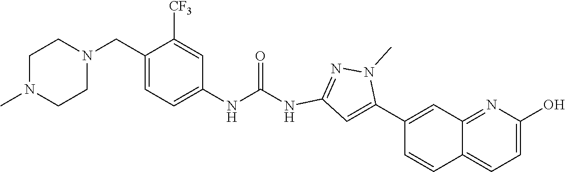 [SMILES: CN1CCN(Cc2ccc(NC(=O)Nc3cc(-c4ccc5ccc(O)nc5c4)n(C)n3)cc2C(F)(F)F)CC1] | 1-(5-(2-hydroxyquinolin-7-yl)-1- methyl-1H-pyralol-3-yl)-3-(4-((4- methylpiperazin-1-yl)methyl)-3- (trifluoromethyl)phenyl)urea |
| 24 | 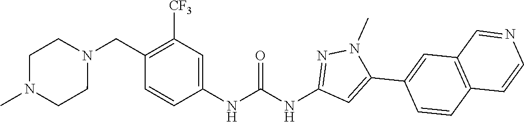 [SMILES: CN1CCN(Cc2ccc(NC(=O)Nc3cc(-c4ccc5ccncc5c4)n(C)n3)cc2C(F)(F)F)CC1] | 1-(5-(isoquinolin-7-yl)-1-methyl- 1H-pyrazol-3-yl)-3-(4-((4- methylpiperazin-1-yl)methyl)-3- (trifluoromethyl)phenyl)urea |
| 25 | 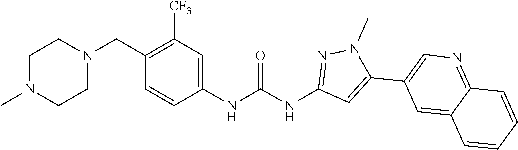 [SMILES: CN1CCN(Cc2ccc(NC(=O)Nc3cc(-c4cnc5ccccc5c4)n(C)n3)cc2C(F)(F)F)CC1] | 1-(1-methyl-5-(quinolin-3-yl)-1H- pyrazol-3-yl)-3-(4-((4- methylpiperazin-1-yl)methyl)-3- (trifluoromethyl)phenyl)urea |
| 26 | 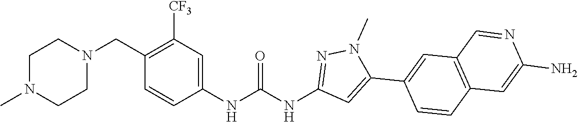 [SMILES: CN1CCN(Cc2ccc(NC(=O)Nc3cc(-c4ccc5cc(N)ncc5c4)n(C)n3)cc2C(F)(F)F)CC1] | 1-(5-(3-aminoisoquinolin-7-yl)-1- methyl-1H-pyrazol-3-yl)-3-(4-((4- methylpiperazin-1-yl)methyl)-3- (trifluoromethyl)phenyl)urea |
| 27 | 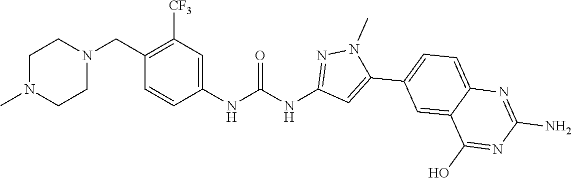 [SMILES: CN1CCN(Cc2ccc(NC(=O)Nc3cc(-c4ccc5nc(N)nc(O)c5c4)n(C)n3)cc2C(F)(F)F)CC1] | 1-(5-(2-amino-4- hydroxyquinazolin-6-yl)-1- methyl-1H-pyrazol-3-yl)-3-(4-((4- methylpiperazin-1-yl)methyl)-3- (trifluoromethyl)phenyl)urea |
| 28 | 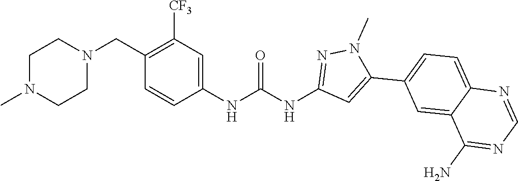 [SMILES: CN1CCN(Cc2ccc(NC(=O)Nc3cc(-c4ccc5ncnc(N)c5c4)n(C)n3)cc2C(F)(F)F)CC1] | 1-(5-(4-aminoguinazolin-6-yl)-1- methyl-1H-pyrazol-yl)-3-(4-((4- methylpiperazin-1-yl)methyl)-3- (trifluoromethyl)phenyl)urea |
| 29 | 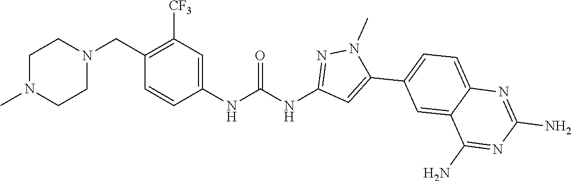 [SMILES: CN1CCN(Cc2ccc(NC(=O)Nc3cc(-c4ccc5nc(N)nc(N)c5c4)n(C)n3)cc2C(F)(F)F)CC1] | 1-(5-(2,4-diaminoquinazolin-6- yl)-1-methyl-1H-pyrazol-3-yl)-3- (4-((4-methylpiperazin-1- yl)methyl)-3- (trifluoromethyl)phenyl)urea |
| 30 | 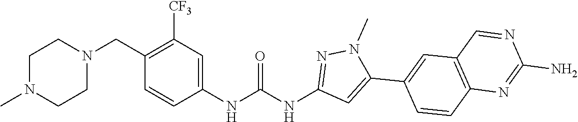 [SMILES: CN1CCN(Cc2ccc(NC(=O)Nc3cc(-c4ccc5nc(N)ncc5c4)n(C)n3)cc2C(F)(F)F)CC1] | 1-(5-(2-aminoquinazolin-6-yl)-1- methyl-1H-pyrazol-3-yl)-3-(4-((4- methylpiperazin-1-yl)methyl)-3- (trifluoromethyl)phenyl)urea |
| 31 | 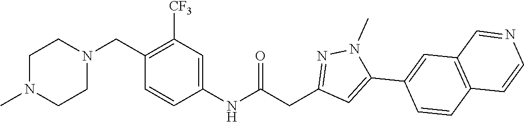 [SMILES: CN1CCN(Cc2ccc(NC(=O)Cc3cc(-c4ccc5ccncc5c4)n(C)n3)cc2C(F)(F)F)CC1] | 2-(5-(isoquinolin-7-yl)-1-methyl- 1H-pyrazol-3-yl)-N-(4-((4- methylpiperazin-1-yl)methyl)-3- (trifluoromethyl)phenyl)acetamide |
| 32 | 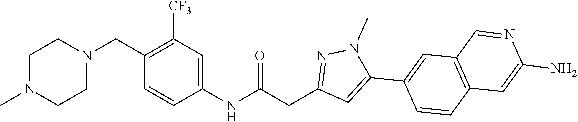 [SMILES: CN1CCN(Cc2ccc(NC(=O)Cc3cc(-c4ccc5cc(N)ncc5c4)n(C)n3)cc2C(F)(F)F)CC1] | 2-(5-(3-aminoisoquinolin-7-yl)-1- methyl-1H-pyrazol-3-yl)-N-(4- ((4-methylpiperazin-1-yl)methyl)- 3-(trifluoromethyl)phenyl)- acetamide |
| 33 | 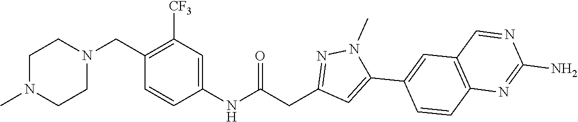 [SMILES: CN1CCN(Cc2ccc(NC(=O)Cc3cc(-c4ccc5nc(N)ncc5c4)n(C)n3)cc2C(F)(F)F)CC1] | 2-(5-(2-aminoquinazolin-6-yl)-1- methyl-1H-pyrazol-3-yl)-N-(4- ((4-methylpiperazin-1-yl)methyl)- 3-(trifluoromethyl)phenyl)- acetamide |
| 34 | 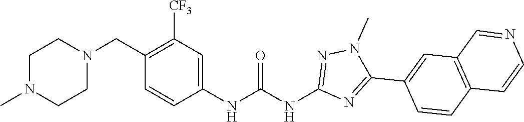 [SMILES: CN1CCN(Cc2ccc(NC(=O)Nc3nc(-c4ccc5ccncc5c4)n(C)n3)cc2C(F)(F)F)CC1] | 1-(5-(isoquinolin-7-yl)-1-methyl- 1H-1,2,4-triazol-3-yl)-3-(4-((4- methylpiperazin-1-yl)methyl)-3- (trifluoromethyl)phenyl)urea |
| 35 | 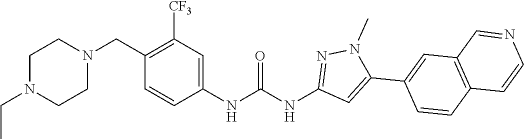 [SMILES: CCN1CCN(Cc2ccc(NC(=O)Nc3cc(-c4ccc5ccncc5c4)n(C)n3)cc2C(F)(F)F)CC1] | 1-(4-((4-ethylpiperazin-1- yl)methyl)-3- (triflueromethyl)phenyl)-3-(5- (isoquinolin-7-yl)-1-methyl-1H- pyrazol-3-yl)urea |
| 36 | 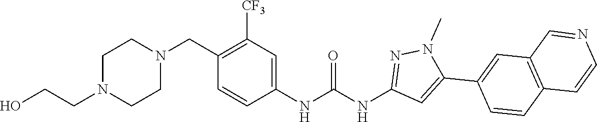 [SMILES: Cn1nc(NC(=O)Nc2ccc(CN3CCN(CCO)CC3)c(C(F)(F)F)c2)cc1-c1ccc2ccncc2c1] | 1-(4-((4-(2- hydroxyethyl)piperazin-1- yl)methyl)-3- (trifluoromethyl)phenyl)-3-(5- (isoquinolin-7-yl)-1-methyl-1H- pyrazol-3-yl)urea |
| 37 | 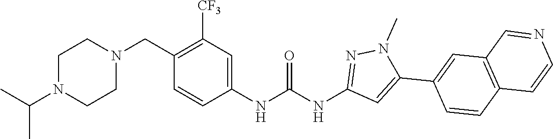 [SMILES: CC(C)N1CCN(Cc2ccc(NC(=O)Nc3cc(-c4ccc5ccncc5c4)n(C)n3)cc2C(F)(F)F)CC1] | 1-(4-((4-isopropylpiperazin-1- yl)methyl)-3- (triflueromethyl)phenyl)-3-(5- (isoquinolin-7-yl)-1-methyl-1H- pyrazol-3-yl)urea |
| 38 | 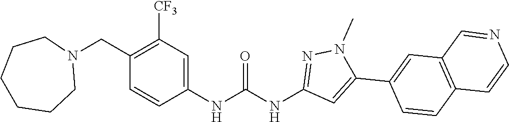 [SMILES: Cn1nc(NC(=O)Nc2ccc(CN3CCCCCC3)c(C(F)(F)F)c2)cc1-c1ccc2ccncc2c1] | 1-(4-(azepan-1-ylmethyl)-3- (trifluoromethyl)phenyl)-3-(5- (isoquinolin-7-yl)-1-methyl-1H- pyrazol-3-yl)urea |
| 39 | 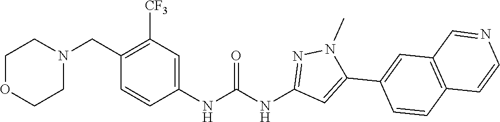 [SMILES: Cn1nc(NC(=O)Nc2ccc(CN3CCOCC3)c(C(F)(F)F)c2)cc1-c1ccc2ccncc2c1] | 1-(5-(isoquinol-7-yl)-1-methyl- 1H-1-pyrazol-3-yl)-3-(4- (morphormomethyl)-3- (trifluoromethyl)phenyl)urea |
| 40 | 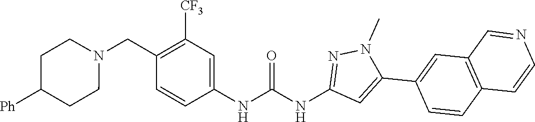 [SMILES: Cn1nc(NC(=O)Nc2ccc(CN3CCC(c4ccccc4)CC3)c(C(F)(F)F)c2)cc1-c1ccc2ccncc2c1] | 1-(5-(isoquinolin-7-yl)-1-methyl- 1H-pyrazol-3-yl)-3-(4-((4- phenylpiperidin-1-yl)methyl)-3- (trifluoromethyl)phenyl)urea |
| 41 | 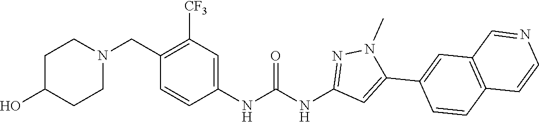 [SMILES: Cn1nc(NC(=O)Nc2ccc(CN3CCC(O)CC3)c(C(F)(F)F)c2)cc1-c1ccc2ccncc2c1] | 1-(4-((4-hydroxypiperidin-1- yl)methyl)-3- (trifluoromethyl)phenyl)-3-(5- (isoquinolin-7-yl)-1-methyl-1H- pyrazol-3-yl)urea |
| 42 | 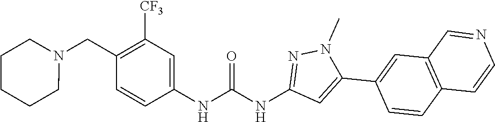 [SMILES: Cn1nc(NC(=O)Nc2ccc(CN3CCCCC3)c(C(F)(F)F)c2)cc1-c1ccc2ccncc2c1] | 1-(5-(isoquinolin-7-yl)-1-methyl- 1H-pyrazol-3-yl)-3-(4-(piperidin- 1-ylmethyl)-3- (trifluoromethyl)phenyl)urea |
| 43 | 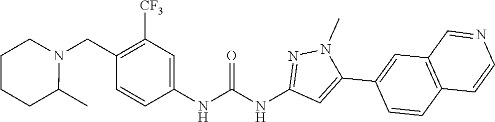 [SMILES: CC1CCCCN1Cc1ccc(NC(=O)Nc2cc(-c3ccc4ccncc4c3)n(C)n2)cc1C(F)(F)F] | 1-(5-(isoquinolin-7-yl)-1-methyl- 1H-pyrazol-3-yl)-3-(4-((2- methylpiperidin-1-yl)methyl)-3- (trifluoromethyl)phenyl)urea |
| 44 | 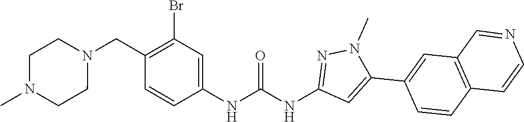 [SMILES: CN1CCN(Cc2ccc(NC(=O)Nc3cc(-c4ccc5ccncc5c4)n(C)n3)cc2Br)CC1] | 1-(3-bromo-4-((4- methylpiperazin-1- yl)methyl)phenyl)-3-(5- (isoquinolin-7-yl)-1-methyl-1H- pyrazol-3-yl)urea |
| 45 | 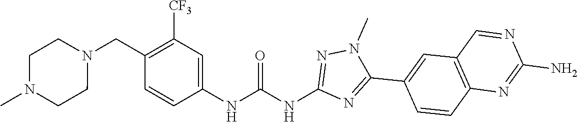 [SMILES: CN1CCN(Cc2ccc(NC(=O)Nc3nc(-c4ccc5nc(N)ncc5c4)n(C)n3)cc2C(F)(F)F)CC1] | 1-(5-(2-aminoquinazolin-6-yl)-1- methyl-1H-1,2,4-triazol-3-yl)-3- (4-((4-methylpiperazin-1- yl)methyl)-3- (trifluoromethyl)phenyl)urea |
| 46 | 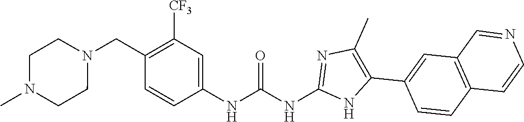 [SMILES: Cc1nc(NC(=O)Nc2ccc(CN3CCN(C)CC3)c(C(F)(F)F)c2)[nH]c1-c1ccc2ccncc2c1] | 1-(5-(isoquinolin-7-yl)-4-methyl- 4H-imidazol-2-yl)-3-(4-((4- methylpiperazin-1-yl)methyl)-3- (trifluoromethyl)phenyl)urea |
| 47 | 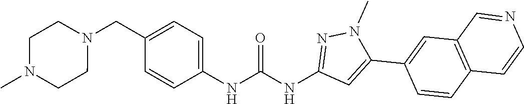 [SMILES: CN1CCN(Cc2ccc(NC(=O)Nc3cc(-c4ccc5ccncc5c4)n(C)n3)cc2)CC1] | 1-(5-(isoquinolin-7-yl)-1-methyl- 1H-pyrazol-3-yl)-3-(4-((4- methylpiperazin-1- yl)methyl)phenyl)urea |
| 48 | 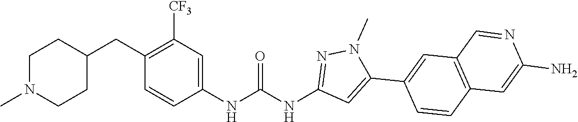 [SMILES: CN1CCC(Cc2ccc(NC(=O)Nc3cc(-c4ccc5cc(N)ncc5c4)n(C)n3)cc2C(F)(F)F)CC1] | 1-(5-(3-aminoisoquinolin-7-yl)-1- methyl-1H-pyrazol-3-yl)-3-(4-((1- methylpiperidin-4-yl)methyl)-3- (triflueromethyl)phenyl)urea |
| 49 | 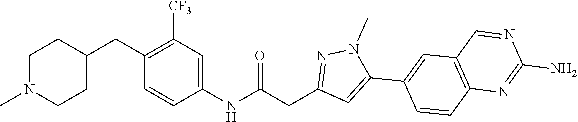 [SMILES: CN1CCC(Cc2ccc(NC(=O)Cc3cc(-c4ccc5nc(N)ncc5c4)n(C)n3)cc2C(F)(F)F)CC1] | 2-(5-(2-aminoquinazolin-6-yl)-1- methyl-1H-pyrazol-3-yl)-N-(4- ((1-methylpiperidin-4-yl)methyl)- 3-(trifluoromethyl)phenyl)- acetamide |
| 50 | 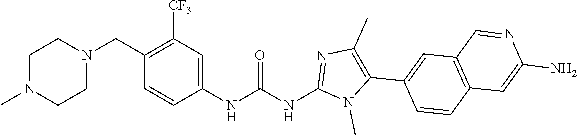 [SMILES: Cc1nc(NC(=O)Nc2ccc(CN3CCN(C)CC3)c(C(F)(F)F)c2)n(C)c1-c1ccc2cc(N)ncc2c1] | 1-(5-(3-aminoisoquinolin-7-yl)- 1,4-dimethyl-1H-imidazol-2-yl)- 3-(4-((4-methylpiperazin-1- yl)methyl)-3- (trifluoromethyl)phenyl)urea |
| 51 | 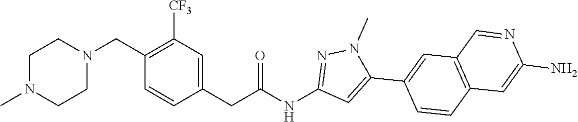 [SMILES: CN1CCN(Cc2ccc(CC(=O)Nc3cc(-c4ccc5cc(N)ncc5c4)n(C)n3)cc2C(F)(F)F)CC1] | N-(5-(3-aminoisoquinolin-7-yl)-1- methyl-1H-pyrazol-3-yl)-2-(4-((4- methylpiperazin-1-yl)methyl)-3- (trifloromethyl)phenyl)acetamide |
| 52 | 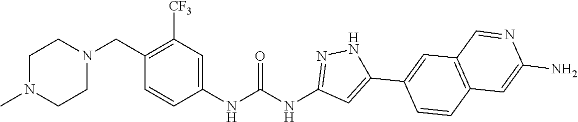 [SMILES: CN1CCN(Cc2ccc(NC(=O)Nc3cc(-c4ccc5cc(N)ncc5c4)[nH]n3)cc2C(F)(F)F)CC1] | 1-(5-(3-aminoisoquinolin-7-yl)- 1H-pyrazol-3-yl)-3-(4-((4- methylpiperazin-1-yl)methyl)-3- (trifluoromethyl)phenyl)urea |

TABLE 4

| Compound | | |
|---|---|---|
| No. | Structure | Name |
| 53 | 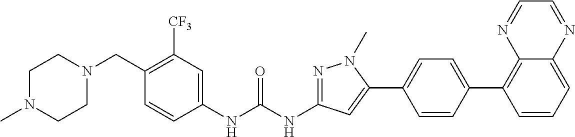 [SMILES: CN1CCN(Cc2ccc(NC(=O)Nc3cc(-c4ccc(-c5cccc6nccnc56)cc4)n(C)n3)cc2C(F)(F)F)CC1] | 1-(1-methyl-5-(4-(quinoxalin-5- yl)phenyl)-1H-pyrazol-3-yl)-3-(4- ((4-methylpiperazin-1-yl)methyl)- 3-(trifluoromethyl)phenyl)urea |
| 54 | 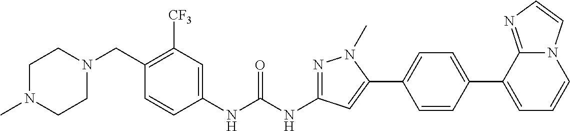 [SMILES: CN1CCN(Cc2ccc(NC(=O)Nc3cc(-c4ccc(-c5cccn6ccnc56)cc4)n(C)n3)cc2C(F)(F)F)CC1] | 1-(5-(4-(imidazo[1,2-a]pyridin-8- yl)phenyl)-1-methyl-1H-pyrazol- 3-yl)-3-(4-((4-methylpiperazin-1- yl)methyl)-3- (trifluoromethyl)phenyl)urea |
| 55 | 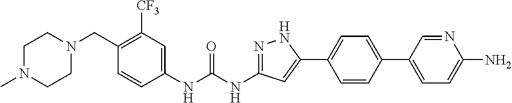 [SMILES: CN1CCN(Cc2ccc(NC(=O)Nc3cc(-c4ccc(-c5ccc(N)nc5)cc4)[nH]n3)cc2C(F)(F)F)CC1] | 1-(5-(4-(6-aminopyridin-3- yl)phenyl)-1H-pyrazol-3-yl)-3-(4- ((4-methylpiperazin-1-yl)methyl)- 3-(trifluoromethyl)phenyl)urea |
| 56 | 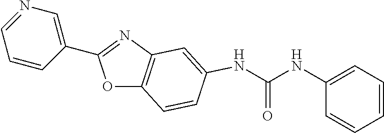 [SMILES: O=C(Nc1ccccc1)Nc1ccc2oc(-c3cccnc3)nc2c1] | 1-phenyl-3-(2-(pyridin-3- yl)benzoic[d]oxazol-5-yl)urea |
| 57 | 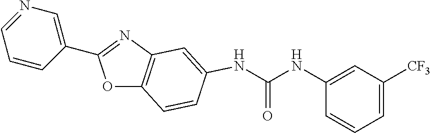 [SMILES: O=C(Nc1cccc(C(F)(F)F)c1)Nc1ccc2oc(-c3cccnc3)nc2c1] | 1-(2-(pyridin-3- yl)benzo[d]oxazol-5-yl)-3-(3- (trifluoromethyl)phenyl)urea |
| 58 | 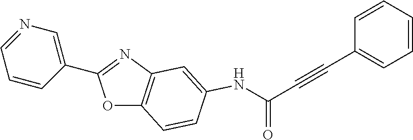 [SMILES: O=C(C#Cc1ccccc1)Nc1ccc2oc(-c3cccnc3)nc2c1] | 3-phenyl-N-(2-(pyridin-3- yl)benzo[d]oxazol-5- yl)propiolamide |
| 59 | 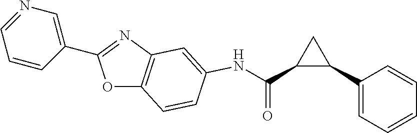 [SMILES: O=C(Nc1ccc2oc(-c3cccnc3)nc2c1)[C@H]1C[C@H]1c1ccccc1] | (1S,2R)-2-phenyl-N-(2-(pyridin- 3-yl)benzo[d]oxazol-5- yl)cyclopropane-1-carboxamide |
| 60 | 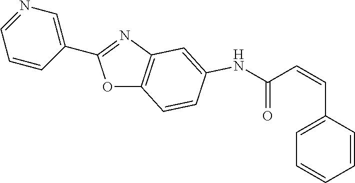 [SMILES: O=C(/C=C\c1ccccc1)Nc1ccc2oc(-c3cccnc3)nc2c1] | (Z)-3-phenyl-N-(2-(pyridin-3- yl)benzo[d]oxazol-5- yl)acrylamide |
| 61 | 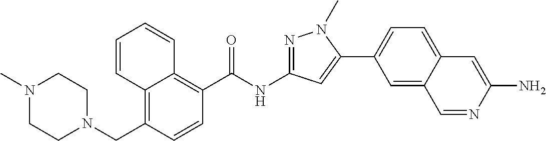 [SMILES: CN1CCN(Cc2ccc(C(=O)Nc3cc(-c4ccc5cc(N)ncc5c4)n(C)n3)c3ccccc23)CC1] | N-(5-(3-aminoisoquinolin-7-yl)-1- methyl-1H-pyrazol-3-yl)-4-((4- methylpiperazin-1-yl)methyl)-1- naphthamide |
| 62 | 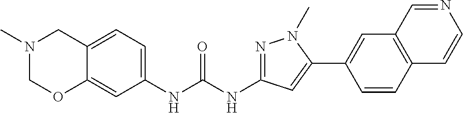 [SMILES: CN1COc2cc(NC(=O)Nc3cc(-c4ccc5ccncc5c4)n(C)n3)ccc2C1] | 1-(5-(isoquinolin-7-yl)-1-ethyl- 1H-pyrazol-3-yl)-3-(3-methyl-3,4- dihydro-2H-benzo[e][1,3]oxazin- 7-yl)urea |
| 63 | 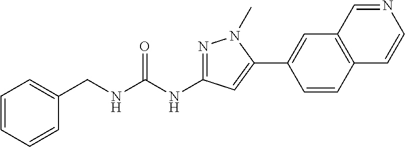 [SMILES: Cn1nc(NC(=O)NCc2ccccc2)cc1-c1ccc2ccncc2c1] | 1-benzyl-3-(5-(isoquinolin-7-yl)- 1-methyl-pyrazol-3-yl)urea |
| 64 | 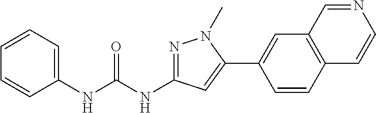 [SMILES: Cn1nc(NC(=O)Nc2ccccc2)cc1-c1ccc2ccncc2c1] | 1-(5-(isoquinolin-7-yl)-1-methyl- 1H-pyrazol-3-yl)-3-phenylurea |
| 65 | 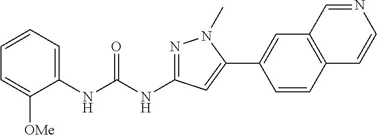 [SMILES: COc1ccccc1NC(=O)Nc1cc(-c2ccc3ccncc3c2)n(C)n1] | 1-(5-(isoquinolin-7-yl)-1-methyl- 1H-pyrazol-3-yl)-3-(2- methoxyphenyl)urea |
| 66 | 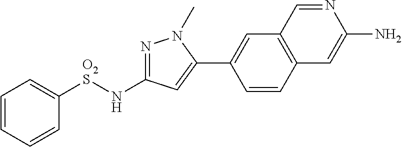 [SMILES: Cn1nc(NS(=O)(=O)c2ccccc2)cc1-c1ccc2cc(N)ncc2c1] | N-(5-(3-aminoisoquinolin-7-yl)-1- methyl-1H-pyrazol-3- yl)benzenesulfonamide |
| 67 | 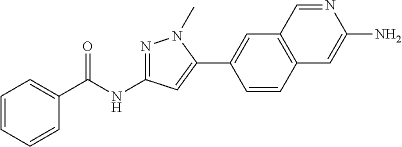 [SMILES: Cn1nc(NC(=O)c2ccccc2)cc1-c1ccc2cc(N)ncc2c1] | N-(5-(3-aminoisoquinolin-7-yl)-1- methyl-1H-pyrazol-3- yl)benzamide |
| 68 | 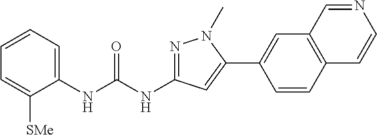 [SMILES: CSc1ccccc1NC(=O)Nc1cc(-c2ccc3ccncc3c2)n(C)n1] | 1-(5-(isoquinolin-7-yl)-1-methyl- 1H-pyrazol-3-yl)-3-(2- (methylthio)phenyl)urea |
| 69 | 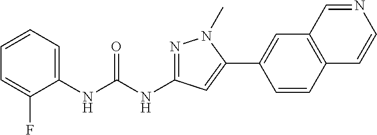 [SMILES: Cn1nc(NC(=O)Nc2ccccc2F)cc1-c1ccc2ccncc2c1] | 1-(2-fluorophenyl)-3-(5- (isoquinolin-7-yl)-1-methyl-1H- pyrazol-3-yl)urea |
| 70 | 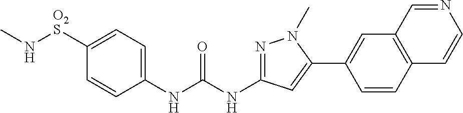 [SMILES: CNS(=O)(=O)c1ccc(NC(=O)Nc2cc(-c3ccc4ccncc4c3)n(C)n2)cc1] | (3-(5-(isoquinolin-7-yl)-1- methyl-1H-pyrazol-3-yl)ureido)- N-methylbenzenesulfonamide |
| 71 | 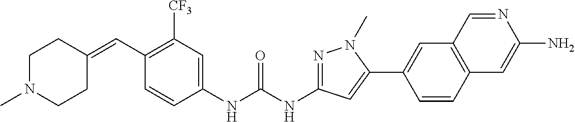 [SMILES: CN1CCC(=Cc2ccc(NC(=O)Nc3cc(-c4ccc5cc(N)ncc5c4)n(C)n3)cc2C(F)(F)F)CC1] | 1-(5-(3-aminoisoquinolin-7-yl)-1- methyl-1H-pyrazol-3-yl)-3-(4-((1- methylpiperidin-4- ylidene)methyl)-3- (trifluoromethyl)phenyl)urea |
| 72 | 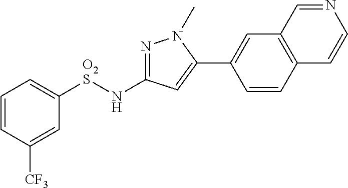 [SMILES: Cn1nc(NS(=O)(=O)c2cccc(C(F)(F)F)c2)cc1-c1ccc2ccncc2c1] | N-(5-(isoquinolin-7-yl)-1-methyl- 1H-pyrazol-3-yl)-3- (trifluoromethyl)benzenesulfonamide |
| 73 | 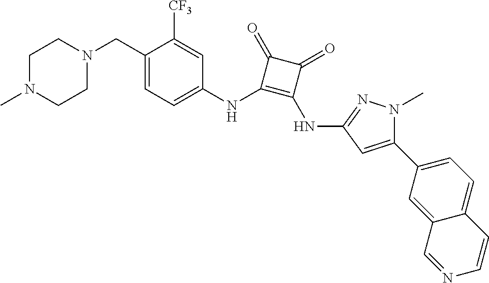 [SMILES: CN1CCN(Cc2ccc(Nc3c(Nc4cc(-c5ccc6ccncc6c5)n(C)n4)c(=O)c3=O)cc2C(F)(F)F)CC1] | 3-((5-(isoquinolin-7-yl)-1-methyl- 1H-pyrazol-3-yl)amino)-4-((4-((4- methylpiperazin-1-yl)methyl)-3- (trifluoromethyl)phenyl)amino)- cyclobut-3-ene-1,2-dione |
| 74 | 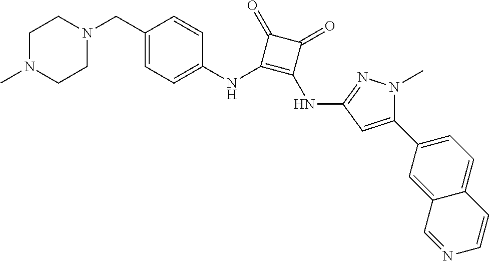 [SMILES: CN1CCN(Cc2ccc(Nc3c(Nc4cc(-c5ccc6ccncc6c5)n(C)n4)c(=O)c3=O)cc2)CC1] | 3-((5-(isoquinolin-1-yl)-1-methyl- 1H-pyrazol-3-yl)amino)-4-((4-((4- methylpiperazin-1- yl)methyl)phenyl)amino)cyclobut- 3-ene-1,2-dione |
| 75 | 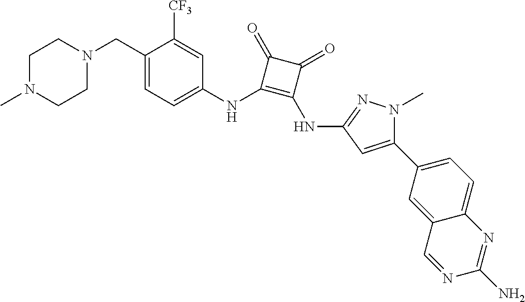 [SMILES: CN1CCN(Cc2ccc(Nc3c(Nc4cc(-c5ccc6nc(N)ncc6c5)n(C)n4)c(=O)c3=O)cc2C(F)(F)F)CC1] | 3-((5-(2-aminoquinazolin-6-yl)-1- methyl-1H-pyrazol-3-yl)amino)-4- ((4-((4-methylpiperazin-1- yl)methyl)-3- (trifluoromethyl)phenyl)amino) cyclobut-3-ene-1,2-dione |
| 76 | 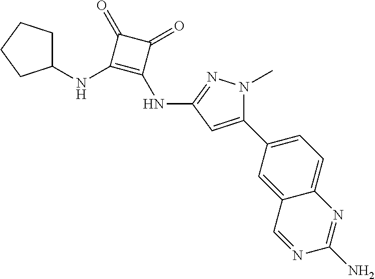 [SMILES: Cn1nc(Nc2c(NC3CCCC3)c(=O)c2=O)cc1-c1ccc2nc(N)ncc2c1] | 3-((5-(2-aminoquinazolin-6-yl)-1- methyl-1H-pyrazol-3-yl)amino)-4- (cyclopentylamino)cyclobut-3- ene-1,2-dione |

TABLE 5

| Compound | |
|---|---|
| No | Structure |
| 77 | 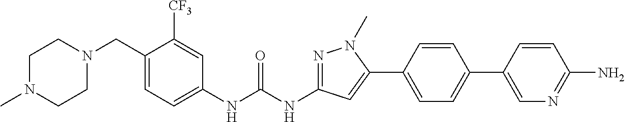 [SMILES: CN1CCN(Cc2ccc(NC(=O)Nc3cc(-c4ccc(-c5ccc(N)nc5)cc4)n(C)n3)cc2C(F)(F)F)CC1] |
| 78 | 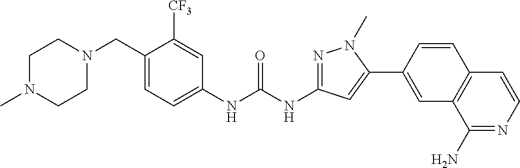 [SMILES: CN1CCN(Cc2ccc(NC(=O)Nc3cc(-c4ccc5ccnc(N)c5c4)n(C)n3)cc2C(F)(F)F)CC1] |
| 79 | 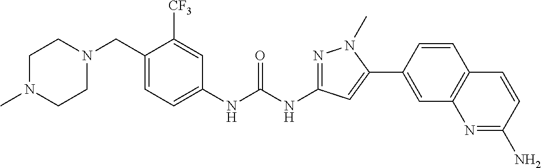 [SMILES: CN1CCN(Cc2ccc(NC(=O)Nc3cc(-c4ccc5ccc(N)nc5c4)n(C)n3)cc2C(F)(F)F)CC1] |
| 80 | 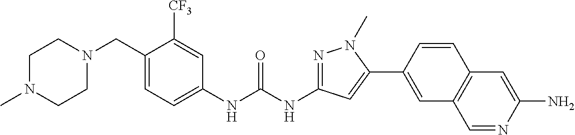 [SMILES: CN1CCN(Cc2ccc(NC(=O)Nc3cc(-c4ccc5cc(N)ncc5c4)n(C)n3)cc2C(F)(F)F)CC1] |
| 81 | 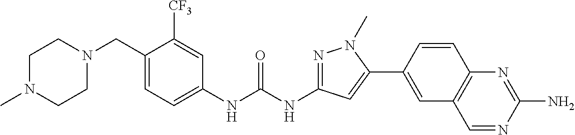 [SMILES: CN1CCN(Cc2ccc(NC(=O)Nc3cc(-c4ccc5nc(N)ncc5c4)n(C)n3)cc2C(F)(F)F)CC1] |
| 82 | 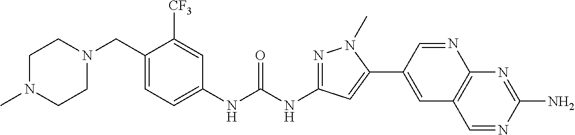 [SMILES: CN1CCN(Cc2ccc(NC(=O)Nc3cc(-c4cnc5nc(N)ncc5c4)n(C)n3)cc2C(F)(F)F)CC1] |
| 83 | 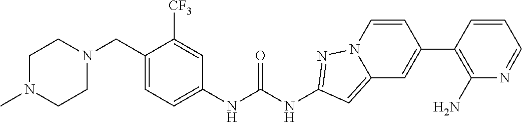 [SMILES: CN1CCN(Cc2ccc(NC(=O)Nc3cc4cc(-c5cccnc5N)ccn4n3)cc2C(F)(F)F)CC1] |
| 84 | 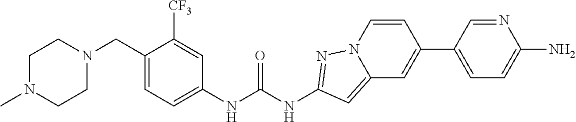 [SMILES: CN1CCN(Cc2ccc(NC(=O)Nc3cc4cc(-c5ccc(N)nc5)ccn4n3)cc2C(F)(F)F)CC1] |
| 85 | 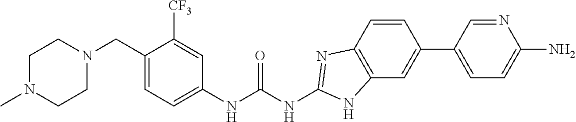 [SMILES: CN1CCN(Cc2ccc(NC(=O)Nc3nc4ccc(-c5ccc(N)nc5)cc4[nH]3)cc2C(F)(F)F)CC1] |
| 86 | 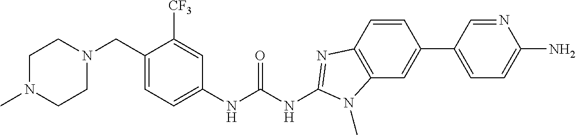 [SMILES: CN1CCN(Cc2ccc(NC(=O)Nc3nc4ccc(-c5ccc(N)nc5)cc4n3C)cc2C(F)(F)F)CC1] |
| 87 | 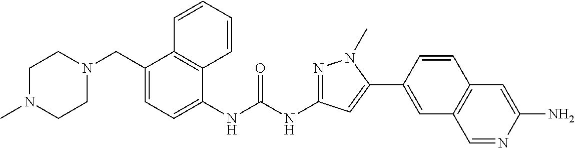 [SMILES: CN1CCN(Cc2ccc(NC(=O)Nc3cc(-c4ccc5cc(N)ncc5c4)n(C)n3)c3ccccc23)CC1] |
| 88 | 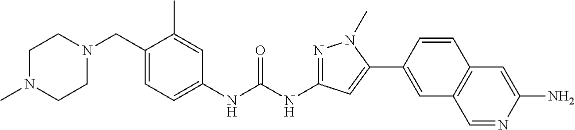 [SMILES: Cc1cc(NC(=O)Nc2cc(-c3ccc4cc(N)ncc4c3)n(C)n2)ccc1CN1CCN(C)CC1] |
| 89 | 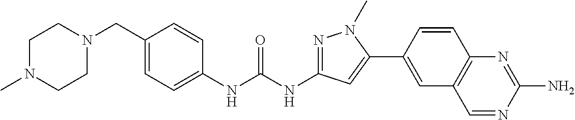 [SMILES: CN1CCN(Cc2ccc(NC(=O)Nc3cc(-c4ccc5nc(N)ncc5c4)n(C)n3)cc2)CC1] |
| 90 | 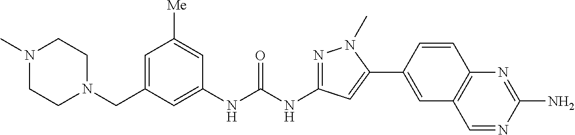 [SMILES: Cc1cc(CN2CCN(C)CC2)cc(NC(=O)Nc2cc(-c3ccc4nc(N)ncc4c3)n(C)n2)c1] |
| 91 | 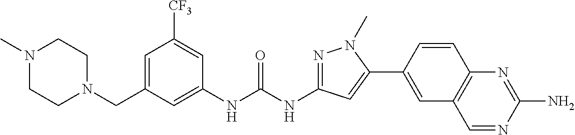 [SMILES: CN1CCN(Cc2cc(NC(=O)Nc3cc(-c4ccc5nc(N)ncc5c4)n(C)n3)cc(C(F)(F)F)c2)CC1] |
| 92 | 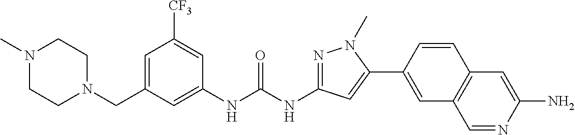 [SMILES: CN1CCN(Cc2cc(NC(=O)Nc3cc(-c4ccc5cc(N)ncc5c4)n(C)n3)cc(C(F)(F)F)c2)CC1] |
| 93 | 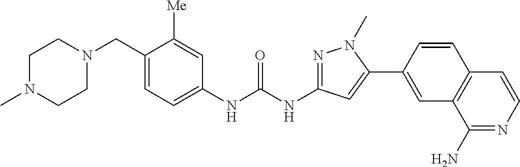 [SMILES: Cc1cc(NC(=O)Nc2cc(-c3ccc4ccnc(N)c4c3)n(C)n2)ccc1CN1CCN(C)CC1] |
| 94 | 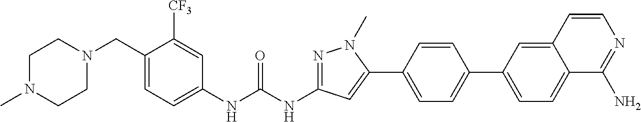 [SMILES: CN1CCN(Cc2ccc(NC(=O)Nc3cc(-c4ccc(-c5ccc6c(N)nccc6c5)cc4)n(C)n3)cc2C(F)(F)F)CC1] |
| 95 | 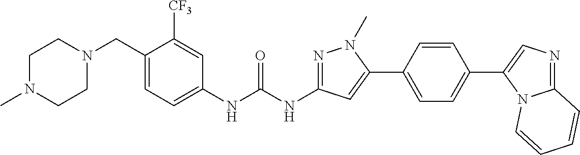 [SMILES: CN1CCN(Cc2ccc(NC(=O)Nc3cc(-c4ccc(-c5cnc6ccccn56)cc4)n(C)n3)cc2C(F)(F)F)CC1] |
| 96 | 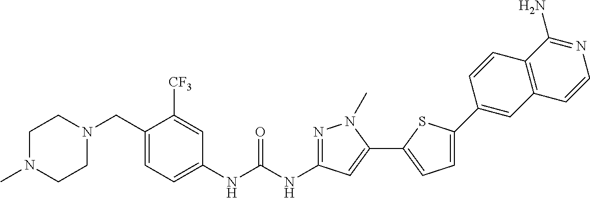 [SMILES: CN1CCN(Cc2ccc(NC(=O)Nc3cc(-c4ccc(-c5ccc6c(N)nccc6c5)s4)n(C)n3)cc2C(F)(F)F)CC1] |
| 97 | 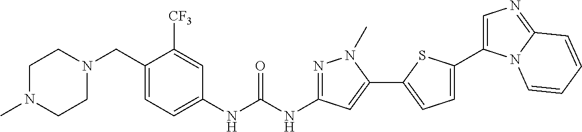 [SMILES: CN1CCN(Cc2ccc(NC(=O)Nc3cc(-c4ccc(-c5cnc6ccccn56)s4)n(C)n3)cc2C(F)(F)F)CC1] |
| 98 | 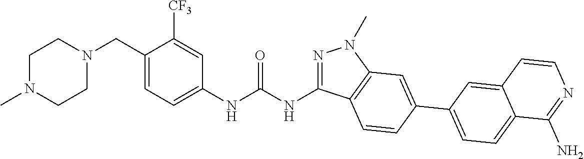 [SMILES: CN1CCN(Cc2ccc(NC(=O)Nc3nn(C)c4cc(-c5ccc6c(N)nccc6c5)ccc34)cc2C(F)(F)F)CC1] |
| 99 | 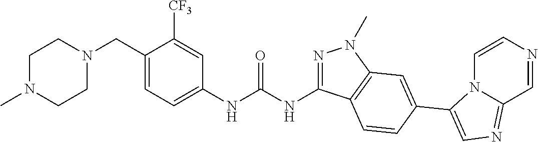 [SMILES: CN1CCN(Cc2ccc(NC(=O)Nc3nn(C)c4cc(-c5cnc6cnccn56)ccc34)cc2C(F)(F)F)CC1] |

TABLE 6

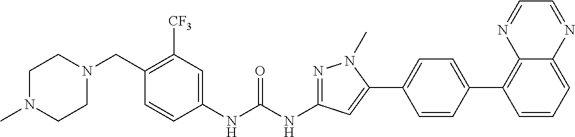 [SMILES: CN1CCN(Cc2ccc(NC(=O)Nc3cc(-c4ccc(-c5cccc6nccnc56)cc4)n(C)n3)cc2C(F)(F)F)CC1]

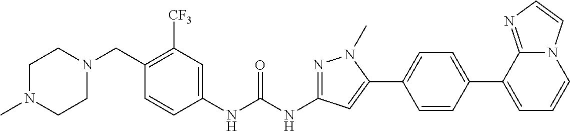 [SMILES: CN1CCN(Cc2ccc(NC(=O)Nc3cc(-c4ccc(-c5cccn6ccnc56)cc4)n(C)n3)cc2C(F)(F)F)CC1]

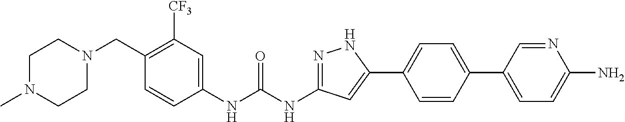 [SMILES: CN1CCN(Cc2ccc(NC(=O)Nc3cc(-c4ccc(-c5ccc(N)nc5)cc4)[nH]n3)cc2C(F)(F)F)CC1]

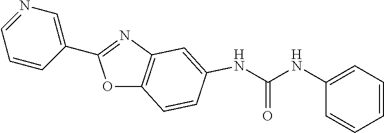 [SMILES: O=C(Nc1ccccc1)Nc1ccc2oc(-c3cccnc3)nc2c1]

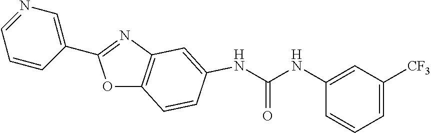 [SMILES: O=C(Nc1cccc(C(F)(F)F)c1)Nc1ccc2oc(-c3cccnc3)nc2c1]

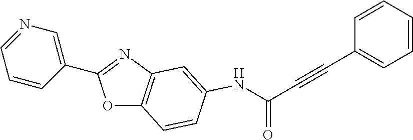 [SMILES: O=C(C#Cc1ccccc1)Nc1ccc2oc(-c3cccnc3)nc2c1]

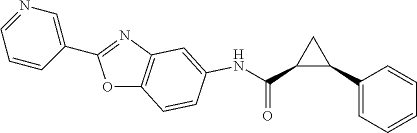 [SMILES: O=C(Nc1ccc2oc(-c3cccnc3)nc2c1)[C@H]1C[C@H]1c1ccccc1]

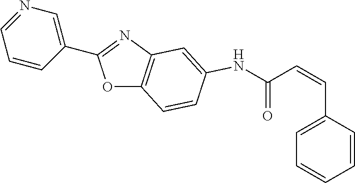 [SMILES: O=C(/C=C\c1ccccc1)Nc1ccc2oc(-c3cccnc3)nc2c1]

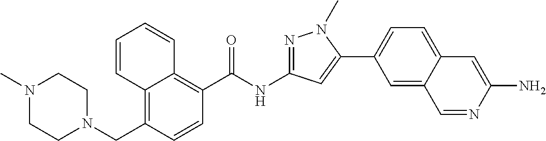 [SMILES: CN1CCN(Cc2ccc(C(=O)Nc3cc(-c4ccc5cc(N)ncc5c4)n(C)n3)c3ccccc23)CC1]

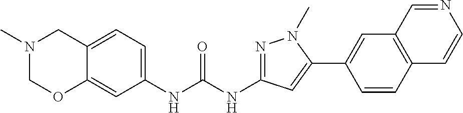 [SMILES: CN1COc2cc(NC(=O)Nc3cc(-c4ccc5ccncc5c4)n(C)n3)ccc2C1]

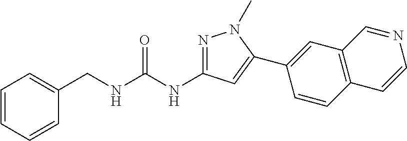 [SMILES: Cn1nc(NC(=O)NCc2ccccc2)cc1-c1ccc2ccncc2c1]

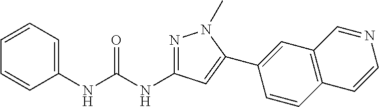 [SMILES: Cn1nc(NC(=O)Nc2ccccc2)cc1-c1ccc2ccncc2c1]

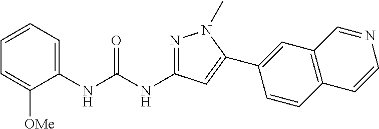 [SMILES: COc1ccccc1NC(=O)Nc1cc(-c2ccc3ccncc3c2)n(C)n1]

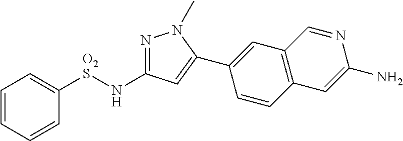 [SMILES: Cn1nc(NS(=O)(=O)c2ccccc2)cc1-c1ccc2cc(N)ncc2c1]

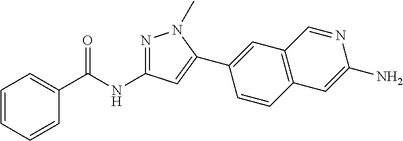 [SMILES: Cn1nc(NC(=O)c2ccccc2)cc1-c1ccc2cc(N)ncc2c1]

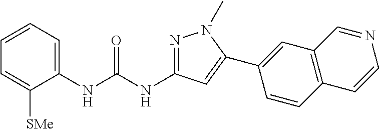 [SMILES: CSc1ccccc1NC(=O)Nc1cc(-c2ccc3ccncc3c2)n(C)n1]

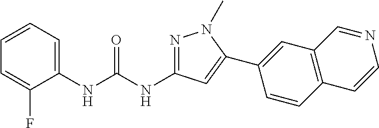 [SMILES: Cn1nc(NC(=O)Nc2ccccc2F)cc1-c1ccc2ccncc2c1]

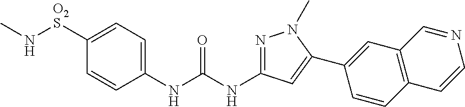 [SMILES: CNS(=O)(=O)c1ccc(NC(=O)Nc2cc(-c3ccc4ccncc4c3)n(C)n2)cc1]

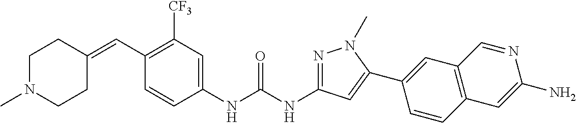 [SMILES: CN1CCC(=Cc2ccc(NC(=O)Nc3cc(-c4ccc5cc(N)ncc5c4)n(C)n3)cc2C(F)(F)F)CC1]

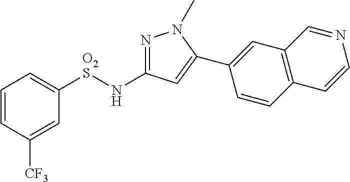 [SMILES: Cn1nc(NS(=O)(=O)c2cccc(C(F)(F)F)c2)cc1-c1ccc2ccncc2c1]

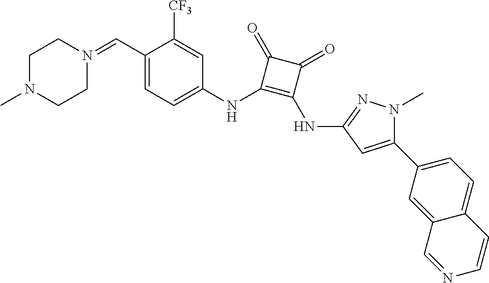

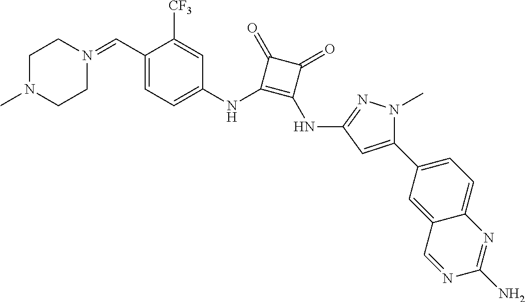

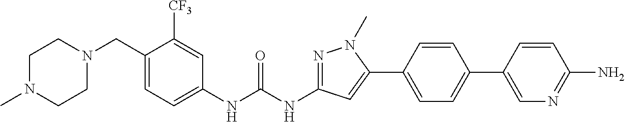 [SMILES: CN1CCN(Cc2ccc(NC(=O)Nc3cc(-c4ccc(-c5ccc(N)nc5)cc4)n(C)n3)cc2C(F)(F)F)CC1]

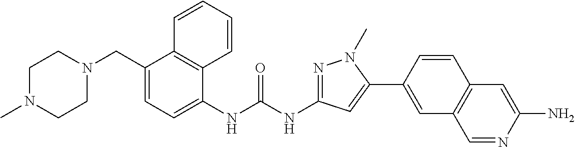 [SMILES: CN1CCN(Cc2ccc(NC(=O)Nc3cc(-c4ccc5cc(N)ncc5c4)n(C)n3)c3ccccc23)CC1]

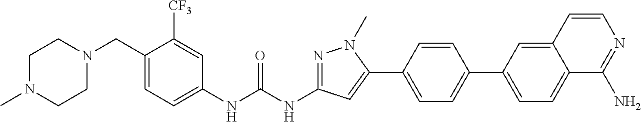 [SMILES: CN1CCN(Cc2ccc(NC(=O)Nc3cc(-c4ccc(-c5ccc6c(N)nccc6c5)cc4)n(C)n3)cc2C(F)(F)F)CC1]

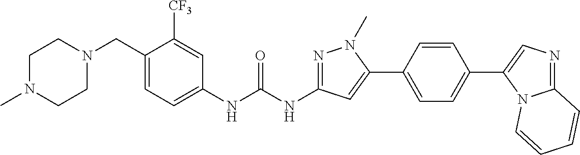 [SMILES: CN1CCN(Cc2ccc(NC(=O)Nc3cc(-c4ccc(-c5cnc6ccccn56)cc4)n(C)n3)cc2C(F)(F)F)CC1]

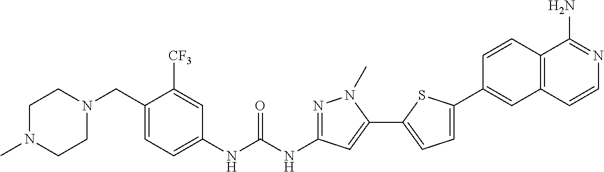 [SMILES: CN1CCN(Cc2ccc(NC(=O)Nc3cc(-c4ccc(-c5ccc6c(N)nccc6c5)s4)n(C)n3)cc2C(F)(F)F)CC1]

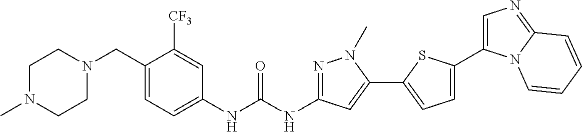 [SMILES: CN1CCN(Cc2ccc(NC(=O)Nc3cc(-c4ccc(-c5cnc6ccccn56)s4)n(C)n3)cc2C(F)(F)F)CC1]

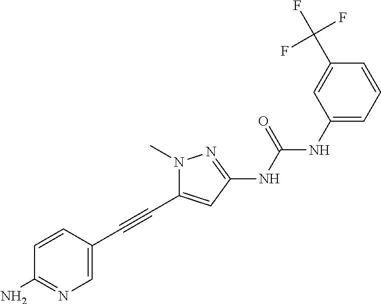 [SMILES: Cn1nc(NC(=O)Nc2cccc(C(F)(F)F)c2)cc1C#Cc1ccc(N)nc1]

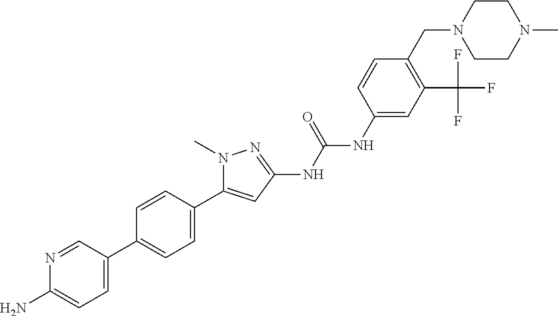 [SMILES: CN1CCN(Cc2ccc(NC(=O)Nc3cc(-c4ccc(-c5ccc(N)nc5)cc4)n(C)n3)cc2C(F)(F)F)CC1]

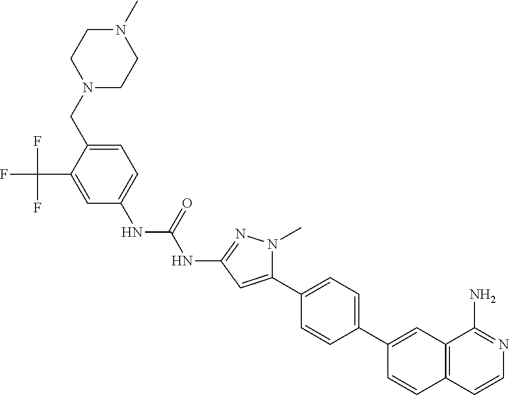 [SMILES: CN1CCN(Cc2ccc(NC(=O)Nc3cc(-c4ccc(-c5ccc6ccnc(N)c6c5)cc4)n(C)n3)cc2C(F)(F)F)CC1]

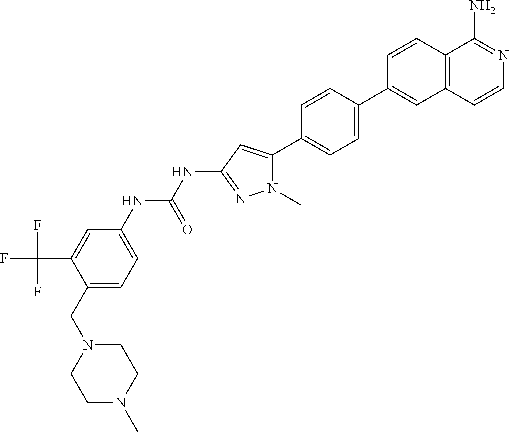 [SMILES: CN1CCN(Cc2ccc(NC(=O)Nc3cc(-c4ccc(-c5ccc6c(N)nccc6c5)cc4)n(C)n3)cc2C(F)(F)F)CC1]

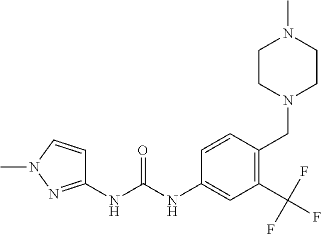 [SMILES: CN1CCN(Cc2ccc(NC(=O)Nc3ccn(C)n3)cc2C(F)(F)F)CC1]

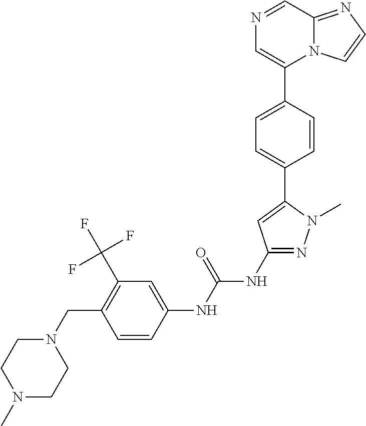 [SMILES: CN1CCN(Cc2ccc(NC(=O)Nc3cc(-c4ccc(-c5cncc6nccn56)cc4)n(C)n3)cc2C(F)(F)F)CC1]

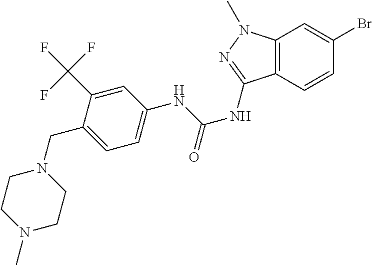 [SMILES: CN1CCN(Cc2ccc(NC(=O)Nc3nn(C)c4cc(Br)ccc34)cc2C(F)(F)F)CC1]

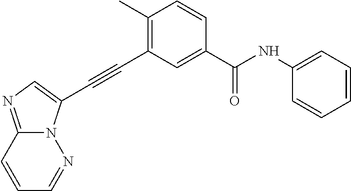 [SMILES: Cc1ccc(C(=O)Nc2ccccc2)cc1C#Cc1cnc2cccnn12]

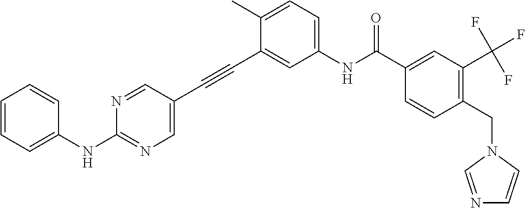 [SMILES: Cc1ccc(NC(=O)c2ccc(Cn3ccnc3)c(C(F)(F)F)c2)cc1C#Cc1cnc(Nc2ccccc2)nc1]

TABLE 7

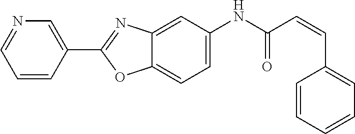 [SMILES: O=C(/C=C\c1ccccc1)Nc1ccc2oc(-c3cccnc3)nc2c1]

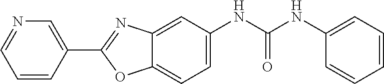 [SMILES: O=C(Nc1ccccc1)Nc1ccc2oc(-c3cccnc3)nc2c1]

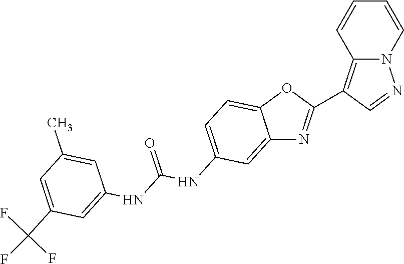 [SMILES: Cc1cc(NC(=O)Nc2ccc3oc(-c4cnn5ccccc45)nc3c2)cc(C(F)(F)F)c1]

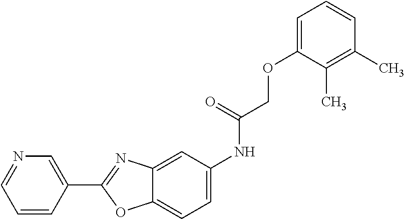 [SMILES: Cc1cccc(OCC(=O)Nc2ccc3oc(-c4cccnc4)nc3c2)c1C]

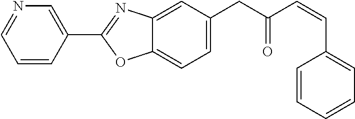 [SMILES: O=C(/C=C\c1ccccc1)Cc1ccc2oc(-c3cccnc3)nc2c1]

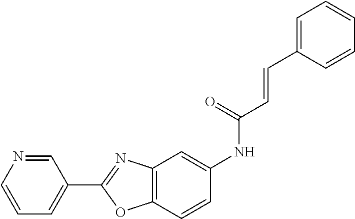 [SMILES: O=C(/C=C/c1ccccc1)Nc1ccc2oc(-c3cccnc3)nc2c1]

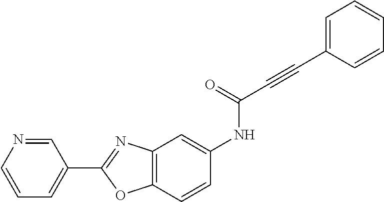 [SMILES: O=C(C#Cc1ccccc1)Nc1ccc2oc(-c3cccnc3)nc2c1]

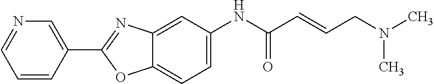 [SMILES: CN(C)C/C=C/C(=O)Nc1ccc2oc(-c3cccnc3)nc2c1]

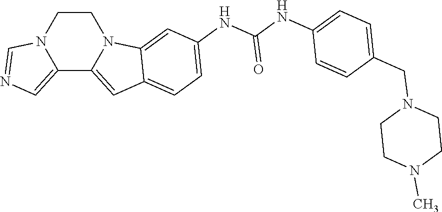 [SMILES: CN1CCN(Cc2ccc(NC(=O)Nc3ccc4cc5n(c4c3)CCn3cncc3-5)cc2)CC1]

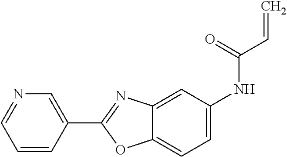 [SMILES: C=CC(=O)Nc1ccc2oc(-c3cccnc3)nc2c1]

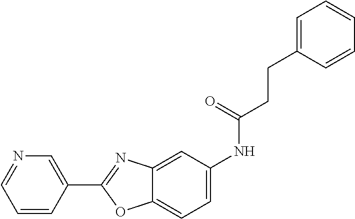 [SMILES: O=C(CCc1ccccc1)Nc1ccc2oc(-c3cccnc3)nc2c1]

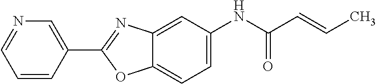 [SMILES: C/C=C/C(=O)Nc1ccc2oc(-c3cccnc3)nc2c1]

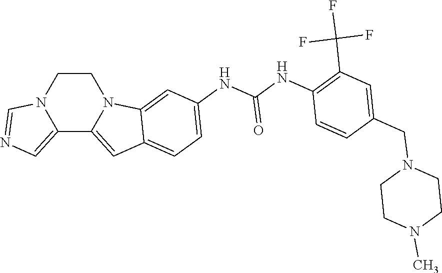 [SMILES: CN1CCN(Cc2ccc(NC(=O)Nc3ccc4cc5n(c4c3)CCn3cncc3-5)c(C(F)(F)F)c2)CC1]

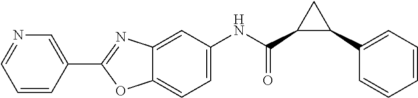 [SMILES: O=C(Nc1ccc2oc(-c3cccnc3)nc2c1)[C@H]1C[C@H]1c1ccccc1]

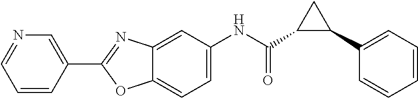 [SMILES: O=C(Nc1ccc2oc(-c3cccnc3)nc2c1)[C@@H]1C[C@H]1c1ccccc1]

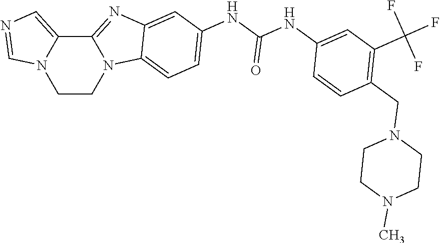 [SMILES: CN1CCN(Cc2ccc(NC(=O)Nc3ccc4c(c3)nc3n4CCn4cncc4-3)cc2C(F)(F)F)CC1]

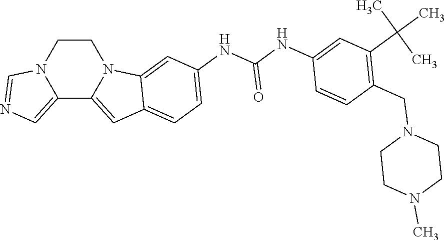 [SMILES: CN1CCN(Cc2ccc(NC(=O)Nc3ccc4cc5n(c4c3)CCn3cncc3-5)cc2C(C)(C)C)CC1]

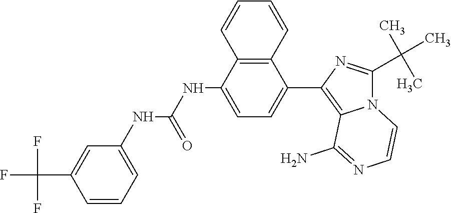 [SMILES: CC(C)(C)c1nc(-c2ccc(NC(=O)Nc3cccc(C(F)(F)F)c3)c3ccccc23)c2c(N)nccn12]

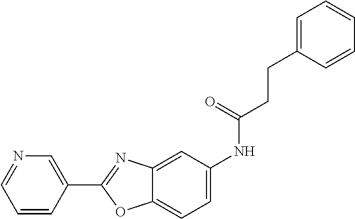 [SMILES: O=C(CCc1ccccc1)Nc1ccc2oc(-c3cccnc3)nc2c1]

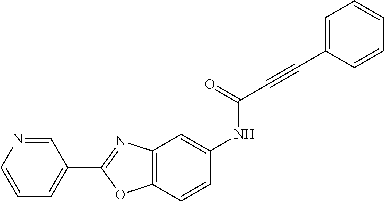 [SMILES: O=C(C#Cc1ccccc1)Nc1ccc2oc(-c3cccnc3)nc2c1]

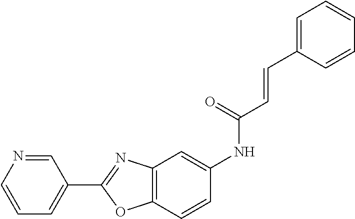 [SMILES: O=C(/C=C/c1ccccc1)Nc1ccc2oc(-c3cccnc3)nc2c1]

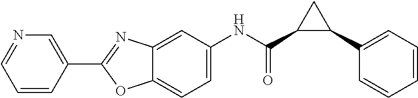 [SMILES: O=C(Nc1ccc2oc(-c3cccnc3)nc2c1)[C@H]1C[C@H]1c1ccccc1]

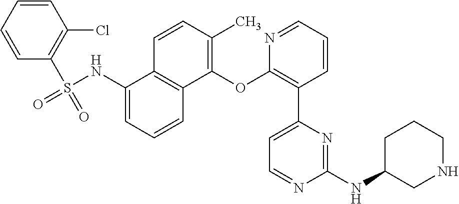 [SMILES: Cc1ccc2c(NS(=O)(=O)c3ccccc3Cl)cccc2c1Oc1ncccc1-c1ccnc(N[C@H]2CCCNC2)n1]

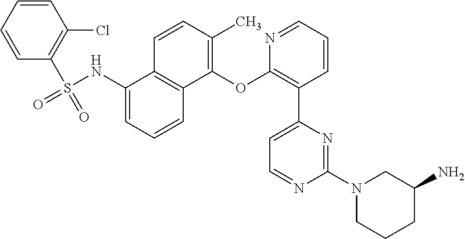 [SMILES: Cc1ccc2c(NS(=O)(=O)c3ccccc3Cl)cccc2c1Oc1ncccc1-c1ccnc(N2CCC[C@H](N)C2)n1]

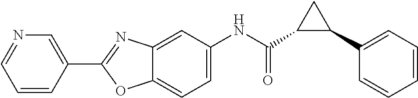 [SMILES: O=C(Nc1ccc2oc(-c3cccnc3)nc2c1)[C@@H]1C[C@H]1c1ccccc1]

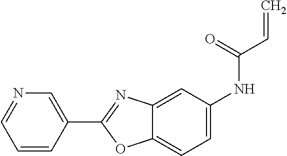 [SMILES: C=CC(=O)Nc1ccc2oc(-c3cccnc3)nc2c1]

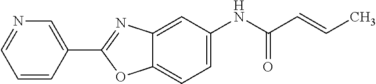 [SMILES: C/C=C/C(=O)Nc1ccc2oc(-c3cccnc3)nc2c1]

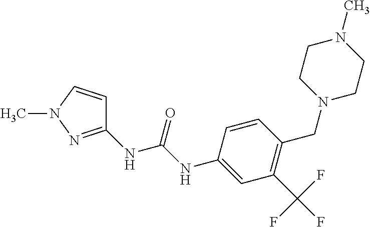 [SMILES: CN1CCN(Cc2ccc(NC(=O)Nc3ccn(C)n3)cc2C(F)(F)F)CC1]

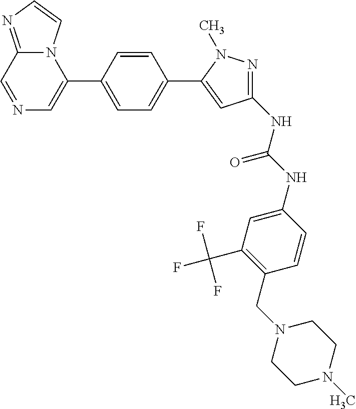 [SMILES: CN1CCN(Cc2ccc(NC(=O)Nc3cc(-c4ccc(-c5cncc6nccn56)cc4)n(C)n3)cc2C(F)(F)F)CC1]

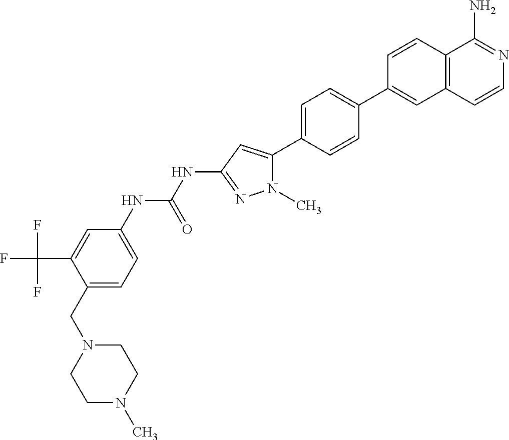 [SMILES: CN1CCN(Cc2ccc(NC(=O)Nc3cc(-c4ccc(-c5ccc6c(N)nccc6c5)cc4)n(C)n3)cc2C(F)(F)F)CC1]

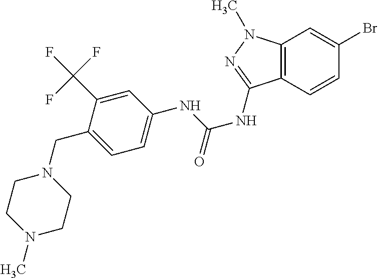 [SMILES: CN1CCN(Cc2ccc(NC(=O)Nc3nn(C)c4cc(Br)ccc34)cc2C(F)(F)F)CC1]

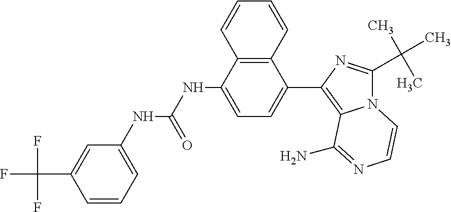 [SMILES: CC(C)(C)c1nc(-c2ccc(NC(=O)Nc3cccc(C(F)(F)F)c3)c3ccccc23)c2c(N)nccn12]

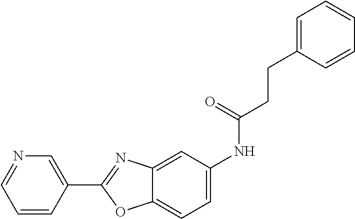 [SMILES: O=C(CCc1ccccc1)Nc1ccc2oc(-c3cccnc3)nc2c1]

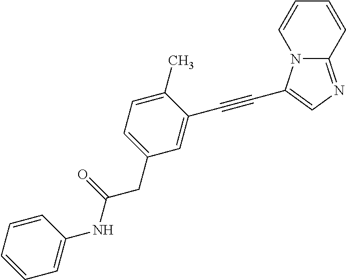 [SMILES: Cc1ccc(CC(=O)Nc2ccccc2)cc1C#Cc1cnc2ccccn12]

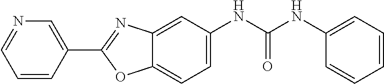 [SMILES: O=C(Nc1ccccc1)Nc1ccc2oc(-c3cccnc3)nc2c1]

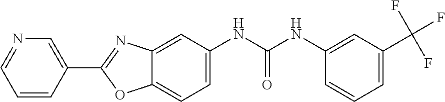 [SMILES: O=C(Nc1cccc(C(F)(F)F)c1)Nc1ccc2oc(-c3cccnc3)nc2c1]

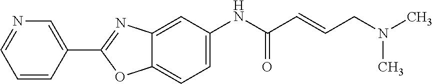 [SMILES: CN(C)C/C=C/C(=O)Nc1ccc2oc(-c3cccnc3)nc2c1]

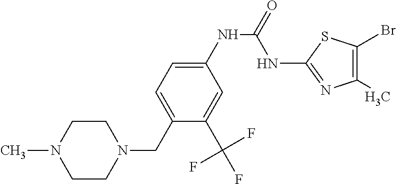 [SMILES: Cc1nc(NC(=O)Nc2ccc(CN3CCN(C)CC3)c(C(F)(F)F)c2)sc1Br]

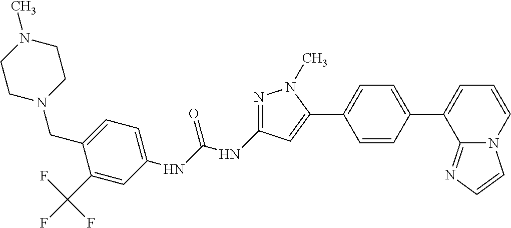 [SMILES: CN1CCN(Cc2ccc(NC(=O)Nc3cc(-c4ccc(-c5cccn6ccnc56)cc4)n(C)n3)cc2C(F)(F)F)CC1]

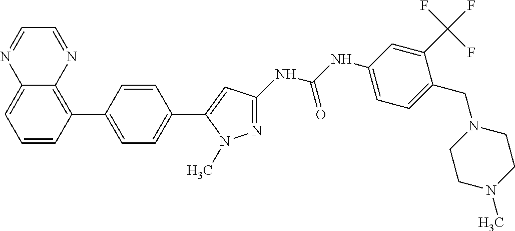 [SMILES: CN1CCN(Cc2ccc(NC(=O)Nc3cc(-c4ccc(-c5cccc6nccnc56)cc4)n(C)n3)cc2C(F)(F)F)CC1]

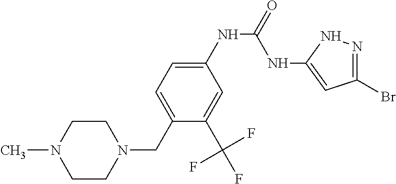 [SMILES: CN1CCN(Cc2ccc(NC(=O)Nc3cc(Br)n[nH]3)cc2C(F)(F)F)CC1]

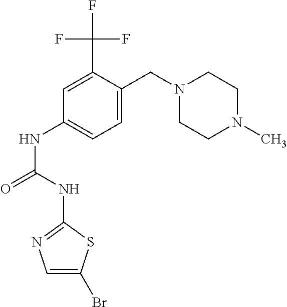 [SMILES: CN1CCN(Cc2ccc(NC(=O)Nc3ncc(Br)s3)cc2C(F)(F)F)CC1]

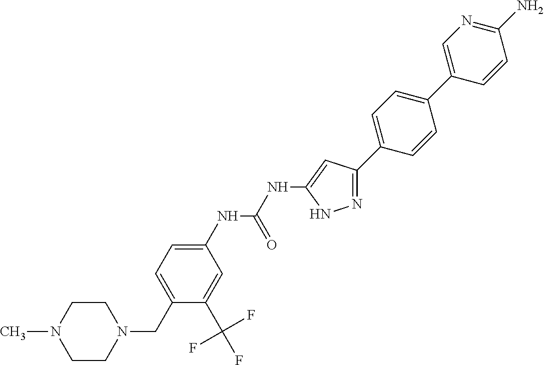 [SMILES: CN1CCN(Cc2ccc(NC(=O)Nc3cc(-c4ccc(-c5ccc(N)nc5)cc4)n[nH]3)cc2C(F)(F)F)CC1]

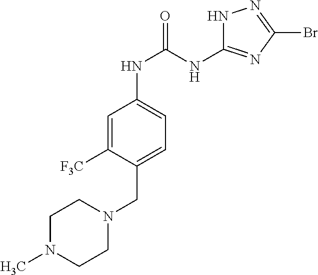 [SMILES: CN1CCN(Cc2ccc(NC(=O)Nc3nc(Br)n[nH]3)cc2C(F)(F)F)CC1]

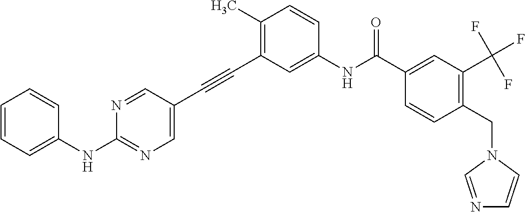 [SMILES: Cc1ccc(NC(=O)c2ccc(Cn3ccnc3)c(C(F)(F)F)c2)cc1C#Cc1cnc(Nc2ccccc2)nc1]

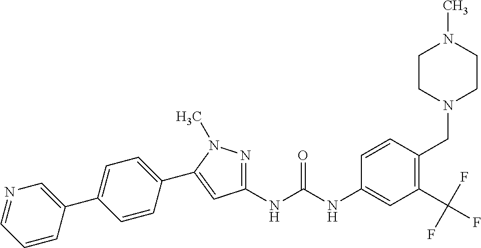 [SMILES: CN1CCN(Cc2ccc(NC(=O)Nc3cc(-c4ccc(-c5cccnc5)cc4)n(C)n3)cc2C(F)(F)F)CC1]

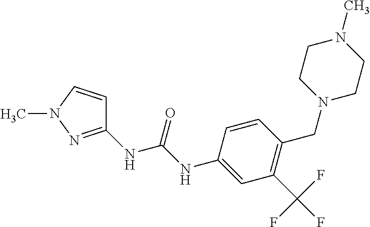 [SMILES: CN1CCN(Cc2ccc(NC(=O)Nc3ccn(C)n3)cc2C(F)(F)F)CC1]

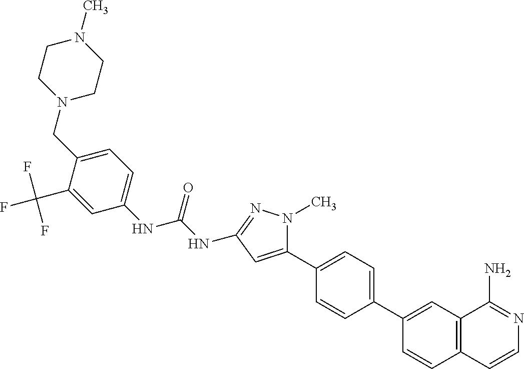 [SMILES: CN1CCN(Cc2ccc(NC(=O)Nc3cc(-c4ccc(-c5ccc6ccnc(N)c6c5)cc4)n(C)n3)cc2C(F)(F)F)CC1]

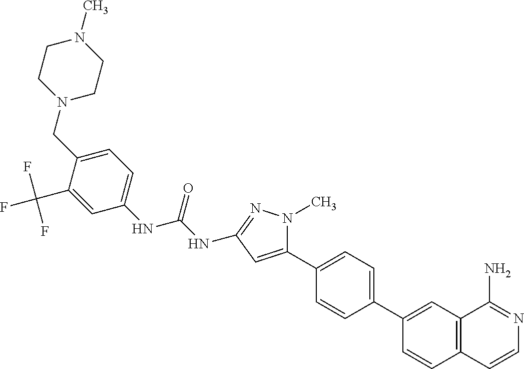 [SMILES: CN1CCN(Cc2ccc(NC(=O)Nc3cc(-c4ccc(-c5ccc6ccnc(N)c6c5)cc4)n(C)n3)cc2C(F)(F)F)CC1]

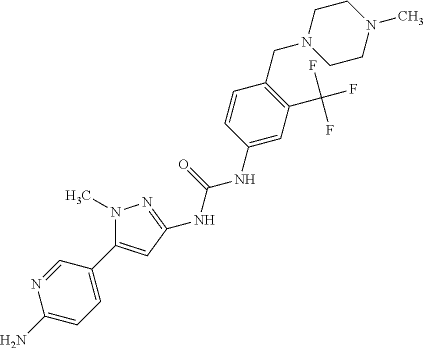 [SMILES: CN1CCN(Cc2ccc(NC(=O)Nc3cc(-c4ccc(N)nc4)n(C)n3)cc2C(F)(F)F)CC1]

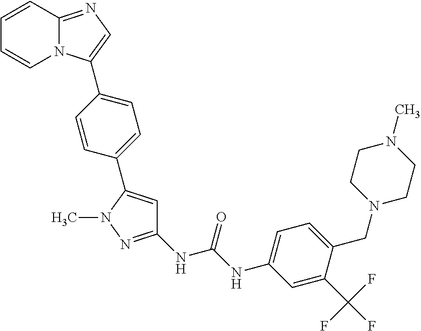 [SMILES: CN1CCN(Cc2ccc(NC(=O)Nc3cc(-c4ccc(-c5cnc6ccccn56)cc4)n(C)n3)cc2C(F)(F)F)CC1]

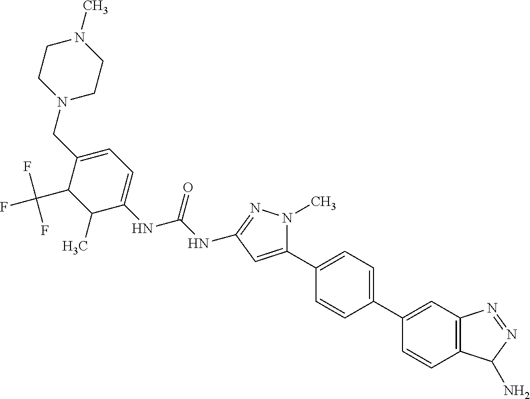 [SMILES: CC1C(NC(=O)Nc2cc(-c3ccc(-c4ccc5c(c4)N=NC5N)cc3)n(C)n2)=CC=C(CN2CCN(C)CC2)C1C(F)(F)F]

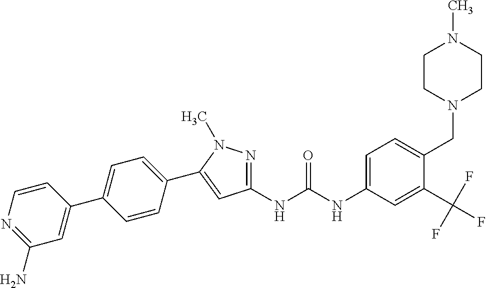 [SMILES: CN1CCN(Cc2ccc(NC(=O)Nc3cc(-c4ccc(-c5ccnc(N)c5)cc4)n(C)n3)cc2C(F)(F)F)CC1]

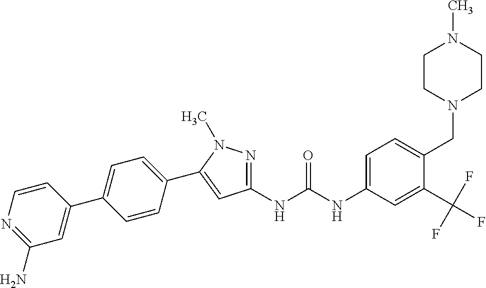 [SMILES: CN1CCN(Cc2ccc(NC(=O)Nc3cc(-c4ccc(-c5ccnc(N)c5)cc4)n(C)n3)cc2C(F)(F)F)CC1]

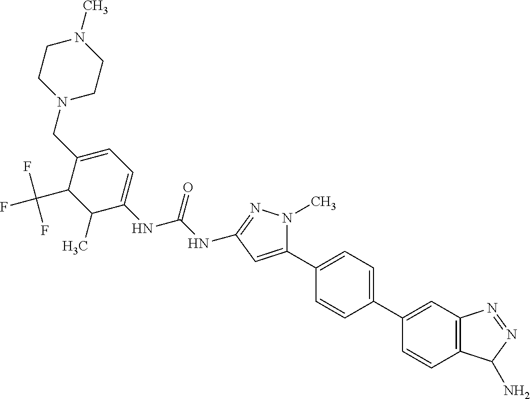 [SMILES: CC1C(NC(=O)Nc2cc(-c3ccc(-c4ccc5c(c4)N=NC5N)cc3)n(C)n2)=CC=C(CN2CCN(C)CC2)C1C(F)(F)F]

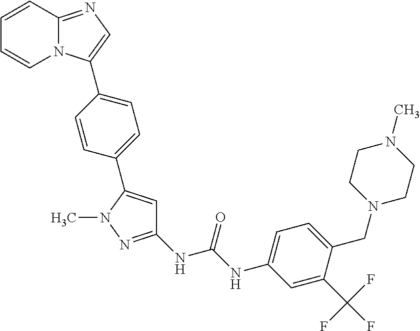 [SMILES: CN1CCN(Cc2ccc(NC(=O)Nc3cc(-c4ccc(-c5cnc6ccccn56)cc4)n(C)n3)cc2C(F)(F)F)CC1]

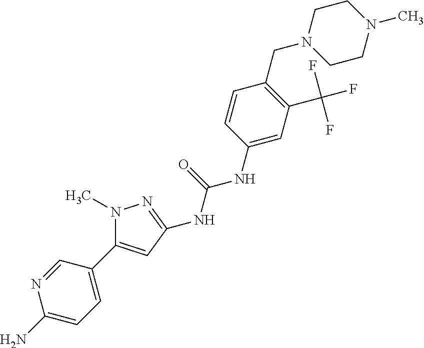 [SMILES: CN1CCN(Cc2ccc(NC(=O)Nc3cc(-c4ccc(N)nc4)n(C)n3)cc2C(F)(F)F)CC1]

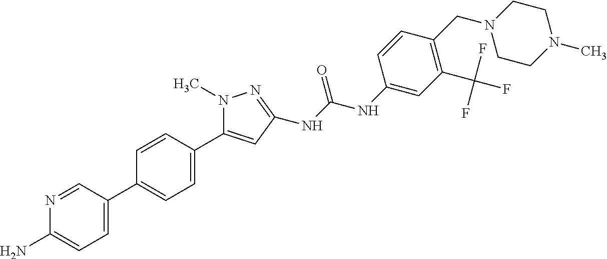 [SMILES: CN1CCN(Cc2ccc(NC(=O)Nc3cc(-c4ccc(-c5ccc(N)nc5)cc4)n(C)n3)cc2C(F)(F)F)CC1]

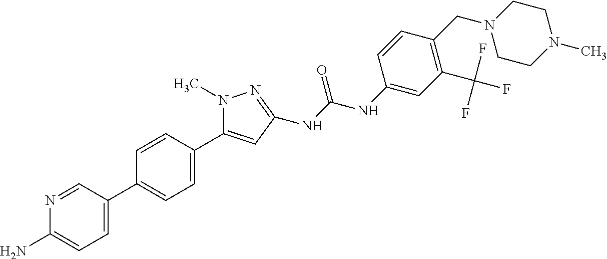 [SMILES: CN1CCN(Cc2ccc(NC(=O)Nc3cc(-c4ccc(-c5ccc(N)nc5)cc4)n(C)n3)cc2C(F)(F)F)CC1]

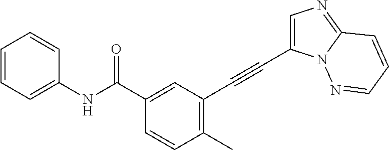 [SMILES: Cc1ccc(C(=O)Nc2ccccc2)cc1C#Cc1cnc2cccnn12]

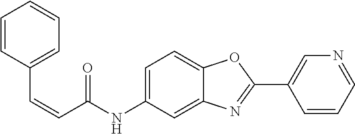 [SMILES: O=C(/C=C\c1ccccc1)Nc1ccc2oc(-c3cccnc3)nc2c1]

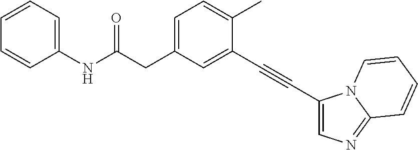 [SMILES: Cc1ccc(CC(=O)Nc2ccccc2)cc1C#Cc1cnc2ccccn12]

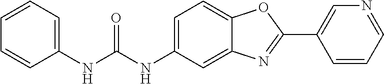 [SMILES: O=C(Nc1ccccc1)Nc1ccc2oc(-c3cccnc3)nc2c1]

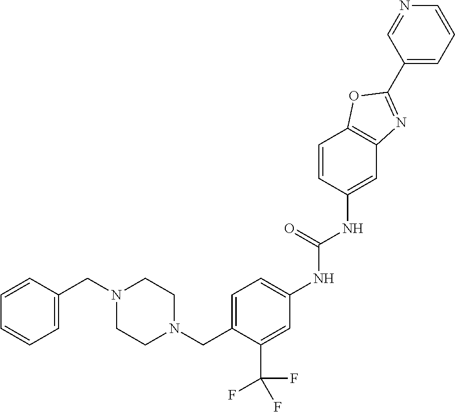 [SMILES: O=C(Nc1ccc(CN2CCN(Cc3ccccc3)CC2)c(C(F)(F)F)c1)Nc1ccc2oc(-c3cccnc3)nc2c1]

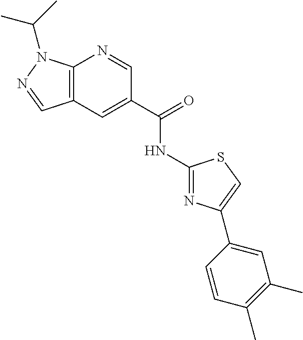 [SMILES: Cc1ccc(-c2csc(NC(=O)c3cnc4c(cnn4C(C)C)c3)n2)cc1C]

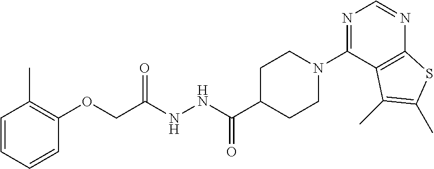 [SMILES: Cc1ccccc1OCC(=O)NNC(=O)C1CCN(c2ncnc3sc(C)c(C)c23)CC1]

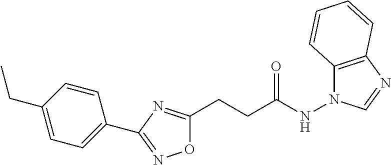 [SMILES: CCc1ccc(-c2noc(CCC(=O)Nn3cnc4ccccc43)n2)cc1]

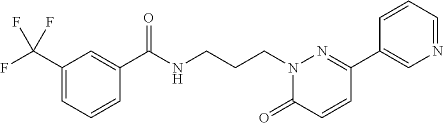 [SMILES: O=C(NCCCn1nc(-c2cccnc2)ccc1=O)c1cccc(C(F)(F)F)c1]

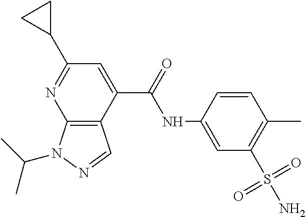 [SMILES: Cc1ccc(NC(=O)c2cc(C3CC3)nc3c2cnn3C(C)C)cc1S(N)(=O)=O]

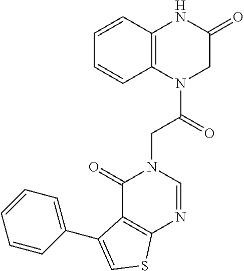 [SMILES: O=C1CN(C(=O)Cn2cnc3scc(-c4ccccc4)c3c2=O)c2ccccc2N1]

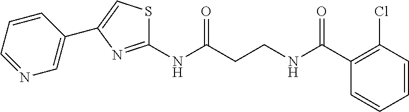 [SMILES: O=C(CCNC(=O)c1ccccc1Cl)Nc1nc(-c2cccnc2)cs1]

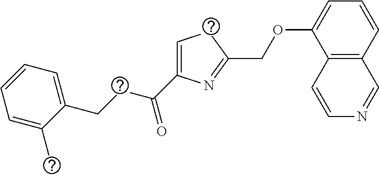 [SMILES: Cc1ccccc1CCC(=O)C1=CCC(COc2cccc3cnccc23)=N1]

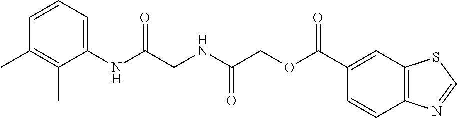 [SMILES: Cc1cccc(NC(=O)CNC(=O)COC(=O)c2ccc3ncsc3c2)c1C]

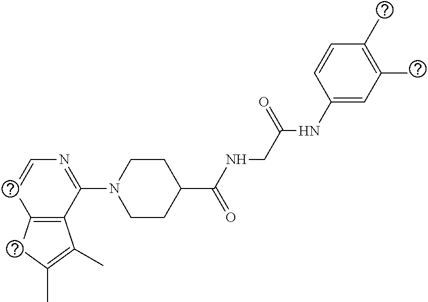 [SMILES: CC1=C(C)c2c(ccnc2N2CCC(C(=O)NCC(=O)Nc3ccc(C)c(C)c3)CC2)C1]

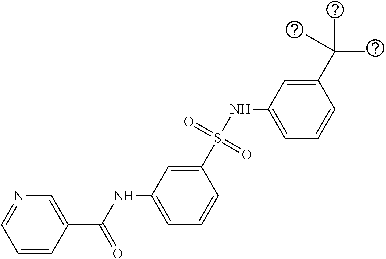 [SMILES: CC(C)(C)c1cccc(NS(=O)(=O)c2cccc(NC(=O)c3cccnc3)c2)c1]

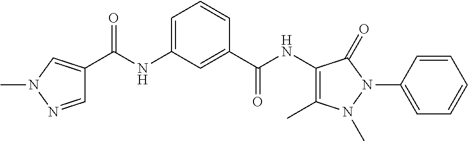 [SMILES: Cc1c(NC(=O)c2cccc(NC(=O)c3cnn(C)c3)c2)c(=O)n(-c2ccccc2)n1C]

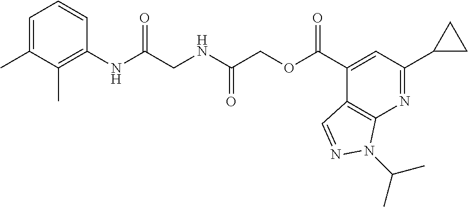 [SMILES: Cc1cccc(NC(=O)CNC(=O)COC(=O)c2cc(C3CC3)nc3c2cnn3C(C)C)c1C]

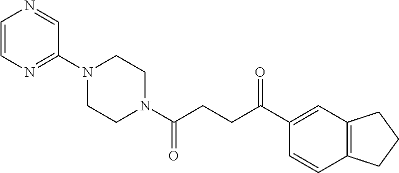 [SMILES: O=C(CCC(=O)N1CCN(c2cnccn2)CC1)c1ccc2c(c1)CCC2]

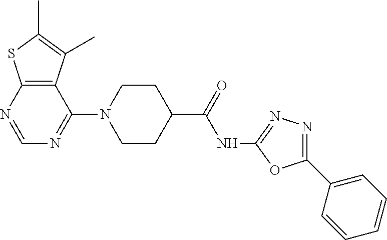 [SMILES: Cc1sc2ncnc(N3CCC(C(=O)Nc4nnc(-c5ccccc5)o4)CC3)c2c1C]

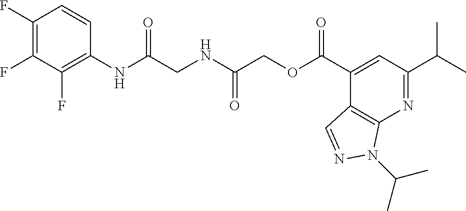 [SMILES: CC(C)c1cc(C(=O)OCC(=O)NCC(=O)Nc2ccc(F)c(F)c2F)c2cnn(C(C)C)c2n1]

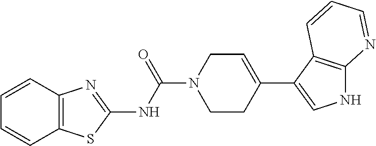 [SMILES: O=C(Nc1nc2ccccc2s1)N1CC=C(c2c[nH]c3ncccc23)CC1]

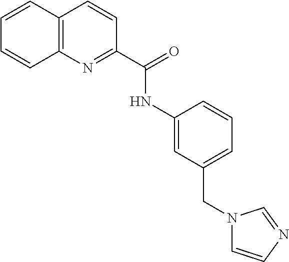 [SMILES: O=C(Nc1cccc(Cn2ccnc2)c1)c1ccc2ccccc2n1]

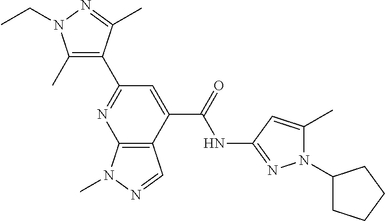 [SMILES: CCn1nc(C)c(-c2cc(C(=O)Nc3cc(C)n(C4CCCC4)n3)c3cnn(C)c3n2)c1C]

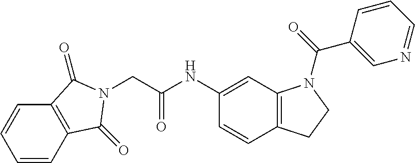 [SMILES: O=C(CN1C(=O)c2ccccc2C1=O)Nc1ccc2c(c1)N(C(=O)c1cccnc1)CC2]

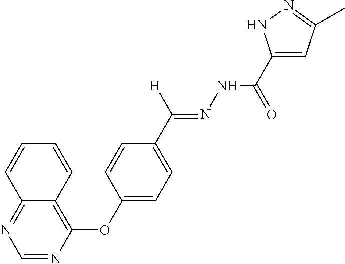 [SMILES: Cc1cc(C(=O)N/N=C/c2ccc(Oc3ncnc4ccccc34)cc2)[nH]n1]

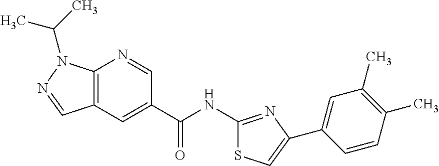 [SMILES: Cc1ccc(-c2csc(NC(=O)c3cnc4c(cnn4C(C)C)c3)n2)cc1C]

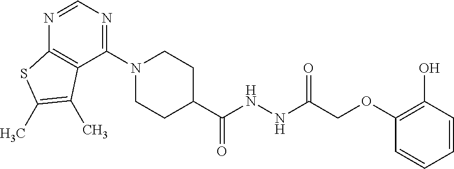 [SMILES: Cc1sc2ncnc(N3CCC(C(=O)NNC(=O)COc4ccccc4O)CC3)c2c1C]

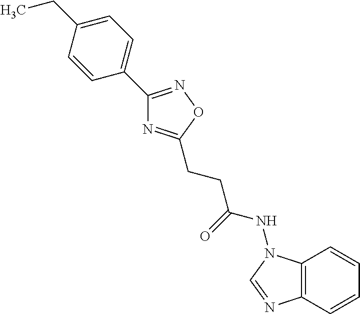 [SMILES: CCc1ccc(-c2noc(CCC(=O)Nn3cnc4ccccc43)n2)cc1]

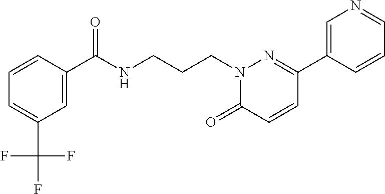 [SMILES: O=C(NCCCn1nc(-c2cccnc2)ccc1=O)c1cccc(C(F)(F)F)c1]

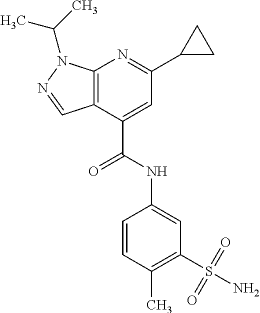 [SMILES: Cc1ccc(NC(=O)c2cc(C3CC3)nc3c2cnn3C(C)C)cc1S(N)(=O)=O]

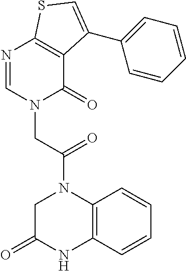 [SMILES: O=C1CN(C(=O)Cn2cnc3scc(-c4ccccc4)c3c2=O)c2ccccc2N1]

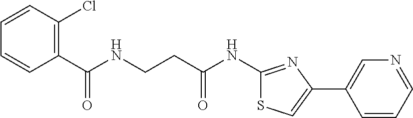 [SMILES: O=C(CCNC(=O)c1ccccc1Cl)Nc1nc(-c2cccnc2)cs1]

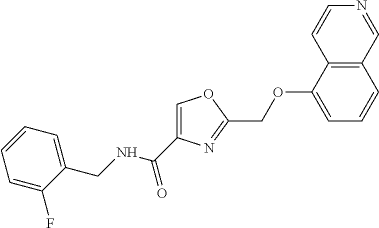 [SMILES: O=C(NCc1ccccc1F)c1coc(COc2cccc3cnccc23)n1]

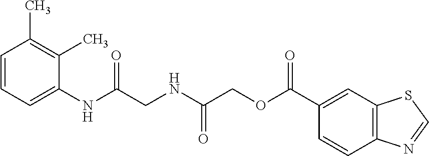 [SMILES: Cc1cccc(NC(=O)CNC(=O)COC(=O)c2ccc3ncsc3c2)c1C]

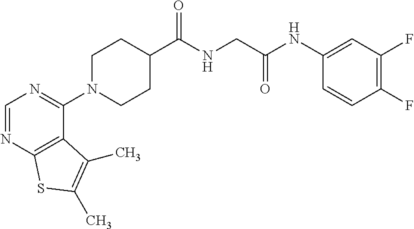 [SMILES: Cc1sc2ncnc(N3CCC(C(=O)NCC(=O)Nc4ccc(F)c(F)c4)CC3)c2c1C]

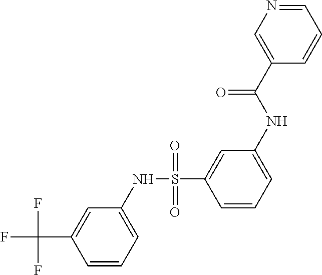 [SMILES: O=C(Nc1cccc(S(=O)(=O)Nc2cccc(C(F)(F)F)c2)c1)c1cccnc1]

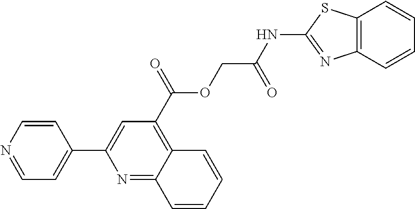 [SMILES: O=C(COC(=O)c1cc(-c2ccncc2)nc2ccccc12)Nc1nc2ccccc2s1]

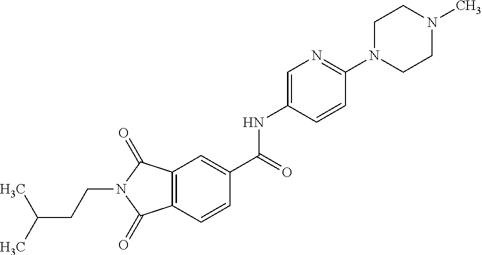 [SMILES: CC(C)CCN1C(=O)c2ccc(C(=O)Nc3ccc(N4CCN(C)CC4)nc3)cc2C1=O]

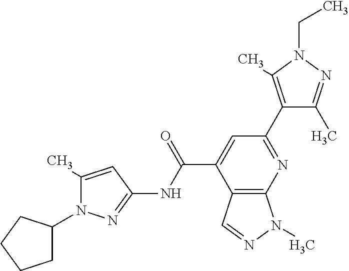 [SMILES: CCn1nc(C)c(-c2cc(C(=O)Nc3cc(C)n(C4CCCC4)n3)c3cnn(C)c3n2)c1C]

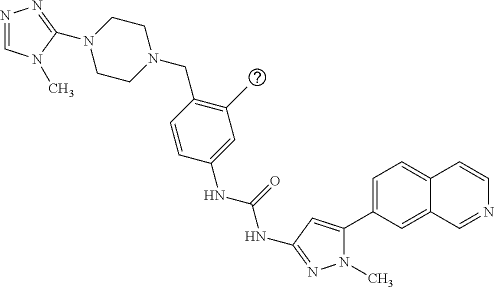 [SMILES: Cc1cc(NC(=O)Nc2cc(-c3ccc4ccncc4c3)n(C)n2)ccc1CN1CCN(c2nncn2C)CC1]

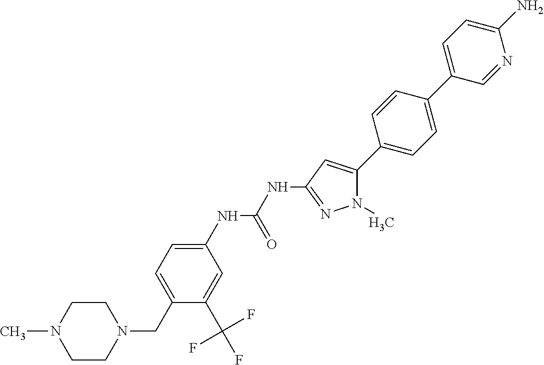 [SMILES: CN1CCN(Cc2ccc(NC(=O)Nc3cc(-c4ccc(-c5ccc(N)nc5)cc4)n(C)n3)cc2C(F)(F)F)CC1]

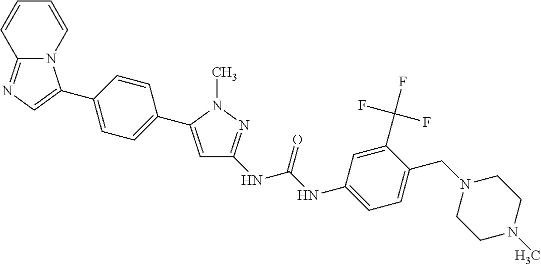 [SMILES: CN1CCN(Cc2ccc(NC(=O)Nc3cc(-c4ccc(-c5cnc6ccccn56)cc4)n(C)n3)cc2C(F)(F)F)CC1]

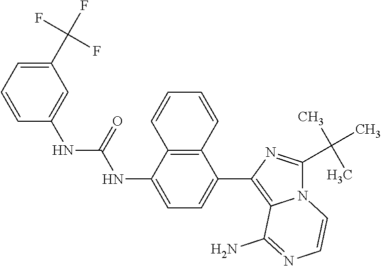 [SMILES: CC(C)(C)c1nc(-c2ccc(NC(=O)Nc3cccc(C(F)(F)F)c3)c3ccccc23)c2c(N)nccn12]

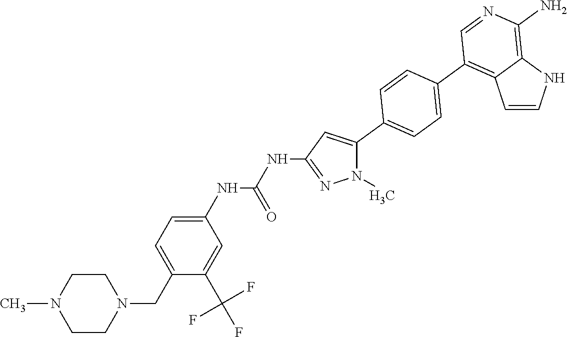 [SMILES: CN1CCN(Cc2ccc(NC(=O)Nc3cc(-c4ccc(-c5cnc(N)c6[nH]ccc56)cc4)n(C)n3)cc2C(F)(F)F)CC1]

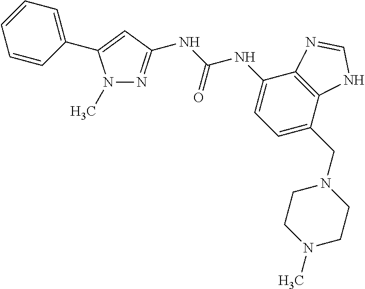 [SMILES: CN1CCN(Cc2ccc(NC(=O)Nc3cc(-c4ccccc4)n(C)n3)c3nc[nH]c23)CC1]

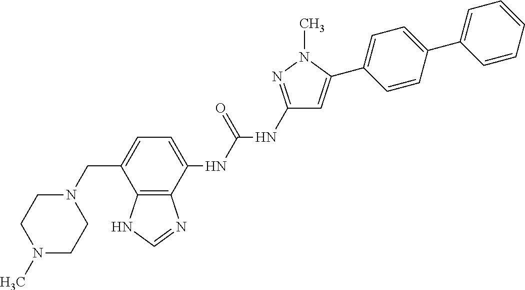 [SMILES: CN1CCN(Cc2ccc(NC(=O)Nc3cc(-c4ccc(-c5ccccc5)cc4)n(C)n3)c3nc[nH]c23)CC1]

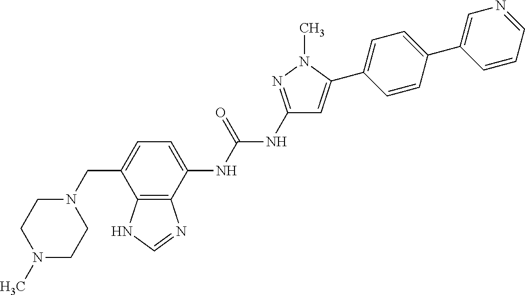 [SMILES: CN1CCN(Cc2ccc(NC(=O)Nc3cc(-c4ccc(-c5cccnc5)cc4)n(C)n3)c3nc[nH]c23)CC1]

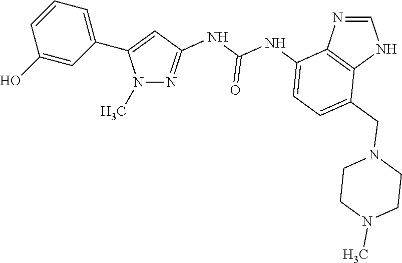 [SMILES: CN1CCN(Cc2ccc(NC(=O)Nc3cc(-c4cccc(O)c4)n(C)n3)c3nc[nH]c23)CC1]

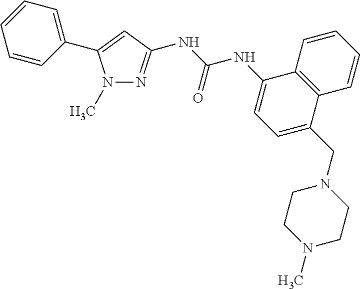 [SMILES: CN1CCN(Cc2ccc(NC(=O)Nc3cc(-c4ccccc4)n(C)n3)c3ccccc23)CC1]

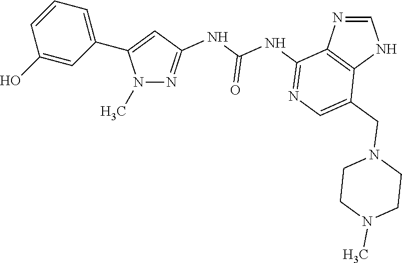 [SMILES: CN1CCN(Cc2cnc(NC(=O)Nc3cc(-c4cccc(O)c4)n(C)n3)c3nc[nH]c23)CC1]

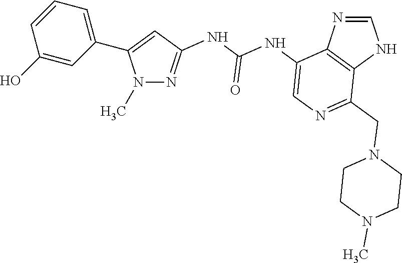 [SMILES: CN1CCN(Cc2ncc(NC(=O)Nc3cc(-c4cccc(O)c4)n(C)n3)c3nc[nH]c23)CC1]

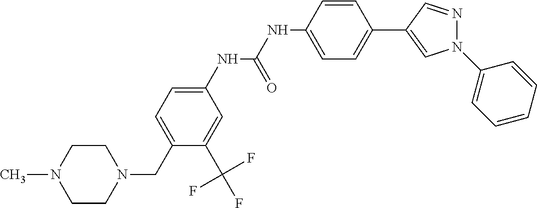 [SMILES: CN1CCN(Cc2ccc(NC(=O)Nc3ccc(-c4cnn(-c5ccccc5)c4)cc3)cc2C(F)(F)F)CC1]

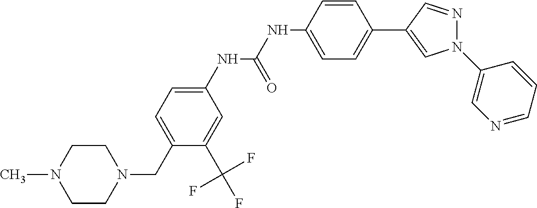 [SMILES: CN1CCN(Cc2ccc(NC(=O)Nc3ccc(-c4cnn(-c5cccnc5)c4)cc3)cc2C(F)(F)F)CC1]

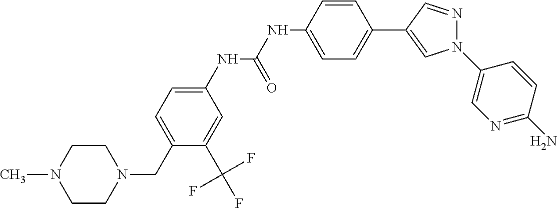 [SMILES: CN1CCN(Cc2ccc(NC(=O)Nc3ccc(-c4cnn(-c5ccc(N)nc5)c4)cc3)cc2C(F)(F)F)CC1]

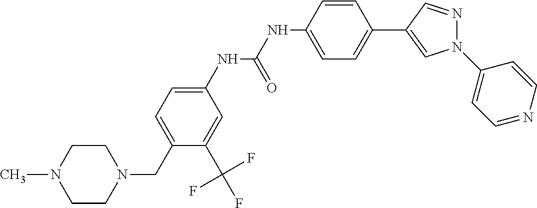 [SMILES: CN1CCN(Cc2ccc(NC(=O)Nc3ccc(-c4cnn(-c5ccncc5)c4)cc3)cc2C(F)(F)F)CC1]

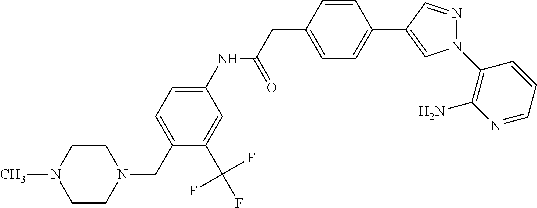 [SMILES: CN1CCN(Cc2ccc(NC(=O)Cc3ccc(-c4cnn(-c5cccnc5N)c4)cc3)cc2C(F)(F)F)CC1]

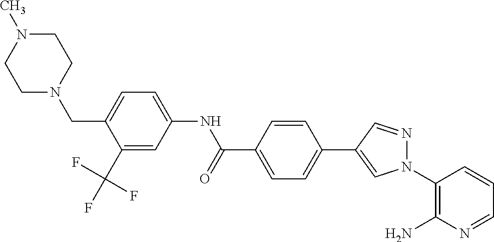 [SMILES: CN1CCN(Cc2ccc(NC(=O)c3ccc(-c4cnn(-c5cccnc5N)c4)cc3)cc2C(F)(F)F)CC1]

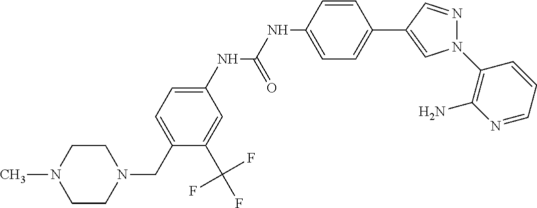 [SMILES: CN1CCN(Cc2ccc(NC(=O)Nc3ccc(-c4cnn(-c5cccnc5N)c4)cc3)cc2C(F)(F)F)CC1]

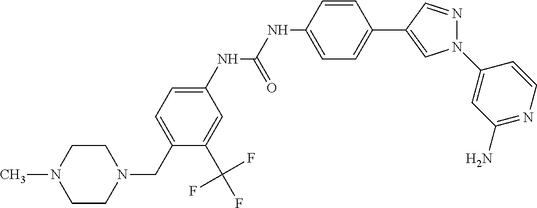 [SMILES: CN1CCN(Cc2ccc(NC(=O)Nc3ccc(-c4cnn(-c5ccnc(N)c5)c4)cc3)cc2C(F)(F)F)CC1]

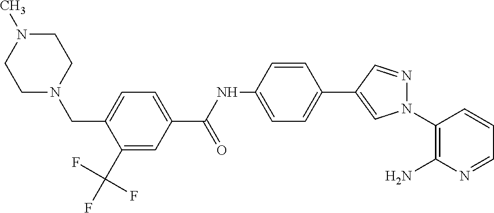 [SMILES: CN1CCN(Cc2ccc(C(=O)Nc3ccc(-c4cnn(-c5cccnc5N)c4)cc3)cc2C(F)(F)F)CC1]

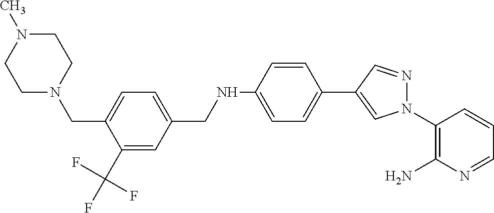 [SMILES: CN1CCN(Cc2ccc(CNc3ccc(-c4cnn(-c5cccnc5N)c4)cc3)cc2C(F)(F)F)CC1]

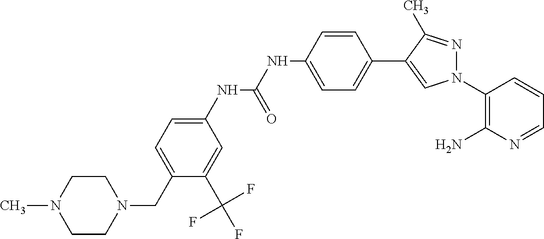 [SMILES: Cc1nn(-c2cccnc2N)cc1-c1ccc(NC(=O)Nc2ccc(CN3CCN(C)CC3)c(C(F)(F)F)c2)cc1]

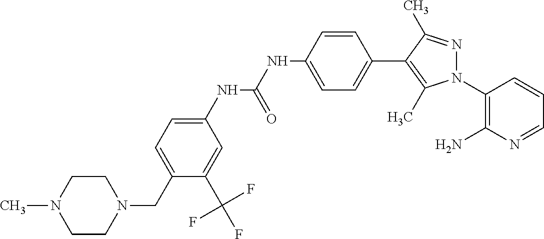 [SMILES: Cc1nn(-c2cccnc2N)c(C)c1-c1ccc(NC(=O)Nc2ccc(CN3CCN(C)CC3)c(C(F)(F)F)c2)cc1]

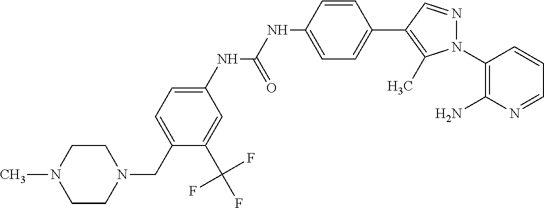 [SMILES: Cc1c(-c2ccc(NC(=O)Nc3ccc(CN4CCN(C)CC4)c(C(F)(F)F)c3)cc2)cnn1-c1cccnc1N]

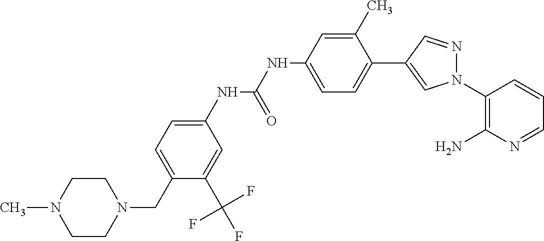 [SMILES: Cc1cc(NC(=O)Nc2ccc(CN3CCN(C)CC3)c(C(F)(F)F)c2)ccc1-c1cnn(-c2cccnc2N)c1]

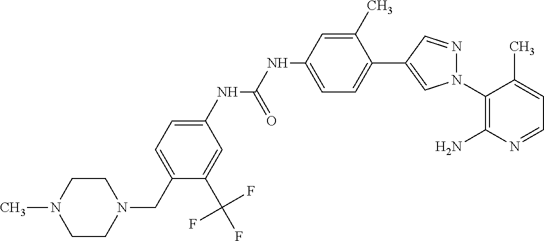 [SMILES: Cc1cc(NC(=O)Nc2ccc(CN3CCN(C)CC3)c(C(F)(F)F)c2)ccc1-c1cnn(-c2c(C)ccnc2N)c1]

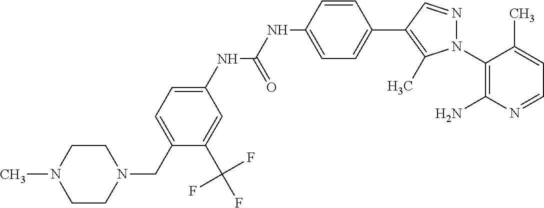 [SMILES: Cc1ccnc(N)c1-n1ncc(-c2ccc(NC(=O)Nc3ccc(CN4CCN(C)CC4)c(C(F)(F)F)c3)cc2)c1C]

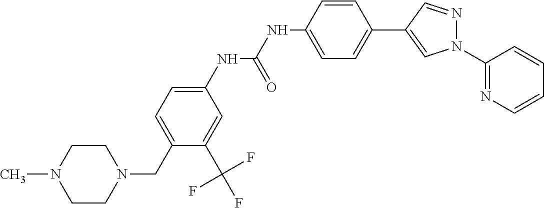 [SMILES: CN1CCN(Cc2ccc(NC(=O)Nc3ccc(-c4cnn(-c5ccccn5)c4)cc3)cc2C(F)(F)F)CC1]

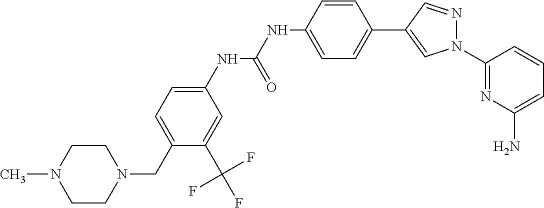 [SMILES: CN1CCN(Cc2ccc(NC(=O)Nc3ccc(-c4cnn(-c5cccc(N)n5)c4)cc3)cc2C(F)(F)F)CC1]

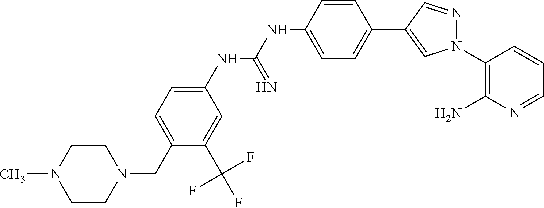 [SMILES: CN1CCN(Cc2ccc(NC(=N)Nc3ccc(-c4cnn(-c5cccnc5N)c4)cc3)cc2C(F)(F)F)CC1]

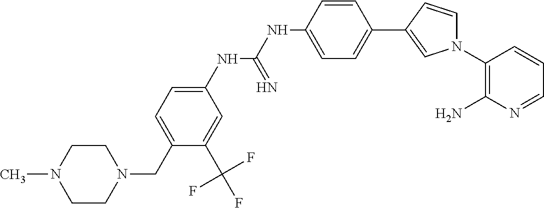 [SMILES: CN1CCN(Cc2ccc(NC(=N)Nc3ccc(-c4ccn(-c5cccnc5N)c4)cc3)cc2C(F)(F)F)CC1]

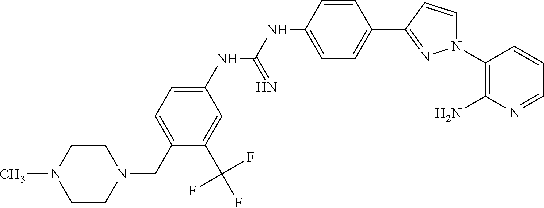 [SMILES: CN1CCN(Cc2ccc(NC(=N)Nc3ccc(-c4ccn(-c5cccnc5N)n4)cc3)cc2C(F)(F)F)CC1]

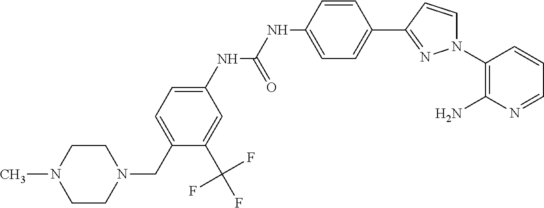 [SMILES: CN1CCN(Cc2ccc(NC(=O)Nc3ccc(-c4ccn(-c5cccnc5N)n4)cc3)cc2C(F)(F)F)CC1]

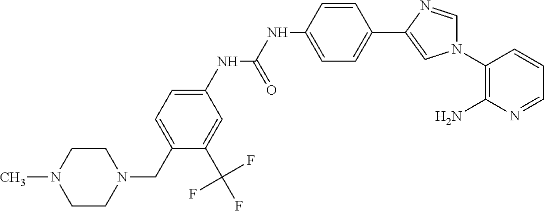 [SMILES: CN1CCN(Cc2ccc(NC(=O)Nc3ccc(-c4cn(-c5cccnc5N)cn4)cc3)cc2C(F)(F)F)CC1]

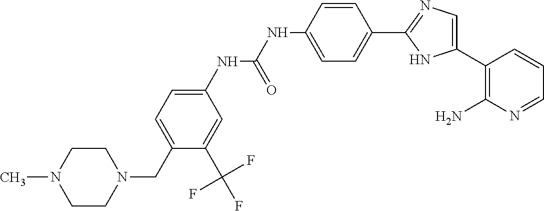 [SMILES: CN1CCN(Cc2ccc(NC(=O)Nc3ccc(-c4ncc(-c5cccnc5N)[nH]4)cc3)cc2C(F)(F)F)CC1]

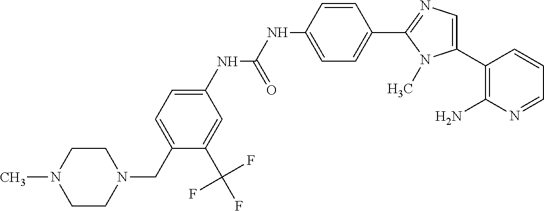 [SMILES: CN1CCN(Cc2ccc(NC(=O)Nc3ccc(-c4ncc(-c5cccnc5N)n4C)cc3)cc2C(F)(F)F)CC1]

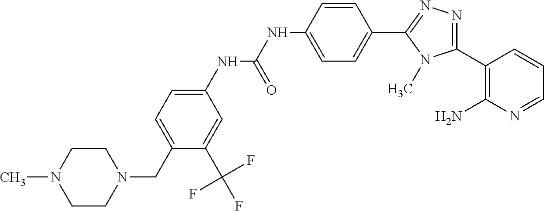 [SMILES: CN1CCN(Cc2ccc(NC(=O)Nc3ccc(-c4nnc(-c5cccnc5N)n4C)cc3)cc2C(F)(F)F)CC1]

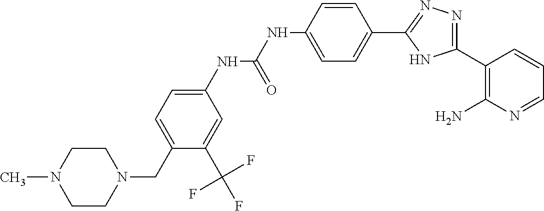 [SMILES: CN1CCN(Cc2ccc(NC(=O)Nc3ccc(-c4nnc(-c5cccnc5N)[nH]4)cc3)cc2C(F)(F)F)CC1]

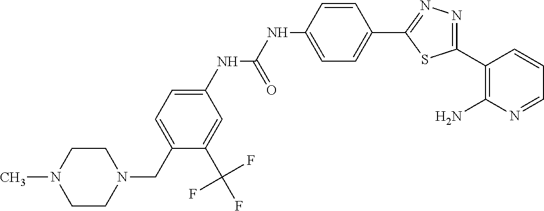 [SMILES: CN1CCN(Cc2ccc(NC(=O)Nc3ccc(-c4nnc(-c5cccnc5N)s4)cc3)cc2C(F)(F)F)CC1]

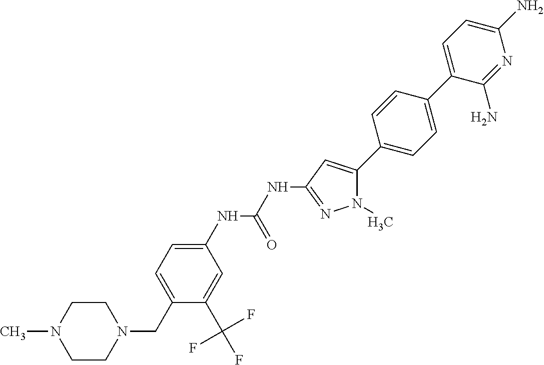 [SMILES: CN1CCN(Cc2ccc(NC(=O)Nc3cc(-c4ccc(-c5ccc(N)nc5N)cc4)n(C)n3)cc2C(F)(F)F)CC1]

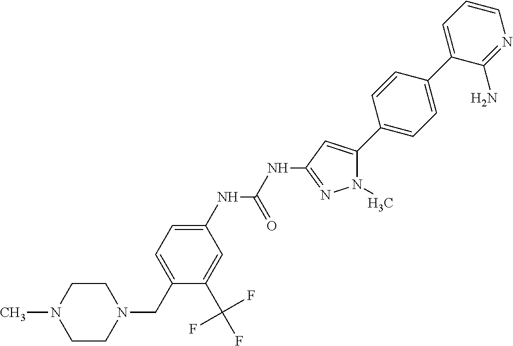 [SMILES: CN1CCN(Cc2ccc(NC(=O)Nc3cc(-c4ccc(-c5cccnc5N)cc4)n(C)n3)cc2C(F)(F)F)CC1]

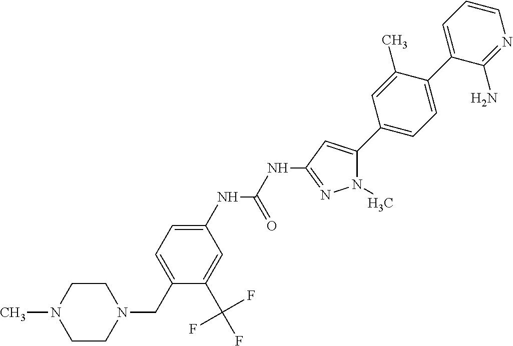 [SMILES: Cc1cc(-c2cc(NC(=O)Nc3ccc(CN4CCN(C)CC4)c(C(F)(F)F)c3)nn2C)ccc1-c1cccnc1N]

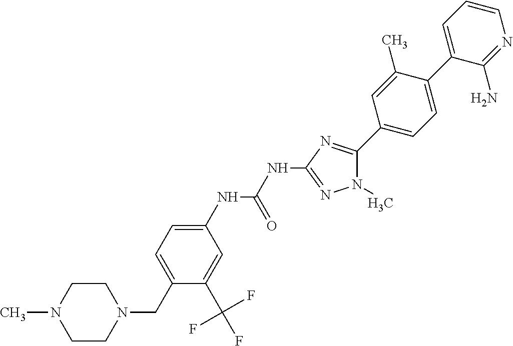 [SMILES: Cc1cc(-c2nc(NC(=O)Nc3ccc(CN4CCN(C)CC4)c(C(F)(F)F)c3)nn2C)ccc1-c1cccnc1N]

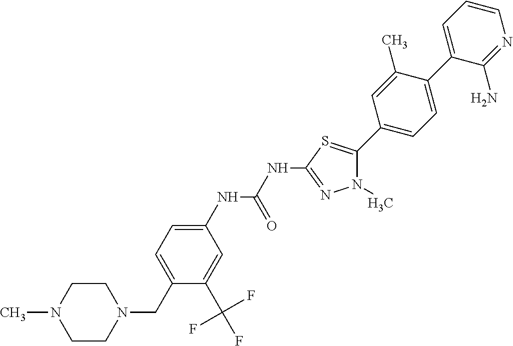

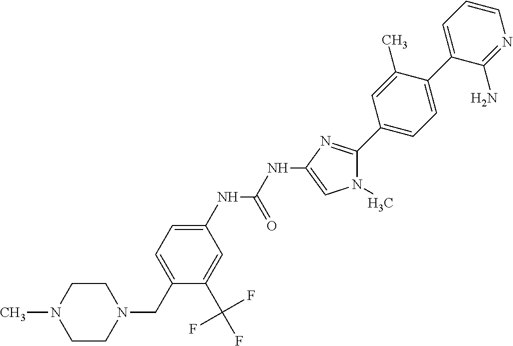 [SMILES: Cc1cc(-c2nc(NC(=O)Nc3ccc(CN4CCN(C)CC4)c(C(F)(F)F)c3)cn2C)ccc1-c1cccnc1N]

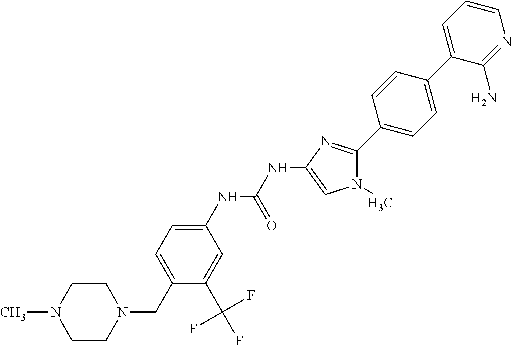 [SMILES: CN1CCN(Cc2ccc(NC(=O)Nc3cn(C)c(-c4ccc(-c5cccnc5N)cc4)n3)cc2C(F)(F)F)CC1]

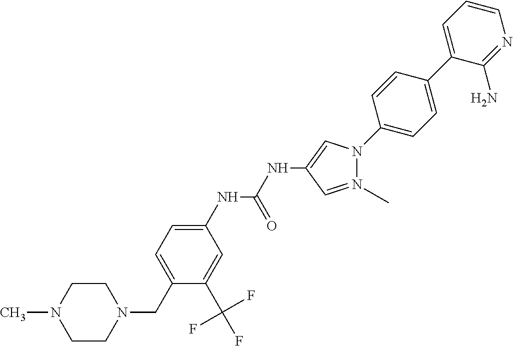

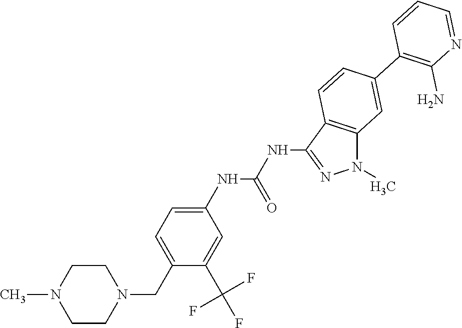 [SMILES: CN1CCN(Cc2ccc(NC(=O)Nc3nn(C)c4cc(-c5cccnc5N)ccc34)cc2C(F)(F)F)CC1]

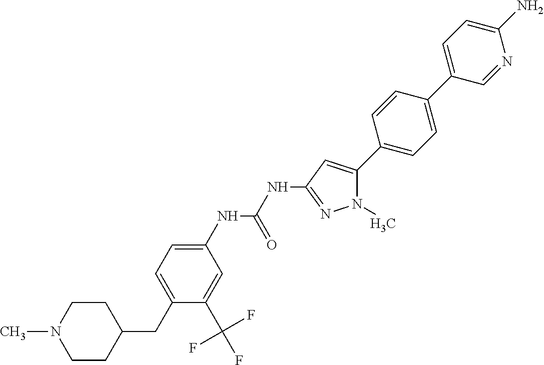 [SMILES: CN1CCC(Cc2ccc(NC(=O)Nc3cc(-c4ccc(-c5ccc(N)nc5)cc4)n(C)n3)cc2C(F)(F)F)CC1]

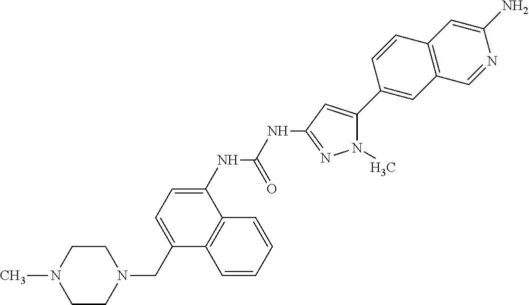 [SMILES: CN1CCN(Cc2ccc(NC(=O)Nc3cc(-c4ccc5cc(N)ncc5c4)n(C)n3)c3ccccc23)CC1]

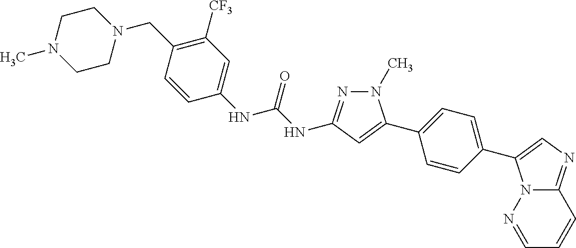 [SMILES: CN1CCN(Cc2ccc(NC(=O)Nc3cc(-c4ccc(-c5cnc6cccnn56)cc4)n(C)n3)cc2C(F)(F)F)CC1]

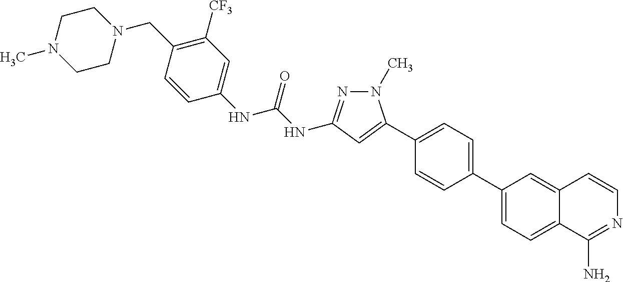 [SMILES: CN1CCN(Cc2ccc(NC(=O)Nc3cc(-c4ccc(-c5ccc6c(N)nccc6c5)cc4)n(C)n3)cc2C(F)(F)F)CC1]

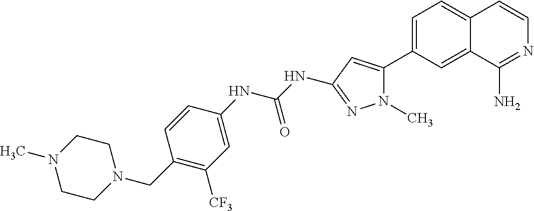 [SMILES: CN1CCN(Cc2ccc(NC(=O)Nc3cc(-c4ccc5ccnc(N)c5c4)n(C)n3)cc2C(F)(F)F)CC1]

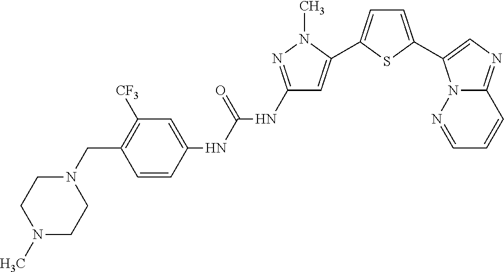 [SMILES: CN1CCN(Cc2ccc(NC(=O)Nc3cc(-c4ccc(-c5cnc6cccnn56)s4)n(C)n3)cc2C(F)(F)F)CC1]

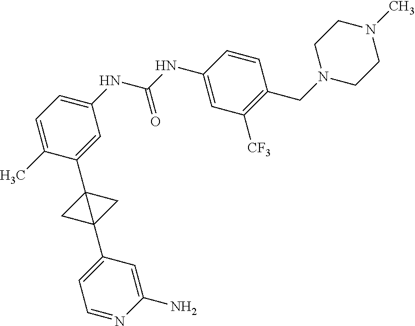

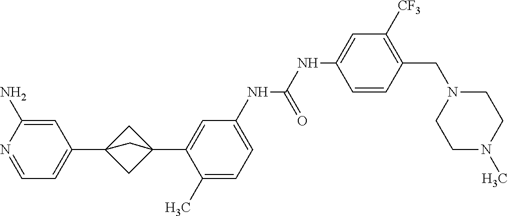 [SMILES: Cc1ccc(NC(=O)Nc2ccc(CN3CCN(C)CC3)c(C(F)(F)F)c2)cc1C12CC(c3ccnc(N)c3)(C1)C2]

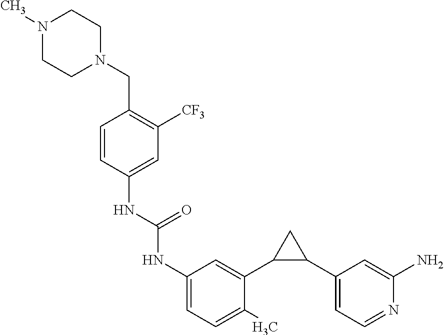 [SMILES: Cc1ccc(NC(=O)Nc2ccc(CN3CCN(C)CC3)c(C(F)(F)F)c2)cc1C1CC1c1ccnc(N)c1]

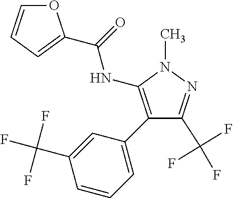 [SMILES: Cn1nc(C(F)(F)F)c(-c2cccc(C(F)(F)F)c2)c1NC(=O)c1ccco1]

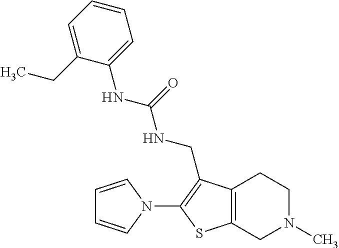 [SMILES: CCc1ccccc1NC(=O)NCc1c(-n2cccc2)sc2c1CCN(C)C2]

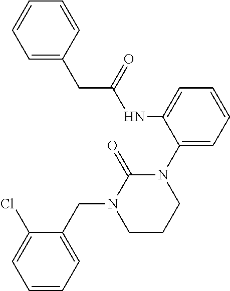 [SMILES: O=C(Cc1ccccc1)Nc1ccccc1N1CCCN(Cc2ccccc2Cl)C1=O]

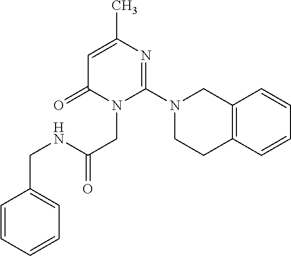 [SMILES: Cc1cc(=O)n(CC(=O)NCc2ccccc2)c(N2CCc3ccccc3C2)n1]

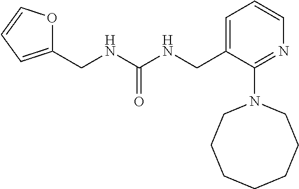 [SMILES: O=C(NCc1ccco1)NCc1cccnc1N1CCCCCCC1]

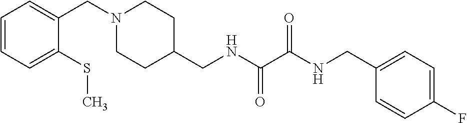 [SMILES: CSc1ccccc1CN1CCC(CNC(=O)C(=O)NCc2ccc(F)cc2)CC1]

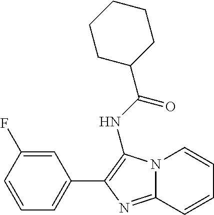 [SMILES: O=C(Nc1c(-c2cccc(F)c2)nc2ccccn12)C1CCCCC1]

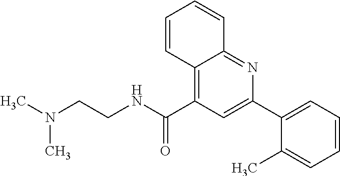 [SMILES: Cc1ccccc1-c1cc(C(=O)NCCN(C)C)c2ccccc2n1]

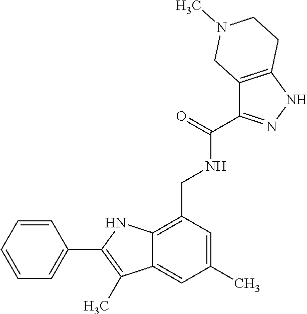 [SMILES: Cc1cc(CNC(=O)c2n[nH]c3c2CN(C)CC3)c2[nH]c(-c3ccccc3)c(C)c2c1]

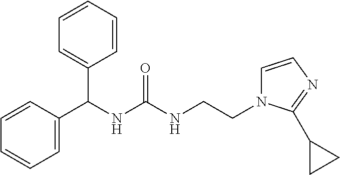 [SMILES: O=C(NCCn1ccnc1C1CC1)NC(c1ccccc1)c1ccccc1]

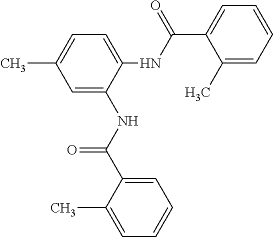 [SMILES: Cc1ccc(NC(=O)c2ccccc2C)c(NC(=O)c2ccccc2C)c1]

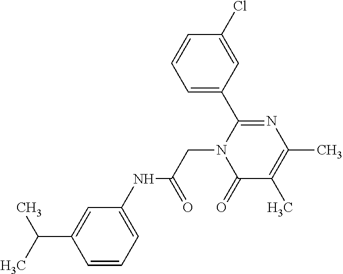 [SMILES: Cc1nc(-c2cccc(Cl)c2)n(CC(=O)Nc2cccc(C(C)C)c2)c(=O)c1C]

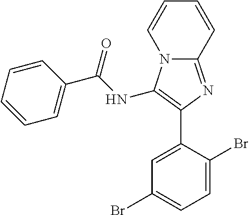 [SMILES: O=C(Nc1c(-c2cc(Br)ccc2Br)nc2ccccn12)c1ccccc1]

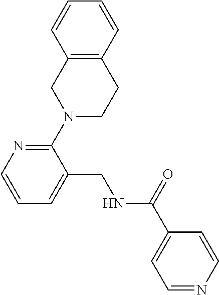 [SMILES: O=C(NCc1cccnc1N1CCc2ccccc2C1)c1ccncc1]

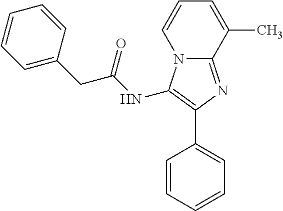 [SMILES: Cc1cccn2c(NC(=O)Cc3ccccc3)c(-c3ccccc3)nc12]

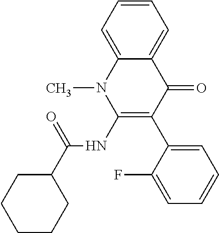 [SMILES: Cn1c(NC(=O)C2CCCCC2)c(-c2ccccc2F)c(=O)c2ccccc21]

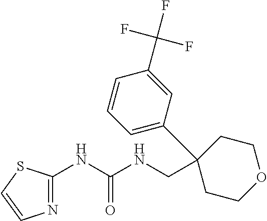 [SMILES: O=C(NCC1(c2cccc(C(F)(F)F)c2)CCOCC1)Nc1nccs1]

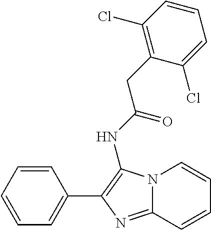 [SMILES: O=C(Cc1c(Cl)cccc1Cl)Nc1c(-c2ccccc2)nc2ccccn12]

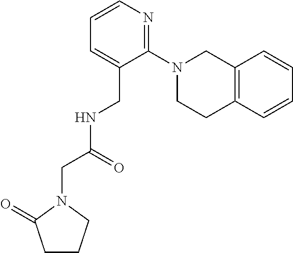 [SMILES: O=C(CN1CCCC1=O)NCc1cccnc1N1CCc2ccccc2C1]

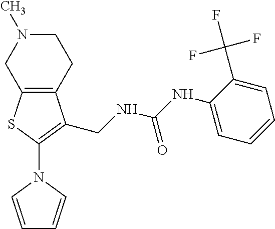 [SMILES: CN1CCc2c(sc(-n3cccc3)c2CNC(=O)Nc2ccccc2C(F)(F)F)C1]

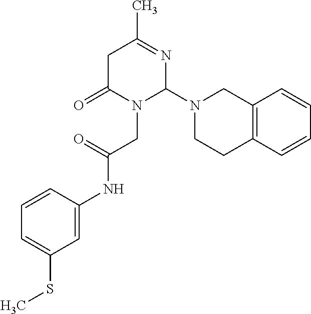 [SMILES: CSc1cccc(NC(=O)CN2C(=O)CC(C)=NC2N2CCc3ccccc3C2)c1]

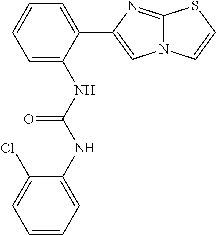 [SMILES: O=C(Nc1ccccc1Cl)Nc1ccccc1-c1cn2ccsc2n1]

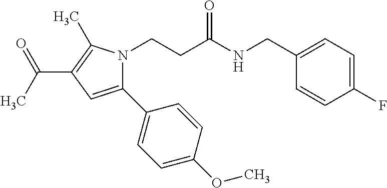 [SMILES: COc1ccc(-c2cc(C(C)=O)c(C)n2CCC(=O)NCc2ccc(F)cc2)cc1]

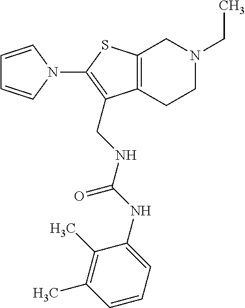 [SMILES: CCN1CCc2c(sc(-n3cccc3)c2CNC(=O)Nc2cccc(C)c2C)C1]

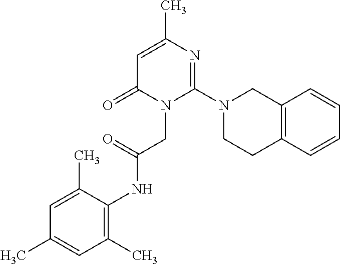 [SMILES: Cc1cc(C)c(NC(=O)Cn2c(N3CCc4ccccc4C3)nc(C)cc2=O)c(C)c1]

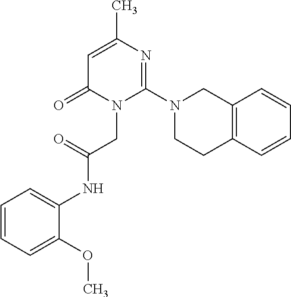 [SMILES: COc1ccccc1NC(=O)Cn1c(N2CCc3ccccc3C2)nc(C)cc1=O]

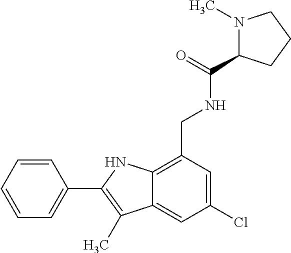 [SMILES: Cc1c(-c2ccccc2)[nH]c2c(CNC(=O)[C@@H]3CCCN3C)cc(Cl)cc12]

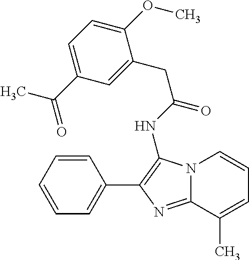 [SMILES: COc1ccc(C(C)=O)cc1CC(=O)Nc1c(-c2ccccc2)nc2c(C)cccn12]

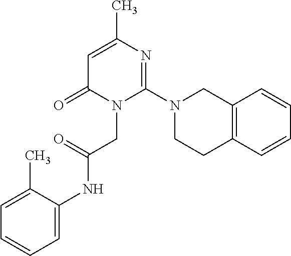 [SMILES: Cc1cc(=O)n(CC(=O)Nc2ccccc2C)c(N2CCc3ccccc3C2)n1]

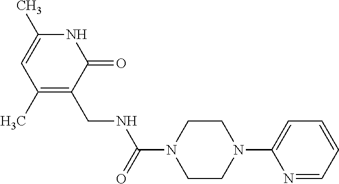 [SMILES: Cc1cc(C)c(CNC(=O)N2CCN(c3ccccn3)CC2)c(=O)[nH]1]

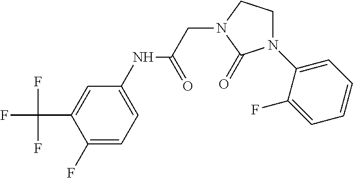 [SMILES: O=C(CN1CCN(c2ccccc2F)C1=O)Nc1ccc(F)c(C(F)(F)F)c1]

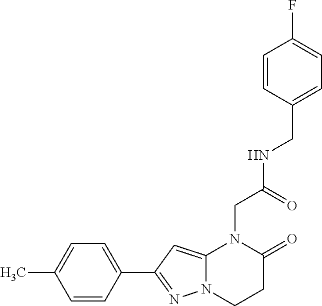 [SMILES: Cc1ccc(-c2cc3n(n2)CCC(=O)N3CC(=O)NCc2ccc(F)cc2)cc1]

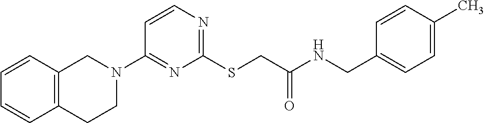 [SMILES: Cc1ccc(CNC(=O)CSc2nccc(N3CCc4ccccc4C3)n2)cc1]

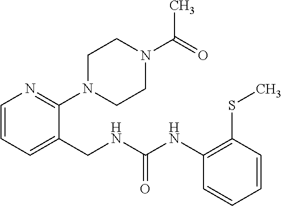 [SMILES: CSc1ccccc1NC(=O)NCc1cccnc1N1CCN(C(C)=O)CC1]

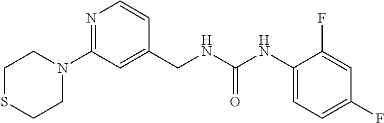 [SMILES: O=C(NCc1ccnc(N2CCSCC2)c1)Nc1ccc(F)cc1F]

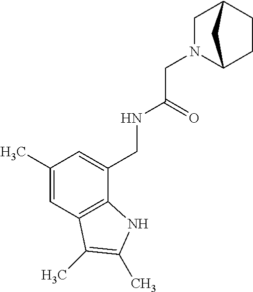 [SMILES: Cc1cc(CNC(=O)CN2C[C@@H]3CC[C@H]2C3)c2[nH]c(C)c(C)c2c1]

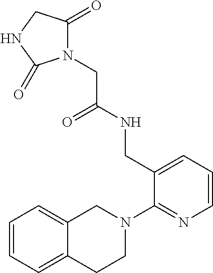 [SMILES: O=C(CN1C(=O)CNC1=O)NCc1cccnc1N1CCc2ccccc2C1]

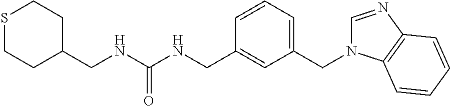 [SMILES: O=C(NCc1cccc(Cn2cnc3ccccc32)c1)NCC1CCSCC1]

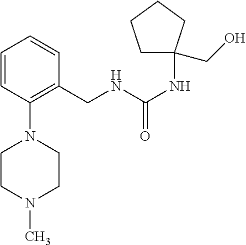 [SMILES: CN1CCN(c2ccccc2CNC(=O)NC2(CO)CCCC2)CC1]

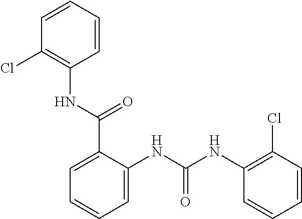 [SMILES: O=C(Nc1ccccc1Cl)Nc1ccccc1C(=O)Nc1ccccc1Cl]

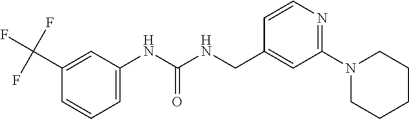 [SMILES: O=C(NCc1ccnc(N2CCCCC2)c1)Nc1cccc(C(F)(F)F)c1]

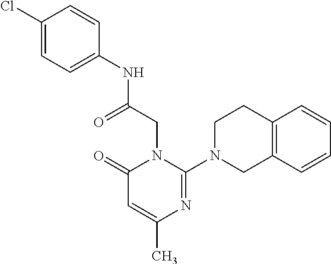 [SMILES: Cc1cc(=O)n(CC(=O)Nc2ccc(Cl)cc2)c(N2CCc3ccccc3C2)n1]

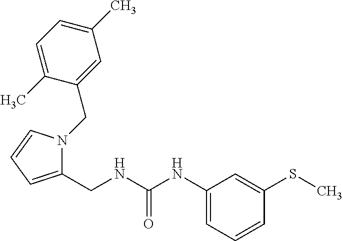 [SMILES: CSc1cccc(NC(=O)NCc2cccn2Cc2cc(C)ccc2C)c1]

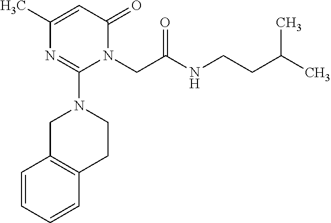 [SMILES: Cc1cc(=O)n(CC(=O)NCCC(C)C)c(N2CCc3ccccc3C2)n1]

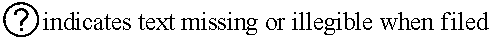

When a substituent is specified to be an atom or atoms of specified identity, “or a bond”, a configuration is referred to when the substituent is “a bond” that the groups that are immediately adjacent to the specified substituent are directly connected to each other by a chemically feasible bonding configuration.

In general, “optionally substituted” and “substituent” refers to an organic group as defined herein in which one or more bonds to a hydrogen atom contained therein are optionally replaced by one or more bonds to a non-hydrogen atom such as, but not limited to, a halogen (i.e., “halo” selected from F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboyxlate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR′, OC(O)N(R′)₂, CN, CF₃, OCF₃, R′, O, S, C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, (CH₂)₀₋₂N(R′)N(R′)₂, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further substituted. In some cases, the R′ group is a hydrogen, C₁-C₆ alkyl, or phenyl.

In many of the compounds described herein, the optional substituents are selected from amino, C₁-C₃ alkyl, ether, alkoxy, oxy, CF₃, and cyano C₁-C₃ alkoxy, benzyl, and benzaldehyde. The ether and alkoxy groups can have 1-6 carbon atoms.

Substituted alkyl, alkenyl, alkynyl, cycloalkyl, and cycloalkenyl groups as well as other substituted groups also include groups in which one or more bonds to a hydrogen atom are replaced by one or more bonds, including double or triple bonds, to a carbon atom, or to a heteroatom such as, but not limited to, oxygen in carbonyl (oxo), carboxyl, ester, amide, imide, urethane, and urea groups; and nitrogen in imines, hydroxyimines, oximes, hydrazones, amidines, guanidines, and nitriles.

Substituted ring groups such as substituted aryl, heterocyclyl and heteroaryl groups also include rings and fused ring systems in which a bond to a hydrogen atom is replaced with a bond to a carbon atom. Therefore, substituted aryl, heterocyclyl and heteroaryl groups can also be substituted with alkyl, alkenyl, cycloalkyl, aryl, heteroaryl, and alkynyl groups as defined herein, which can themselves be further substituted.

The term “heteroatoms” as used herein refers to non-carbon and non-hydrogen atoms, capable of forming covalent bonds with carbon, and is not otherwise limited. Typical heteroatoms are N, O, and S. When sulfur (S) is referred to, it is understood that the sulfur can be in any of the oxidation states in which it is found, thus including sulfoxides (R₃₀—S(O)—R₃₁) and sulfones (R₃₀—S(O)₂—R₃₁), unless the oxidation state is specified; thus, the term “sulfone” encompasses only the sulfone form of sulfur; the term “sulfide” encompasses only the sulfide (R₃₀—S—R₃₁) form of sulfur. When the phrases such as “heteroatoms selected from the group consisting of O, NH, NR₃₂ and S,” or “[variable] is O, S . . . ” are used, they are understood to encompass all of the sulfide, sulfoxide and sulfone oxidation states of sulfur.

Alkyl groups include straight chain and branched alkyl groups and cycloalkyl groups having from 1 to about 20 carbon atoms, and typically from 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, isobutyl, sec-butyl, t-butyl, neopentyl, isopentyl, and 2,2-dimethylpropyl groups. Representative substituted alkyl groups can be substituted one or more times with any of the groups listed above, for example, amino, hydroxy, cyano, carboxy, nitro, thio, alkoxy, and halogen groups.

An “alkylene” group refers to a divalent alkyl radical. Any of the above-mentioned monovalent alkyl groups may be an alkylene by abstraction of a second hydrogen atom from the alkyl. In some embodiments, an alkylene is a C₁-C₆alkylene. In some embodiments, an alkylene is a C₁-C₄alkylene. Examples of alkylene groups include, but are not limited to, —CH₂—, —CH₂CH₂—, —CH₂CH₂CH₂—, —CH₂CH₂CH₂CH₂—, and the like.

Cycloalkyl groups are alkyl groups forming a ring structure, which can be substituted or unsubstituted. Examples of cycloalkyl include, but are not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above.

Representative substituted cycloalkyl groups can be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups.

The terms “carbocyclic” and “carbocycle” denote a ring structure wherein the atoms of the ring are carbon. In some embodiments, the carbocycle has 3 to 8 ring members, whereas in other embodiments the number of ring carbon atoms is 4, 5, 6, or 7. Unless specifically indicated to the contrary, the carbocyclic ring can be substituted with as many as N substituents, wherein N is the number of atoms in the carbocyclic ring. Such substituents can, for example, be amino, hydroxy, cyano, carboxy, nitro, thio, alkoxy, and halogen groups.

(Cycloalkyl)alkyl groups, also denoted cycloalkylalkyl, are alkyl groups as defined above in which a hydrogen or carbon bond of the alkyl group is replaced with a bond to a cycloalkyl group as defined above.

Alkenyl groups include straight and branched chain and cyclic alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to —CH═CH(CH₃), —CH═C(CH₃)₂, —C(CH₃)═CH₂, —C(CH₃)═CH(CH₃), —C(CH₂CH₃)═CH₂, vinyl, cyclohexenyl, cyclopentenyl, cyclohexadienyl, butadienyl, pentadienyl, and hexadienyl among others.

The term “cycloalkenyl” alone or in combination denotes a cyclic alkenyl group wherein at least one double bond is present in the ring structure. Cycloalkenyl groups include cycloalkyl groups having at least one double bond between two adjacent carbon atoms. Thus, for example, cycloalkenyl groups include but are not limited to cyclohexenyl, cyclopentenyl, and cyclohexadienyl groups.

(Cycloalkenyl)alkyl groups are alkyl groups as defined above in which a hydrogen or carbon bond of the alkyl group is replaced with a bond to a cycloalkenyl group as defined above.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃), among others.

Aryl groups are cyclic aromatic hydrocarbons that do not contain heteroatoms. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain 6-14 carbons in the ring portions of the groups. The phrase “aryl groups” includes groups containing fused rings, such as fused aromatic-aliphatic ring systems (e.g., indanyl, tetrahydronaphthyl, and the like), and also includes substituted aryl groups that have other groups, including but not limited to alkyl, halo, amino, hydroxy, cyano, carboxy, nitro, thio, or alkoxy groups, bonded to one of the ring atoms. Representative substituted aryl groups can be mono-substituted or substituted more than once, such as, but not limited to, 2-, 3-, 4-, 5-, or 6-substituted phenyl or naphthyl groups, which can be substituted with groups including but not limited to those listed above.

Aralkyl groups are alkyl groups as defined above in which a hydrogen or carbon bond of an alkyl group is replaced with a bond to an aryl group as defined above. Representative aralkyl groups include benzyl and phenylethyl groups and fused (cycloalkylaryl)alkyl groups such as 4-ethyl-indanyl. The aryl moiety or the alkyl moiety or both are optionally substituted with other groups, including but not limited to alkyl, halo, amino, hydroxy, cyano, carboxy, nitro, thio, or alkoxy groups. Aralkenyl group are alkenyl groups as defined above in which a hydrogen or carbon bond of an alkyl group is replaced with a bond to an aryl group as defined above.

Heterocyclyl groups include aromatic and non-aromatic ring compounds containing 3 or more ring members, of which one or more is a heteroatom such as, but not limited to, N, O, S, or P. Heteroaryl and heterocyclicalkyl groups are included in the definition of heterocyclyl. In some embodiments, heterocyclyl groups include 3 to 20 ring members, whereas other such groups have 3 to 15 ring members. At least one ring contains a heteroatom, but every ring in a polycyclic system need not contain a heteroatom. For example, a dioxolanyl ring and a benzdioxolanyl ring system (methylenedioxyphenyl ring system) are both heterocyclyl groups within the meaning herein. A heterocyclyl group designated as a C₂-heterocyclyl can be a 5-ring with two carbon atoms and three heteroatoms, a 6-ring with two carbon atoms and four heteroatoms and so forth. Likewise, a C₄-heterocyclyl can be a 5-ring with one heteroatom, a 6-ring with two heteroatoms, and so forth. The number of carbon atoms plus the number of heteroatoms sums up to equal the total number of ring atoms. In some cases, the heterocyclyl is a single ring. In other cases, the heterocyclyl is a fusion of two or three rings. The phrase “heterocyclyl group” includes fused ring species including those having fused aromatic and non-aromatic groups. The phrase also includes polycyclic ring systems containing a heteroatom such as, but not limited to, quinuclidyl and also includes heterocyclyl groups that have substituents, including but not limited to alkyl, halo, amino, hydroxy, cyano, carboxy, nitro, thio, or alkoxy groups, bonded to one of the ring members. A heterocyclyl group as defined herein can be a heteroaryl group or a partially or completely saturated cyclic group including at least one ring heteroatom. Heterocyclyl groups include, but are not limited to, pyrrolidinyl, furanyl, tetrahydrofuranyl, dioxolanyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heterocyclyl groups can be substituted. Representative substituted heterocyclyl groups can be mono-substituted or substituted more than once, including but not limited to, rings containing at least one heteroatom which are mono, di, tri, tetra, penta, hexa, or higher-substituted with substituents such as those listed above, including but not limited to alkyl, halo, amino, hydroxy, cyano, carboxy, nitro, thio, and alkoxy groups.

Heteroaryl groups are aromatic ring compounds containing 5 or more ring members, of which, one or more is a heteroatom such as, but not limited to, N, O, and S. A heteroaryl group designated as a C₂-heteroaryl can be a 5-ring with two carbon atoms and three heteroatoms, a 6-ring with two carbon atoms and four heteroatoms and so forth. Likewise, a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, tetrahydroisoquinolinyl, quinoxalinyl, and quinazolinyl groups. The terms “heteroaryl” and “heteroaryl groups” include fused ring compounds such as wherein at least one ring, but not necessarily all rings, are aromatic, including tetrahydroquinolinyl, tetrahydroisoquinolinyl, indolyl and 2,3-dihydro indolyl. The term also includes heteroaryl groups that have other groups bonded to one of the ring members, including but not limited to alkyl, halo, amino, hydroxy, cyano, carboxy, nitro, thio, or alkoxy groups. Representative substituted heteroaryl groups can be substituted one or more times with groups such as those listed above.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

Heterocyclylalkyl groups are cyclic alkyl groups as defined above in which a hydrogen or carbon bond of an alkyl group is replaced with a bond to a heterocyclyl group as defined above. Representative heterocyclyl alkyl groups include, but are not limited to, furan-2-yl methyl, furan-3-yl methyl, pyridine-2-yl methyl (α-picolyl), pyridine-3-yl methyl (β-picolyl), pyridine-4-yl methyl (γ-picolyl), tetrahydrofuran-2-yl ethyl, and indol-2-yl propyl. Heterocyclylalkyl groups can be substituted on the heterocyclyl moiety, the alkyl moiety, or both.

Heteroarylalkyl groups are alkyl groups as defined above in which a hydrogen or carbon bond of an alkyl group is replaced with a bond to a heteroaryl group as defined above. Heteroarylalkyl groups can be substituted on the heteroaryl moiety, the alkyl moiety, or both.

By a “ring system” or “ring,” as the term is used herein, is meant a moiety comprising one, two, three or more rings, which can be substituted with non-ring groups or with other ring systems, or both, which can be fully saturated, partially unsaturated, fully unsaturated, or aromatic, and when the ring system includes more than a single ring, the rings can be fused, bridging, or spirocyclic. By “spirocyclic” is meant the class of structures wherein two rings are fused at a single tetrahedral carbon atom, as is well known in the art.

A “monocyclic, bicyclic or polycyclic, aromatic or partially aromatic ring” as the term is used herein refers to a ring system including an unsaturated ring possessing 4n+2 pi electrons, or a partially reduced (hydrogenated) form thereof. The aromatic or partially aromatic ring can include additional fused, bridged, or spiro rings that are not themselves aromatic or partially aromatic. For example, naphthalene and tetrahydronaphthalene are both a “monocyclic, bicyclic or polycyclic, aromatic or partially aromatic ring” within the meaning herein. Also, for example, a benzo-[2.2.2]-bicyclooctane is also a “monocyclic, bicyclic or polycyclic, aromatic or partially aromatic ring” within the meaning herein, containing a phenyl ring fused to a bridged bicyclic system. A fully saturated ring has no double bonds therein and is carbocyclic or heterocyclic depending on the presence of heteroatoms within the meaning herein.

The term “alkoxy” refers to an oxygen atom connected to an alkyl group, including a cycloalkyl group, as are defined above. Examples of linear alkoxy groups include but are not limited to methoxy, ethoxy, n-propoxy, n-butoxy, n-pentyloxy, n-hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like.

The terms “aryloxy” and “arylalkoxy” refer to, respectively, an aryl group bonded to an oxygen atom and an aralkyl group bonded to the oxygen atom at the alkyl moiety. Examples include but are not limited to phenoxy, naphthyloxy, and benzyloxy.

An “acyl” group as the term is used herein refers to a group containing a carbonyl moiety wherein the group is bonded via the carbonyl carbon atom. The carbonyl carbon atom is also bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. In cases where the carbonyl carbon atom is bonded to a hydrogen, the group is a “formyl” group, an acyl group as the term is defined herein. An acyl group can include 0 to about 12-20 additional carbon atoms bonded to the carbonyl group. An acyl group can include double or triple bonds within the meaning herein. An acryloyl group is an example of an acyl group. An acyl group can also include heteroatoms within the meaning here. A nicotinoyl group (pyridyl-3-carbonyl) group is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “amine” or “amino” includes primary, secondary, and tertiary amines having. e.g., the formula N(group)₃ wherein each group can independently be H or non-H, such as alkyl, aryl, and the like. Amines include but are not limited to R₄₀—NH₂, for example, alkylamines, arylamines, alkylarylamines; R₄₀NH wherein each R₄₀ is independently selected, such as dialkylamines, diarylamines, aralkylamines, heterocyclylamines and the like; and R₄₀N wherein each R₄₀ is independently selected, such as trialkylamines, dialkylarylamines, alkyldiarylamines, triarylamines, and the like. The term “amine” also includes ammonium ions as used herein.

An “amino” group is a substituent of the form —NH₂, —NHR₄₁, —N(R₄₁)₂, —N(R₄₁)₃⁺, wherein each R₄₁ is independently selected, and protonated forms of each. Accordingly, any compound substituted with an amino group can be viewed as an amine.

An “ammonium” ion includes the unsubstituted ammonium ion NH₄⁺, but unless otherwise specified, it also includes any protonated or quaternarized forms of amines. Thus, trimethylammonium hydrochloride and tetramethylammonium chloride are both ammonium ions, and amines, within the meaning herein.

The term “amide” (or “amido”) includes C- and N-amide groups, i.e., —C(O)N(R₄₂)₂, and —NRC(O)R₄₂— groups, respectively. Amide groups therefore include but are not limited to carbamoyl groups (—C(O)NH₂) and formamide groups (—NHC(O)H). A “carboxamido” group is a group of the formula C(O)N(R₄₂)₂, wherein R₄₂ can be H, alkyl, aryl, etc.

The term “urethane” (or “carbamyl”) includes N- and O-urethane groups, i.e., —NRC(O)OR₄₃ and —OC(O)N(R₄₃)₂ groups, respectively.

The term “sulfonamide” (or “sulfonamido”) includes S- and N-sulfonamide groups, i.e., —SO₂NR₄₄ and —NRSO₂R₄₄ groups, respectively. Sulfonamide groups therefore include but are not limited to sulfamoyl groups (—SO₂NH₂).

The term “amidine” or “amidino” includes groups of the formula —C(NR)N(R₄₅)₂. Typically, an amidino group is —C(NH)NH₂.

The term “guanidine” or “guanidino” includes groups of the formula —NRC(NR₄₆)N(R₄₆)₂. Typically, a guanidino group is —NHC(NH)NH₂.

“Halo,” “halogen.” and “halide” include fluorine, chlorine, bromine and iodine.

The terms “comprising.” “including,” “having.” “composed of,” are open-ended terms as used herein, and do not preclude the existence of additional elements or components. In a claim element, use of the forms “comprising,” “including,” “having,” or “composed of” means that whatever element is comprised, had, included, or composes is not necessarily the only element encompassed by the subject of the clause that contains that word.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein. The compounds of the present invention may take the form of salts. The term “salts” embraces addition salts of free acids or free bases which are compounds of the invention. Salts can be “pharmaceutically-acceptable salts.” The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention.

Suitable pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. Although pharmaceutically unacceptable salts are not generally useful as medicaments, such salts may be useful, for example as intermediates in the synthesis of compounds, for example in their purification by recrystallization. Any of these salts may be prepared from the corresponding compound by reacting, for example, the appropriate acid or base with the compound. The term “pharmaceutically acceptable salts” refers to nontoxic inorganic or organic acid and/or base addition salts, see, for example, Lit et al., Salt Selection for Basic Drugs (1986), Int J. Pharm., 33, 201-217, incorporated by reference herein.

A “hydrate” is a compound that exists in a composition with water molecules. The composition can include water in stoichiometric quantities, such as a monohydrate or a dihydrate, or can include water in random amounts. As the term is used herein a “hydrate” refers to a solid form, i.e., a compound in water solution, while it may be hydrated, is not a hydrate as the term is used herein.

A “solvate” is a similar composition except that a solvent other that water replaces the water. For example, methanol or ethanol can form an “alcoholate”, which can again be stoichiometric or non-stoichiometric. As the term is used herein a “solvate” refers to a solid form, i.e., a compound in solution in a solvent, while it may be solvated, is not a solvate as the term is used herein.

A “prodrug” as is well known in the art is a substance that can be administered to a patient where the substance is converted in vivo by the action of biochemicals within a mammal's body (e.g., in a patient's body), such as enzymes, to the active pharmaceutical ingredient. Examples of prodrugs include esters of carboxylic acid groups, which can be hydrolyzed by endogenous esterases as are found in the bloodstream of humans and other mammals.

In addition, where features or aspects of the invention are described in terms of Markush groups, those skilled in the art will recognize that the invention is also thereby described in terms of any individual member or subgroup of members of the Markush group. For example, if a variable (e.g., variable M) is described as selected from the group consisting of bromine, chlorine, and iodine, claims for M being bromine and claims for M being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if M is described as selected from the group consisting of bromine, chlorine, and iodine, and M₁ is described as selected from the group consisting of methyl, ethyl, and propyl, claims for M being bromine and M₁ being methyl are fully described.

In various embodiments, the compound or set of compounds, either per se or as are used in practice of embodiments of the inventive methods, can be any one of any of the combinations and/or sub-combinations of the various embodiments recited.

Provisos may apply to any of the disclosed categories or embodiments wherein any one or more of the other above disclosed embodiments or species may be excluded from such categories or embodiments.

### XBP1 and IRE1α

XBP1 is believed to sustain dendritic cell immunosuppressive activity within the tumor microenvironment by directly upregulating enzymes involved in triglyceride biosynthesis (Cubillos-Ruiz, et al., Cell 161(7): 1527-38 (2015)). XBP1, also known as X-box binding protein 1, is a transcription factor that regulates the expression of genes involved in the proper functioning of the immune system and in the cellular stress response. The inventors demonstrated that IRE1α-mediated XBP1 activation was fueled by the induction of reactive oxygen species and subsequent formation of peroxidized lipids.

The most conserved arm of the endoplasmic reticulum (ER) stress response is the dual enzyme, IRE1α. Activated during periods of ER stress, the IRE1α endoribonuclease domain excises a short nucleotide fragment from Xbp1 mRNA to generate the functional transcription factor, XBP1. This potent, multitasking protein promotes cell survival by upregulating expression of a broad range of critical genes involved in protein folding and quality control.

Unexpectedly, the inventors have demonstrated that modulating IRE1α or XBP1 can regulate the two rate limiting enzymes, Cox-2 and mPGES-1 in the prostaglandin biosynthetic pathway, which leads to a dramatic reduction in the production of prostaglandins such as PGE₂. Moreover, targeting IRE1α or XBP1 can also lead to reduction in cytokines like IL-6, IL-10, CXCL1 and RANTES. These features place IRE1α in a unique position to target diseases like pain, arthritis, fever, vascular permeability, hepatic lipogenesis, response to hypoxia, angiogenesis, atherosclerosis, allergies, and anti-tumor immunity. Moreover, targeting of this pathway in the setting of the tumor microenvironment also leads to reduction in PGE₂ biosynthesis. Additionally, IRE1α-mediated XBP1 signaling is also involved in production of prostaglandins such as prostaglandin E2 (PGE2).

Novel small-molecule IRE1α inhibitors are described herein with the ability to modulate prostaglandin levels and reduce pain responses. For example, small-molecule IRE1α inhibitors can be used to treat or inhibit pain in the animal. The pain that is treated or inhibited can be chronic pain, acute pain, inflammatory pain, somatic pain, visceral pain, neuropathic pain, and combinations thereof. In some embodiments, the pain that is treated is inflammatory pain. In other embodiments, the pain that is treated is somatic pain or visceral pain. In further embodiments, the origin of pain that is treated is unknown or arises from a combination of causes or pain types.

The disclosure also includes novel uses for vitamin E and hydralazine derivatives, which indirectly reduce IRE1α activation.

Hence, a method is described herein that includes administering any of the compounds or the composition described herein. The mammal can be in need of administration of the composition. For example, the mammal can have pain, inflammation, arthritis, liver dysfunction, brain ischemia, heart ischemia, or an autoimmune disease.

### Compositions

The IRE1α inhibitor compounds, their pharmaceutically acceptable salts or hydrolyzable esters of the present disclosure may be combined with a pharmaceutically acceptable carrier to provide pharmaceutical compositions useful for treating the biological conditions or disorders noted herein in mammalian species, and more preferably, in humans. The particular carrier employed in these pharmaceutical compositions may vary depending upon the type of administration desired (e.g. intravenous, oral, topical, suppository, or parenteral).

In preparing the compositions in oral liquid dosage forms (e.g. suspensions, elixirs and solutions), typical pharmaceutical media, such as water, glycols, oils, alcohols, flavoring agents, preservatives, coloring agents and the like can be employed. Similarly, when preparing oral solid dosage forms (e.g. powders, tablets and capsules), carriers such as starches, sugars, diluents, granulating agents, lubricants, binders, disintegrating agents and the like can be employed.

The instant disclosure provides compositions of the compounds, alone or in combination with another IRE1α inhibitor or another type of therapeutic agent, or both. For example, the compositions and methods described herein can include one or more agents such as vitamin E, an antioxidant, and/or hydralazine. Such compositions can be effective treatments for controlling pain and ER stress responses.

As set forth herein, compounds include stereoisomers, tautomers, solvates, hydrates, salts including pharmaceutically acceptable salts, and mixtures thereof. Compositions containing a compound can be prepared by conventional techniques, e.g. as described in Remington: The Science and Practice of Pharmacy, 19th Ed., 1995, incorporated by reference herein. The compositions can appear in conventional forms, for example capsules, tablets, aerosols, solutions, suspensions or topical applications.

Typical compositions include one or more compounds and a pharmaceutically acceptable excipient which can be a carrier or a diluent. For example, the active compound will usually be mixed with a carrier, or diluted by a carrier, or enclosed within a carrier which can be in the form of an ampoule, capsule, sachet, paper, or other container. When the active compound is mixed with a carrier, or when the carrier serves as a diluent, it can be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, tale, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent can include any sustained release material known in the art, such as glyceryl monostearate or glyceryl distearate, alone or mixed with a wax.

The formulations can be nixed with auxiliary agents which do not deleteriously react with the active compounds. Such additives can include wetting agents, emulsifying and suspending agents, salt for influencing osmotic pressure, buffers and/or coloring substances preserving agents, sweetening agents or flavoring agents. The compositions can also be sterilized if desired.

The route of administration can be any route which effectively transports the active compound which inhibits the activity of the IRE1α to the appropriate or desired site of action, such as oral, nasal, pulmonary, buccal, subdermal, intradermal, transdermal or parenteral, e.g., rectal, depot, subcutaneous, intravenous, intraurethral, intramuscular, intranasal, ophthalmic solution or an ointment, the oral route being preferred.

For parenteral administration, the carrier will typically comprise sterile water, although other ingredients that aid solubility or serve as preservatives can also be included. Furthermore, injectable suspensions can also be prepared, in which case appropriate liquid carriers, suspending agents and the like can be employed.

For topical administration, the compounds described herein can be formulated using bland, moisturizing bases such as ointments or creams.

If a solid carrier is used for oral administration, the preparation can be tableted, placed in a hard gelatin capsule in powder or pellet form or it can be in the form of a troche or lozenge. If a liquid carrier is used, the preparation can be in the form of a syrup, emulsion, soft gelatin capsule or sterile injectable liquid such as an aqueous or non-aqueous liquid suspension or solution.

Injectable dosage forms generally include aqueous suspensions or oil suspensions which can be prepared using a suitable dispersant or wetting agent and a suspending agent Injectable forms can be in solution phase or in the form of a suspension, which is prepared with a solvent or diluent. Acceptable solvents or vehicles include sterilized water, Ringer's solution, or an isotonic aqueous saline solution. Alternatively, sterile oils can be employed as solvents or suspending agents. Preferably, the oil or fatty acid is non-volatile, including natural or synthetic oils, fatty acids, mono-, di- or tri-glycerides.

For injection, the formulation can also be a powder suitable for reconstitution with an appropriate solution as described above. Examples of these include, but are not limited to, freeze dried, rotary dried or spray dried powders, amorphous powders, granules, precipitates, or particulates. For injection, the formulations can optionally contain stabilizers. pH modifiers, surfactants, bioavailability modifiers and combinations of these. The compounds can be formulated for parenteral administration by injection such as by bolus injection or continuous infusion. A unit dosage form for injection can be in ampoules or in multi-dose containers.

The formulations can be designed to provide quick, sustained, or delayed release of the active ingredient after administration to the patient by employing procedures well known in the art. Thus, the formulations can also be formulated for controlled release or for slow release.

Compositions contemplated herein can include, for example, micelles or liposomes, or some other encapsulated form, or can be administered in an extended release form to provide a prolonged storage and/or delivery effect. Therefore, the formulations can be compressed into pellets or cylinders and implanted intramuscularly or subcutaneously as depot injections. Such implants can employ known inert materials such as silicones and biodegradable polymers, e.g., polylactide-polyglycolide. Examples of other biodegradable polymers include poly(orthoesters) and poly(anhydrides).

For nasal administration, the preparation can contain a compound which inhibits the enzymatic activity of the focal adhesion kinase, dissolved or suspended in a liquid carrier, preferably an aqueous carrier, for aerosol application. The carrier can contain additives such as solubilizing agents, e.g., propylene glycol, surfactants, absorption enhancers such as lecithin (phosphatidylcholine) or cyclodextrin, or preservatives such as parabens.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet that can be prepared by conventional tableting techniques can contain, for example, components listed in Table 8.

TABLE 8
Example of a Tablet Formulation
Core:

| | | |
|---|---|---|
| Active compound (as free compound or | 100-500 | mg |
| salt thereof) | | |
| Colloidal silicon dioxide (Aerosil) ® | 1.5 | mg |
| Cellulose, microcryst. (Avicel) ® | 70 | mg |
| Modified cellulose gum (Ac-Di-Sol) ® | 7.5 | mg |

| | |
|---|---|
| Magnesium stearate | Ad. |

Coating:

| | | |
|---|---|---|
| HPMC approx. | 9 | mg |
| *Mywacett 9-40 T approx. | 0.9 | mg |

*Acylated monoglyceride used as plasticizer for film coating

A typical capsule for oral administration contains compounds (250 mg), lactose (75 mg) and magnesium stearate (15 mg). The mixture is passed through a 60-mesh sieve and packed into a No, 1 gelatin capsule. A typical injectable preparation is produced by aseptically placing 100-500 mg (e.g., 250 mg) of one or more compounds into a vial, aseptically freeze-drying and sealing. For use, the contents of the vial are mixed with 2 mL of sterile physiological saline, to produce an injectable preparation.

The compounds can be administered to an animal or a human in need of such treatment, prevention, elimination, alleviation or amelioration of a malcondition that is mediated through the action of IRE1α, for example, pain, fever, vascular permeability, inflammation, arthritis, cancer, neurodegenerative diseases, metabolic disorders, liver dysfunction, brain ischemia, or heart ischemia.

The pharmaceutical compositions and compounds described herein can generally be administered in the form of a dosage unit (e.g. tablet, capsule, etc.) in an amount from about 1 ng/kg of body weight to about 0.5 g/kg of body weight, or from about 1 μ/kg of body weight to about 500 mg/kg of body weight, or from about 10 μ/kg of body weight to about 250 mg/kg of body weight, most preferably from about 20 μ/kg of body weight to about 100 mg/kg of body weight. Those skilled in the art will recognize that the particular quantity of pharmaceutical composition and/or compounds described herein administered to an individual will depend upon a number of factors including, without limitation, the biological effect desired, the condition of the individual and the individual's tolerance for the compound.

The compounds are effective over a wide dosage range. For example, in the treatment of adult humans, dosages from about 0.05 to about 5000 mg, preferably from about 1 to about 2000 mg, and more preferably between about 2 and about 2000 mg per day can be used. A typical dosage is about 10 mg to about 1000 mg per day. In choosing a regimen for patients it can frequently be necessary to begin with a higher dosage and when the condition is under control to reduce the dosage. The exact dosage will depend upon the activity of the compound, mode of administration, on the therapy desired, form in which administered, the subject to be treated and t body weight of the subject to be treated, and the preference and experience of the physician or veterinarian in charge. IRE1α inhibitor bioactivity of the compounds can be determined by use of an in vitro assay system which measures the activity of IRE1α, which can be expressed as EC₅₀ or IC₅₀ values, as are well known in the art inhibitors can be determined by the method described in the Examples.

Generally, the compounds are dispensed in unit dosage form including from about 0.05 mg to about 1000 mg of active ingredient together with a pharmaceutically acceptable carrier per unit dosage.

Usually, dosage forms suitable for oral, nasal, pulmonal or transdermal administration include from about 125 μg to about 1250 mg, preferably from about 250 μg to about 500 mg, and more preferably from about 2.5 mg to about 250 mg, of the compounds admixed with a pharmaceutically acceptable carrier or diluent.

Dosage forms can be administered daily, or more than once a day, such as twice or thrice daily. Alternatively, dosage forms can be administered less frequently than daily, such as every other day, or weekly, if found to be advisable by a prescribing physician.

Prodrugs of a compound which, on administration, undergo chemical conversion by metabolic or other physiological processes before becoming active pharmacological substances are contemplated herein. Conversion by metabolic or other physiological processes includes without limitation enzymatic (e.g., specific enzymatically catalyzed) and non-enzymatic (e.g., general or specific acid or base induced) chemical transformation of the prodrug into the active pharmacological substance. In general, such prodrugs will be functional derivatives of a compound which are readily convertible in vivo into a compound. Conventional procedures for the selection and preparation of suitable prodrug derivatives are described, for example, in Design of Prodrugs, ed. H. Bundgaard, Elsevier, 1985.

There are provided methods of making a composition of a compound described herein including formulating a compound with a pharmaceutically acceptable carrier or diluent. The pharmaceutically acceptable carrier or diluent is suitable for oral administration. The methods can further include the step of formulating the composition into a tablet or capsule. Or the pharmaceutically acceptable carrier or diluent is suitable for parenteral administration. The methods can further include the step of lyophilizing the composition to form a lyophilized preparation.

The compounds can be used therapeutically in combination with i) one or more other IRE1α inhibitors and/or ii) one or more other types of protein kinase inhibitors and/or one or more other types of therapeutic agents which can be administered orally in the same dosage form, in a separate oral dosage form (e.g., sequentially or non-sequentially) or by injection together or separately (e.g., sequentially or non-sequentially).

The disclosure provides combinations, comprising:

a) a compound as described herein; and

b) one or more compounds comprising:

i) other compounds described herein.

ii) other agents or medicaments adapted for treatment of a disease or malcondition for which inhibition of IRE1α is medically indicated, for example, vitamin E, an antioxidant, hydralazine, or any combination thereof. Such compounds, agents or medicaments can be medically indicated for treatment of inflammation, cancers, neurodegenerative diseases, metabolic disorders, liver dysfunction, autoimmune diseases, brain ischemia, or heart ischemic.

Combinations include mixtures of compounds from (a) and (b) in a single formulation and compounds from (a) and (b) as separate formulations. Some combinations can be packaged as separate formulations in a kit. Two or more compounds from (b) can be formulated together while another compound can be formulated separately.

The dosages and formulations for the other agents to be employed, where applicable, will be as set out in the latest edition of the Physicians' Desk Reference, incorporated herein by reference.

### Pain

The compositions and methods herein are useful for treating and/or reducing pain. All types of pain can be treated with the compositions and methods, including chronic pain, acute pain (e.g., nociceptive pain), inflammatory pain, somatic pain, visceral pain, neuropathic pain, and combinations thereof.

There are primarily three types of pain: somatic, visceral and neuropathic, all of which can be acute and chronic.

Somatic pain is typically caused by the activation of pain receptors in either the cutaneous or musculoskeletal tissues. In contrast to surface somatic pain which is usually described as sharp and may have a burning or pricking quality, deep somatic pain is usually characterized as a dull, aching but localized sensation. Somatic pain may include fractures in the vertebrae, joint pain (deep somatic pain) and postsurgical pain from a surgical incision (surface pain). Thus, the pain to be treated can be a form of somatic pain.

Visceral pain is caused by activation of pain receptors in internal areas of the body that are enclosed within a cavity. Visceral pain is usually described as pressure-like, poorly localized and deep. Therefore, the pain to be treated can be a form of visceral pain.

Neuropathic pain, caused by neural damage, is usually described as burning, tingling, shooting or stinging but can also manifest itself as sensory loss either as a result of compression, infiltration, chemical or metabolic damage or is idiopathic. Examples of neuropathic pain are heterogenous and include medication-induced neuropathy and nerve compression syndromes such as carpal tunnel, radiculopathy due to vertebral disk herniation, post-amputation syndromes such as stump pain and phantom limb pain, metabolic disease such as diabetic neuropathy, neurotropic viral disease from herpes zoster and human immunodeficiency virus (HIV) disease, tumor infiltration leading to irritation or compression of nervous tissue, radiation neuritis, as after cancer radiotherapy, and autonomic dysfunction from complex regional pain syndrome (CRPS). Thus, the pain to be treated can be a form of neuropathic pain.

Inflammatory pain is related to tissue damage which can occur in the form of penetration wounds, burns, extreme cold, fractures, inflammatory arthropathies as seen in many autoimmune conditions, excessive stretching, infections, vasoconstriction and cancer. The pain to be treated can therefore be a form of inflammatory pain.

The chronic pain can be due to problems such as arthritis, cancer, injuries, HIV, and the like. According to the invention, the compositions and methods can treat chronic pain.

Acute pain, termed nociception, is the instantaneous onset of a painful sensation in response to a noxious stimulus. It is considered to be adaptive because it can prevent an organism from damaging itself. For example, removing a hand from a hot stove as soon as pain is felt can prevent serious burns. The second type of pain is persistent pain. Unlike acute pain, it usually has a delayed onset but can last for hours to days. It is predominately considered adaptive because the occurrence of persistent pain following injury can prevent further damage to the tissue. For example, the pain associated with a sprained ankle will prevent the patient from using the foot, thereby preventing further trauma and aiding healing. A third category of pain is chronic pain. It has a delayed onset and can last for months to years. In contrast to acute and persistent pain, chronic pain is considered maladaptive and is associated with conditions such as arthritis, nerve injury, AIDS and diabetes. Yet another type of pain can be termed breakthrough pain. This is a brief flare-up of severe pain lasting from minutes to hours that can occur in the presence or absence of a preceding or precipitating factor even while the patient is regularly taking pain medication. Many patients experience a number of episodes of breakthrough pain each day. The pain to be treated with the compositions and methods described herein can be acute pain.

According to the invention, pain can be treated or inhibited in an animal. As used herein an animal is a mammal or a bird. Thus, animals that can be treated using the compositions and/or methods of the invention include humans, domesticated animals, experimental animals and zoo animals. For example, animals that can be treated using the compositions and/or methods of the invention include humans, dogs, cats, horses, pigs, cattle, goats, mice, rats, rabbits, and the like.

The Examples illustrate some of experimental work performed in the development of the invention.

### Example 1: Materials and Methods

This Example describes some of the materials and methods employed in the development of the invention.

### RNA Isolation, Quantitative RT-PCR and Xbp1 Splicing Assays

Total RNA was isolated using RNeasy Mini kit or QIAzol lysis reagent (Qiagen) according to the manufacturer's instructions. RNA (0.1-1 μg) was reverse-transcribed to generate cDNA using the qScript cDNA synthesis kit (Quantabio). Quantitative RT-PCR was performed using PerfeCTa SYBR green fastmix (Quantabio) and TaqMan Universal PCR master mix (Life Technologies) on a QuantStudio 6 Flex real-time PCR system (Applied Biosystems). Normalized gene expression was calculated by comparative threshold cycle method using ACTB or Actb as a control. Xbp1 splicing assays were performed as described by Lee et al. (Proc Natl Acad Sci USA 100, 9946 (Aug. 19, 2003)). PCR products were separated by electrophoresis through a 2.5% agarose gel and visualized by ethidium bromide staining. Primers used in this study are described in Table 9.

TABLE 9
Primer Sequences

| | | Primer | Sequence |
|---|---|---|---|
| Species | Gene | direction | 5′-3′ |
| mouse | Actb | Forward | CTCAG |
| | | | GAGGA |
| | | | CCAAT |
| | | | GATCT |
| | | | TGAT |
| | | | (SEQ ID |
| | | | NO: 1) |
| | | Reverse | TACCA |
| | | | CCATG |
| | | | TACCC |
| | | | AGGCA |
| | | | (SEQ ID |
| | | | NO: 2) |
| mouse | Xbp1s | Forward | AAGAA |
| | | | CACGC |
| | | | TTGGG |
| | | | AATGG |
| | | | (SEQ ID |
| | | | NO: 3) |
| | | Reverse | CTGCA |
| | | | CCTGC |
| | | | TGCGG |
| | | | AC |
| | | | (SEQ ID |
| | | | NO: 4) |
| mouse | Xbp1 | Forward | ACACG |
| | | | TTTGG |
| | | | GAATG |
| | | | OACAC |
| | | | (SEQ ID |
| | | | NO: 5) |
| | | Reverse | CCATG |
| | | | GGAAG |
| | | | ATGTT |
| | | | CTGGG |
| | | | (SEQ ID |
| | | | NO: 6) |
| mouse | Ptgs1 | Forward | CITAA |
| | | | GTACC |
| | | | AGGTG |
| | | | CTGGA |
| | | | CG |
| | | | (SEQ ID |
| | | | NO: 7) |
| | | Reverse | GGTGG |
| | | | GTAGC |
| | | | GCATC |
| | | | AACAC |
| | | | (SEQ ID |
| | | | NO: 8) |
| mouse | Ptgs2 | Forward | TGGCT |
| | | | GTGAA |
| | | | GGGAA |
| | | | ATAAG |
| | | | GAG |
| | | | (SEQ ID |
| | | | NO: 9) |
| | | Reverse | ATTTG |
| | | | AGCCT |
| | | | TGGGG |
| | | | GTCAG |
| | | | (SEQ ID |
| | | | NO: 10) |
| mouse | Ptges | Forward | AGCAC |
| | | | ACTGC |
| | | | TGGTC |
| | | | ATCAA |
| | | | (SEQ ID |
| | | | NO: 11) |
| | | Reverse | TTGGC |
| | | | AAAAG |
| | | | CCTTC |
| | | | TTCCG |
| | | | C(SEQ ID |
| | | | NO:12) |
| mouse | Ptges2 | Forward | CTTGC |
| | | | TGACC |
| | | | TGGCA |
| | | | GTGTA |
| | | | TG |
| | | | (SEQ ID |
| | | | NO: 13) |
| | | Reverse | TGTGA |
| | | | GTGTC |
| | | | GCATC |
| | | | AGGTC |
| | | | (SEQ ID |
| | | | NO: 14) |
| HUMAN | ACTB | Forward | GCGAG |
| | | | AAGAT |
| | | | GACCC |
| | | | AGATC |
| | | | (SEQ ID |
| | | | NO: 15) |
| | | Reverse | CCAGT |
| | | | GGTAC |
| | | | GGCCA |
| | | | GAGG |
| | | | (SEQ ID |
| | | | NO: 16) |
| HUMAN | XBP1s | Forward | AACCA |
| | | | GGAGT |
| | | | TAAGA |
| | | | CAGCG |
| | | | CTT |
| | | | (SEQ ID |
| | | | NO: 17) |
| | | Reverse | CTGCA |
| | | | CCCTC |
| | | | TGCGG |
| | | | ACT |
| | | | (SEQ ID |
| | | | NO: 18) |
| HUMAN | PTGS2 | Forward | GAATG |
| | | | GGGTG |
| | | | ATGAG |
| | | | CAGTT |
| | | | (SEQ ID |
| | | | NO: 19) |
| | | Reverse | CAGAA |
| | | | GGGCA |
| | | | GGATA |
| | | | CAGC |
| | | | (SEQ ID |
| | | | NO: 20) |
| HUMAN | PTGES | Forward | CCTAA |
| | | | CCCTT |
| | | | TTGTC |
| | | | GCCTG |
| | | | (SEQ ID |
| | | | NO: 21) |
| | | Reverse | CAGGT |
| | | | AGGCC |
| | | | ACGGT |
| | | | GTGT (SEQ ID |
| | | | NO: 22) |
| HUMAN | PTGS2 | Forward | TCCTA |
| | X2-Box | | TGAAG |
| | distal | | GGCTA |
| | (−984/−829) | | GTAAC |
| | | | CAA |
| | | | (SEQ ID |
| | | | NO: 23) |
| | | Reverse | TCCAC |
| | | | GGGTC |
| | | | ACCAA |
| | | | TATAA |
| | | | A |
| | | | (SEQ ID |
| | | | NO: 24) |
| HUMAN | PTGS2 | Forward | AACCT |
| | X2-Box | | TACTC |
| | proximal | | GCCCC |
| | (−531/−376) | | AGTCT |
| | | | (SEQ ID |
| | | | NO: 25) |
| | | Reverse | CAGAA |
| | | | GGACA |
| | | | CTTGG |
| | | | CTTCC |
| | | | (SEQ ID |
| | | | NO: 26) |
| HUMAN | PTGES | Forward | TCTTT |
| | X2-Box | | CGGGG |
| | distal | | AGATC |
| | (−1088/−882) | | TTGTG |
| | | | (SEQ ID |
| | | | NO: 27) |
| | | Reverse | TOAGA |
| | | | CCCAT |
| | | | TTCAG |
| | | | GCTTC |
| | | | (SEQ ID |
| | | | NO: 28) |
| HUMAN | PTGES | Forward | CTCCA |
| | X2-Box | | TTGTC |
| | medial | | CAGGC |
| | (−435/−209) | | TGAGT |
| | | | (SEQ ID |
| | | | NO: 29) |
| | | Reverse | TTCCA |
| | | | GGCAA |
| | | | ATCCT |
| | | | CAAAC |
| | | | (SEQ ID |
| | | | NO: 30) |
| HUMAN | GFPT1 | Forward | GAGTT |
| | X2-Box | | TCTCC |
| | (−254/−52) | | CTCCC |
| | | | TCTC |
| | | | (SEQ ID |
| | | | NO: 31) |
| | | Reverse | GCTCC |
| | | | ATTGA |
| | | | ACCGC |
| | | | TCAC |
| | | | (SEQ ID |
| | | | NO: 32) |
| HUMAN | Pri-miR-21 | Forward | CATTG |
| | (2725/2920) | | TGGGT |
| | | | TTTGA |
| | | | AAAGG |
| | | | TTA |
| | | | (SEQ ID |
| | | | NO: 33) |
| | | Reverse | ATGAA |
| | | | CCACG |
| | | | ACTAG |
| | | | AGGCT |
| | | | GACTT |
| | | | (SEQ ID |
| | | | NO: 34) |

### Transgenic Mice

Atf6^{f/f}, Eij2ak3^{f/f}, Vav1^{cre} and CD11c^{cre} mice were obtained from The Jackson Laboratory. Xbp1^{f/f} and Ern1^{f/f} mice have been previously described by the inventors (Lee et al. Science 320, 1492 (Jun. 13, 2008); Iwawaki et al. Proc Natl Acad Sci USA 106, 16657 (Sep. 29, 2009)). Conditional knockout mice lacking XBP1, IRE1α or ATF6 in leukocytes were generated by crossing Xbp1^{f/f}, Ern1^{f/f} or Atf6^{f/f} animals, respectively, with the Vav1cre strain that allows selective gene deletion in hematopoietic cells (de Boer et al. Eur J Immunol 33, 314 (February 2003)). Crossing Eif2ak3^{f/f} mice with CD11c^{cre} animals generated mice devoid of PERK in dendritic cells (DC). All mouse strains had a full C57BL/6 background. Mice were housed in specific pathogen-free animal facilities at Weill Cornell Medical College. Memorial Sloan Kettering Cancer Center, and Wake Forest University. Mice were handled in compliance with Weill Cornell Institutional Animal Care and Use Committees procedures. Mice used for behavioral pain tests were housed at Wake Forest School of Medicine, in accordance with the Wake Forest University Guidelines on the ethical use of animals. The Institutional Animal Care and Use Committee of Wake Forest University approved all pain-related experiments. Animals were housed under a 12-h light-dark cycle, with food and water ad libitum.

### Primary Cell Isolation and Generation

Murine dendritic cells were generated by incubation of flushed, single suspended, bone marrow cells isolated from mice of the indicated genotypes in complete RPMI media (RPMI+L-glutamine+10% FBS+HEPES+Sodium Pyruvate+non-essential amino acids+β mercaptoethanol+Pen/strep) containing 10% FBS and 20 ng/ml of recombinant granulocyte macrophage colony-stimulating factor (GM-CSF) (Gemini or Peprotech). Media was replenished on day 6, and cells were harvested on day 7 and used directly for subsequent in vitro functional assays.

Human monocyte-derived DC were generated by isolating CD14⁺ cells (Miltenyi, catalog number 130-050-201) from blood/buffy coats using a Ficoll-gradient centrifugation and plated in complete RPMI media containing 10% FBS and human recombinant GM-CSF (Peprotech) at 1000 IU/ml and IL-4 (Peprotech) at 500 IU/ml for 7 days. Cells were then harvested and used for subsequent in vitro assays (Nair et al. Curr Protoc Immunol Chapter 7, Unit7 32 (November 2012)).

Mouse primary macrophages were generated by incubation of flushed, single suspended, bone marrow cells from mice of the indicated genotypes in media (DMEM F12 50/50 mix+L-glutamine+10% FBS+Pen/strep) with 20 ng/ml recombinant M-CSF (Peprotech) and 1 ng/ml recombinant IL-3 (Peprotech) for 3 days in bacteriological plates. On day 4, non-adherent cells were washed and plated in tissue culture-treated dishes at 1×105 cells/ml in media containing 20 ng/ml recombinant M-CSF. On day 6, media was replaced, and cells were harvested and used for stimulation on day 7.

Primary neutrophils were isolated directly from the bone marrow of Ern1^{f/f} or Ern1^{f/f} Vav1cre mice using negative selection (Miltenyi, catalog #130-097-658) according to manufacturer's protocol. In all cases, isolation purity was greater than 80%. All stimulations were done in 96 well plates in a volume of 200 μl of media and supernatants were collected after the indicated time points.

### Flow Cytometry-Based Analysis

Murine bone marrow-derived dendritic cells (DC) were washed with PBS, Fc-gamma receptor-blocked using TruStain fcX™ (anti-mouse CD16/32, Biolegend, clone 93) and then stained with antibodies specific for CD11c (Biolegend, clone N418) and MHC-II (Tonbo biotech, clone M5/114.15.2), along with staining to detect live/dead cells using DAPI. Data was acquired on an LSR II instrument (BD biosciences).

Single cell suspensions from ipsilateral paws (described below) were washed, Fc-gamma receptor-blocked using TruStain fcX™ and stained with antibodies specific for CD45 (BD biosciences, clone 30-F11), CD11c (Biolegend, clone N418), MHC-II (Tonbo biotech, clone M5/I 14.15.2), Ly-6G (Tonbo, clone 1A8), CD11b (Tonbo, clone M1/70), F4/80 (Biolegend, clone BM8) along with live/dead staining using DAPI. Live CD45⁺ cells were sorted using BD Aria 11 SORP cell sorter at the Flow Cytometry Core facility of Weill Cornell Medicine. All FACS data were analyzed with FlowJo software (TreeStar).

### Lipidomic Analyses

Either Ern1^{WT} or Ern1^{KO} dendritic cells (5×10⁶) were stimulated with 50 ng/ml LPS in 6 well plates. Cells were collected after 6 hours, washed with ice-cold PBS and cell pellets were frozen at −80° C. until further analysis. Cell pellets were suspended in 850 μl of ice-cold PBS and homogenized using a probe sonicator (3×10 sec each on ice). The homogenate was diluted with 150 μl methanol containing 10 ng each of prostaglandin E1-d4, resolvin D1-d5, leukotriene B4-d4, 15-HETE-d8, arachidonic acid-d8, and 100 ng each of cholesteryl heptadecanoate and triheptadecanoyl glycerol (all served as internal standards for the LC-MS analysis). The samples were applied to C18 solid phase extraction cartridge (StrataX C18, Phenomenex) and the lipids were extracted following procedures described by (Maddipati et al. Prostaglandins Other Lipid Mediators 94, 59 (February 2011); Markworth et al. Am J Physiol Regul Integr Comp Physiol 305, R1281 (December 2013)) with following modifications: The SPE cartridges were eluted with isooctane-ethyl acetate (9:1) first for non-polar lipids (sterol esters, neutral sphingolipids, and triglycerides) before eluting the fatty acyl lipidome with methanol containing 0.1% formic acid. The lipidomic analysis was performed by the Lipidomics Core Facility at Wayne State University by LC-MS using standard protocols. The procedures followed were essentially as described earlier for eicosanomic analysis (Maddipati et al. FASEB J 28, 4835 (November 2014); Maddipati et al. The FASEB Journal 30, 3296 (Oct. 3, 2016, 2016); Maddipati et al. J. Lipid Res. 57, 1906 (Oct. 1, 2016)) and by other published procedures for fatty acids, sterol esters, triacylglycerols, and sphingolipids (Shaner et al. J Lipid Res 50, 1692 (August 2009); Hellmuth et al. Anal. Chem. 84, 1483 (2012); Hutchins et al. J. Lipid Res. 49, 804 (April 2008)).

### Immunoblot Assays

Dendritic cells (DC) were washed twice in 1× cold PBS and cell pellets were lysed using RIPA lysis buffer (150 mM Sodium Chloride, 1% Triton X100, 0.5% Sodium Deoxycholate, 0.1% SDS and 50 mM Tris pH8.0) supplemented with protease and phosphatase inhibitors (Roche). Homogenates were centrifuged at 14,000 rpm for 30 min at 4° C., and the supernatants were collected. Protein concentrations were determined using BCA protein assay kit (Thermo Fisher Scientific). Equivalent amounts of protein were separated via SDS-PAGE and transferred to PVDF membranes (Immobilon, Millipore). Membranes were blotted with primary antibodies like anti-Cox-2 (cell signaling, catalog #12282), anti-mPGES-1 (Cayman chemicals, catalog #160140) and anti-b actin (cell signaling, catalog #4967) antibody; and anti-rabbit secondary antibody conjugated with HRP (Thermo Fischer Scientific). SuperSignal West Pico and Femto chemiluminescent substrates (Thermo Fisher Scientific) were used to image blots in a FlourChemE instrument (ProteinSimple).

### PGE2 ELISA

Cells (2.5×10⁵) were stimulated with selected compounds and at the indicated time points. PGE2 was measured in the supernatants using PGE2 ELISA kit (Enzo, Cat #ADI-900-001). If different number of cells were plated, PGE2 levels were normalized to 2.5×105 cells/well. Cell viability counts were comparable in all cases. Peritoneal lavages were obtained by flushing the abdominal cavity with 10 ml of 1× PBS (pH 7.4). The wash was centrifuged at 1500 rpm for 5 min and supernatants were stored at −80° C. until analyzed using the PGE₂ ELISA kit described above. Plates were read at 405 nm using Vairoskan (Thermo Fischer Scientific).

### ChIP Assays

Human monocyte-derived DC were incubated in complete RPMI medium (1 mM glucose and 4 mM L-glutamine) in the presence and absence of 1 mM 2-DG and treated with 1 mg/ml zymosan, as described by Marquez et al. Frontiers in Immunology 8, 639 (2017). Cells were then washed and fixed in 1% formaldehyde for ChIP assays. Cross-linking was terminated using 0.125 M glycine. Nuclear extracts were collected and resuspended in a lysis buffer containing a high salt concentration. Chromatin sonication was carried out using a Bioruptor device from Diagenode (Liege, Belgium). The chromatin solution was precleared by adding Protein A/G PLUS-Agarose for 30 min at 4° C. under continuous rotation. After elimination of the beads, antibody was added for overnight incubation at 4° C., and then incubation with Protein A/G PLUS-Agarose was carried out for 2 hours at 4° C. Beads were pelleted by centrifugation at 12,000 rpm and sequentially washed with lysis buffer high salt, wash buffer, and elution buffer. Cross-links were reversed by heating at 67° C. in a water bath, and the DNA bound to the beads isolated by extraction with phenol/chloroform/isoamylalcohol. Irrelevant antibody (Ab) and sequences of the Pri-miR-21 promoter were used as control of binding specificity. The IRE1α specific inhibitor utilized in these assays was MKC8866 (Mankind Pharmaceutical) and was obtained under an MTA with M.S.C. Results are expressed as percentage of input. Primer sequences used for ChIP-PCR are shown in Table 9, with numbering in base pairs (bp) from the transcription initiation site. However, in the case of Pri-miR-21, numbering was from the mRNA sequence, which is encoded in chromosome 17, GRCh38.p7. This was selected because of its lack of putative XBP1s-binding sequences.

### RNA Sequencing and Bioinformatic Analyses

RNA was isolated using RNeasy MinElute kit (Qiagen) from LPS-stimulated or zymosan-stimulated murine bone marrow-derived dendritic cells (DC). All samples passed RNA quality control examined by Agilent Bioanalyzer 2100, and mRNA libraries were generated and sequenced at the Weill Cornell Epigenomics Core Facility. RNA-sequence data was aligned using bowtie2 (Langmead & Salzberg, Nat Methods 9, 357 (Mar. 4, 2012)) against hg19 genome and RSEM v1.2.12 software (Li & Dewey, BMC bioinformatics 12, 323 (2011)) was used to estimate gene-level read counts using Ensemble transcriptome information. DESeq2 (Love et al. Genome Biol 15, 550 (2014)) was used to estimate significance of differential expression difference between any two experimental groups and gene expression changes of at least 1.2-fold were considered significant if passed false discover rate (FDR)<5% thresholds. Gene set enrichment analysis was done using QIAGEN's Ingenuity® Pathway Analysis software (IPA®, QIAGEN Redwood City, see website qiagen.com/ingenuity) using “Canonical Pathways,” “Diseases & Functions.” and “Upstream Regulators” options. Enrichment results with at least 10 deregulated genes were considered and pathways that passed FDR<5%, functions with p-value<10⁻⁷ and regulators with p-value<0.001 were considered significant. Only functions and regulators with significant predicted activation states (|Z-score|>2) were reported. Functions were additionally filtered to remove entries specific to cancer cell lines and immune cell types. Significance of overlap was calculated with hypergeometric test.

### Gene Editing in Human DC

The 20-nucleotide crRNA targeting human XBP1 (Homo sapiens chromosome 22. GRCh38.p12, NC_00022.11) is directed at the genomic sequence TGCACGTAGTCTGAGTCCTGCGG (SEQ ID NO:35), the 3 additional nucleotides highlighted in bold represent the protospacer adjacent motif, or PAM). This target sequence corresponds to exon 4 of the human XBP1 transcript and was manually chosen by identifying a 20-base pair fragment immediately upstream of the highlighted PAM (Ran et al. Nat Protoc 8, 2281 (November 2013)). The PAM was selected such that Cas9-mediated target DNA cleavage would occur within the 26 nucleotides of XBP1u that are recognized and spliced by activated IRE1α (Yoshida et al. Cell 107: 881 (Dec. 28, 2001) Calfon et al. Nature 415: 92 (Jan. 3, 2002)). The on-target and off-target effects of the manually selected CRISPR sequence were then analyzed using the Broad Institute's Genetic Perturbation Platform (see website at portals.broadinstitute.org/gpp/public/analysis-tools/sgrna-design). To validate the genomic editing capacity of the crRNA, RT-qPCR was performed on total RNA isolated from cells transfected with sgRNA-Cas9 complexes containing the XBP1 crRNA described above. The reverse primer for XBP1s quantification via RT-qPCR anneals to the same nucleotides as the XBP1 crRNA target site. Therefore, the primers can only efficiently amplify intact, unperturbed XBP1s cDNAs. The primers for evaluating deletion efficacy are listed in Table 9. The genomic target sequence for the crRNA directed at human ERN1 (Homo sapiens chromosome 17, GRCh38.p12, NC_000017.11) is ATGTAGAGGATTCCATCTGACCC (SEQ ID NO:36). This sequence was generated and chosen using the Zhang Lab's crRNA design tool (see website at crispr.mit.edu). To validate the genomic editing capacity of this crRNA, RT-qPCR was performed on total RNA isolated from cells transfected with sgRNA-Cas9 complexes containing ERN1 crRNA. XBP1s levels were used to assess the genetic perturbation of IRE1α, using the primer pair specified in Table 9. The scrambled crRNA contains a 20-nucleotide sequence that is computationally designed to be nontargeting within the human genome (see website at sfvideo.blob.core.windows.net/sitefinity/docs/default-source/user-guide-manual/alt-r-crisprcas9-user-guide-ribonucleoprotein-transfections-recommended.pdf?sfvrsn=1c43407_12.). The RNA sequence for this non-targeting control was CGUUAAUCGCGUAUAAUACG (SEQ ID NO:37).

Human CD14+ monocytes were isolated from peripheral blood and plated at a density of 5×10⁶ cells in 3 mL RPMI supplemented with human recombinant GMCSF at 1000 IU/mL and IL-4 at 500 IU/mL as described above. On day 6, dendritic cells (DC) were prepared for transfection by washing with serum-free PBS and re-suspending in RPMI medium supplemented with human recombinant GM-CSF and IL-4, at the same concentrations mentioned above. DC were then reverse-transfected on a 96-well plate by adding 2.5×10⁵ cells in suspension onto 150 nM complexes containing lipofectamine CRISPRMAX transfection reagent (Invitrogen). All materials for sgRNA-Cas9 complex generation were purchased from Integrated DNA Technologies and prepared as instructed (see website at sfvideo.blob.core. windows.net/sitefinity/docs/default-source/user-guide-manual/alt-r-crisprcas9-user-guide-ribonucleoprotein-transfections-recommended.pdf9sfvrsn=1c43407_12. The final sgRNA-Cas9 and CRISPRMAX complex concentrations per well were 50 nM and 1% (vol/vol), respectively. Forty-eight hours post-transfection, genetic ablation of target genes was assessed via RT-qPCR.

### Plasmid Constructs and Luciferase Reporter Assays

Expression constructs used for luciferase-based assays are pcDNA3.1 XBP1s (NM_001079539.1), pcDNA3.1 CHOP (NM_001195053.1) while reporter constructs used are pGL3-PTGS2 promoter (at −1.2 kb/+137) and pGL3-PTGES promoter (at −1.3 kb/+35). All plasmids were generated at VectorBuilder.

For dual luciferase assays, 2×10⁴ HEK293FT cells were plated overnight in a 96-well plate and were transfected with the indicated plasmids using Lipofectamine 3000 (Thermo Fischer Scientific). 18 ng of reporter and 2 ng of renilla plasmid were co-transfected with various amounts of expression plasmids (1:1, 1:3 or 1:5 reporter: expression plasmid ratios) and pcDNA3.1, which was added to reach a total of 200 ng of DNA/well. After 36-48 hours, cells were washed with 1×PBS and were lysed in 1× Passive Lysis Buffer according to the manufacturer's protocol (Dual luciferase reporter assay system, Promega, catalog #E1960) (Lee et al. Mol Cell Biol 23, 7448 (November 2003)). Luciferase and Renilla activity were measured in 96-well plates using an automated luminometer (Luminoskan Ascent, Thermo Fischer). Luciferase activity was normalized to Renilla.

Single-Cell Suspensions from Mouse Paws

Mice were perfused transcardially with 20 mL of 0.1 M phosphate buffer one day after paw incision. Both anterior and posterior parts of the injured or non-injured paw were dissected in a petri dish containing 2 mL of RPMI 164 medium (Gibco). Tissue was dissected into small pieces using surgical scissors, then transferred to a tube containing 2 mL of 0.5 mg/mL of Type II collagenase (Worthington Biochemical Corporation. Lakewood, N.J.) in RPMI 1640 (Gibco) and incubated for 2 hours at 37° C. shaking at 700 rpm. Enzymatic reaction was stopped by adding 4 mL of 2% fetal bovine serum (FBS, Sigma) in 0.1 M phosphate buffer. Digested tissue was passed through a 40 μm nylon mesh (BD Biosciences) using a syringe plunger. Cell suspension was centrifuged at 450 G for 5 min at 4° C. and resuspended in 1 mL of 2% fetal bovine serum in 0.1 M phosphate buffer. Total cell number and cellular viability were determined using trypan blue staining and a hemocytometer. Cells were stored at −80° C. in FBS containing 10% DMSO until subsequent flow cytometry analyses were performed.

### Animals and Paw Incision Surgery

Plantar incision surgery was performed as described by Pogatzki & Raja (Anesthesiology 99, 1023 (October 2003)). Briefly, mice were anesthetized with isoflurane in oxygen (4% induction, 1.5%-2% for maintenance) and the right hind paw was aseptically cleaned with 10% povidone-iodine solution. Then, a 5 mm incision was made in the glabrous hind-paw skin from the heel to the base of the toes using a No. 11 scalpel and sterile technique. The underlying muscle and ligaments were elevated with a curved forceps and stretched for 6-8 seconds, without incising them. The incision was closed using 5.0 nylon mattress sutures.

### Paw Inflammation

Paw perimeter was measured in both left and right hind paws before the surgery and after every behavioral evaluation. The procedure was performed in a consistent manner using a 4.0 silk thread that was placed around the center of the surgery in the right paw and at the same level in the paw contralateral to surgery. An increase in the paw perimeter was considered as inflammation of the affected paw.

### Behavioral Tests

All behavioral measurements were performed by a blinded observer before and after surgery (postoperative days 1-21) or acetic acid intraperitoneal injection (15-30 min). Animals were acclimated to the testing devices and/or places for 3 days, and baseline measurements were taken for at least 4 consecutive days before the surgery or acetic acid.

Mice were placed in individual acrylic chambers on an elevated mesh floor for 30-45 min before testing. Two spontaneous pain-relate behaviors were evaluated, rearings and paw flinches. Following the acclimatization period, the number of total vertical rearings and paw flinches were quantified during a 2-min period. Vertical rearings were defined as the number of times that the animal stood supporting its weight on both hind limbs. Vertical rearings are a normal behavior in rodents, thus a reduction of this behavior is indicative of a protective way to prevent pain due to movement, which mimics pain induced by surgeries in humans. Spontaneous flinching of the affected paw was quantified every time that the animal shacked the affected paw without any stimulation. Flinches of the injured paw is a pain-related behavior that is indicative of breakthrough pain, similar to intense spontaneous spike of pain in humans with postoperative pain.

Mechanical hypersensitivity was assessed after quantification of vertical rearings and spontaneous flinching. Mechanical withdrawal thresholds were calculated using the up-down method and applying force with calibrated Von Frey filaments (0.07-g, 0.17-g, 0.40-g, 0.60-g, 1.04-g, 1.37-g, and 2.0-g, Stoeling, Wood Dale, Ill., USA) to the plantar aspect of the paw for 5 seconds. Paw withdraws or flinching in response to a given applied force was noted as a positive response.

Hind paw weight bearing distribution was determined using an incapacitance tester apparatus (Stoelting, Ill., version 5.64). This is a test for non-reflexive behaviors that represents a spontaneous pain-related behavior that mimics postoperative pain behaviors in humans (protection of the surgery site from normal activities). Before surgery, animals were habituated for at least 3 days to the apparatus, in which animals stand with each hind paw resting on individual weight plates inside an acrylic chamber. The apparatus measures the body weight distributed between the two hind paws over a 3 second period and provides the average measurement. The average value of each hind paw was used to determine the weight distribution ratio (ispsilateral/contralateral side). A ratio below one indicates a greater weight bearing on the contralateral paw and was therefore considered as a pain-related behavior.

Writhing spontaneous pain behaviors were evaluated after intraperitoneal injection of 0.9% acetic acid (v/v, 5 ml/kg). The number of writhing responses was quantified immediately after acetic acid injection for 30 min in 5 min intervals by an observer blinded to genotype. Writings induced acetic acid are overt stretching behaviors indicative of abdominal pain, a phenomenon that is dependent upon mPGES-1 and PGE2 (Kamei et al. J Biol Chem 279, 33684 (Aug. 6, 2004); Trebino et al. Proc Nat Acad Sci USA 100, 9044 (Jul. 22, 2003)).

### Immunohistochemistry

Mice were anesthetized with isoflurane (34% in oxygen) and perfused transcardially with 20 ml of filtered solution 0.1 M phosphate-buffered saline (PBS) followed by 20 ml of 4% formaldehyde. Tissue around the injured paw was collected by making a rectangular incision around the injury about 1.5 mm apart from the center of the surgery. Skin and muscle associated with the incision were collected and post-fixed for 3 hours in 4% formaldehyde at 4° C. Tissue was stored at 4° C. in 30% sucrose solution for 72 h before sectioning. Slices of tissue were cut at 18 μm using optimal cutting temperature compound (Sakura Finetek, Torrence, Calif. USA) in a Leica cryostat and placed in coated slides. Slides were then washed three times for five minutes with 0.1 M PBS and blocked using a solution of 3% normal donkey serum (NDS)+0.3% triton X-100 in 0.1 M PBS for 1 h at room temperature. Primary antibodies used were rabbit anti-Cox-2 (1:500, Cell Signaling, catalog #12282) and rat anti-CD45 (1:100, BioRad, catalog #MCA1388). Tissues with primary antibodies were incubated overnight at 4° C. Then, tissues were washed three times for five minutes with 0.1 M PBS and incubated 2 hours at room temperature with corresponding secondary antibodies: donkey anti-rabbit Cyanine 2 (1:400) and donkey anti-rat Cyanine 3 (1:400) (Jackson Immuno Research Labs, West grove, PA, USA). Finally, slides were rinsed three times and mounted using anti-fade medium containing 4′,6-diamidino-2-phenylindole dihydrochloride hydrate (DAPI, Invitrogen) to allow visualization of cell nuclei.

At least three pictures per slide were taken at 20× at areas adjacent to the incision using a Nikon Eclipse Ni fluorescent microscope system (Nikon, Japan). In each picture, the quantification of CD45+ or Cox-2+ cells was made in three random squares of 100 μm2 each. The percent of Cox-2+ was then calculated in relationship to the total CD45+ cells by a blind observer. For co-localization studies, images were acquired with an Olympus FV1200 confocal microscope and images were prepared with Olympus Fluoview Version 4.2b software and Adobe Photoshop software. All images were taken from adjacent areas of the surgical wound ipsilateral to paw incision.

### Statistical Analysis

All statistical analyses were performed using GraphPad Prism 6 software. Comparisons between two groups were assessed using unpaired two-tailed Student's t-test, unless otherwise stated. All grouped data (time course) were analyzed using two-way ANOVA and Sidak's multiple comparisons test. Data are presented as mean±s.e.m. and P values of <0.05 were considered to be statistically significant.

### Example 2: Wild Type Vs. IRE1α-Deficient Transcriptional Analyses

This Example describes transcriptional analysis of IRE1α-deficient bone marrow-derived dendritic cells (DC).

Unbiased transcriptional analyses were performed of wild type vs. IRE1α-deficient bone marrow-derived dendritic cells (DC) stimulated with bacterial LPS (TLR4 agonist) or fungal zymosan (TLR2 and Dectin-1 agonist).

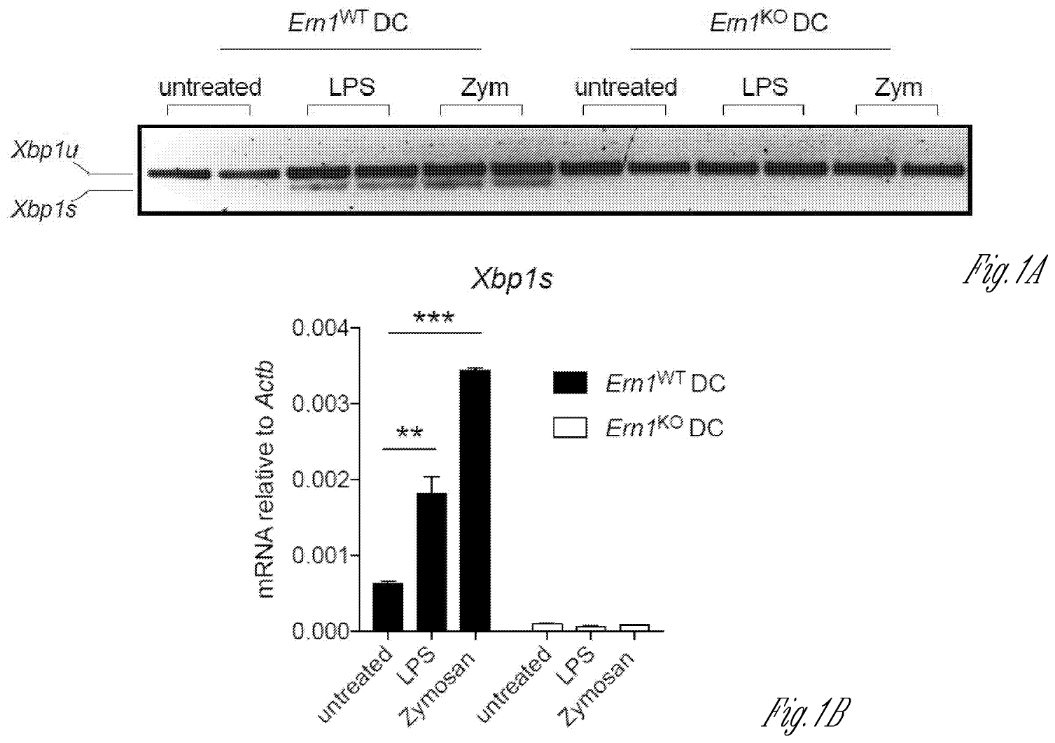

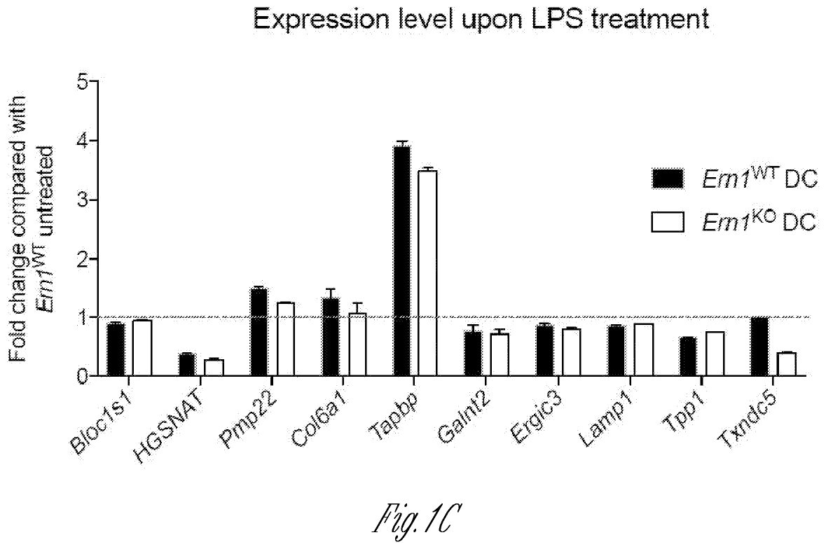

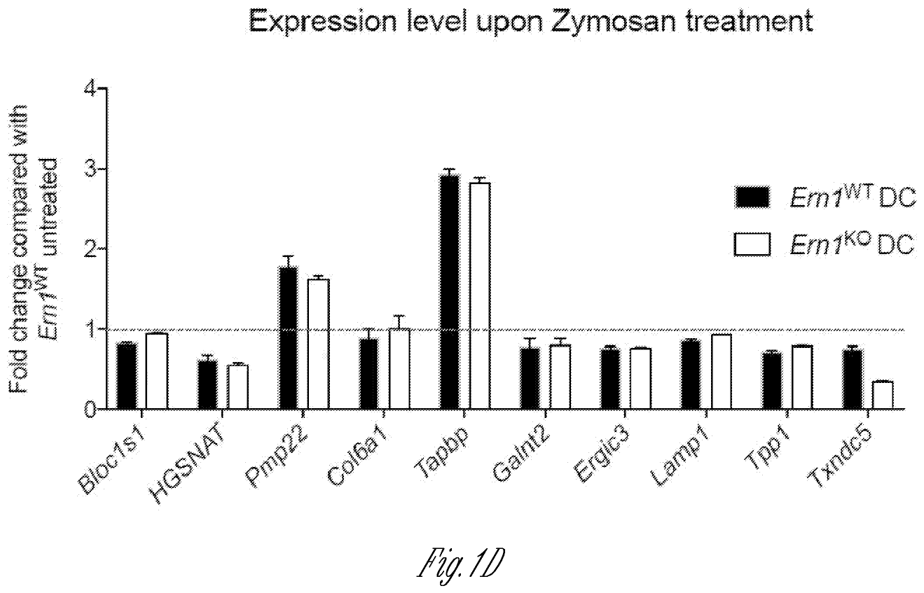

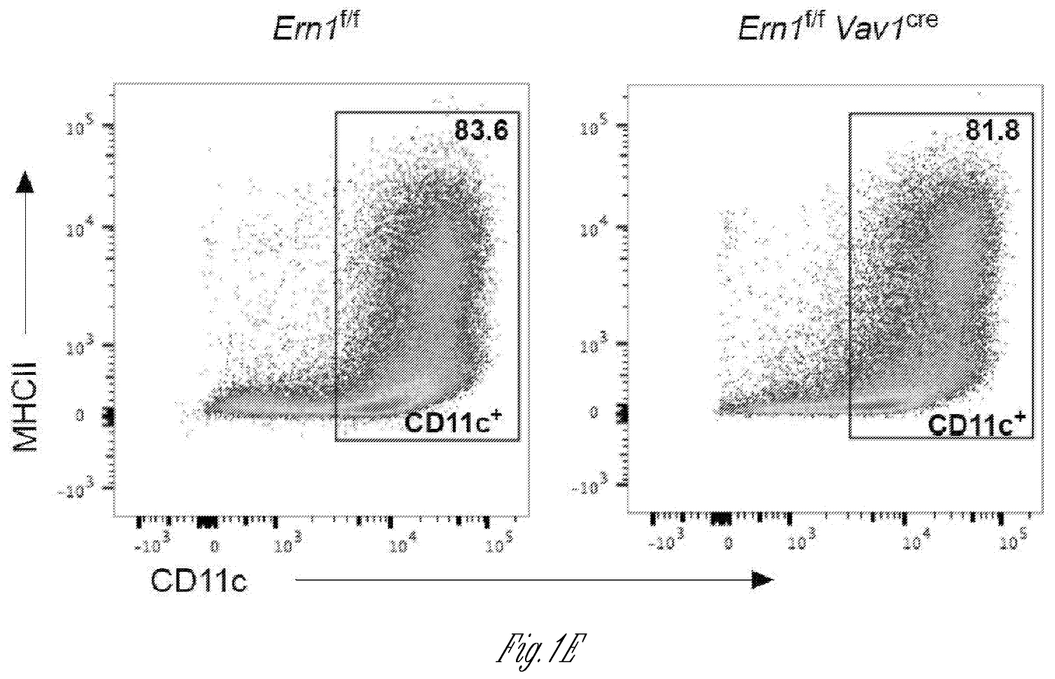

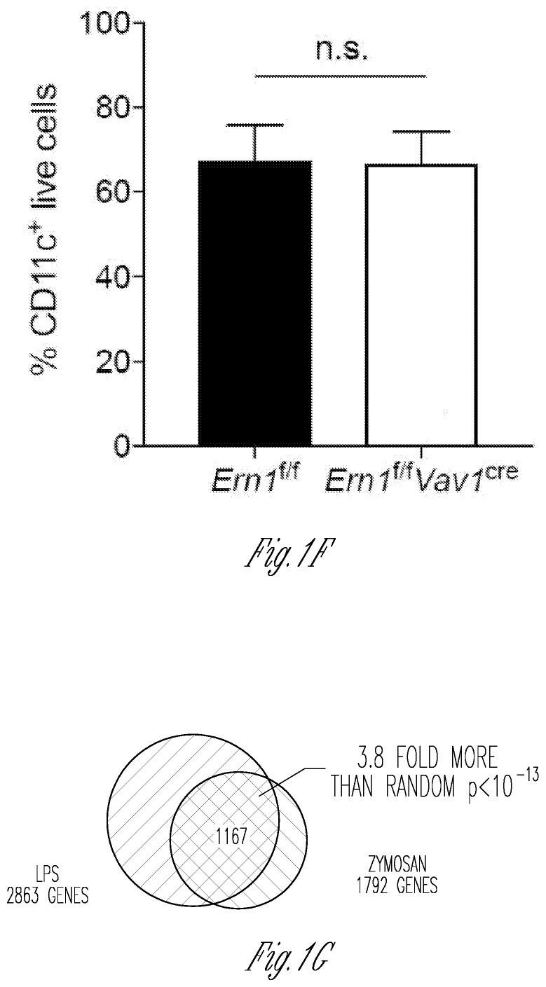

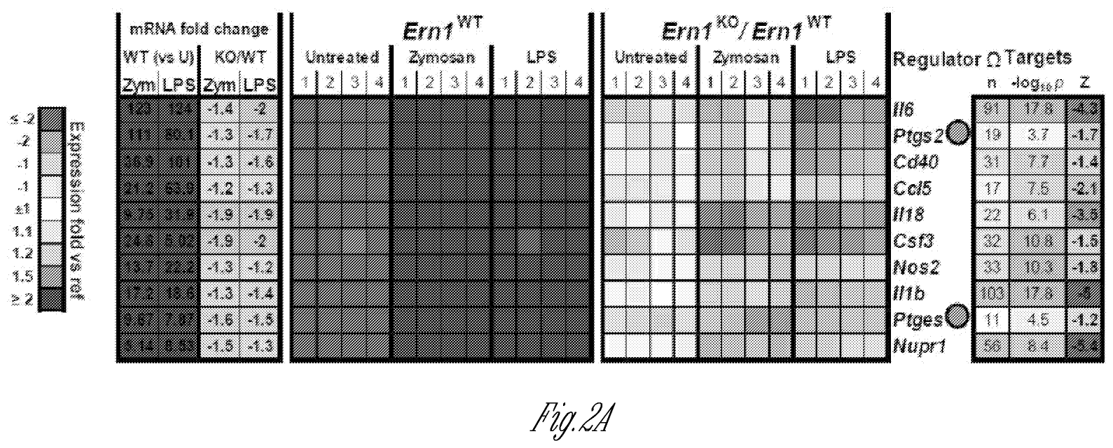

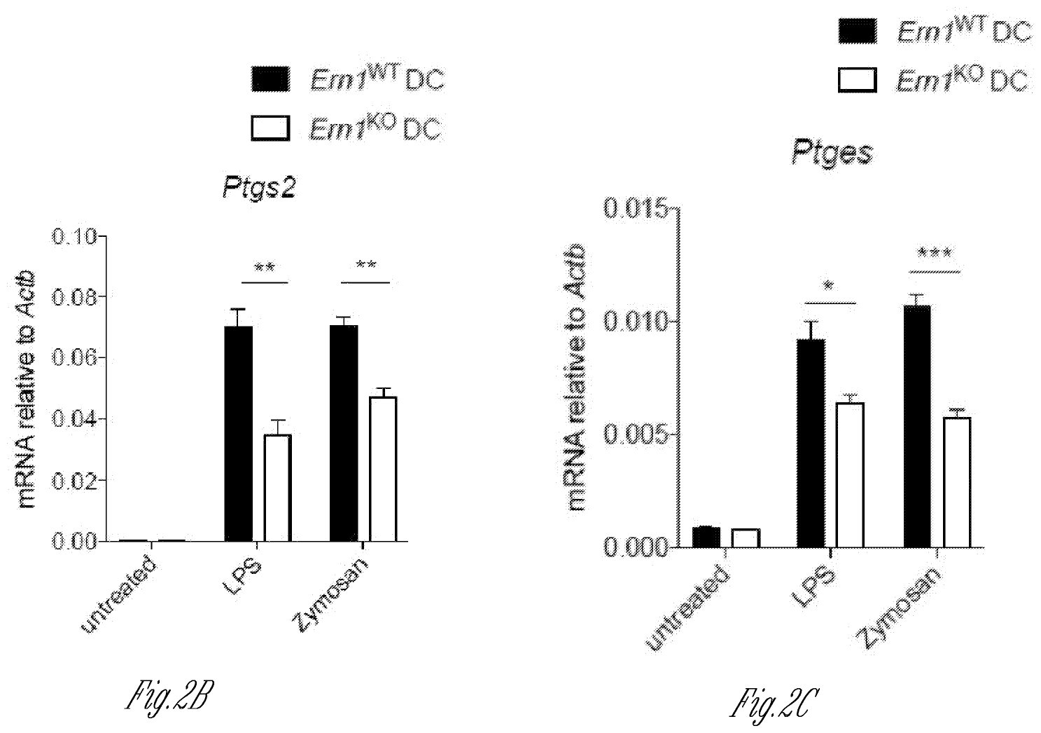

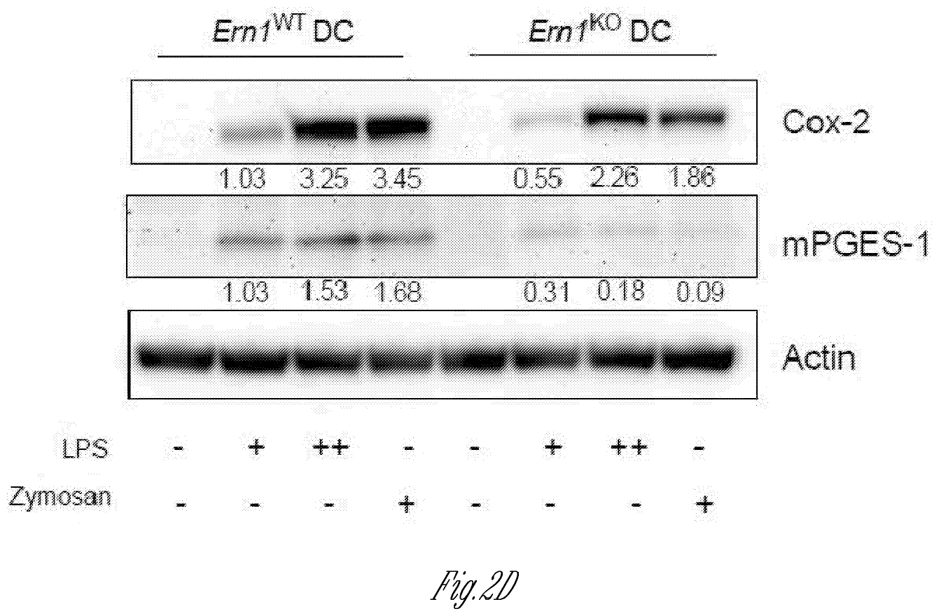

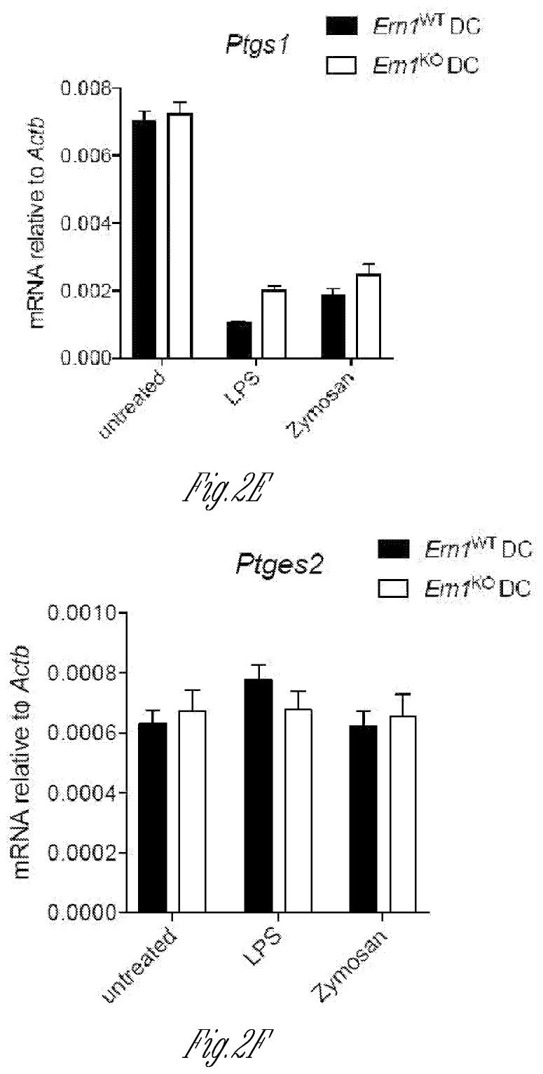

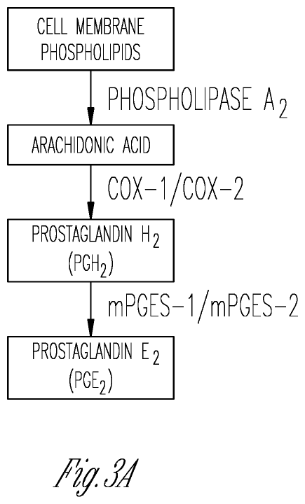

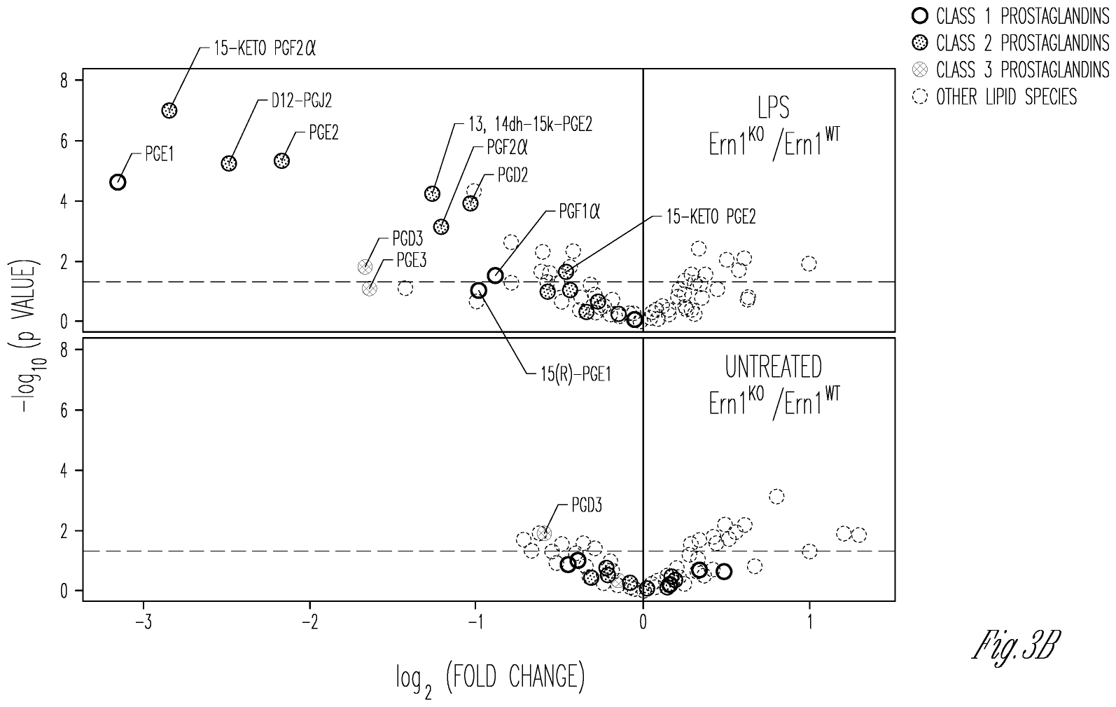

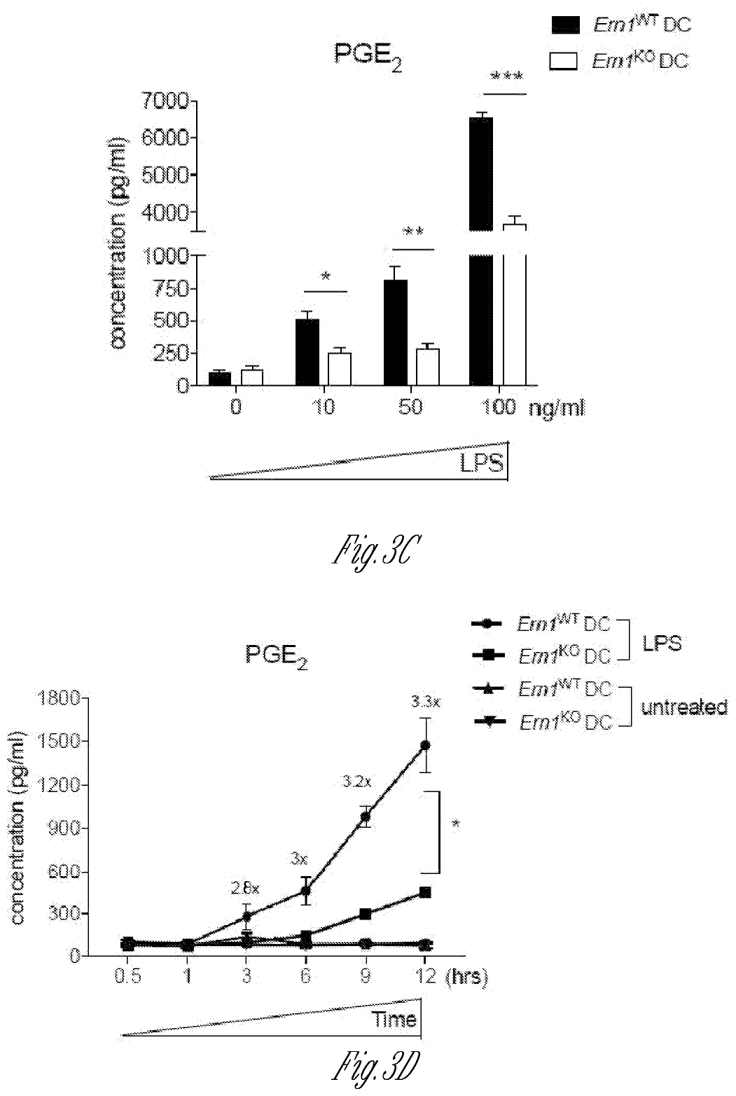

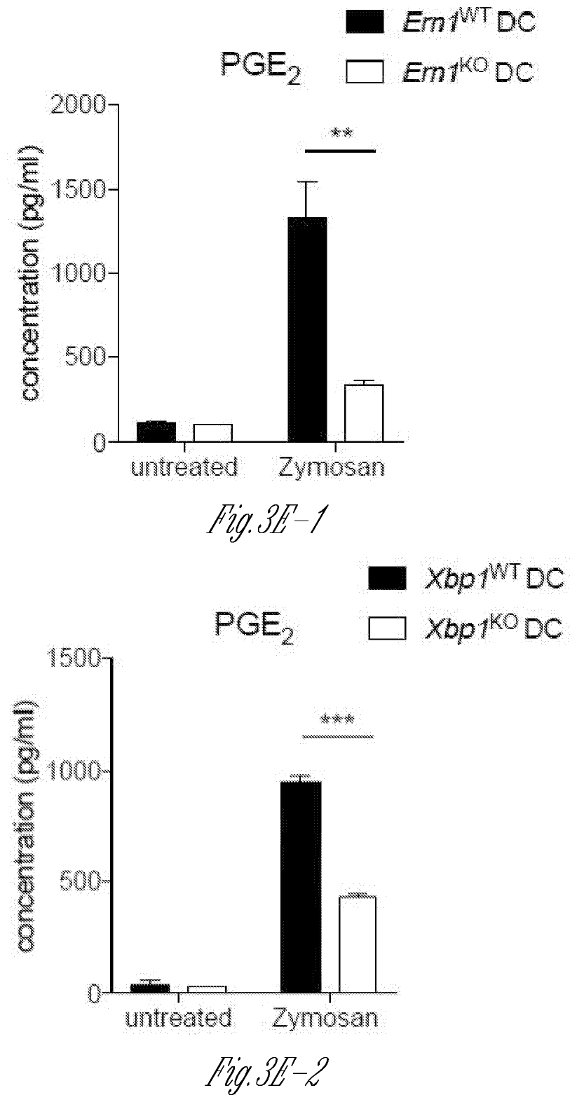

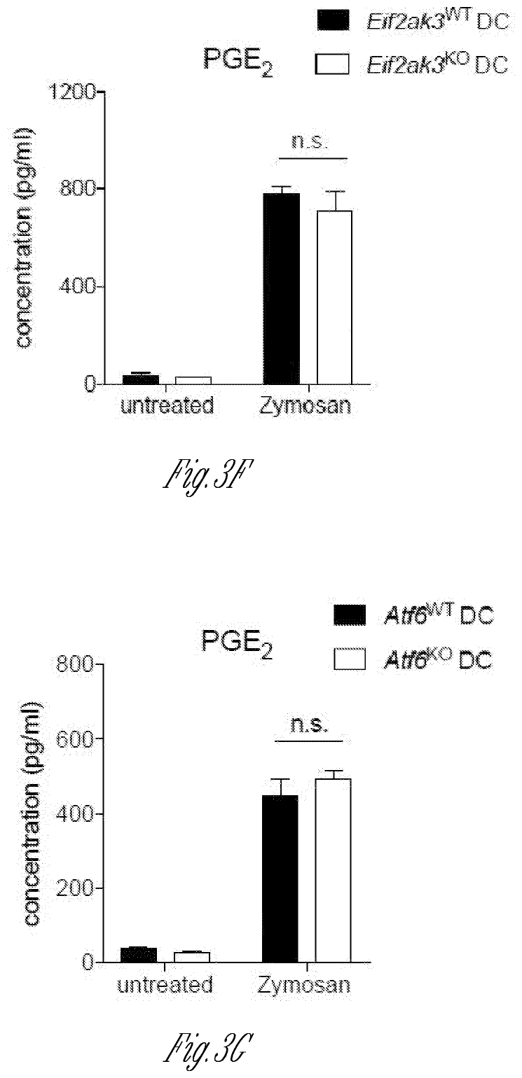

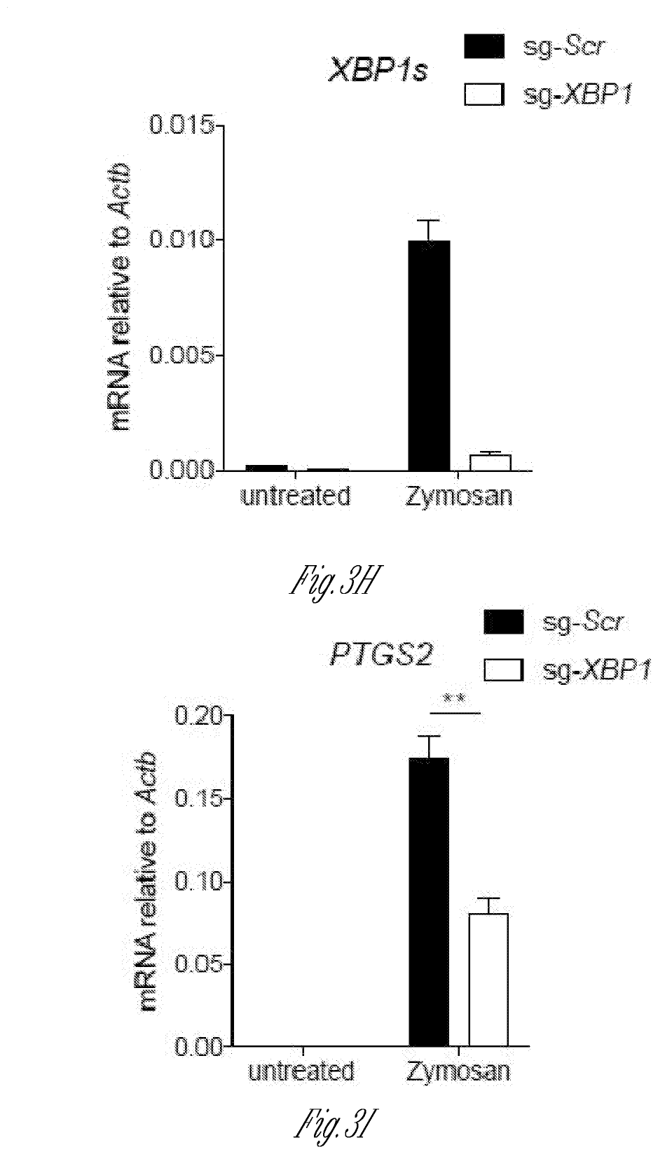

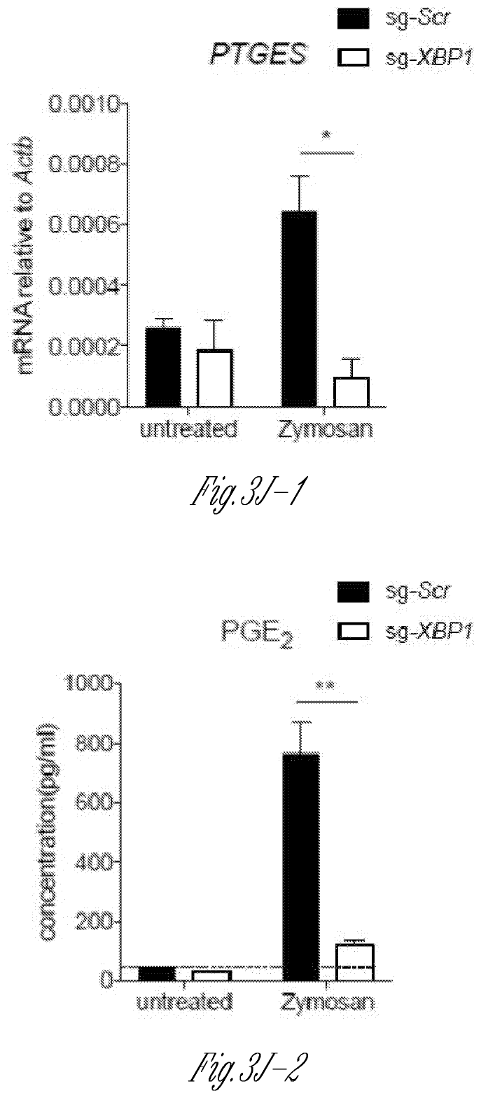

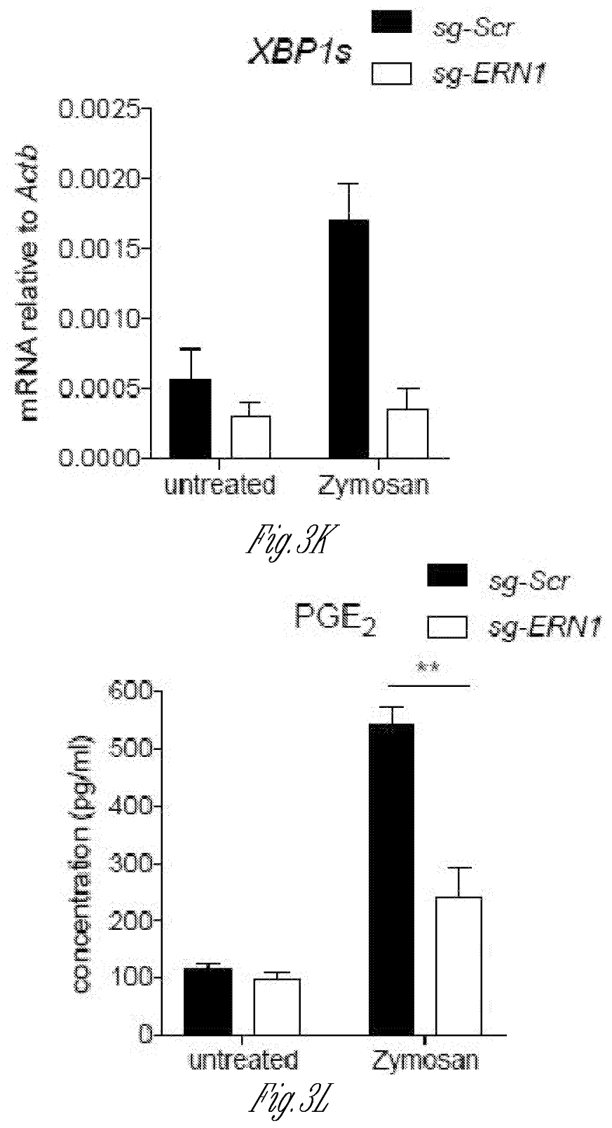

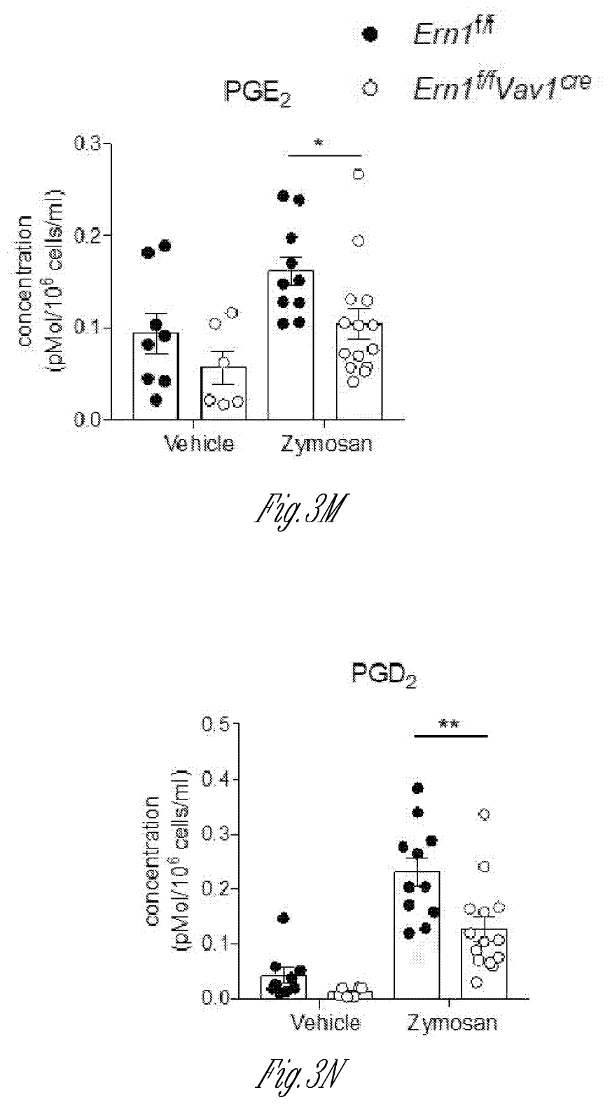

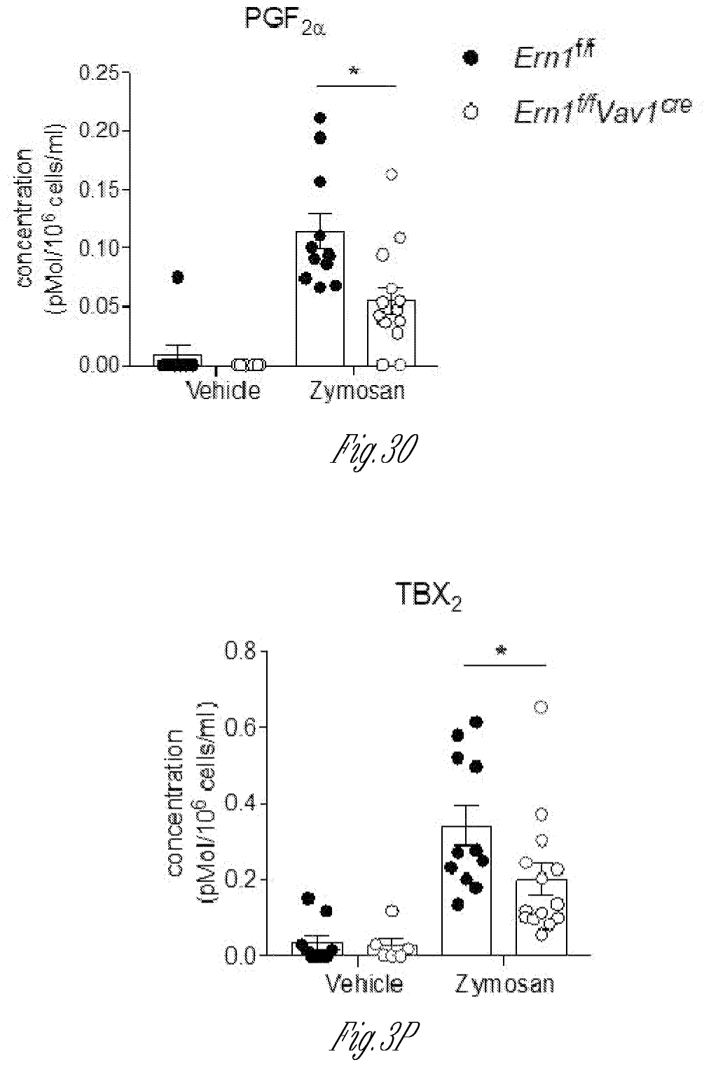

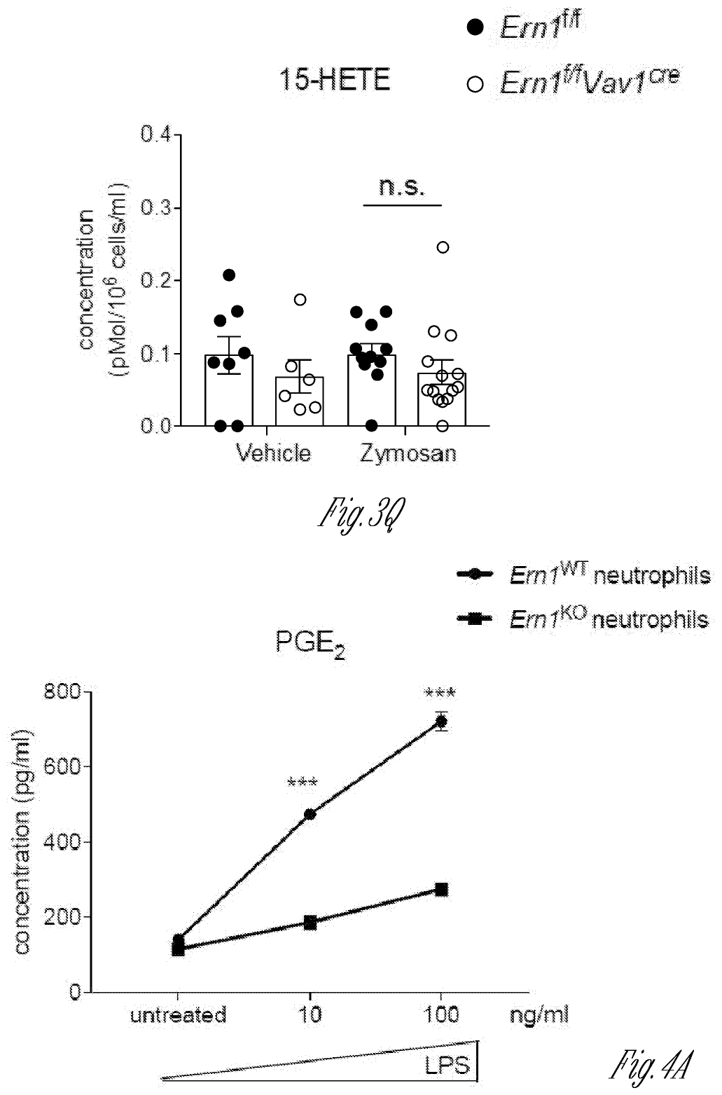

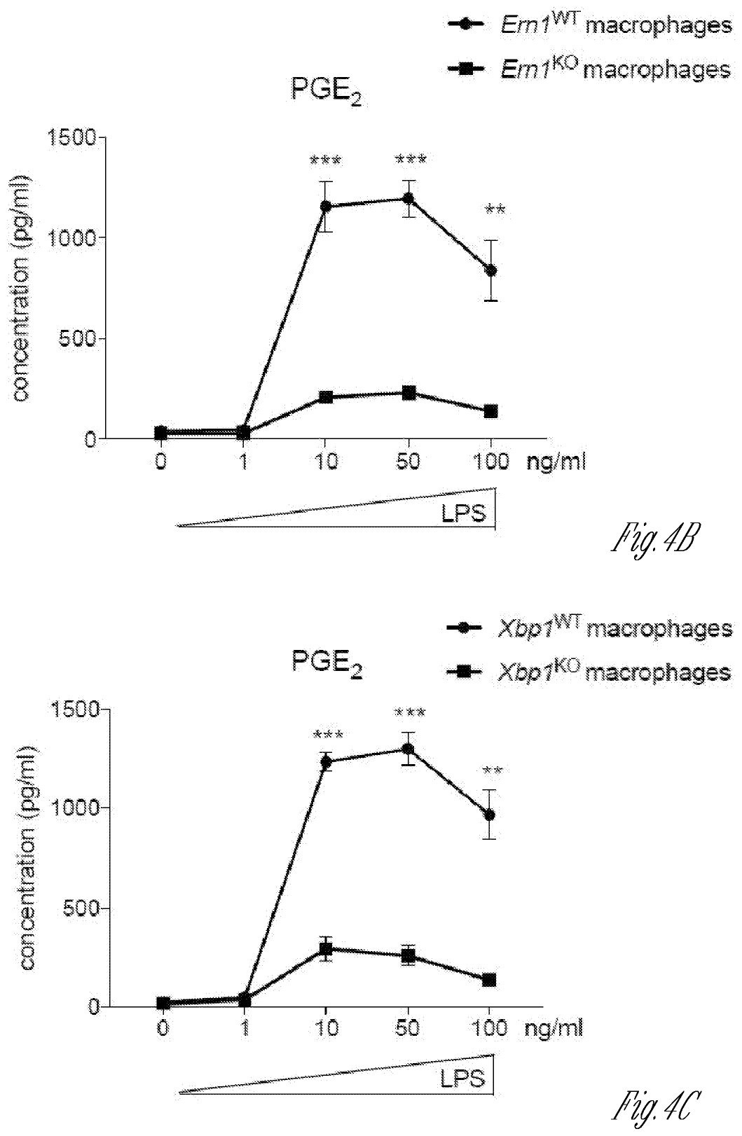

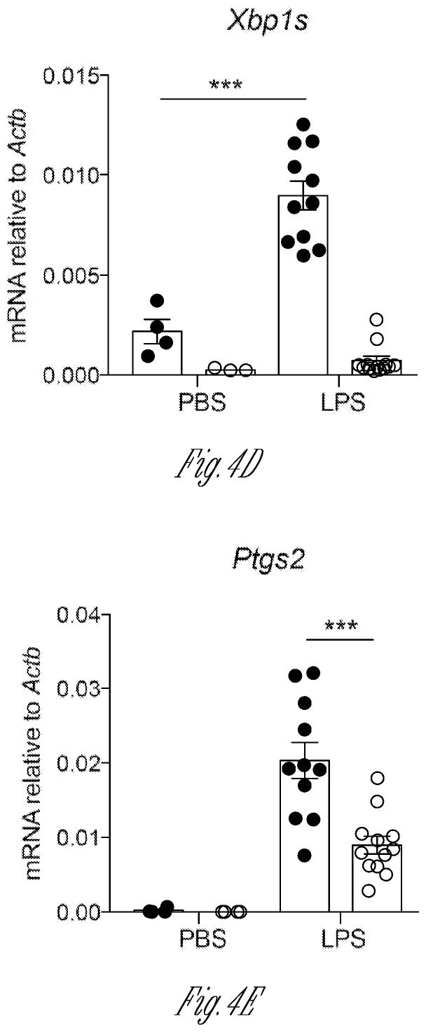

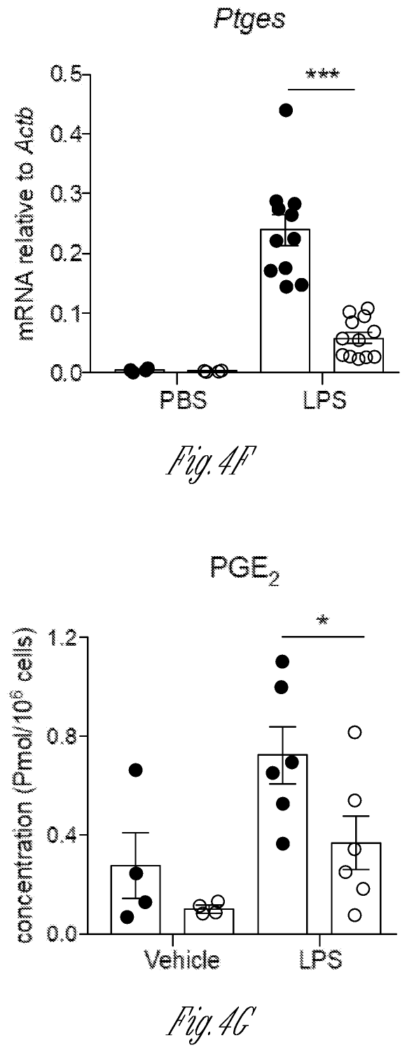

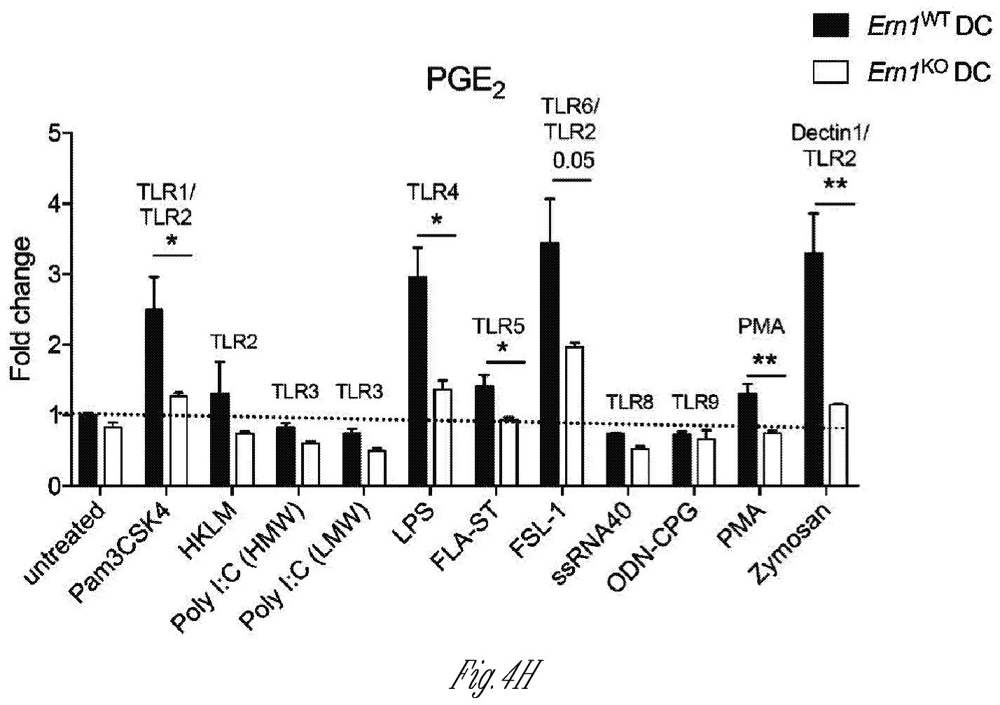

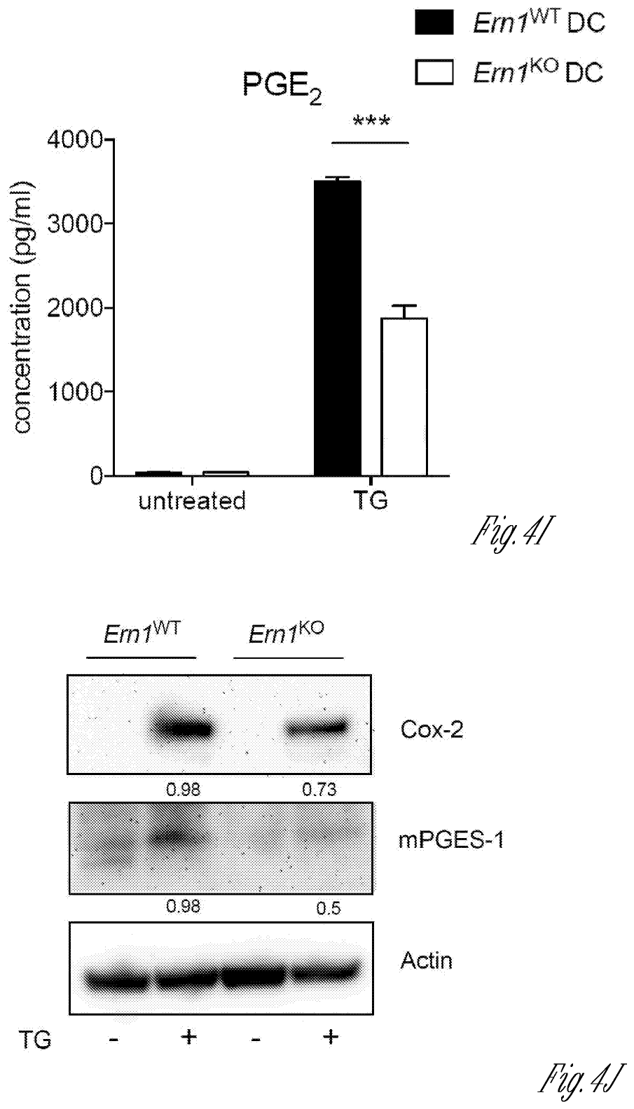

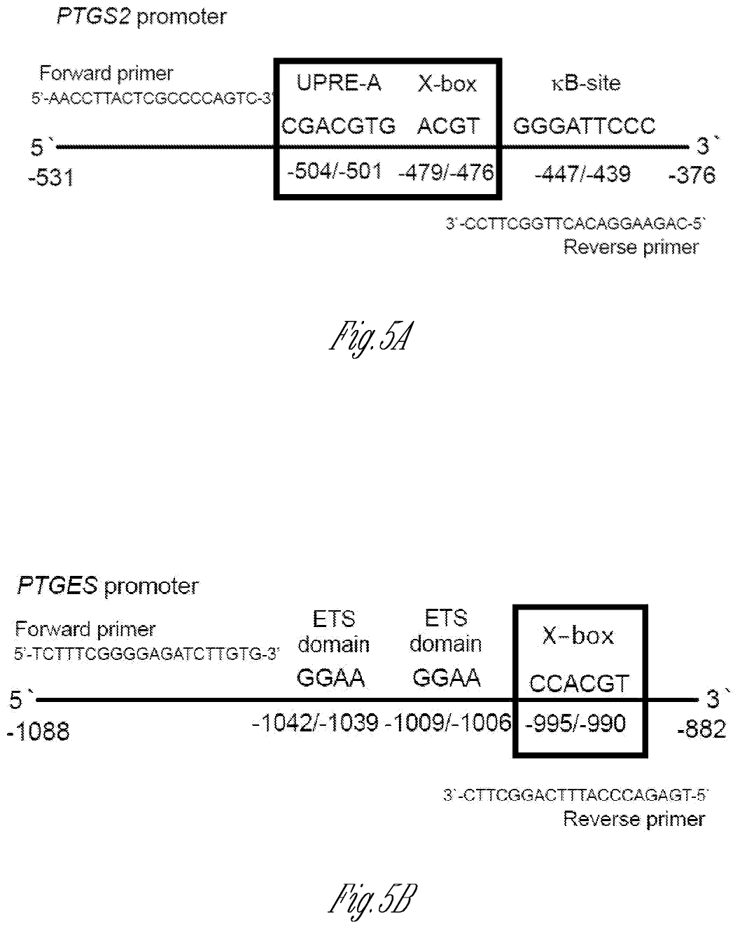

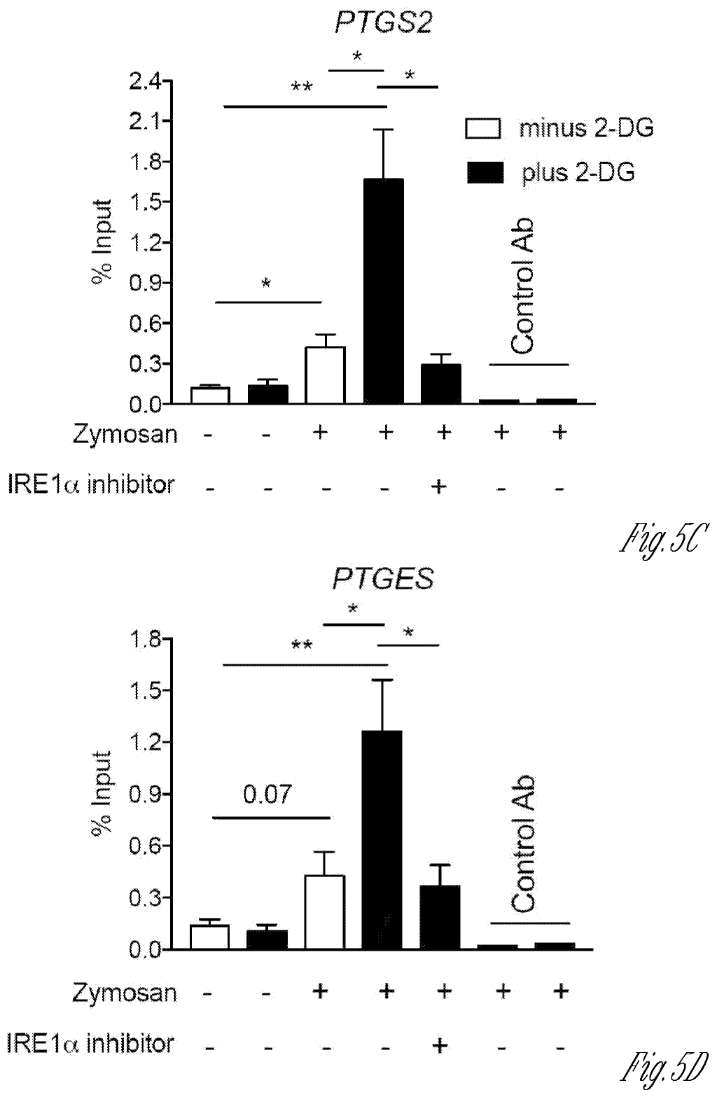

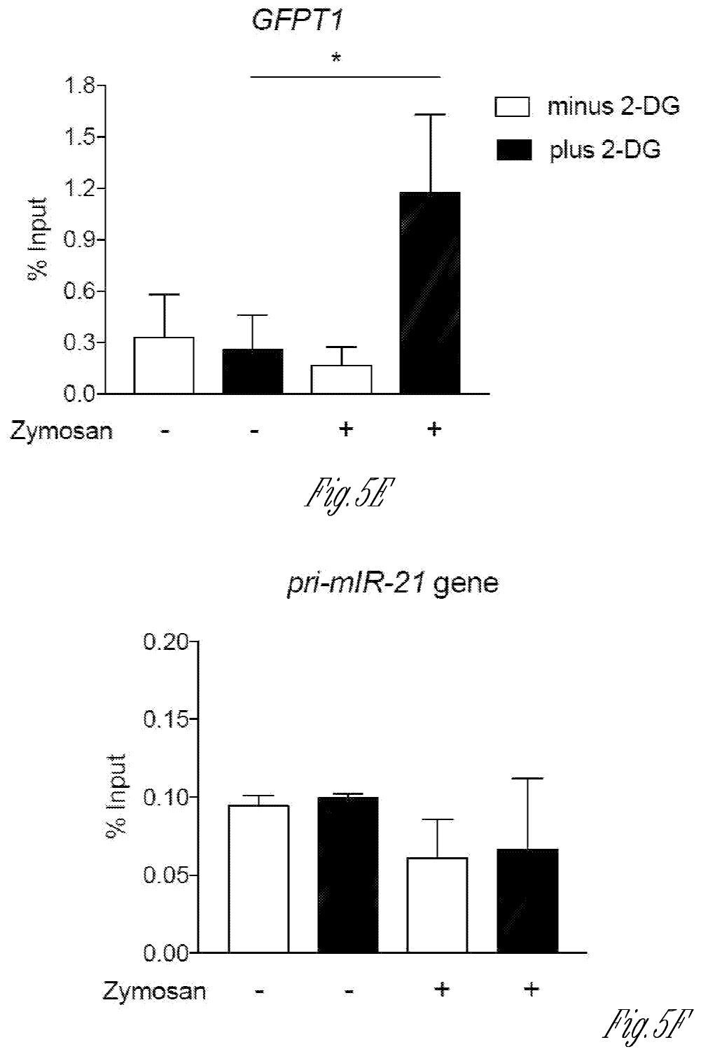

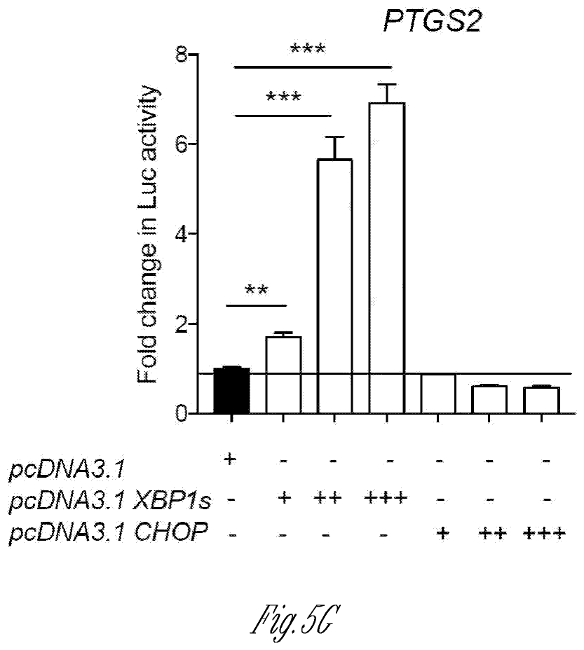

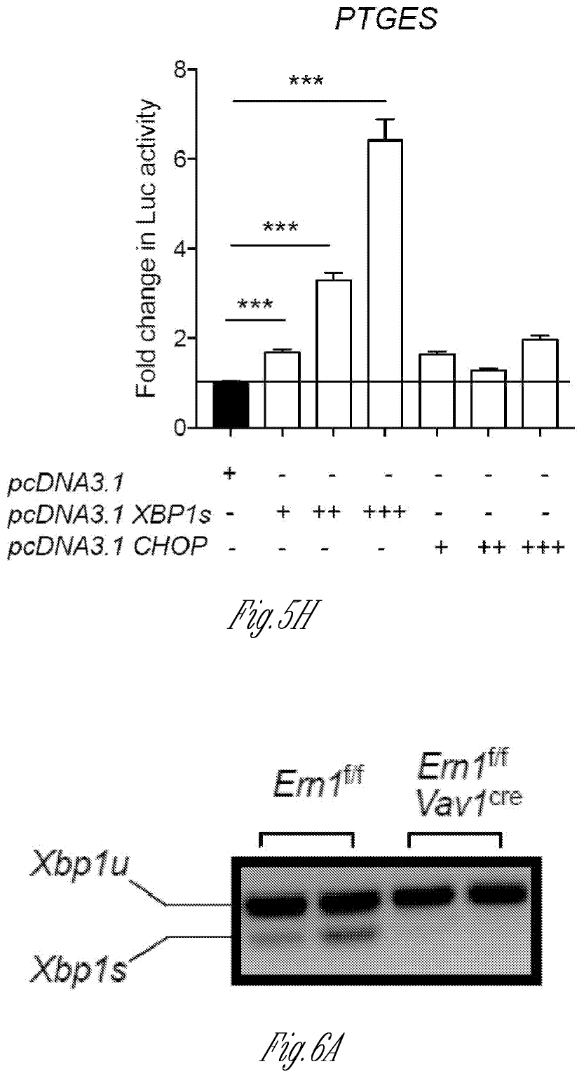

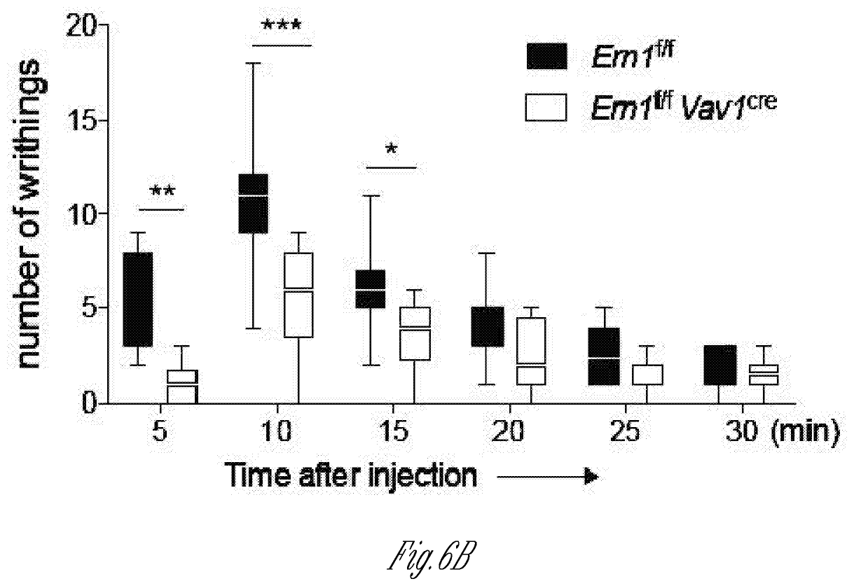

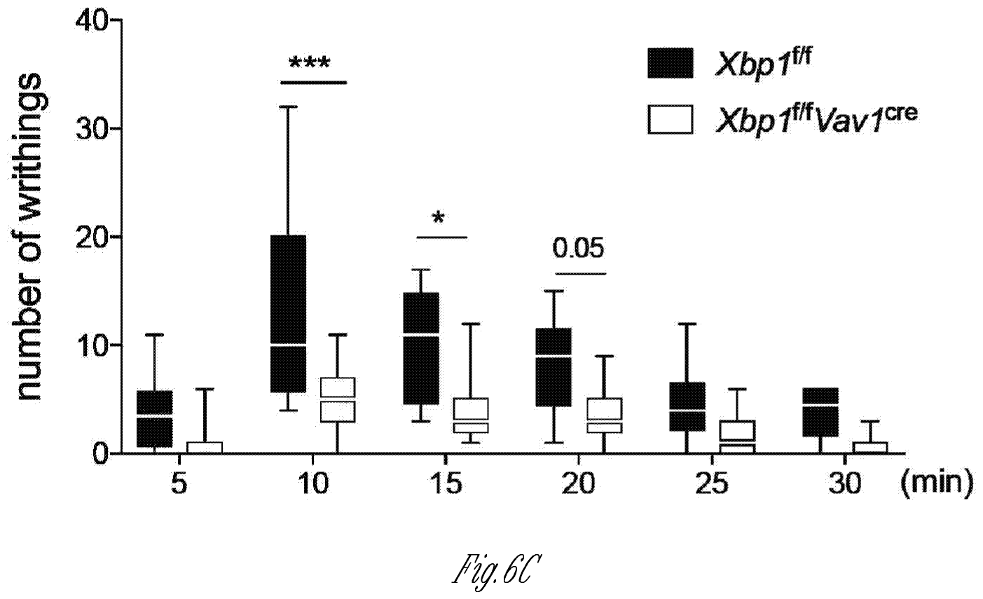

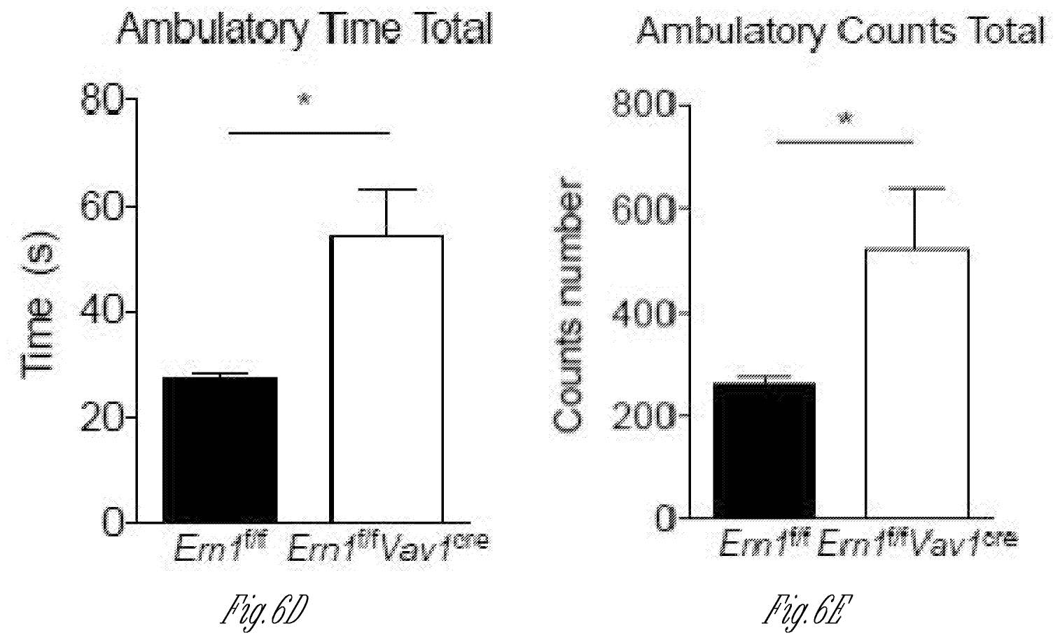

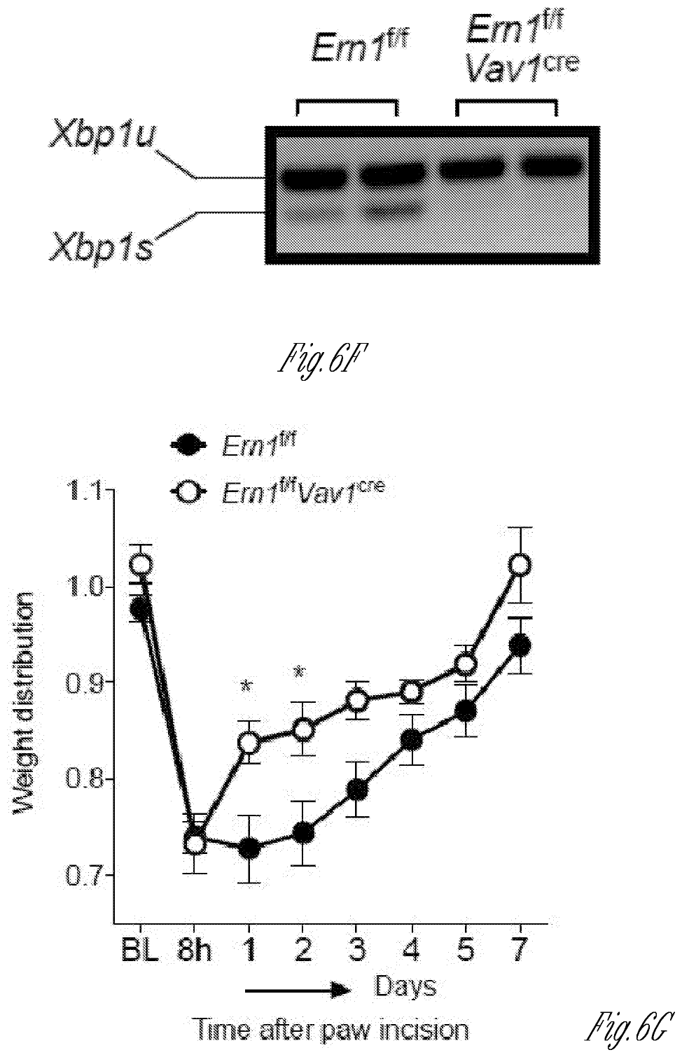

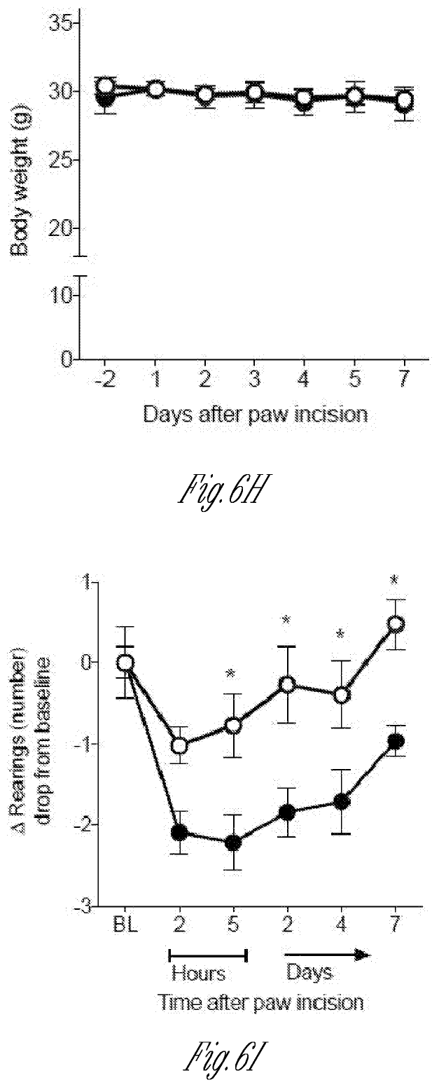

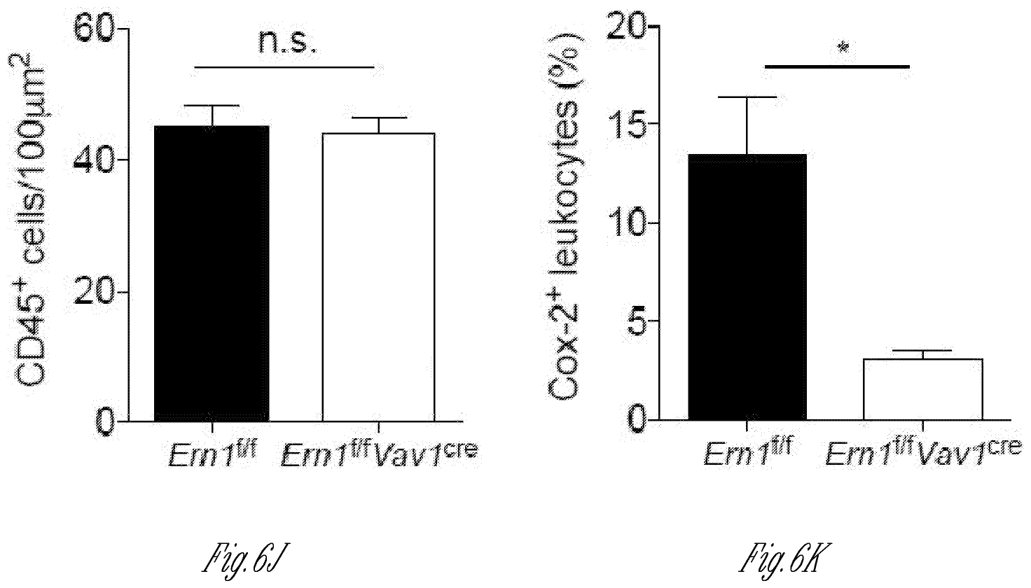

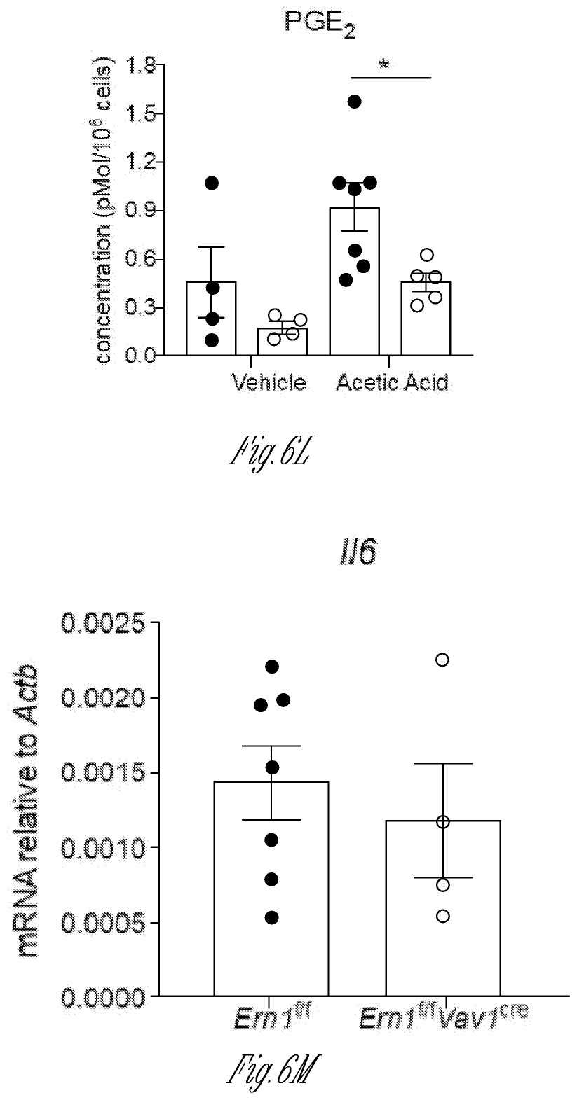

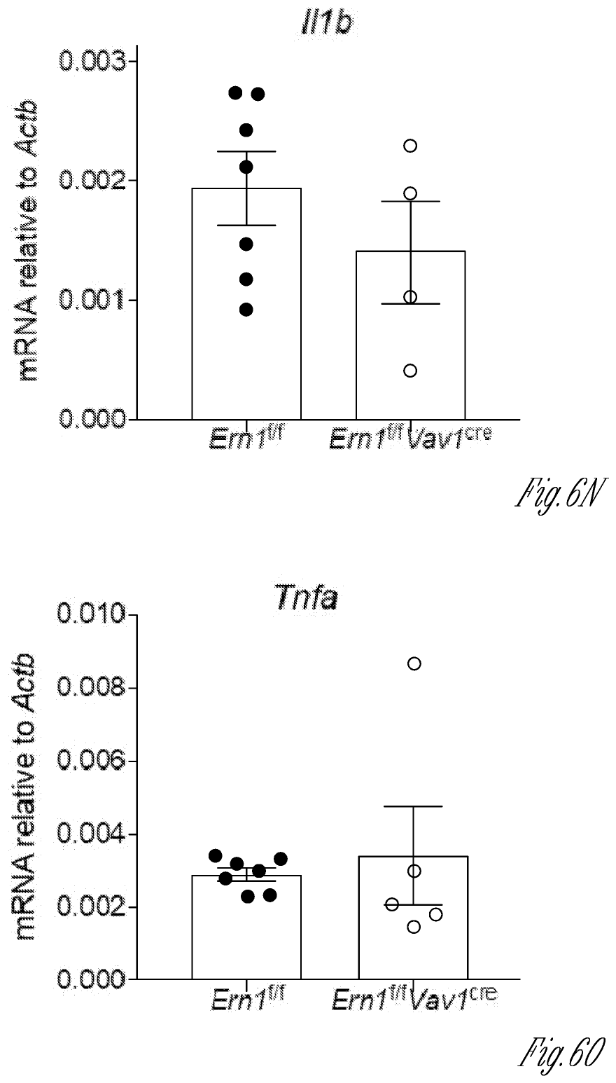

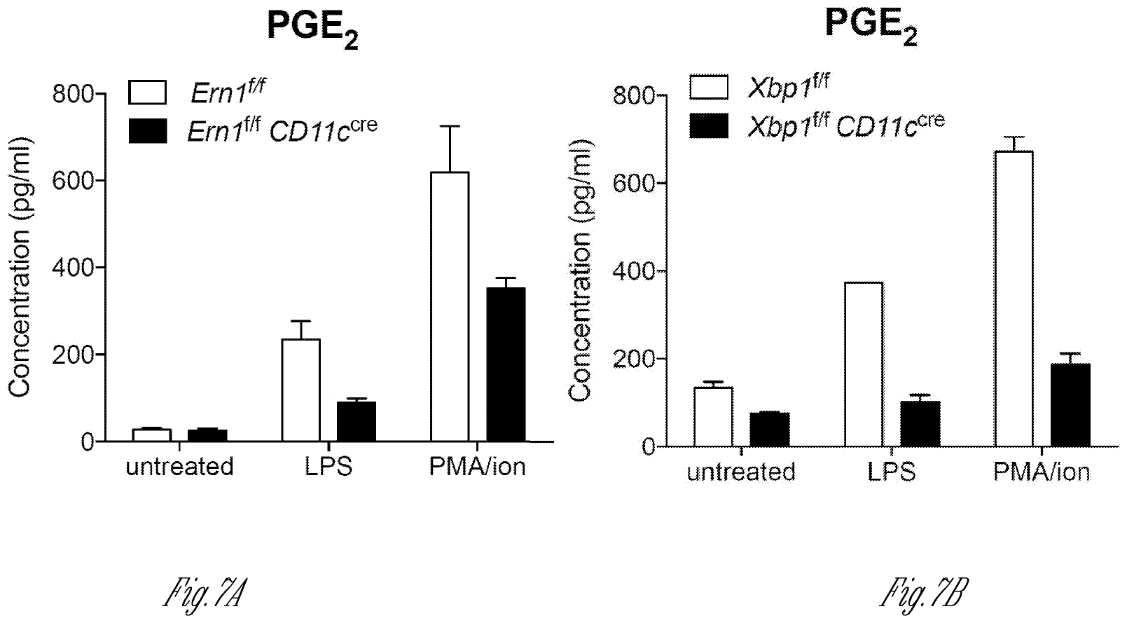

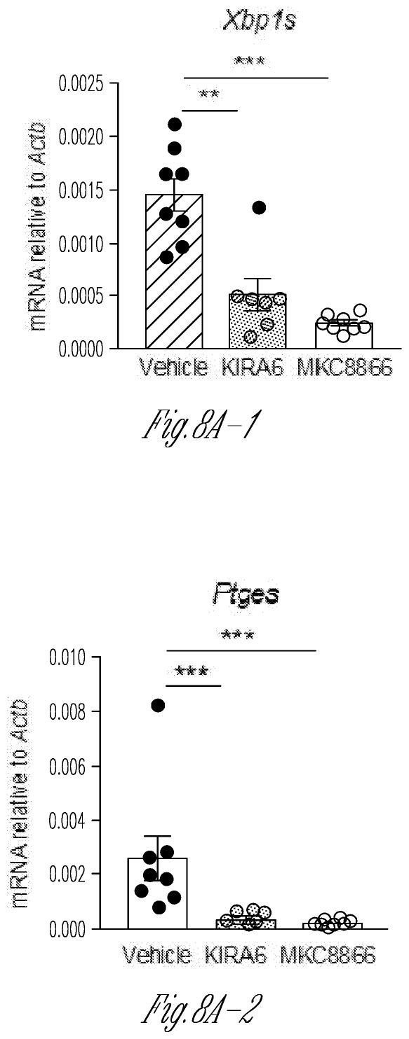

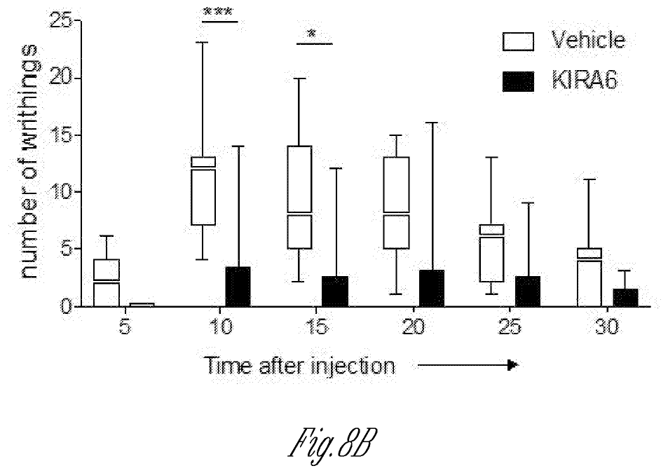

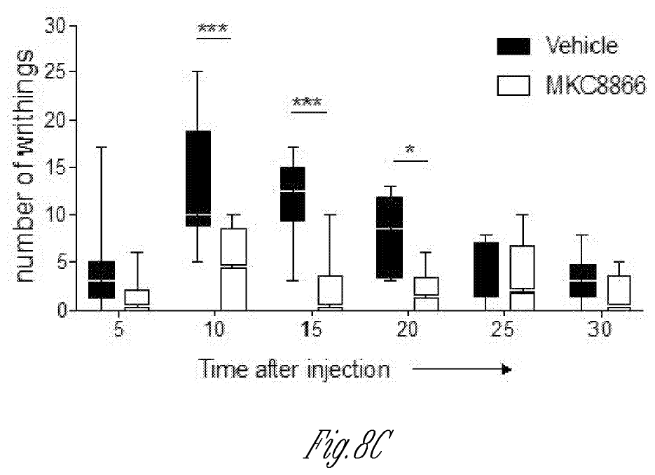

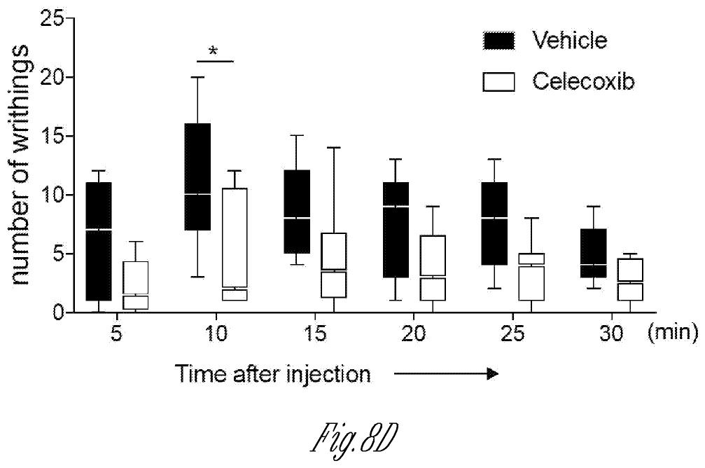

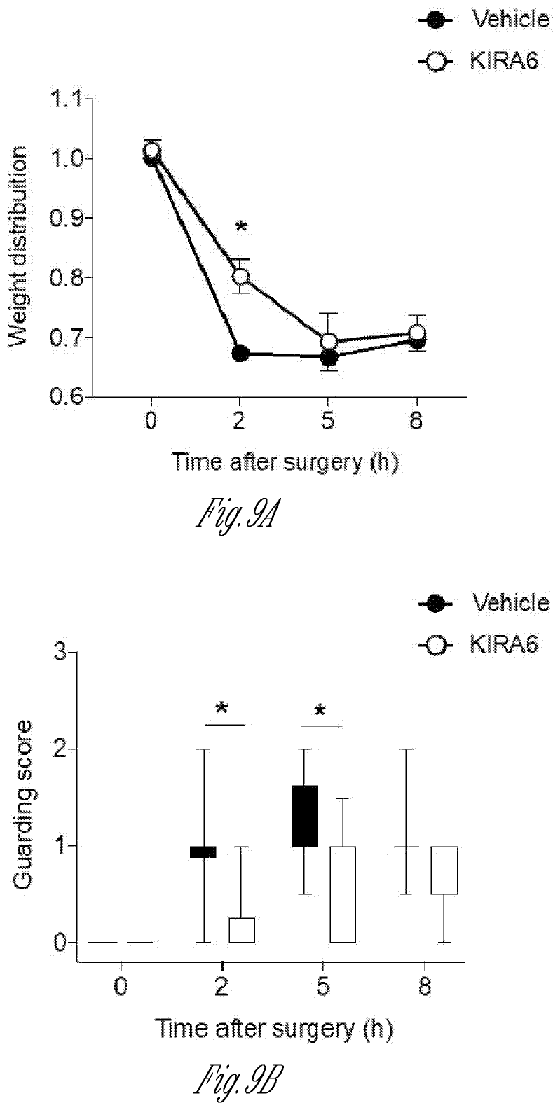

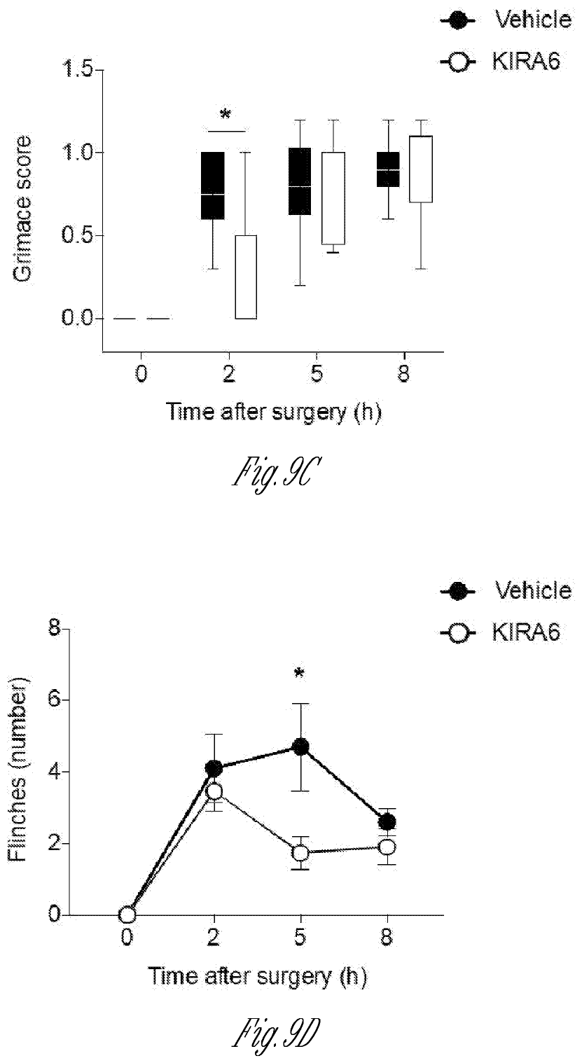

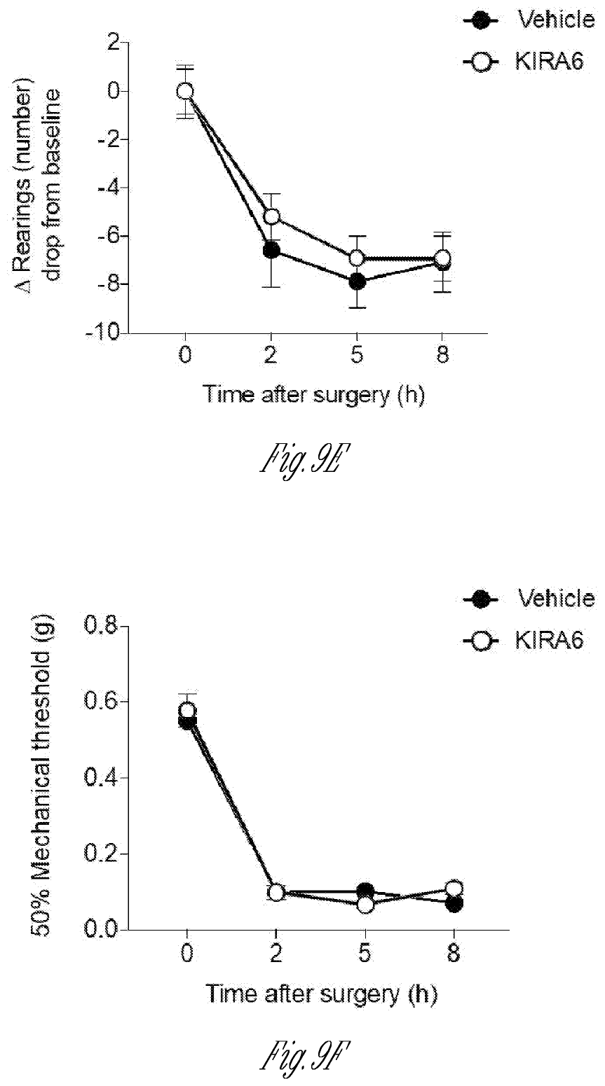

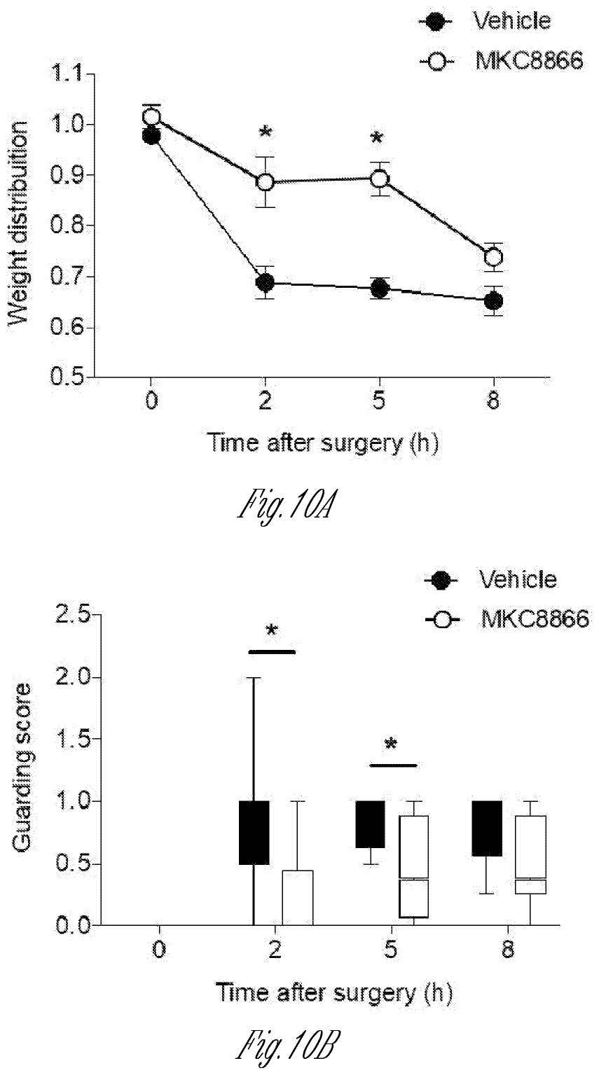

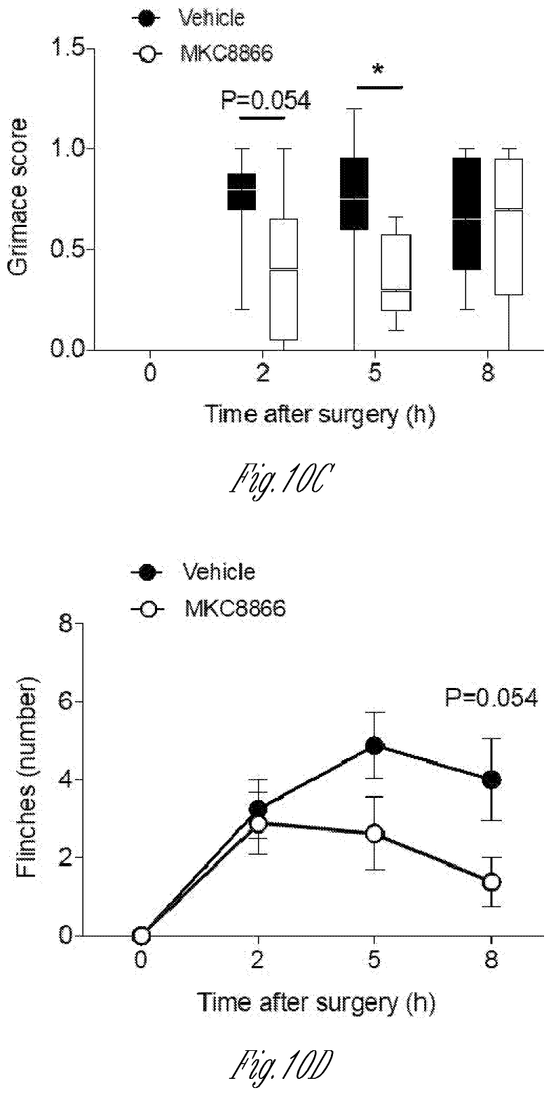

Wild type DC exposed to these microbial products exhibited IRE1α-dependent Xbp1 splicing (FIG. 1A-1B) but did not show robust induction of canonical XBP1 target genes in the ER stress response or activation of other UPR branches. No signs of regulated IRE1α-dependent decay (RIDD) (So et al. Cell Metab 16, 487 (Oct. 3, 2012); Hollien et al. J Cell Biol 186, 323 (Aug. 10, 2009)) were observed upon LPS or zymosan stimulation, as the expression levels of several genes reported to be potentially modulated by this process remained unaltered in DC lacking IRE1α (FIG. 1C-1D).

IRE1α-deficiency did not compromise normal DC generation or survival in response to GM-CSF (FIG. 1E-1F). However, 1,792 and 2,863 genes whose expression was significantly altered were identified in IRE1α-deficient dendritic cells stimulated with either zymosan or LPS, respectively, compared with their wild type counterparts. There was a significant overlap of 1,167 differentially regulated genes between the two stimuli (FIG. 1G), indicating a common effect of IRE1α-deficiency independently of the agonist used. Ingenuity Pathway Analysis (IPA) for these commonly regulated genes revealed enrichment of nine biological categories (Table 10).

TABLE 10
Ingenuity Pathway Analysis (IPA) of RNA-sequences

| Category | Function | N | P value | Z | |
|---|---|---|---|---|---|
| Cell death/ | Survival of organism | 101 | 9 × 10⁻¹² | −3.7 | Decreased |
| Survival | Cell death | 363 | 2 × 10⁻¹⁶ | −2.7 | |
| | Necrosis | 305 | 3 × 10⁻¹⁸ | −2.1 | |
| Cellular | Cell movement | 266 | 4 × 10⁻²⁰ | −3.5 | Decreased |
| Movement | Cellular infiltration | 65 | 2 × 10⁻⁹ | −2.6 | |
| Cellular | Cellular homeostasis | 191 | 5 × 10⁻¹³ | −3.4 | Decreased |
| Maintenance | | | | | |
| Lipid | Synthesis of | 37 | 2 × 10⁻⁸ | −3.0 | Decreased |
| Metabolism | eicosanoid | | | | |
| | Metabolism of | 41 | 3 × 10⁻⁹ | −2.0 | |
| | eicosanoid | | | | |
| | Synthesis of of lipid | 93 | 1 × 10⁻⁹ | −2.2 | |
| Post- | Phosphorylation of | 89 | 1 × 10⁻⁸ | −2.3 | Decreased |
| translational | protein | | | | |
| Modification | | | | | |
| Nucleic Acid | Metabolism of | 73 | 1 × 10⁻¹⁰ | −2.0 | Decreased |
| Metabolism | nucleic acid | | | | |
| Organismal | Organismal death | 253 | 9 × 10⁻¹¹ | 3.2 | Increased |
| Survival | Morbidity or | 261 | 6 × 10⁻¹² | 3.0 | |
| | mortality | | | | |
| Infectious | Infection of | 58 | 5 × 10⁻¹² | 3.0 | Increased |
| Diseases | mammalia | | | | |
| DNA | Alignment of | 13 | 2 × 10⁻¹⁰ | 2.1 | Increased |
| recombination | chromosomes | | | | |
| and repair | | | | | |

IRE1α-deficiency influenced transcriptional processes involved in post-translational protein modification as well as cellular maintenance and survival. As illustrated in Table 10, biosynthesis and metabolism of eicosanoids surprisingly emerged as a major cellular function potentially regulated by IRE1α in DC stimulated with LPS or zymosan.

### Example 3: IRE1α Regulates Expression of Ptgs2 and Ptges

This Example describes analysis of IRE1α transcriptional regulators in bone marrow-derived dendritic cells (DC) and provides experimental evidence that IRE1α Regulates Expression of Ptgs2 and Ptges.

Searches were performed for key regulators that could be responsible for a significant number of the observed transcriptional changes. Twenty-seven regulators were identified that not only changed expression at the mRNA level, but also had a significant number of known targets enriched in selected genes. Expression of 116 and its associated target genes was significantly decreased in TLR-stimulated DC lacking IRE1α, compared with their wild type counterparts (FIG. 2A).

Additionally, and corresponding with IPA analyses denoting altered eicosanoid metabolism, prostaglandinendoperoxide synthase 2 (Ptgs2/Cox-2) and prostaglandin E synthase (Ptges/mPGES-1) emerged as potential regulators that were markedly decreased in IRE1α-deficient dendritic cells exposed to LPS or zymosan (FIG. 2A). Down-regulation of these two enzymes was confirmed at the mRNA and protein levels in stimulated dendritic cells devoid of IRE1α using RT-qPCR and immunoblot assays (FIG. 2B-2D). Importantly. IRE1α deficiency did not affect the constitutive expression of Ptgs1/Cox-1 or Ptges2 (FIG. 2E-2F), indicating that this ER stress sensor primarily mediates the rapid induction of Ptgs2/Cox-2 and Ptges/mPGES-1 in response to inflammatory stimuli. These findings indicate that IRE1α is required for optimal eicosanoid production by myeloid cells.

### Example 4: IRE1α Promotes Prostaglandin Production

This Example illustrates that reduction in IRE1α reduces prostaglandin levels.

Prostaglandins are a major class of eicosanoids whose inducible biosynthesis depends on the rapid metabolism of arachidonic acid by Cox-2 (FIG. 3A). These bioactive lipids participate in the regulation of diverse physiological processes such as allergy, fever, vascular permeability, and pain, amongst many others. Lipidomic analyses revealed that IRE1α deficiency did not influence basal prostaglandin levels in untreated DC (FIG. 3B). However, a profound decrease was detected in the intracellular levels of several prostaglandins, including PGE1, 15-keto PGF2a, D12-PGJ2, PGD3, PGE2, PGF2a, 13,14dh-15k PGE2, PGD2, PGD3 and PGF1a, in LPS-stimulated dendritic cells devoid of IRE1α when compared with their wild type counterparts (FIG. 3B, Table 11).

TABLE 11
Prostanoid species significantly dysregulated
in IRE1a-deficient DC stimulated with LPS

| | Log Fold Change | |
|---|---|---|
| Lipid Species | (Ern1-KO/Ern1-WT) | P-value |
| PGE1 | −3.15 | 2.4E−05 |
| 15-keto PGF2a | −2.84 | 9.5E−08 |
| D12-PGJ2 | −2.48 | 5.5E−06 |
| PGE2 | −2.17 | 4.5E−06 |
| PGD3 | −1.67 | 1.5E−02 |
| 13,14dh-15k-PGE2 | −1.26 | 5.6E−05 |
| PGF2a | −1.21 | 6.9E−04 |
| PGD2 | −1.03 | 1.2E−04 |
| PGF1a | −0.88 | 3.0E−02 |
| 15-keto PGE2 | −0.46 | 2.3E−02 |

Cox-2 converts arachidonic acid to prostaglandin endoperoxide H₂ (PGH₂), which is subsequently metabolized by mPGES-1 to generate the potent lipid mediator prostaglandin E₂ (PGE₂) (FIG. 3A). Corresponding with decreased induction of both Cox-2 and mPGES-1 in IRE1α-deficient dendritic cells stimulated with LPS (FIG. 2), a marked reduction in PGE2 production by these cells was observed in comparison with their wild type counterparts (FIG. 3C-3D). Additional IRE1α-deficient myeloid cell subsets, including primary neutrophils and macrophages, also demonstrated defective PGE2 synthesis upon LPS stimulation. To further confirm these findings in vivo. LPS was administered intraperitoneally (i.p) to transgenic mice specifically lacking IRE1α in leukocytes (Ern1^{f/f} Vav1^{cre}) and PGE2 production was quantified in situ. As shown in FIG. 4A-4C, PGE2 synthesis was reduced in Ern1^{f/f} Vav1^{cre} leucocytes and Ern1^{f/f} Vav1^{cre} macrophages, as well as in Xbp1^{KO} macrophages.

LPS exposure triggered Xbp1 splicing and concomitant IRE1α-dependent induction of both Ptgs2 and Ptges in peritoneal leukocytes (FIG. 4D-4F). Strikingly, mice devoid of IRE1a in leukocytes were incapable of inducing PGE2 production upon peritoneal LPS administration (FIG. 4G). Confirming such transcriptional profiling using an independent agonist (FIG. 2), PGE2 synthesis was also diminished in zymosan-exposed DC lacking IRE1α (FIGS. 3E-1 and 3E-2). Similar results were observed in vivo after i.p. administration of zymosan to mice lacking IRE1α in leukocytes. Lipidomic analyses revealed that production of all Cox-2-dependent prostaglandins (PGE₂, PGD₂, PGF₂α and TBX₂) was reduced, while lipoxygenase-dependent 15-HETE was unaltered, in cell-free peritoneal lavage from Ern1^{f/f} Vav1^{cre} compared with Ern1^{f/f} mice (FIG. 3M-3Q). Of note. XBP1 deletion phenocopied the same defects observed in IRE1α-deficient myeloid cells (FIGS. 3E-1, 3E-2, 4C), while ablation of other ER stress sensors such as PERK (encoded by Eif2ak3) and ATF6a did not compromise inducible PGE2 generation (FIGS. 3F-3G). These data indicate that the IRE1α-XBP1 arm of the ER stress response is selectively required for optimal PGE2 production by LPS-stimulated or zymosan-stimulated myeloid cells. Interestingly, IRE1α-dependent induction of PGE2 was also observed in DC treated with TLR1, TLR2, TLR4, TLR5 and TLR6 agonists, while stimulation via TLR3, TLR8 or TLR9 had no effect (FIG. 4H). These results are consistent with previous reports demonstrating predominant IRE1α-XBP1 activation by agonists engaging membrane-bound, but not endosomal TLRs. Reduced PGE2 induction was also found in IRE1α-deficient DC activated with phorbol myristate acetate (PMA) (FIG. 4H), thus ruling out the possibility that IRE1α ablation could compromise proximal TLR signaling. Importantly, we also found diminished PGE2 production, accompanied by reduced expression of both Cox-2 and mPGES-1, in IRE1α-deficient DC treated with the pharmacological ER stressor thapsigargin (FIG. 4I-4J). Taken together, these data indicate that optimal PGE2 synthesis by murine myeloid cells undergoing ER stress, or stimulated via membrane-bound TLRs, requires IRE1α-XBP1 activation that promotes expression of Cox-2 and mPGES-1.

To define whether IRE1α-XBP1 signaling also controlled inducible PGE₂ production in human myeloid cells, monocyte-derived DC were generated from peripheral blood of healthy volunteers. The IRE1α-XBP1 signaling pathway was then abrogated from the dendritic cells using gene-editing techniques (see Example 1 section on Gene Editing in Human DC for details). Transient transfection of primary human DC with sgRNA-Cas9 complexes targeting XBP1 effectively edited this gene and prevented the generation of its spliced (active) form upon zymosan treatment (FIG. 3H).

Notably, induction of PTGS2 and PTGES, as well as PGE2 production, were significantly diminished in zymosan-exposed human DC devoid of XBP1, compared with their wild type counterparts transfected with scrambled sgRNA-Cas9 complexes (FIG. 3I-3J). Importantly, similar effects were observed when ERN1-deficient human dendritic cells were treated with zymosan (FIG. 3K-3L), thus confirming a conserved role for IRE1α-XBP1 signaling as a key mediator of inducible PGE₂ production in human DC.

### Example 5: Analysis of Promoter Binding Sites for IRE1α-Activated XBP1s

This Example describes experiments designed to determine the molecular mechanism by which IRE1α-activated XBP1 (XBP1s) mediates inducible PGE₂ production in human myeloid cells.

The promoter regions of PTGS2 and PTGES were analyzed for potential IRE1□-activated XBP1 (XBP1s) binding sites using methods described by Acosta-Alvear et al. (Mol Cell 27, 53-66 (2007); Clauss et al. Nucleic Acids Res 24, 1855-1864 (1996). Putative X-box-binding and Unfolded Protein Responses Element A (UPRE-A) sequences were found on the PTGS2 promoter (FIG. 5A). Additionally, an X-box-binding region and two ETS domain-binding sites were identified in the PTGES promoter (FIG. 5B). These results indicated that XBP1s could operate as a driver of PTGS2 and PTGES transcription.

ChIP-PCR was used to evaluate direct XBP1s binding to the promoter regions identified. Human primary DC were stimulated with zymosan alone or in combination with 2-deoxy-D-glucose (2-DG), which inhibits N-linked protein glycosylation and hence causes ER stress and robust IRE1α-XBP1 activation (Marquez et al. Frontiers in immunology 8, 639 (2017)). Zymosan exposure provoked an increase in XBP1s binding to the predicted PTGS2 and PTGES promoter regions, and concomitant treatment with the ER stressor 2-DG substantially enhanced these effects (FIG. 5C-5D). Importantly, disabling the IRE1α RNAse domain using a selective pharmacological inhibitor abrogated XBP1s binding to these promoters in zymosan-stimulated human DC undergoing ER stress (FIG. 5C-5D). XBP1s binding to the GFPT1 promoter was also observed, as previously reported (Marquez et al. Frontiers in immunology 8, 639 (2017)), whereas promoter regions of pri-miR-21 devoid of XBP1s-binding sites were not enriched in these assays (FIG. 5E-5F). Furthermore, luciferase reporter assays using HEK293 cells demonstrated that XBP1s was sufficient to dose-dependently transactivate the PTGS2 and PTGES promoters, while the PERK-controlled ER stress transcription factor CHOP had no effects in this reporter system (FIG. 5G-5H). Taken together, these data indicate that IRE1α-activated XBP1s mediates inducible PGE₂ biosynthesis by directly driving transcriptional induction of both PTGS2 and PTGES.

### Example 6: IRE1α Expression in Immune Cells Promotes Pain Behaviors

This Example illustrates experiments designed to evaluate whether loss of IRE1α function can reduce pain.

PGE₂ generated via induction of Cox-2 and mPGES-1 engages EP1-4 receptors on peripheral sensory neurons and the central nervous system to promote pain responses. The inventors postulated that mice lacking IRE1α in leukocytes would demonstrate reduced pain behaviors due to their impaired capacity to induce PGE₂ production in response to inflammatory stimuli (FIG. 4D-4G; 3M-3Q).

Two classical PGE₂-dependent models of pain were used to test this hypothesis. An acetic acid-based model as used for inflammatory visceral pain (Kamei et al. J Biol Chem 279, 33684-33695 (2004); Trebino et al. Proc Natl Acad Sci USA 100, 9044-9049 (2003); Collier et al. Br J Pharmacol Chemother 32, 295-310 (1968); Lu et al. Acta Pharmacol Sin 26, 1505-1511 (2005)). A paw incision model of post-surgical pain was also employed (Pogatzki & Raja, Anesthesiology 99, 1023-1027 (2003)).

Acetic acid (0.9% v/v) was inject i.p. into either Ern1^{f/f} or Ern1^{f/f} Vav1^{cre} male mice and writhing behaviors were monitored over time by a blinded observer. Peritoneal leukocytes demonstrated IRE1α-dependent Xbp1 splicing upon acetic acid administration (FIG. 6A). Strikingly, the number of writhing events recorded within the first 30 minutes were significantly reduced in Ern1^{f/f} Vav1^{cre} male mice compared with their IRE1α-sufficient counterparts (FIG. 6B). Reduced pain behaviors were also evidenced in Ern1^{f/f} Vav1^{cre} female mice in a separate experiment upon acetic acid administration, indicating that IRE1α expression in leukocytes does not differentially. Similar effects were observed in mice selectively lacking XBP1 in leukocytes (Xbp1^{f/f} Vav1^{cre}) (FIG. 6C), thus confirming a key role for canonical IRE1α-XBP1 signaling in controlling this behavioral process.

Automated unbiased and blinded tests were also performed after acetic acid injection showing that the total ambulatory times and counts, indicative of displacement ability, were normally preserved in Ern1^{f/f} Vav1^{cre} mice, whereas control Ern1^{f/f} animals displayed a significant reduction in inflammatory visceral pain (FIG. 6D-6E).

PGE2 levels in cell-free peritoneal lavage samples from mice lacking IRE1α in leukocytes upon administration of acetic acid was also reduced (FIG. 6L).

Together, these data demonstrate that the activation of IRE1α and its downstream XBP1s in leukocytes promotes inflammatory visceral pain in this model.

Similar levels of IL-6, IL-1β or TNFα were found in the peritoneal lavage of Ern1^{f/f} Vav1^{cre} vs. Ern1^{f/f} mice upon acidic acid administration (FIG. 6M-6O). These data indicate that, in the context of acetic acid administration, IRE1α does not control cytokines such as IL-6, IL-1β and TNFα, but drives the production of PGE₂.

Next, experiments were performed to evaluate whether IRE1α deficiency in leukocytes could also influence post-operative pain, which is a PGE₂-mediated process commonly treated with COX-2 inhibitors. A surgical incision was made in the left hind paw of either Ern1^{f/f} or Ern1^{f/f} Vav1^{cre} mice, and non-reflexive pain-related behaviors such as hind paw weight distribution, as well as spontaneous rearing activity, were monitored over time and analyzed in comparison with baseline measurements prior to surgery. IRE1α-dependent Xbp1 splicing was observed in CD45⁺ leukocytes sorted from the injury site 24 hours post-surgery (FIG. 6F). The proportion of neutrophils, macrophages and dendritic cells infiltrating the lesions at this time point was not altered in Ern1^{f/f} vs. Ern1^{f/f} Vav1^{cre} mice. However, a significant reduction in the number of Cox-2-expressing leukocytes infiltrating the injured tissues was observed in the surgical site of Ern1^{f/f} Vav1^{cre} mice, compared with their littermate controls (FIG. 6J-6K). Accordingly, weight bearing distribution tests indicated that Ern1^{f/f} Vav1^{cre} mice had superior capacity to use the injured paw 24-48 hours after surgery, compared with their IRE1α-sufficient counterparts (FIG. 6G), and these effects were not caused by differential body weight in the two genotypes FIG. 6H). Ern1^{f/f} Vav1^{cre} mice also displayed reduced impairment and more rapid recovery of rearing activity in comparison with Ern1^{f/f} animals, a phenotype that appeared as early as 5 hours post-surgery and was maintained for up to 7 days after surgery (FIG. 6I). In contrast, mechanical hypersensitivity and paw perimeter were comparable in Ern1^{f/f} vs. Ern1^{f/f} Vav1^{cre} mice post-surgery. Taken together, these data indicate that mice lacking IRE1α-XBP1 in leukocytes exhibit reduced behavioral pain responses in two distinct PGE₂-dependent models of pain.

Evidence is therefore provided herein demonstrating an unexpected new function for the ER stress sensor IRE1α as a central mediator of prostaglandin biosynthesis and behavioral pain responses in mice. The data provided herein indicate that a previously unappreciated mechanism exists whereby IRE1α activates transcription factor XBP1 to promote optimal expression of two rate-limiting enzymes that are necessary for inducible prostaglandin biosynthesis, namely Cox-2 and mPGES-1. Novel and more effective pain management strategies can be provided by pharmacological modulation of IRE1α-XBP1 signaling. Such pharmacological modulation of IRE1α-XBP1 signaling is an alternative approach for pain control that can provide better analgesia, diminished opioid requirements, and reduced opioid side effects. IRE1α-XBP1 signaling can also regulate processes driven by prostaglandins, including pregnancy, fever, vascular permeability, allergy and immunosuppression in cancer hosts will be of substantial interest.

### Example 7: Reduction of Pain by Inhibitors of IRE1α

To determine if pharmacological disabling of IRE1α-XBP1 signaling could reduce inflammatory visceral pain, two commercially available inhibitors of IRE1α were employed: the kinase domain-specific inhibitor KIRA6 (25 mg/kg) and the RNAse domain-specific inhibitor MKC8866 (20 mg/kg).

These inhibitors were independently administered intraperitoneally to C57BL/6J mice 6 hours and 30 minutes before acetic acid injection, and writhing behaviors were recorded for 30 minutes. Treatment with both compounds significantly reduced Xbp1s and Ptges expression in peritoneal leukocytes (FIGS. 8A-1 and 8A-2) obtained from the mice after acetic acid injection. The number of writhings also decreased after acetic acid injection (FIG. 8B-8C) when KIRA6 (25 mg/kg) and MKC8866 (20 mg/kg) were administered. Hence, pharmacologic inhibition of IRE1α-XBP1 can reduce pain in mammalian subjects.

Treatment with equimolar amounts (20 mg/kg) of a selective Cox-2 inhibitor, Celecoxib, also decreased the number of writhings, further indicating that the foregoing behavioral response depends on an intact Cox-2-PGE₂ axis (FIG. 8D).

Together, these data demonstrate that the activation of IRE1α-XBP1 in leukocytes promotes inflammatory visceral pain in the acetic acid-based model and that inhibition of IRE1α-XBP1 can reduce pain.

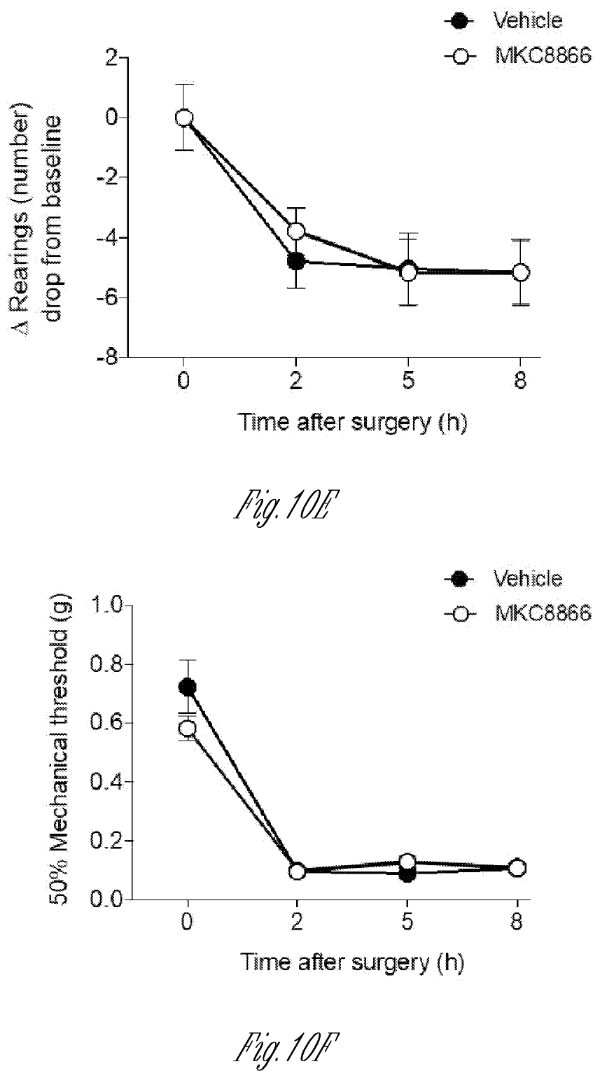

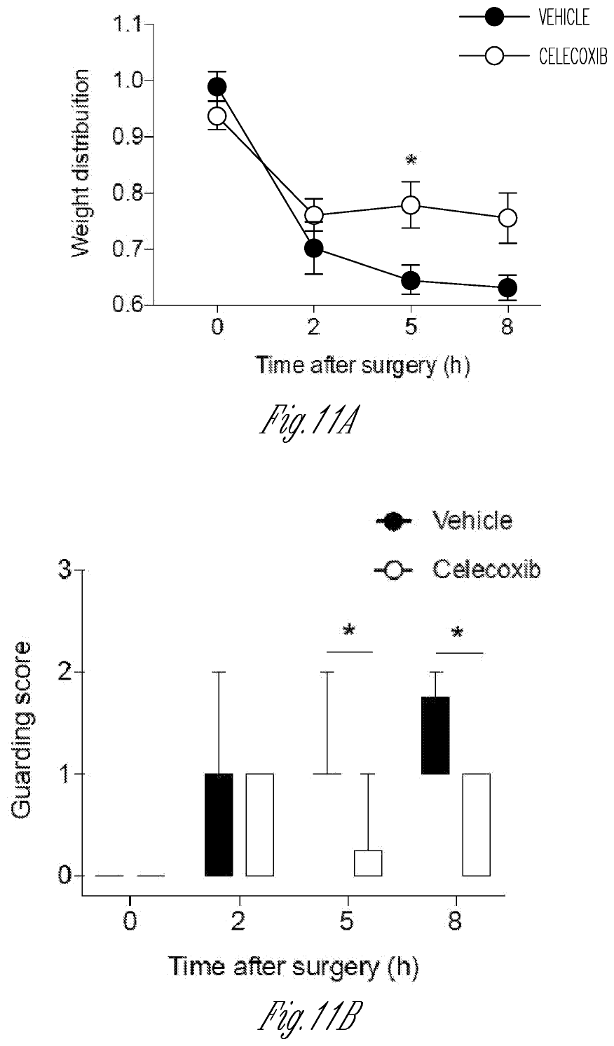

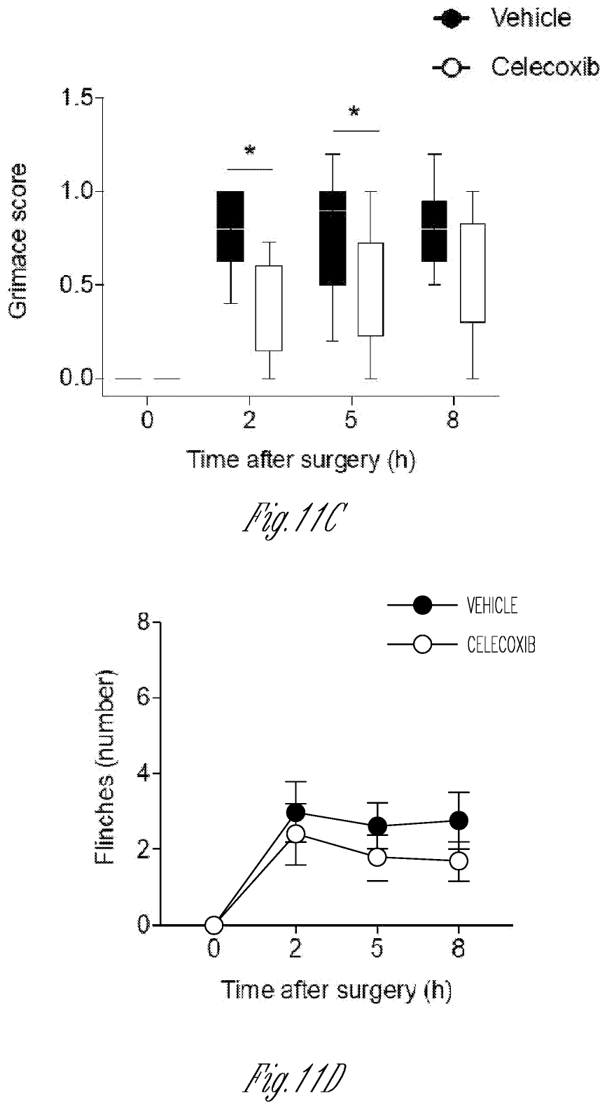

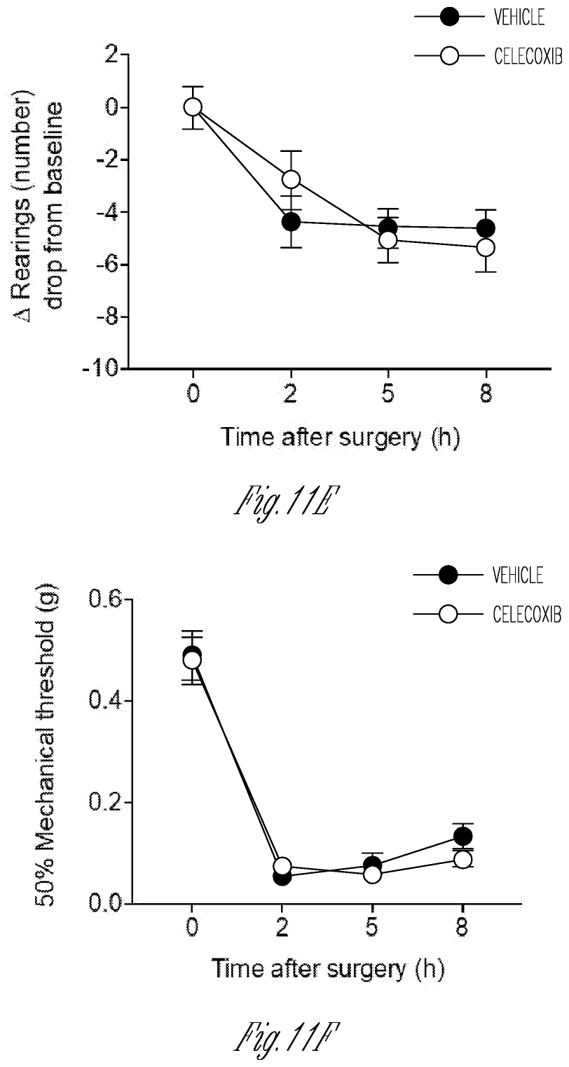

To determine whether pharmacological targeting of IRE1α could also modulate post-surgical pain. KIRA6 (FIG. 9A-9F) or MKC8866 (FIG. 10) was administered i.p. 6 hours and 30 minutes prior to paw incision surgery, and pain responses were monitored thereafter. IRE1α inhibition in vivo improved nociceptive functional behaviors, as demonstrated by a more balanced weight distribution when compared to vehicle treated mice (FIGS. 9A and 10A). Grimace and guarding scales post-surgery were also significantly reduced in mice receiving either KIRA6 (FIG. 9B-9C) or MKC8866 (FIG. 10B-10C). Interestingly, in contrast to our observations using Ern1^{f/f} Vav1^{cre} mice, we found reduced flinching activity after paw incision in KIRA6- or MKC8866-administered groups (FIGS. 9D and 10D), suggesting a pro-algesic role for IRE1α in additional non-leukocyte cells in this setting. Rearing activity was unchanged upon IRE1α inhibition (FIGS. 9E and 10E), indicating that complete inhibition of IRE1α might be required for altering this specific behavior after paw incision. Consistent with our results using conditional IRE1α-deficient mice, mechanical hypersensitivity remained unaltered upon administration of IRE1α inhibitors (FIGS. 9F and 10F). As a positive control, we administered equimolar amounts (20 mg/kg) of Celecoxib following the same scheme and route described above. Similar to IRE1α inhibition, we observed a more balanced weight bearing distribution as wells as diminished guarding and grimace scores after paw incision in mice receiving Celecoxib, compared with vehicle treated mice (FIG. 11A-11C). Flinches, rearing activity and mechanical threshold after paw incision also remained unaffected upon Celecoxib treatment (FIG. 11D-11F). These data indicate that mice lacking IRE1α-XBP1 in leukocytes exhibit reduced behavioral pain responses in two distinct PGE2-dependent models of pain, and that targeting IRE1α pharmacologically can modulate these pain behaviors in vivo.

### Example 8: PGE2 Production by Ovarian Cancer-Associated Dendritic Cells

This Example illustrates experiments on prostaglandin (PGE₂) concentrations in ovarian cancer-associated dendritic cells.

Ovarian cancer cells were introduced into Ern1^{f/f} and Ern1^{f/f} CD11c^{cre} mice as well as into Xbp1^{f/f} and Xbp1^{f/f} CD11c^{cre} mice. After 24-28 days, tumor-associated dendritic cells were isolated from metastatic ovarian cancer ascites samples using flow cytometry and the cells were cultured in the presence of LPS or phorbol myristate acetate (PMA).

As shown in FIG. 7A, LPS- or PMA-stimulated dendritic cells lacking CD11c (Ern1^{f/f} CD11c^{cre} cells) exhibited reduced PGE₂ production compared to cells that do express CD11c. Similarly, FIG. 7B also shows that LPS- or PMA-stimulated dendritic cells lacking Xbp1 (Xbp1^{f/f} CD11c^{cre} cells) exhibited reduced PGE₂ production compared to cells that do express Xbp1.

### Example 9: Evaluating Compounds for Inhibition of Xbp1 Splicing

This Example describes methods for evaluating whether test compounds can inhibit Xbp1 splicing.

Dendritic cells, or any other myeloid cell type, can be incubated in 96 well plates, each well containing one or more of the compounds described herein. As controls, dendritic cells can be incubated without any test compounds (negative control) or Ern1^{f/f} Vav1^{cre} (Ern1^{KO}) cells can be incubated with compounds as a positive control for IRE1α inhibition.

Total RNA can be isolated using RNeasy Mini kit or QIAzol lysis reagent (Qiagen) according to the manufacturer's instructions. RNA (0.1-1 μg) can be reverse-transcribed to generate cDNA using the qScript cDNA synthesis kit (Quantabio). Quantitative RT-PCR can be performed using PerfeCTa SYBR green fastmix (Quantabio) and TaqMan Universal PCR master mix (Life Technologies) on a QuantStudio 6 Flex real-time PCR system (Applied Biosystems). Normalized gene expression can be calculated by comparative threshold cycle method using ACTB or Actb as a control. Xbp1 splicing assays can be performed as described by Lee et al. (Proc Natl Acad Sci USA 100, 9946 (Aug. 19, 2003)). PCR products may be separated by electrophoresis through a 2.5% agarose gel and visualized by ethidium bromide staining. Primers that can be used in this study are described in Table 9.

Compounds that inhibit the formation of the Xbp1s (e.g., shown in FIG. 1A) are inhibitors of Xbp1 splicing. Other cell types can be similarly tested for Xbp1 splicing and inhibition thereof by the compounds described herein.

### Example 10: Evaluating Compounds for In Vitro Inhibition of PGE₂ Production

This Example describes methods for evaluating whether test compounds can inhibit PGE₂ production in cell culture.

Dendritic cells (2.5×10⁵), or any other cell type described herein, can be incubated in 96 well plates, each well containing one or more of the compounds described herein. As controls, dendritic cells can be incubated without any test compounds (negative control) or Ern1^{f/f} Vav1^{cre} (Ern1^{KO}) cells can be incubated with compounds as a positive control for IRE1α inhibition. Cells can be stimulated with LPS or any other TLR or CLR (C-type lectin) agonist or PMA. PGE2 can be measured in the supernatants using PGE2 ELISA kit (Enzo, Cat #ADI-900-001) or by mass spectrometry. If different number of cells were plated. PGE2 levels can be normalized to 2.5×105 cells/well. Cell viability counts can be observed to evaluate the toxicity of test compounds.

Compounds that inhibit the formation of PGE₂ (e.g., shown in FIG. 3C-3D) compared to negative controls are inhibitors of PGE₂ production by dendritic cells. Other cell types can be similarly tested for PGE₂ production and inhibition thereof by the compounds described herein.

### Example 11: Evaluating Compounds for In Vivo Inhibition of PGE2 Production

This Example describes methods for evaluating whether test compounds can inhibit PGE₂ production in vivo.

One or more of the compounds described herein can be administered daily to wild type mice for 1-7 days. As controls, some wild type mice may not receive any test compounds, and compounds may also be administered to Ern1KO mice. Neutrophils, macrophages, dendritic cells, or other cell types can be collected from the mice. The cells can be washed and stimulated with LPS, then analyzed for PGE₂ production using the PGE₂ ELISA kit described in Example 1. Plates can be read at 405 nm using Vairoskan (Thermo Fischer Scientific).

Compounds that inhibit the formation of PGE₂ (e.g., as shown FIGS. 4A-4C, 4I) compared negative controls are inhibitors of PGE₂ production by myeloid cells. Other cell types can be similarly tested for PGE₂ production and inhibition thereof by the compounds described herein.

### Example 12: Evaluating Compounds for Pain Reduction

This Example describes methods for evaluating whether test compounds can inhibit pain in vivo.

After surgery or upon use of any related stimulation causing local, peripheral or systemic tissue injury, wild type or Ern1^{KO} mice can be placed in individual acrylic chambers on an elevated mesh floor for 3045 min before testing. Two spontaneous pain-relate behaviors can be evaluated: rearings and paw flinches. Following the acclimatization period, the number of total vertical rearings and paw flinches can be quantified during a 2-min period. Vertical rearings can be defined as the number of times that the animal stood supporting its weight on both hind limbs. Vertical rearings are a normal behavior in rodents, thus a reduction of this behavior is indicative of a protective way to prevent pain due to movement, which mimics pain induced by surgeries in humans. Spontaneous flinching of the affected paw can be quantified every time that the animal shacked the affected paw without any stimulation. Flinches of the injured paw are pain-related behaviors indicative of breakthrough pain, similar to intense spontaneous spikes of pain in humans with postoperative pain.

Mechanical hypersensitivity can be assessed after quantification of vertical rearings and spontaneous flinching. Mechanical withdrawal thresholds can be calculated using the up-down method and applying force with calibrated Von Frey filaments (0.07-g, 0.17-g, 0.40-g, 0.60-g, 1.04-g, 1.37-g, and 2.0-g, Stoeling, Wood Dale, Ill., USA) to the plantar aspect of the paw for 5 seconds. Paw withdraws or flinching in response to a given applied force can be noted as a positive pain response.

Hind paw weight bearing distribution can be determined using an incapacitance tester apparatus (Stoelting, Ill., version 5.64). This is a test for non-reflexive behaviors that represents a spontaneous pain-related behavior that mimics postoperative pain behaviors in humans (protection of the surgery site from normal activities). Before surgery, animals can be habituated for at least 3 days to the apparatus, in which animals stand with each hind paw resting on individual weight plates inside an acrylic chamber. The apparatus measures the body weight distributed between the two hind paws over a 3 second period and provides the average measurement. The average value of each hind paw can be used to determine the weight distribution ratio (ispsilateral/contralateral side). A ratio below one indicates a greater weight bearing on the contralateral paw and can therefore be considered as a pain-related behavior.

Writhing spontaneous pain behaviors can be evaluated after intraperitoneal injection of 0.9% acetic acid (v/v, 5 ml/kg). The number of writhing responses can be quantified immediately after acetic acid injection for 30 min in 5 min intervals by an observer blinded to genotype. Writhings induced by acetic acid are overt stretching behaviors indicative of abdominal pain, a phenomenon that is dependent upon mPGES-1 and PGE2 (Kamei et al. J Biol Chem 279, 33684 (Aug. 6, 2004); Trebino et al. Proc Natl Acad Sci USA 100, 9044 (Jul. 22, 2003)).

One or more of the compounds described herein can be administered daily to two or more wild type mice for 1-7 days, before, during, and/or after surgery or administration of acetic acid. As controls, some wild type mice may not receive any test compounds, acetic acid or surgery. Other controls can include compounds administered to Ern1KO mice that are subjected to surgery or administration of acetic acid.

Compounds that reduce pain responses (e.g., as shown in FIG. 6A-6E, 6G, 6I, 8B-8C) in mice are useful pain inhibitors.

### REFERENCES

1. S. E. Bettigole, L. H. Glimcher, Endoplasmic reticulum stress in immunity. Annu Rev Immunol 33, 107-138 (2015).
2. H. Yoshida, T. Matsui, A. Yamamoto, T. Okada, K. Mor. XBP1 mRNA is induced by ATF6 and spliced by IRE1 in response to ER stress to produce a highly active transcription factor. Cell 107, 881-891 (2001).
3. A. H. Lee, N. N. Iwakoshi, L. H. Glimcher, XBP-1 regulates a subset of endoplasmic reticulum resident chaperone genes in the unfolded protein response. Mol Cell Biol 23, 7448-7459 (2003).
4. A. H. Lee, E. F. Scapa, D. E. Cohen, L. H. Glimcher, Regulation of hepatic lipogenesis by the transcription factor XBP1. Science 320, 1492-1496 (2008).
5. X. Chen et al., XBP1 promotes triple-negative breast cancer by controlling the HIF1alpha pathway. Nature 508, 103-107 (2014).
6. Q. Duan et al., Deregulation of XBP1 expression contributes to myocardial vascular endothelial growth factor-A expression and angiogenesis during cardiac hypertrophy in vivo. Aging cell 15, 625-633 (2016).
7. A. X. Zhou. I. Tabas, The UPR in atherosclerosis. Seminars in immunopathology 35, 321-332 (2013).
8. O. Tufanli et al., Targeting IRE1 with small molecules counteracts progression of atherosclerosis. Proc Natl Acad Sci USA 114. E1395-E1404 (2017).
9. Q. Qiu et al., Toll-like receptor-mediated IRE1alpha activation as a therapeutic target for inflammatory arthritis. EMBO J 32, 2477-2490 (2013).
10. J. R. Cubillos-Ruiz, S. E. Bettigole, L. H. Glimcher, Tumorigenic and Immunosuppressive Effects of Endoplasmic Reticulum Stress in Cancer. Cell 168, 692-706 (2017).
11. J. R. Cubillos-Ruiz et al., ER Stress Sensor XBP1 Controls Anti-tumor Immunity by Disrupting Dendritic Cell Homeostasis. Cell 161, 1527-1538 (2015).
12. F. Martinon, X. Chen, A. H. Lee, L. H. Glimcher, TLR activation of the transcription factor XBP1 regulates innate immune responses in macrophages. Nat Immunol 11, 411-418 (2010).
13. J. S. So et al., Silencing of lipid metabolism genes through IRE1alpha-mediated mRNA decay lowers plasma lipids in mice. Cell Metab 16, 487-499 (2012).
14. J. Hollien et al., Regulated Ire1-dependent decay of messenger RNAs in mammalian cells. J Cell Biol 186, 323-331 (2009).
15. F. Osorio et al., The unfolded-protein-response sensor IRE-1alpha regulates the function of CD8alpha+ dendritic cells. Nat Immunol 15, 248-257 (2014).
16. C. Hetz, F. R. Papa, The Unfolded Protein Response and Cell Fate Control. Mol Cell 69, 169-181 (2018).
17. A. J. Dannenberg, K. Subbaramaiah, Targeting cyclooxygenase-2 in human neoplasia: rationale and promise. Cancer Cell 4, 431-436 (2003).
18. E. Ricciotti, G. A. FitzGerald, Prostaglandins and inflammation. Arteriosclerosis, thrombosis, and vascular biology 31, 986-1000 (2011).
19. J. de Boer et al., Transgenic mice with hematopoietic and lymphoid specific expression of Cre. Eur J Immunol 33, 314-325 (2003).
20. D. Acosta-Alvear et al., XBP1 controls diverse cell type- and condition-specific transcriptional regulatory networks. Mol Cell 27, 53-66 (2007).
21. I. M. Clauss, M. Chu, J. L. Zhao, L. H. Glimcher. The basic domain/leucine zipper protein hXBP-1 preferentially binds to and transactivates CRE-like sequences containing an ACGT core. Nucleic Acids Res 24, 1855-1864 (1996).
22. S. Marquez et al., Endoplasmic Reticulum Stress Sensor IRE1alpha Enhances IL-23 Expression by Human Dendritic Cells. Frontiers in immunology 8, 639 (2017).
23. C. D. Funk, Prostaglandins and leukotrienes: advances in eicosanoid biology. Science 294, 1871-1875 (2001).
24. J. L. Stock et al., The prostaglandin E2 EP1 receptor mediates pain perception and regulates blood pressure. J Clin Invest 107, 325-331 (2001).
25. A. Kawabata, Prostaglandin E2 and pain—an update. Biol Pharm Bull 34, 1170-1173 (2011).
26. D. Kamei et al., Reduced pain hypersensitivity and inflammation in mice lacking microsomal prostaglandin e synthase-1. J Biol Chem 279, 33684-33695 (2004).
27. C. E. Trebino et al., Impaired inflammatory and pain responses in mice lacking an inducible prostaglandin E synthase. Proc Natl Acad Sci USA 100, 9044-9049 (2003).
28. H. O. Collier, L. C. Dinneen, C. A. Johnson, C. Schneider. The abdominal constriction response and its suppression by analgesic drugs in the mouse. Br J Pharmacol Chemother 32, 295-310 (1968).
29. Z. H. Lu et al., Evaluation of 2 celecoxib derivatives: analgesic effect and selectivity to cyclooxygenase-2/1. Acta Pharmacol Sin 26, 1505-1511 (2005).
30. E. M. Pogatzki, S. N. Raja, A mouse model of incisional pain. Anesthesiology 99, 1023-1027 (2003).
31. E. M. Pogatzki-Zahn, D. Segelcke, S. A. Schug, Postoperative pain—from mechanisms to treatment. Pain reports 2, e588 (2017).
32. G. T. Whiteside et al., Pharmacological characterisation of a rat model of incisional pain. British journal of pharmacology 141, 85-91 (2004).
33. A. Buvanendran et al., Upregulation of prostaglandin E2 and interleukins in the central nervous system and peripheral tissue during and after surgery in humans. Anesthesiology 104, 403-410 (2006).
34. P. S. Ostling et al., America's Opioid Epidemic: a Comprehensive Review and Look into the Rising Crisis. Current pain and headache reports 22, 32 (2018).
35. A. H. Lee, N. N. Iwakoshi, K. C. Anderson, L. H. Glimcher, Proteasome inhibitors disrupt the unfolded protein response in myeloma cells. Proc Natl Acad Sci USA 100, 9946-9951 (2003).
36. T. Iwawaki, R. Akai, S. Yamanaka, K. Kohno, Function of IRE1 alpha in the placenta is essential for placental development and embryonic viability. Proc Natl Acad Sci USA 106, 16657-16662 (2009).
37. S. Nair, G. E. Archer, T. F. Tedder, Isolation and generation of human dendritic cells. Curr Protoc Immunol Chapter 7, Unit7 32 (2012).
38. K. R. Maddipati, S. L. Zhou, Stability and analysis of eicosanoids and docosanoids in tissue culture media. Prostaglandins Other Lipid Mediators 94, 59-72 (2011).
39. J. F. Markworth et al., Human inflammatory and resolving lipid mediator responses to resistance exercise and ibuprofen treatment. Am J Physiol Regul Integr Comp Physiol 305, R1281-1296 (2013).
40. K. R. Maddipati et al., Eicosanomic profiling reveals dominance of the epoxygenase pathway in human amniotic fluid at term in spontaneous labor. FASEB J 28, 4835-4846 (2014).
41. K. R. Maddipati et al., Lipidomic analysis of patients with microbial invasion of the amniotic cavity reveals up-regulation of leukotriene B4. The FASEB Journal 30, 3296-3307 (2016).
42. K. R. Maddipati et al., Clinical chorioamnionitis at term: the amniotic fluid fatty acyl lipidome. J. Lipid Res. 57, 1906-1916 (2016).
43. R. L. Shaner et al., Quantitative analysis of sphingolipids for lipidomics using triple quadrupole and quadrupole linear ion trap mass spectrometers. J Lipid Res 50, 1692-1707 (2009).
44. C. Hellmuth, M. Weber, B. Koletzko, W. Peissner, Nonesterified Fatty Acid Determination for Functional Lipidomics: Comprehensive Ultrahigh Performance Liquid Chromatography-Tandem Mass Spectrometry Quantitation, Qualification, and Parameter Prediction. Anal. Chem. 84, 1483-1490 (2012).
45. P. M. Hutchins. R. M. Barkley, R. C. Murphy, Separation of cellular nonpolar neutral lipids by normal-phase chromatography and analysis by electrospray ionization mass spectrometry. J. Lipid Res. 49, 804-813 (2008).
46. B. Langmead, S. L. Salzberg. Fast gapped-read alignment with Bowtie 2. Nat Methods 9, 357-359 (2012).
47. B. Li, C. N. Dewey, RSEM: accurate transcript quantification from RNA-Seq data with or without a reference genome. BMC bioinformatics 12, 323 (2011).
48. M. I. Love. W. Huber, S. Anders, Moderated estimation of fold change and dispersion for RNA-seq data with DESeq2. Genome Biol 15, 550 (2014).
49. F. A. Ran et al., Genome engineering using the CRISPR-Cas9 system. Nat Protoc 8, 2281-2308 (2013).
50. M. Calfon et al., IRE1 couples endoplasmic reticulum load to secretory capacity by processing the XBP-1 mRNA. Nature 415, 92-96 (2002).
51. Website at portals.broadinstitute.org/gpp/public/analysis-tools/sgrna-design.
52. Website at crispr.mit.edu/.
53. Website at sfvideo.blob.core.windows.net/sitefinity/docs/default-source/user-guide-manual/alt-r-crispr-cas9-user-guide-ribonucleoprotein-transfections-recommended.pdf?sfvrsn=1c43407_12.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various embodiments of the invention according to the foregoing description in the specification.

### Statements:

1. A method comprising administering a composition comprising one or more IRE1α-XBP1 signaling inhibitors to reduce pain in a mammalian or avian subject.
2. The method of statement 1, wherein the composition comprises one or more compounds of formula I:

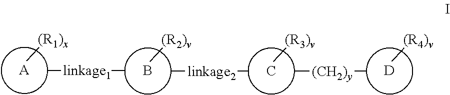

wherein:
   A and B are separately each a heterocyclyl ring or a phenyl group, where the A ring has x R₁ substituents;
   C is phenyl or pyridinyl;
   D is heterocyclyl ring:
   linkage₁ is a single bond between A and B or
   linkage₁ is a C₁-C₅ alkylene, an alkenylene, an alkynylene, an alkylamido, an acyl, or an oxo(carbonyl)alkylene with a first and second terminal atom;
   linkage₂ is a C₁-C₃ alkylamido, amidoalkyl, amino, urea, alkylurea, or ureaalkyl with a first and second terminal atom;
   y is an integer of 0-3, and when y is 0, the linkage between the rings is a single bond;
   x is an integer of 0-4 (e.g. 0-2);
   v is an integer of 0-2 (e.g., 0-1);
   R₁ substituents on the A ring are selected from amino, optionally substituted C₁-C₄ alkyl, optionally substituted ether, optionally substituted C₁-C₄ alkoxy, oxy, hydroxy, —NH—SO₂-phenyl-(R₅), and cyano;
   R₂ substituents on the B ring are selected from amino, and optionally substituted C₁-C₄ alkyl;
   R₃ substituents on the C ring are selected from halo, CF₃, optionally substituted C₁-C₄ alkyl, and optionally substituted heteroaryl; and
   R₄ substituents on the D ring are selected from optionally substituted C₁-C₄ alkyl, optionally substituted C₁-C₄ alkoxy, (optionally substituted C₁-C₄ alkylene)-OH, hydroxy, optionally substituted aryl, optionally substituted benzyl, and optionally substituted benzaldehyde;
   R₅ is halo; or
   a pharmaceutically acceptable salt thereof.
3. The method of statement 1, wherein the composition comprises one or more compounds of formula II:

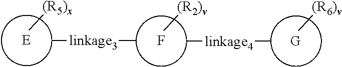

wherein:
   E is phenyl;
   F is phenyl, naphthalene, tetrahydronaphthalene, or a bicyclic heterocycle;
   G is phenyl, or a heterocyclyl ring; heterocycle indene, dihydroindene, or benzodioxole;
   linkage₃ is a C₁-C₃ alkyl, alkylamino, aminoalkyl, alkylaminoalkylene, or amino;
   linkage₄ is alkylamido, amidoalkyl, alkylamidoalkylene;
   R₂ is amino, or C₁-C₃ alkyl;
   R₅ is halo;
   R₆ is C₁-C₃ alkyl, C₁-C₃ alkoxy, or hydroxy;
   x is an integer of 0-2;
   v is an integer of 0-1; or
   a pharmaceutically acceptable salt thereof.
4. The method of statement 1 or 2, wherein the composition comprises one or more compounds of formula Ia:

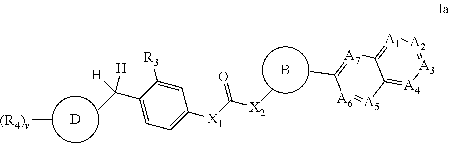

wherein:
   A₁ is N, CH, or CR₁; A₂ is N, CH, or CR₁; A₃ is N, CH, or CR₁; A₄ is N, CH, or CR₁; A₅ is N, CH, or CR₁; A₆ is N, CH, or CR₁; A₇ is N CH, or CR₁;
   v is an integer of 0-2;
   each R₁ is NH₂ or OH; provided that the number of R₁ on the A ring does not exceed 4;
   B is selected from:

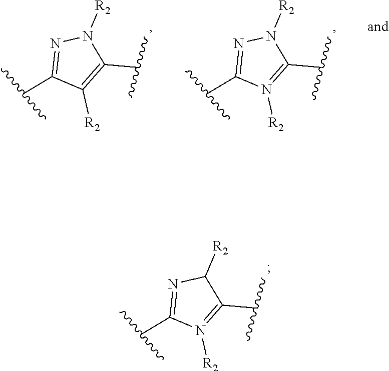

each R₂ is independently selected from H and optionally substituted C₁-C₄ alkyl;
X₁ and X₂ are each independently CH₂ or NH; with the provision that X₁ and X₂ are not each CH₂;
R₃ is selected from H, halo, CF₃, optionally substituted C₁-C₄ alkyl, and optionally substituted heteroaryl;
D is heterocyclyl ring containing at least one N atom;
each R₄ is selected from H, optionally substituted C₁-C₄ alkyl, optionally substituted C₁-C₄ alkoxy, (optionally substituted C₁-C₄ alkylene)-OH, hydroxy, optionally substituted aryl, and optionally substituted benzyl; or
a pharmaceutically acceptable salt thereof.
5. The method of statement 1-3 or 4, wherein the composition comprises one or more compounds of formula Ib:

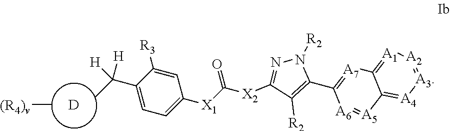

6. The method of statement 1-4 or 5, wherein the composition comprises one or more compounds of formula Ic:

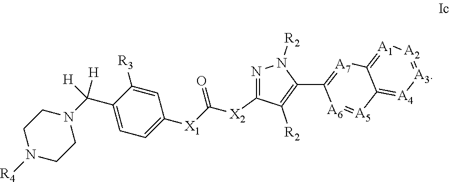

7. The method of statement 1-5 or 6, wherein the composition comprises one or more compounds of formula by formula Id:

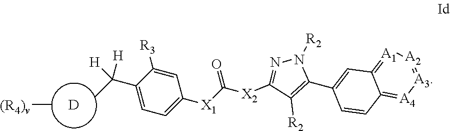

8. The method of statement 1-6 or 7, wherein the composition comprises one or more compounds of formula Ie:

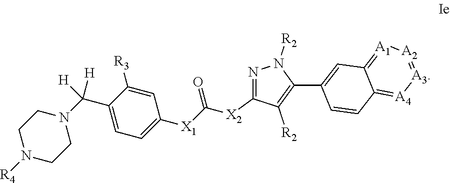

9. The method of statement 1-7 or 8, wherein the composition comprises one or more compounds of Formula III:

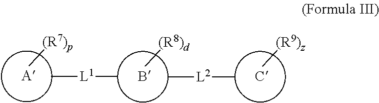

wherein:
   the A′ ring is a heterocyclyl or aryl;
   p is an integer of 0-2;
   R⁷ is independently amino, C₁-C₄ alkyl, C₁-C₄ alkoxy, hydroxy, C₁-C₄ hydroxyalkyl, arylsulfonyl, cyano, halogen, or trifluoromethyl;
   L¹ is a single bond, C₁-C₃ alkyl, C₂-C₃ alkenyl or C₂-C₃ alkynyl;
   the B′ ring is a heterocyclyl or aryl;
   d is an integer of 0-1;
   R⁸ is independently amino, C₁-C₄ alkyl, halogen or trifluoromethyl;
   L² is amino, urea, amido, alkylamido, alkenylamido, amidoalkyl, amidoalkenyl, alkylurea, or alkenylurea;
   the C′ ring is a heterocyclyl or aryl;
   z is an integer of 0-2:
   R⁹ is independently amino, C₁-C₄ alkyl, C₁-C₄ alkoxy, hydroxy, C₁-C₄ hydroxyalkyl, cyano, halogen, trifluoromethyl, difluoromethyl, monofluoroalkyl, benzyl, dialkylaminosulfonyl, alkylsulfonyl, boronic ester, boronic acid, dialkylphosphine, C₁-C₄ alkylcarboxyl, dialkylamido, cycloalkylalkyl, or heterocyclylalkyl;
   or a pharmaceutically acceptable salt thereof.
10. The method of statement 1-8 or 9, wherein the composition comprises one or more compounds of Formula III:
   IV,

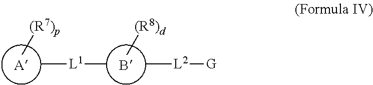

wherein:
   the A′ ring is a heterocyclyl or aryl;
   p is an integer of 0-2;
   R⁷ is independently amino, C₁-C₄ alkyl, C₁-C₄ alkoxy, hydroxy, C₁-C₄ hydroxyalkyl, arylsulfonyl, cyano, halogen, or trifluoromethyl;
   L¹ is a single bond, C₁-C₃ alkyl, C₂-C₃ alkenyl or C₂-C₃ alkynyl;
   the B′ ring is a heterocyclyl or aryl;
   d is an integer of 0-1;
   R⁸ is independently amino, C₁-C₄ alkyl, halogen or trifluoromethyl;
   L² is amino, urea, amido, alkylamido, alkenylamido, amidoalkyl, amidoalkenyl, alkylurea, or alkenylurea;
   G is dialkylamino or H,
   or a pharmaceutically acceptable salt thereof.
11. The method of statement 1-9 or 10, wherein the composition comprises one or more compounds of Formula V,

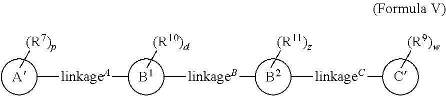

wherein:
   the A′ ring is a heterocyclyl or aryl;
   p is an integer of 0-2;
   R⁷ is independently amino, C₁-C₄ alkyl, C₁-C₄ alkoxy, hydroxy, C₁-C₄ hydroxyalkyl, arylsulfonyl, cyano, halogen, trifluoromethyl or a group having the structure

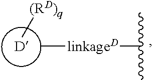

wherein the D′ ring is a heterocyclyl;
q is an integer of 0-2;
R^{D} is amino, C₁-C₄ alkyl, C₁-C₄ alkoxy, hydroxy, C₁-C₄ hydroxyalkyl, arylsulfonyl, cyano, halogen, or trifluoromethyl; and
the linkage^{D} is a single bond, amino or C₁-C₃ alkyl;
the B¹ ring is a heterocyclyl or aryl;
d is an integer of 0-1;
R¹⁰ is independently amino, C₁-C₃ alkyl, halogen or trifluoromethyl;
the B² ring is phenyl, pyridinyl, naphthyl or a bicyclic heterocyclyl;
z is an integer of 0-1;
R¹¹ is independently amino, C₁-C₄ alkyl, halogen or trifluoromethyl;
the C′ ring is a heterocyclyl ring;
w is an integer of 0-2;
R⁹ is independently C₁-C₄ alkyl, C₁-C₄ alkoxy, C₁-C₄ hydroxyalkyl, hydroxy, aryl, benzyl, benzaldehyde, halogen, cyano, amino, heterocyclyl, heterocyclylalkyl, cycloalkyl, cycloalkylalkyl, trifluoromethyl, difluoromethyl, monofluoroalkyl, dialkylaminosulfonyl, alkylsulfonyl, dialkylphosphine, C₁-C₄ alkylcarboxyl, dialkylamido, or dialkylamino;
the linkage^{A} is a single bond, is a C₁-C₅ alkyl, alkenyl, alkynyl, alkylamido, acyl, or oxo(carbonyl)alkyl;
the linkage^{B} is alkylamido, alkenylamido, amidoalkyl, amidoalkenyl, urea, alkylurea, or alkenylurea;
the linkage^{C} is CH or (CH₂)ₙ, where n is an integer of 0-3, and when n is 0, the linkage between the B² ring and the C ring is a single bond; and
or a pharmaceutically acceptable salt thereof.
12. The method of statement 1-10 or 11, wherein the composition comprises one or more compounds of any of Tables 1-7.
13. The method of statement 1-11 or 12, wherein the composition reduces prostaglandinendoperoxide synthase 2 (Ptgs2/Cox-2) expression in cells of the subject by a least 5%, or at least 10%, or at least 20%, or at least 30%, or at least 40%, or at least 50%, or at least 60%.
14. The method of statement 1-11 or 12, wherein the composition reduces prostaglandin E synthase (Ptges/mPGES-1) expression in cells of the subject by a least 5%, or at least 10%, or at least 20%, or at least 30%, or at least 40%, or at least 50%, or at least 60%, or at least 70%, or at least 75%, or at least 80%, or at least 90%, or at least 95%, or at least 98%.
15. The method of statement 13 or 14, wherein the composition does not affect expression of prostaglandin-endoperoxide synthase 1 (also known as COX1; COX3; PHS1; PCOX1; PES-1; PGHS1; PTGHS; PGG/HS; PGHS-1 and referred to as Ptgs1/Cox-1) in cells of the subject.
16. The method of statement 13, 14 or 15, wherein the composition does not affect expression of or prostaglandin E synthase 2 (also known as GBF1; GBF-1; PGES2; C9orf15; mPGES-2; and referred to as Ptges2) in cells of the subject.
17. The method of statement 13-15 or 16, wherein the cells are myeloid cells such as dendritic cells, neutrophils, macrophages, or a combination thereof.
18. The method of statement 13-16 or 17, wherein the cells are exposed to LPS, zymosan or ER stress inducers such as thapsigargin during or before measurement of the expression in vitro.
19. The method of statement 13-16 or 17, wherein the phorbol myristate acetate (PMA), lipopolysaccharide (LPS), zymosan, or acetic acid are administered to the subject and expression is measured in vivo.
20. The method of statement 1-18 or 19, wherein the composition reduces concentrations of one or more prostaglandin, arachidonic acid, or a combination thereof in cells of the subject by a least 5%, or at least 10%, or at least 20%, or at least 30%, or at least 40%, or at least 50%, or at least 60%, or at least 70%, or at least 75%, or at least 80%, or at least 90%, or at least 95%, or at least 98%.
21. The method of statement 19, wherein the prostaglandin is PGE₁, 15-keto PGF₂α, D12-PGJ₂, PGD₃, PGE₂, PGF₂α, 13,14dh-15k PGE₂, PGD₂, PGD₃, PGF1α, or a combination thereof.
22. The method of statement 20 or 21, wherein the composition reduces concentrations of PGE₂ in cells of the subject.
23. The method of statement 20, 21 or 22, wherein the cells are dendritic cells, neutrophils, macrophages, or a combination thereof.
24. The method of statement 1-22 or 23, wherein pain is reduced in the subject by a least 5%, or at least 10%, or at least 20%, or at least 30%, or at least 40%, or at least 50%, or at least 60%, or at least 70%, or at least 75%, or at least 80%, or at least 90%, or at least 95%, or at least 98%.
25. The method of statement 24, wherein pain is measured by the subject's number of writhings per selected time-period, the number of changes in weight distribution per selected time-period, the number of ambulatory counts per selected time-period, the total ambulatory time per time-period, or a combination thereof.
26. The method of statement 1-24 or 25, wherein the composition does not exhibit side effects selected from stomach pain, heartburn, ulcers, or reduced blood clotting compared to a control subject that did not receive administration of the composition.
27. The method of statement 1-25 or 26, wherein the subject does not exhibit side effects selected from stomach pain, heartburn, ulcers, or reduced blood clotting compared to a control subject that did not receive administration of the composition.
28. The method of statement 1-26 or 27, wherein the composition is administered once per day, twice per day, three times per day, four times per day, or five times per day.
29. The method of statement 1-27 or 28, wherein the composition comprises about 1 ng/kg of body weight to about 0.5 g/kg of body weight of at least one compound.
30. The method of statement 1-28 or 29, wherein the composition comprises about 10 μ/kg of body weight to about 250 mg/kg of body weight of at least one compound.
31. The method of statement 1-29 or 30, wherein the composition comprises about 20 μ/kg of body weight to about 100 mg/kg of body weight of at least one compound.
32. The method of statement 1-30 or 31, wherein the composition comprises about 0.05 to about 5000 mg of at least one compound.
33. The method of statement 1-31 or 32, wherein the composition comprises about 1 to about 2000 mg of at least one compound.
34. The method of statement 1-32 or 33, wherein the composition comprises about 2 and about 2000 mg of at least one compound.
35. The method of statement 1-32 or 33, wherein the composition reduces hypoxia, allergies, angiogenesis, atherosclerosis, arthritis, fever, immunosuppression, vascular permeability, or symptoms thereof.

The specific compositions and methods described herein are representative, exemplary and not intended as limitations on the scope of the invention. Other objects, aspects, and embodiments will occur to those skilled in the art upon consideration of this specification and are encompassed within the spirit of the invention as defined by the scope of the claims. It may be apparent to one skilled in the art that varying substitutions and modifications may be made to the invention disclosed herein without departing from the scope and spirit of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims and statements of the invention.

The invention illustratively described herein may be practiced in the absence of any element or elements, or limitation or limitations, which is not specifically disclosed herein as essential. The methods and processes illustratively described herein may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “an inhibitor” or “a molecule” or “a cell” includes a plurality of such inhibitors, molecules or cells, and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

The Abstract is provided to comply with 37 C.F.R. § 1.72(b) to allow the reader to quickly ascertain the nature and gist of the technical disclosure. The Abstract is submitted with the understanding that it will not be used to interpret or limit the scope or meaning of the claims.

1. A method comprising administering a composition comprising one or more IRE1α-XBP1 signaling inhibitors to reduce pain in a mammalian or avian subject, wherein the composition comprises one or more compounds of formula I or II: 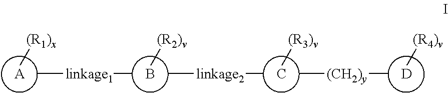
wherein:
A and B are separately each a heterocyclyl ring or a phenyl group, where the A ring has x R₁ substituents;
C is phenyl or pyridinyl;
D is heterocyclyl ring;
linkage₁ is a single bond between A and B or
linkage₁ is a C₁-C₅ alkylene, an alkenylene, an alkynylene, an alkylamido, an acyl, or an oxo(carbonyl)alkylene with a first and second terminal atom;
linkage₂ is a C₁-C₃ alkylamido, amidoalkyl, amino, urea, alkylurea, or ureaalkyl with a first and second terminal atom;
y is an integer of 0-3, and when y is 0, the linkage between the rings is a single bond;
x is an integer of 0-4 (e.g. 0-2);
v is an integer of 0-2 (e.g., 0-1);
R₁ substituents on the A ring are selected from amino, optionally substituted C₁-C₄ alkyl, optionally substituted ether, optionally substituted C₁-C₄ alkoxy, oxy, hydroxy, —NH—SO₂-phenyl-(R₅), and cyano;
R₂ substituents on the B ring are selected from amino, and optionally substituted C₁-C₄alkyl;
R₃ substituents on the C ring are selected from halo, CF₃, optionally substituted C₁-C₄alkyl, and optionally substituted heteroaryl; and
R₄ substituents on the D ring are selected from optionally substituted C₁-C₄ alkyl, optionally substituted C₁-C₄ alkoxy, (optionally substituted C₁-C₄ alkylene)-OH, hydroxy, optionally substituted aryl, optionally substituted benzyl, and optionally substituted benzaldehyde;
R₅ is halo; or
a pharmaceutically acceptable salt thereof; 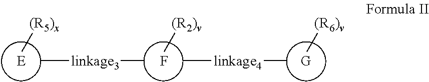
wherein:
E is phenyl;
F is phenyl, naphthalene, tetrahydronaphthalene, or a bicyclic heterocycle;
G is phenyl, or a heterocyclyl ring; heterocycle indene, dihydroindene, or benzodioxole;
linkage₃ is a C₁-C₃ alkyl, alkylamino, aminoalkyl, alkylaminoalkylene, or amino;
linkage₄ is alkylamido, amidoalkyl, alkylamidoalkylene;
R₂ is amino, or C₁-C₃ alkyl;
R₅ is halo;
R₆ is C₁-C₃ alkyl, C₁-C₃ alkoxy, or hydroxy;
x is an integer of 0-2;
v is an integer of 0-1; or
a pharmaceutically acceptable salt thereof.

2. The method of claim 1, wherein the composition comprises one or more compounds of formula Ia: 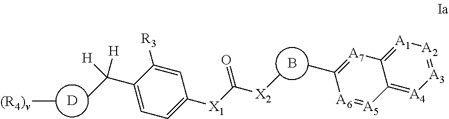
wherein:
A₁ is N, CH, or CR₁; A₂ is N, CH, or CR₁; A₃ is N, CH, or CR₁; A₄ is N, CH, or CR₁; A₅ is N, CH, or CR₁; A₆ is N, CH, or CR₁; A₇ is N CH, or CR₁;
v is an integer of 0-2;
each R₁ is NH₂ or OH; provided that the number of R₁ on the A ring does not exceed 4;
B is selected from: 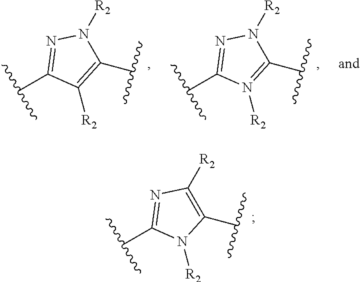
each R₂ is independently selected from H and optionally substituted C₁-C₄ alkyl;
X₁ and X₂ are each independently CH₂ or NH; with the provision that X₁ and X₂ are not each CH₂;
R₃ is selected from H, halo, CF₃, optionally substituted C₁-C₄ alkyl, and optionally substituted heteroaryl;
D is heterocyclyl ring containing at least one N atom;
each R₄ is selected from H, optionally substituted C₁-C₄ alkyl, optionally substituted C₁-C₄ alkoxy, (optionally substituted C₁-C₄ alkylene)-OH, hydroxy, optionally substituted aryl, and optionally substituted benzyl; or
a pharmaceutically acceptable salt thereof.

3. The method of claim 1, wherein the composition comprises one or more compounds of formula Ib:

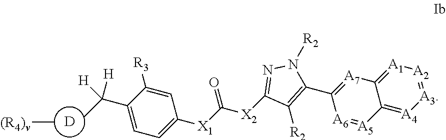

4. The method of claim 1, wherein the composition comprises one or more compounds of formula Ic:

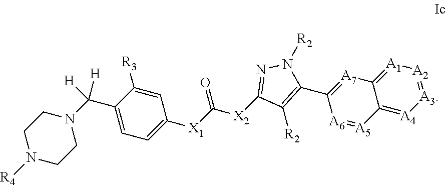

5. The method of claim 1, wherein the composition comprises one or more compounds of formula by formula Id:

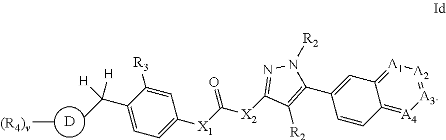

6. The method of claim 1, wherein the composition comprises one or more compounds of formula Ie:

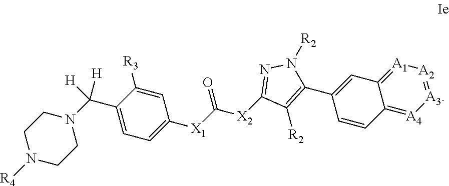

7. The method of claim 1, wherein the composition comprises one or more compounds of Formula III: 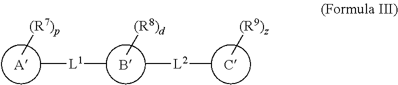
wherein:
the A′ ring is a heterocyclyl or aryl;
p is an integer of 0-2;
R⁷ is independently amino, C₁-C₄ alkyl, C₁-C₄ alkoxy, hydroxy, C₁-C₄ hydroxyalkyl, arylsulfonyl, cyano, halogen, or trifluoromethyl;
L¹ is a single bond, C₁-C₃ alkyl, C₂-C₃ alkenyl or C₂-C₃ alkynyl;
the B′ ring is a heterocyclyl or aryl;
d is an integer of 0-1;
R⁸ is independently amino, C₁-C₄ alkyl, halogen or trifluoromethyl;
L² is amino, urea, amido, alkylamido, alkenylamido, amidoalkyl, amidoalkenyl, alkylurea, or alkenylurea;
the C′ ring is a heterocyclyl or aryl;
z is an integer of 0-2;
R⁹ is independently amino, C₁-C₄ alkyl, C₁-C₄ alkoxy, hydroxy, C₁-C₄ hydroxyalkyl, cyano, halogen, trifluoromethyl, difluoromethyl, monofluoroalkyl, benzyl, dialkylaminosulfonyl, alkylsulfonyl, boronic ester, boronic acid, dialkylphosphine, C₁-C₄ alkylcarboxyl, dialkylamido, cycloalkylalkyl, or heterocyclylalkyl;
or a pharmaceutically acceptable salt thereof.

8. The method of claim 1, wherein the composition comprises one or more compounds of Formula III:
IV, 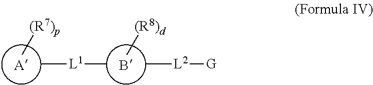
wherein:
the A′ ring is a heterocyclyl or aryl;
p is an integer of 0-2;
R⁷ is independently amino, C₁-C₄ alkyl, C₁-C₄ alkoxy, hydroxy, C₁-C₄ hydroxyalkyl, arylsulfonyl, cyano, halogen, or trifluoromethyl;
L¹ is a single bond, C₁-C₃ alkyl, C₂-C₃ alkenyl or C₂-C₃ alkynyl;
the B′ ring is a heterocyclyl or aryl;
d is an integer of 0-1;
R⁸ is independently amino, C₁-C₄ alkyl, halogen or trifluoromethyl;
L² is amino, urea, amido, alkylamido, alkenylamido, amidoalkyl, amidoalkenyl, alkylurea, or alkenylurea;
G is dialkylamino or H,
or a pharmaceutically acceptable salt thereof.

9. The method of claim 1, wherein the composition comprises one or more compounds of Formula V, 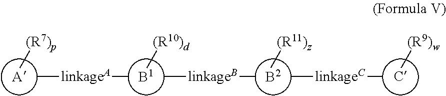
wherein:
the A′ ring is a heterocyclyl or aryl;
p is an integer of 0-2;
R⁷ is independently amino, C₁-C₄ alkyl, C₁-C₄ alkoxy, hydroxy, C₁-C₄ hydroxyalkyl, arylsulfonyl, cyano, halogen, trifluoromethyl or a group having the structure 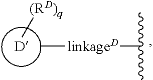
wherein the D′ ring is a heterocyclyl;
q is an integer of 0-2;
R^{D} is amino, C₁-C₄ alkyl, C₁-C₄ alkoxy, hydroxy, C₁-C₄ hydroxyalkyl, arylsulfonyl, cyano, halogen, or trifluoromethyl; and
the linkage^{D} is a single bond, amino or C₁-C₃ alkyl;
the B¹ ring is a heterocyclyl or aryl;
d is an integer of 0-1;
R¹⁰ is independently amino, C₁-C₃ alkyl, halogen or trifluoromethyl;
the B² ring is phenyl, pyridinyl, naphthyl or a bicyclic heterocyclyl;
z is an integer of 0-1;
R¹¹ is independently amino, C₁-C₄ alkyl, halogen or trifluoromethyl;
the C′ ring is a heterocyclyl ring;
w is an integer of 0-2;
R⁹ is independently C₁-C₄ alkyl, C₁-C₄ alkoxy, C₁-C₄ hydroxyalkyl, hydroxy, aryl, benzyl, benzaldehyde, halogen, cyano, amino, heterocyclyl, heterocyclylalkyl, cycloalkyl, cycloalkylalkyl, trifluoromethyl, difluoromethyl, monofluoroalkyl, dialkylaminosulfonyl, alkylsulfonyl, dialkylphosphine, C₁-C₄ alkylcarboxyl, dialkylamido, or dialkylamino;
the linkage^{A} is a single bond, is a C₁-C₅ alkyl, alkenyl, alkynyl, alkylamido, acyl, or oxo(carbonyl)alkyl;
the linkage^{B} is alkylamido, alkenylamido, amidoalkyl, amidoalkenyl, urea, alkylurea, or alkenylurea;
the linkage^{C} is CH or (CH₂)ₙ, where n is an integer of 0-3, and when n is 0, the linkage between the B² ring and the C ring is a single bond; and
or a pharmaceutically acceptable salt thereof.

10. The method of claim 1, wherein the composition comprises one or more compounds of any of Tables 1-7.

11. The method of claim 1, wherein the composition reduces prostaglandinendoperoxide synthase 2 (Ptgs2/Cox-2) expression in cells of the subject by a least 5%, or at least 10%, or at least 20%, or at least 30%, or at least 40%, or at least 50%, or at least 60%.

12. The method of claim 1, wherein the composition reduces prostaglandin E synthase (Ptges/mPGES-1) expression in cells of the subject by a least 5%.

13. The method of claim 11, wherein the composition does not affect expression of prostaglandin-endoperoxide synthase 1 in cells of the subject.

14. The method of claim 11, wherein the composition does not affect expression of or prostaglandin E synthase 2 in cells of the subject.

15. The method of claim 11, wherein the cells are myeloid cells such as dendritic cells, neutrophils, macrophages, or a combination thereof.

16. The method of claim 1, wherein the composition reduces concentrations of one or more prostaglandin, arachidonic acid, or a combination thereof in cells of the subject by a least 5%.

17. The method of claim 16, wherein the prostaglandin is PGE₁, 15-keto PGF₂α, D12-PGJ₂, PGD₃, PGE₂, PGF₂α, 13,14dh-15k PGE₂, PGD₂, PGD₃, PGF1α, or a combination thereof.

18. The method of claim 16, wherein the composition reduces concentrations of PGE₂ in cells of the subject.

19. The method of claim 16, wherein the cells are myeloid cells such as dendritic cells, neutrophils, macrophages, or a combination thereof.

20. The method of claim 1, wherein pain is reduced in the subject by a least 5%.

21. The method of claim 20, wherein pain is measured by the subject's number of writhings per selected time-period, the number of changes in weight distribution per selected time-period, the number of ambulatory counts per selected time-period, the total ambulatory time per time-period, or a combination thereof.

22. The method of claim 1, wherein the composition does not exhibit side effects selected from stomach pain, heartburn, ulcers, or reduced blood clotting compared to a control subject that did not receive administration of the composition.

23. The method of claim 1, wherein the subject does not exhibit side effects selected from stomach pain, heartburn, ulcers, or reduced blood clotting compared to a control subject that did not receive administration of the composition.

24. The method of claim 1, wherein the composition is administered once per day, twice per day, three times per day, four times per day, or five times per day.

25. The method of claim 1, wherein the composition comprises about 1 ng/kg of body weight to about 0.5 g/kg of body weight of at least one compound.

26. The method of claim 1, wherein the composition comprises about 0.05 to about 5000 mg of at least one compound.
